(19) United States
(12) Patent Application Publication
(10) Pub. No.: US 2025/0285756 A1
(43) Pub. Date: Sep. 11, 2025

(54) TWO COMPETING GUILDS AS CORE MICROBIOME SIGNATURE FOR HUMAN DISEASES

(71) Applicants: RUTGERS, THE STATE UNIVERSITY OF NEW JERSEY, New Brunswick, NJ (US); SHANGHAI JIAO TONG UNIVERSITY, Shanghai (CN)

(72) Inventors: Liping ZHAO, New Brunswick, NJ (US); Guojun WU, New Brunswick, NJ (US); Chenhong ZHANG, Shanghai (CN)

(21) Appl. No.: 18/859,524

(22) PCT Filed: Apr. 25, 2023
(86) PCT No.: PCT/US2023/066191

§ 371 Date: Oct. 23, 2024

Related U.S. Application Data
(60) Provisional application No. 63/334,503, filed on Apr. 25, 2022.

(51) Int. Cl. G16H 50/20; C12Q 1/6869; C12Q 1/689; G16B 20/10; G16B 30/10; G16B 40/20

(52) U.S. Cl. CPC G16H 50/20; C12Q 1/6869; C12Q 1/689; G16B 20/10; G16B 30/10; G16B 40/20

(57) ABSTRACT

Methods and systems for determining a disease state by obtaining a first plurality of nucleic acid sequences for genomic DNA from a sample from the gut of a subject. Determine, from the nucleic acid sequences a first plurality of genomic abundance values for a first plurality of gut bacteria and a second plurality of genomic abundance values for a second plurality of at least 20 species of gut bacteria. Apply a model to at least the first plurality of genomic abundance values and the second plurality of genomic abundance values, or one or more combinations thereof, thereby determining the disease state of the subject as an output of the model.

### CROSS REFERENCE TO RELATED APPLICATIONS

This application claims priority to U.S. Provisional Patent Application Ser. No. 63/334,503, filed Apr. 25, 2022, which is hereby incorporated by reference herein in its entirety.

### BRIEF DESCRIPTION OF THE SEQUENCE LISTING

This submission is accompanied by a “Sequence Listing XML” file named ST26_126146_5001_WO.XML containing SEQ ID NOs: 1-99534, created on Apr. 22, 2023, and having a size of 2,491,699 kilobytes, in accordance with 37 CFR §§ 1.831 through 1.835, submitted on a read only optical disc (DVD) as an XML file via mail. 37 CFR § 1.835(a)(1). The Sequence Listing XML is hereby incorporated by reference herein in its entirety.

### BACKGROUND

Over eons of co-evolution, humans have developed a robust symbiotic relationship with their gut microbiome [6,7]. The gut microbiome works as an essential organ to support the host's homeostasis in metabolism, immunity, development, and behavior etc. [8]. The attenuation or loss of such health-relevant functions in dysbiotic gut microbiome has been identified as a risk factor for many chronic diseases including type 2 diabetes (T2DM) [9-11]. A suite of microbiome-wide association studies (MWAS) has attempted to identify the microbiome signatures (including features such as genes, pathways, taxa, etc.) that are associated with disease phenotypes as biomarkers in metagenomic datasets [12-15].

However, the current gene-centric approaches for metagenomic data analysis in most MWAS projects have important limitations. They rely on existing databases for taxonomic and functional annotation of individual genes and exclude novel genes from downstream analysis [12, 14, 15]. More importantly, such approaches treat individual genes as independent units while disregarding the fact that the functionality of bacterial genes is constrained by the ecological behavior of their carriers. For example, two competing bacterial strains may encode the same functional gene. However, these two copies of the same gene will differ in their contribution to the community-wide expression of that gene's function due to opposite growth trajectories of their carrier within the gut habitat [16]. Lumping the same functional gene from different bacterial strains into one unit such as pathway or species will mask or neutralize these opposite changes and lead to spurious correlations [5].

### SUMMARY

Given the background above, a genome-centric MWAS was adopted in which high-quality draft genomes assembled from metagenomic datasets (metagenome-assembled genomes, MAGs) are used as the basic building blocks of the gut ecosystem and the most important microbiome features for correlation analysis with disease phenotypes. MAGs again are not independent microbiome features. They have ecological interactions such as competition or cooperation with each other and organize themselves into a higher-level structure called “guilds” [5]. Each guild is potentially a functional unit in the gut ecosystem and its members may have widely diverse taxonomic background but show co-abundant behavior. Guilds have been shown to be positively or negatively correlated with disease phenotypes [17]. Thus, MAGs and their guild-level aggregation are ecologically meaningful features for identifying microbiome signatures associated with human diseases.

Dysbiosis in the gut microbiome has been linked with an increased risk for a wide range of human diseases [1, 2]. To date, much effort has concentrated on identifying gene- or taxon-based microbial signatures as disease biomarkers. However, such signatures remain controversial [3, 4] and overlook the fact that gut bacterial strains are not independent but rather form coherent functional groups (a.k.a. “guilds”) to interact with each other and affect host health [5]. Therefore, embodiments may propose to search for strain-level microbiome signature in the form of robust guilds, through which the gut microbiome provides stable health-relevant functions to the host. Here embodiments may show that two competing bacterial guilds are organized as two ends of a robustly stable seesaw-like network and their abundance are correlated with a wide range of chronic diseases. 141 out of a total of 1,845 metagenome-assembled genomes (MAGs) formed the two competing guilds given their stable ecological relationships while experiencing profound structural changes in the gut microbiome during a 3-month high fiber intervention and 1-year follow-up in patients with type 2 diabetes (T2DM). The 50 genomes in Guild 1 harbored more genes for plant polysaccharide degradation and butyrate production, while the 91 genomes in Guild 2 included almost all the virulence or antibiotic resistance gene carriers predicted from the 1,845 MAGs. Random Forest regression model showed that the abundance distribution of the 141 genomes were associated with 41 out of 43 bio-clinical parameters. With these 141 MAGs as reference genomes, such a seesaw network was not only detectable but also conducive to machine learning models for predictive classification between case and control of 9 diseases including T2DM, atherosclerosis, hypertension, liver cirrhosis, inflammatory bowel diseases, colorectal cancer, ankylosing spondylitis, schizophrenia, and Parkinson's disease in 12 independent metagenomic datasets from 1,874 participants across ethnicity and geography. The two seesaw networked guilds may work as a core microbiome and their balance can be modulated for disease risk management.

Accordingly, one aspect of the present disclosure provides methods, and systems for performing the disclosed methods, for determining a disease state, in a plurality of disease states, of a subject. The method includes, at a computer system having at least one processor, and memory storing one or more programs for execution by the one or more processors, obtaining, in electronic form, a first plurality of (e.g., at least 100,000) nucleic acid sequences for first genomic DNA from a first biological sample from the gut of the subject. The method also includes determining, from the first plurality of nucleic acid sequences, a first plurality of genomic abundance values comprising, for each respective species of gut bacteria in a first plurality of (e.g., at least 20) species of gut bacteria, a first corresponding abundance value for the genome of the respective species of gut bacteria, in the first plurality of species of gut bacteria, in the first biological sample, and a second plurality of genomic abundance values comprising, for each respective species of gut bacteria in a second plurality of (e.g., at least 20) species of gut bacteria, a first corresponding abundance value for the genome of the respective species of gut bacteria, in the second plurality of species of gut bacteria, in the first biological sample. The method also includes applying, by the at least one processor, a model to at least the first plurality of genomic abundance values and the second plurality of genomic abundance values, or one or more combinations thereof, thereby determining the disease state of the subject as an output of the model.

As disclosed herein, any embodiment disclosed herein when applicable can be applied to any other aspect.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

Accordingly, one aspect of the invention provides a method of identifying a set of gut microorganisms at a computer system having one or more processors, and memory storing one or more programs for execution by the one or more processors.

In some embodiments, the method includes obtaining, in electronic form, for each respective subject in a first plurality of subjects having a first state of a biological characteristic a corresponding plurality of genomic abundance values comprising, for each respective gut microorganism in a plurality of gut microorganisms, a corresponding value for the abundance of the genome of the respective gut microorganism in a biological sample from the gut of the respective subject.

In some such embodiments, for each respective subject in the first plurality of subjects, the biological sample from the gut of the respective subject is a fecal sample.

In some such embodiments, the method includes sequencing, for each respective subject in the first plurality of subjects, genomic DNA from the corresponding biological sample from the gut of the respective subject, thereby obtaining the corresponding first plurality of at least 100,000 nucleic acid sequences.

In some such embodiments, the method includes obtaining, in electronic form, for each respective subject in the first plurality of subjects, a corresponding first plurality of at least 100,000 nucleic acid sequences for genomic DNA from a corresponding biological sample from the gut of the respective subject, and determining, for each respective subject in the first plurality of subjects, the corresponding genomic abundance value for each respective gut microorganism in the plurality of gut microorganisms from the corresponding first plurality of at least 100,000 nucleic acid sequences.

In some such embodiments, the method includes, for each respective subject in the first plurality of subjects, assembling a corresponding first plurality of gut microorganism genomes by metagenomic de novo sequence assembly from the corresponding first plurality of at least 100,000 nucleic acid sequences, and calculating, for each respective gut microorganism genome in the corresponding first plurality of gut microorganism genomes, a corresponding genomic abundance of the respective gut microorganism genome.

In some such embodiments, the method includes, for each respective subject in the first plurality of subjects, assigning each respective nucleic acid sequence in the corresponding first plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding first plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some embodiments, the method includes obtaining, in electronic form, for each respective subject in a second plurality of subjects having a second state of a biological characteristic, a corresponding plurality of genomic abundance values comprising, for each respective gut microorganism in the plurality of gut microorganisms, a corresponding value for the abundance of the genome of the respective gut microorganism in a biological sample from the gut of the respective subject.

In some such embodiments, for each respective subject in the second plurality of subjects, the biological sample from the gut of the respective subject is a fecal sample.

In some such embodiments, the method includes sequencing, for each respective subject in the second plurality of subjects, genomic DNA from the corresponding biological sample from the gut of the respective subject, thereby obtaining the corresponding second plurality of at least 100,000 nucleic acid sequences.

In some such embodiments, the method includes obtaining, in electronic form, for each respective subject in the second plurality of subjects, a corresponding second plurality of at least 100,000 nucleic acid sequences for genomic DNA from a corresponding biological sample from the gut of the respective subject, and determining, for each respective subject in the second plurality of subjects, the corresponding genomic abundance value for each respective gut microorganism in the plurality of gut microorganisms from the corresponding second plurality of at least 100,000 nucleic acid sequences.

In some such embodiments, the method includes, for each respective subject in the first plurality of subjects, assembling a corresponding second plurality of gut microorganism genomes by metagenomic de novo sequence assembly from the corresponding second plurality of at least 100,000 nucleic acid sequences, and calculating, for each respective gut microorganism genome in the corresponding second plurality of gut microorganism genomes, a corresponding genomic abundance of the respective gut microorganism genome.

In some such embodiments, the method includes, for each respective subject in the first plurality of subjects, assigning each respective nucleic acid sequence in the corresponding second plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding second plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some such embodiments, the first state of the biological characteristic is the absence of a disease or disorder and the second state of the biological characteristic is the presence of the disease or disorder, the first state of the biological characteristic is a first severity of a disease or disorder and the second state of the biological characteristic is a second severity of the disease or disorder, the first state of the biological characteristic is an untreated disease or disorder and the second state of the biological characteristic is a treated disease or disorder, the first state of the biological characteristic is a disease or disorder treated with a first therapy and the second state of the biological characteristic is a disease or disorder treated with a second therapy, the first state of the biological characteristic is a first level of a nutrient in a diet and the second state of the biological characteristic is a second level of a nutrient in a diet, or the first state of the biological characteristic is a first age and the second state of the biological characteristic is a second age.

In some such embodiments, the plurality of gut microorganisms comprises at least 20 gut microorganisms selected from Table 1, Table 2, or FIG. 42A-42XX.

In some embodiments, the method includes computing a first plurality of similarity metrics from the corresponding pluralities of genomic abundance values across the first plurality of subjects, where the first plurality of similarity metrics comprises a first corresponding similarity metric for each unique pair of gut microorganisms in the plurality of gut microorganisms, and the first corresponding similarity metric quantifies a similarity between (i) a corresponding first vector formed by the corresponding genomic abundance values of the first microorganism in the unique pair of gut microorganisms across the first plurality of subjects, and (ii) a corresponding second vector formed by the corresponding genomic abundance values of the second microorganism in the unique pair of gut microorganisms across the first plurality of subjects.

In some embodiments, the method includes computing a second plurality of similarity metrics using the corresponding genomic abundance values for the second plurality of subjects, where the second plurality of similarity metrics comprises a second corresponding similarity metric for each unique pair of gut microorganisms in the plurality of gut microorganisms, and the second corresponding similarity metric quantifies a similarity between (i) a corresponding second vector formed by the corresponding genomic abundance values of the first microorganism in the unique pair of gut microorganisms across the second plurality of subjects, and (ii) a corresponding second vector formed by the corresponding genomic abundance values of the second microorganism in the unique pair of gut microorganisms across the second plurality of subjects.

In some embodiments, the method includes determining a set of unique pairs of gut microorganisms in the plurality of gut microorganisms based on the first plurality of similarity metrics and the second plurality of similarity metrics, for each respective unique pair of gut microorganisms in the set of unique pairs of gut microorganisms, where the first corresponding similarity metric and the second corresponding similarity metric both indicate a statistically significant positive correlation between the abundance of the first gut microorganism and the abundance of the second gut microorganism in the respective unique pair of gut microorganisms, or the first corresponding similarity metric and the second corresponding similarity metric both indicate a statistically significant negative correlation between the abundance of the first gut microorganism and the abundance of the second gut microorganism in the respective unique pair of gut microorganisms.

In some such embodiments, the first corresponding similarity metric and the second similarity metric are both a Pearson correlation coefficient, an intraclass correlation coefficient, or a rank correlation coefficient.

In some such embodiments, a statistically significant positive correlation has a P-value of less than 0.001.

In some embodiments, the method includes identifying a set of gut microorganisms comprising respective gut microorganisms represented in the set of unique pairs of gut microorganisms.

In some such embodiments, the method includes clustering the respective gut microorganisms represented in the set of unique pairs of gut microorganisms into one of more networks. Each respective connected network comprising a corresponding plurality of nodes and a corresponding set of one or more edges.

In some such embodiments, each respective node in the corresponding plurality of nodes represents a unique gut microorganism represented in the set of unique pairs of gut microorganisms.

In some such embodiments, each respective edge in the corresponding set of one or more edges connects two nodes represents a respective unique pair of gut microorganisms in the set of unique pairs of gut microorganisms.

In some such embodiments, each respective node in the corresponding plurality of nodes is connected to at least one other respective node in the plurality of nodes through a respective edge in the corresponding set of one or more edges.

In some such embodiments, the method includes identifying the respective network in the one or more networks comprising the most nodes, thereby identifying the set of gut microorganisms represented by the corresponding plurality of nodes in the respective network.

In some such embodiments, the set of gut microorganisms comprises all respective gut microorganisms represented in the set of unique pairs of gut microorganisms.

In some such embodiments, the set of gut microorganisms comprises at least 20 gut microorganisms selected from Table 1, Table 2, or FIG. 42A-42XX.

Another aspect of the present disclosure provides a method of training a model for evaluating human health at a computer system having one or more processors, and memory storing one or more programs for execution by the one or more processors.

In some embodiments, the method includes, obtain, in electronic form, for each respective training subject in a plurality of training subjects: (i) a corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and (ii) a corresponding state of a biological characteristic of the respective training subject.

In some such embodiments, the method includes sequencing, for each respective subject in the plurality of training subjects, genomic DNA from the corresponding biological sample from the gut of the respective training subject, thereby obtaining the corresponding plurality of at least 100,000 nucleic acid sequences.

In some such embodiments, the biological sample from the gut of the respective subject is a fecal sample from the respective training subject.

In some such embodiments, the plurality of gut microorganisms comprise at least 20 gut microorganisms selected from Table 1, Table 2, or FIG. 42A-42XX.

In some such embodiments, the plurality of gut microorganisms comprises at least 20 microorganisms selected from those microorganisms in Table 1, Table 2, or FIG. 42A-42XX having a connectivity of at least 2.

In some such embodiments, the method includes, for each respective training subject in the plurality of training subjects, obtaining, in electronic form, a corresponding plurality of at least 100,000 nucleic acid sequences for genomic DNA from the corresponding biological sample from the gut of the respective training subject, and determining, for each respective gut microorganism in the plurality of gut microorganisms, the corresponding value for the abundance of the genome of the respective gut microorganism from the corresponding first plurality of at least 100,000 nucleic acid sequences.

In some such embodiments, the method includes, for each respective training subject in the plurality of training subjects, assembling, in electronic form, a corresponding plurality of gut microorganism genomes by metagenomic de novo sequence assembly from the corresponding plurality of at least 100,000 nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

In some such embodiments, the method includes, for each respective subject in the plurality of training subjects, assigning each respective nucleic acid sequence in the corresponding plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some such embodiments, the biological characteristic is a disease or disorder, a therapy administered to the subject, or a diet of the subject.

In some such embodiments, the disease or disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD).

In some such embodiments, the disease or disorder is cancer.

In some embodiments, the method includes inputting, for each respective training subject in the plurality of training subjects, information about the respective training subject into a model comprising a plurality of parameters. The model applies the plurality of parameters to the information through at least 10,000 computations to obtain a corresponding output for the respective training subject from the model. The corresponding output comprises an indication of the corresponding state of the biological characteristic of the respective training subject, and the information about the respective training subject comprises the corresponding genomic abundance value for each respective gut microorganism in the plurality of gut microorganisms. The plurality of gut microorganisms is selected from Table 1, Table 2, or FIG. 42A-42XX.

In some such embodiments, the indication of the corresponding state of the biological characteristic is a class output of a respective state, in a plurality of possible states, of the biological characteristic.

In some such embodiments, the indication of the corresponding state of the biological characteristic is a probability output for the corresponding state of the biological characteristic.

In some such embodiments, the model is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

In some such embodiments, the plurality of parameters is at least 1000, at least 10,000, at least 15,000, at least 50,000, at least 100,000, at least 250,000, at least 500,000, or at least 1,000,000 parameters.

In some such embodiments, the model applies the plurality of parameters to the information through at least 25,000, at least 50,000, at least 100,000, at least 250,000, at least 500,000, or at least 1,000,000 computations to obtain a corresponding output for the respective training subject from the model.

In some embodiments, the method includes adjusting the plurality of parameters based on, for each respective training subject in the first plurality of training subjects, one or more differences between (i) the corresponding output from the model, and (ii) the corresponding state of the biological characteristic of the respective training subject.

Another aspect of the present disclosure provides a method for evaluating the health of a subject at a computer system having one or more processors, and memory storing one or more programs for execution by the one or more processors.

In some embodiments, the method includes obtaining, in electronic form, a plurality of genomic abundance values comprising, for each respective gut microorganism in a plurality of at least 20 gut microorganisms selected from Table 1, Table 2, or FIG. 42A-42XX, a corresponding abundance value for the genome of the respective species of gut bacteria, in the plurality of at least 20 gut microorganisms, in a biological sample from the subject.

In some such embodiments, the method includes sequencing genomic DNA from the biological sample from the gut of the subject, thereby obtaining the plurality of at least 100,000 nucleic acid sequences.

In some such embodiments, the biological sample from the gut of the subject is a fecal sample.

In some such embodiments, the plurality of gut microorganisms comprises at least 20 microorganisms selected from those microorganisms in Table 1, Table 2, or FIG. 42A-42XX having a connectivity of at least 2.

In some such embodiments, the method includes obtaining, in electronic form, a plurality of at least 100,000 nucleic acid sequences for genomic DNA from the biological sample from the gut of the subject; and determining, for each respective gut microorganism in the plurality of gut microorganisms, the corresponding value for the abundance of the genome of the respective gut microorganism from the plurality of at least 100,000 nucleic acid sequences.

In some such embodiments, the method includes assembling, in electronic form, a corresponding plurality of gut microorganism genomes by metagenomic de novo sequence assembly from the plurality of at least 100,000 nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

In some such embodiments, the method includes assigning, each respective nucleic acid sequence in the plurality of at least 100,000 sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism.

In some embodiments, the method includes inputting the plurality of genomic abundance values into a model comprising a plurality of parameters. The model applies the plurality of parameters to the plurality of genomic abundance values through at least 10,000 computations to generate as output from the model an indication of the health of the subject.

In some such embodiments, the indication of the health of the subject is an indication of a biological characteristic. The biological characteristic is a disease or disorder, a therapy administered to the subject, or a diet of the subject.

In some such embodiments, the disease or disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD).

In some such embodiments, the disease or disorder is cancer.

In some such embodiments, the indication of the health of the subject is a class output of a respective state, in a plurality of possible states, of the health of the subject.

In some such embodiments, the indication of the health of the subject is a probability output for the corresponding state of the health of the subject.

In some such embodiments, the model is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

In some such embodiments, the plurality of parameters is at least 1000, at least 10,000, at least 15,000, at least 50,000, at least 100,000, at least 250,000, at least 500,000, or at least 1,000,000 parameters.

In some such embodiments, the model applies the plurality of parameters to the information through at least 25,000, at least 50,000, at least 100,000, at least 250,000, at least 500,000, or at least 1,000,000 computations to obtain a corresponding output for the respective training subject from the model.

Another aspect of the present disclosure provides a computer system. The computer system comprises one or more processors and a non-transitory computer-readable medium including computer-executable instructions that, when executed by the one or more processors, cause the processors to perform the method described herein.

Another aspect of the present disclosure provides a non-transitory computer readable storage medium. The non-transitory computer readable storage medium stores instructions, which when executed by a computer system, cause the computer system to perform any of the methods described herein.

### BRIEF DESCRIPTION OF THE DRAWINGS

FIG. 1 illustrates a block diagram of an example computing device, in accordance with some embodiments of the present disclosure.

FIGS. 2A, 2B, 2C, 2D, 2E, 2F, and 2G collectively provide a flow chart of processes and features for identifying a set of gut microorganisms, in accordance with some embodiments of the present disclosure.

FIGS. 3A, 3B, 3C, and 3D collectively provide a flow chart of processes and features for training a model for evaluating human health, in accordance with some embodiments of the present disclosure.

FIGS. 4A, 4B, and 4C collectively provide a flow chart of processes and features evaluating the health of a subject, in accordance with some embodiments of the present disclosure.

FIGS. 5A, 5B, 5C, 5D, 5E, 5F, 5G, and 5H collectively illustrate reversible changes of gut microbiota associates with reversible shifts of metabolic phenotypes in patients with T2DM. (A) Study design. Before Run-in, written informed consent, questionnaire of personal information and measuring HbA1c at screening. After Run-in, medical checkup and sample collection at baseline (M0), three months after on the high fiber intervention or usual diet (M3) and one year after the high fiber intervention stopped (M15). (B) Changes of fiber intake. (C) Global changes of the gut microbiome as shown by the principal coordinate analysis based on the Bray-Curtis distance for the 1845 genomes and (D) Average Bray-Curtis distance between the groups. PERMANOVA test (9,999 permutations) was performed to compare the groups. *P<0.05 and ***P<0.001. The color of the square showed the magnitude of average Bray-Curtis distance. (E) Change of HbA1c, (F) The percentage of participants with adequate glycemic control, (G) Fasting blood glucose, and (H) The glucose area under the curve (AUC) in a meal tolerance test (MTT). For (E), (G) and (H), data shown as percent changes from baseline (±S.E.M). Friedman test followed by Nemenyi post-hoc test was used for comparison in the same group, compact letters reflect significance (P<0.05). n=67 in W group and n=28 in U group. Mann-Whitney test (two-sided) was used for comparison between W and U at the same time point, *P<0.05, **P<0.01 and ***P<0.001. n=74 in W (M0) (For panel H, n=72), n=74 in W (M3), n=67 in W (M15), n=36 in U (M0), n=36 in U (M3) and n=28 in U (M15).

FIGS. 6A, 6B1, 6B2, and 6B3 collectively illustrate that two competing guilds of bacteria constitute a robust seesaw network despite the profound global changes in the gut microbial ecosystem induced by introduction and withdrawal of the high fiber intervention. (A) The distribution of different types of correlations of the genome pairs during the trial. The 3 letters show the correlations of the genome pairs at M0, M3 and M15 subsequently. Stable correlations, NNN and PPP, were highlighted (B) UPGMA clustering of the 141 nodes based on their robust positive and negative correlations showed two clusters (green and purple range). The bar plots show the abundance changes of each node throughout the trial, which is expressed as median abundance with Z-score transformation. The differences of each node over time were tested using the Friedman test followed by Nemenyi post-hoc test. P<0.05 was considered as significant. For (B), the color of the node represents the members in the two guilds: green for Guild 1 and purple for Guild 2.

FIGS. 7A, 7B, 7C1, and 7C2 collectively illustrate the balance between the two competing guilds in the seesaw network was associated with the metabolic health of patients with type 2 diabetes. (A) Change of the total abundance of Guild 1, Guild 2, and their ratio across the trial in the W group. Friedman test followed by Nemenyi test was used to analyze the difference between time points. Compact letters reflect the significance at P<0.05. (B) Random Forest regression with leave-one-out cross-validation was used to explore the associations between the 141 genomes and the clinical parameters. The bar plot shows the Pearson's correlations coefficient between the predicted and measured values. The asterisk before the parameter's name shows the significance of the Pearson's correlations. P values were adjusted by Benjamini & Hochberg's method. * adjusted P<0.05, ** adjusted P<0.01 and adjusted ***P<0.001. BMI, body mass index; SBP, systolic blood pressure; DBP, diastolic blood pressure; WC, waist circumference; HP, hip circumference; TNF-α, tumor necrosis factor-α; WBC, white blood cell count; CRP, C-reactive protein; LBP, lipopolysaccharide-binding protein; TC, total cholesterol; TG, triglyceride; Lpa, lipoprotein a; HDL, high-density lipoprotein; APOA, apolipoprotein A; LDL, low-density lipoprotein; APOB, apolipoprotein B; GFR (MDRR), glomerular filtration rate; CysC, Cystatin C; ACR, urinary microalbumin to creatinine ratio; IMT, intima-media thickness; DAN, diabetic autonomic neuropathy score; MHR, mean heart rate; SDNN, standard deviation of NN intervals; SDANN, standard deviation of the average NN intervals calculated over 5 minutes; SDNNIndex, mean of standard deviation of NN intervals for 5-minute segments; rMSSD, root-mean-square of the differences of successive NN intervals; pNN50, percentage of the interval differences of successive NN intervals greater than 50 ms; TP, total power; VLF, very low frequency power; LF, low frequency power; HF, high frequency power; DPN, diabetic peripheral neuropathy score. (C) Differences in genetic capacity of carbohydrate substrate utilization (CAZy), short-chain fatty acid production (SCFA), number of antibiotic resistance genes (ARG) and number of virulence factor genes (VF). (C) The heatmaps show the proportion (CAZy) or gene copy numbers (SCFA, ARG and VF) of each category in each genome. For carbohydrate substrate utilization, CAZy genes were predicted in each genome. The proportion of CAZy genes for a particular substrate was calculated as the number of the CAZy genes involved in its utilization divided by the total number of the CAZy genes. Arabinoxylan-related CAZy families: CE1, CE2, CE4, CE6, CE7, GH10, GH11, GH115, GH43, GH51, GH67, GH3 and GH5; cellulose-related: GH1, GH44, GH48, GH8, GH9, GH3 and GH5; inulin-related: GH32 and GH91; mucin-related families: GH1, GH2, GH3, GH4, GH18, GH19, GH20, GH29, GH33, GH38, GH58, GH79, GH84, GH85, GH88, GH89, GH92, GH95, GH98, GH99, GH101, GH105, GH109, GH110, GH113, PL6, PL8, PL12, PL13 and PL21; pectin-related: CE12, CE8, GH28, PL1 and PL9; starch-related: GH13, GH31 and GH97. For short chain fatty acid production, FTHFS: formate-tetrahydrofolate ligase for acetate production; ScpC: propionyl-CoA succinate-CoA transferase and Pct: propionate-CoA transferase for propionate production; But: Butyryl-coenzyme A (butyryl-CoA): acetate CoA transferase, Buk: butyrate kinase, 4Hbt: butyryl-CoA: 4-hydroxybutyrate CoA transferase, Ato: butyryl-CoA:acetoacetate CoA transferase (AtoA: alpha subunit, AtoD: beta subunit) for butyrate production. Mann-Whitney test (two-sided) was used to analyze the difference between Guild 1 and Guild 2. #P<0.1, *P<0.05, **P<0.01 and ***P<0.001. Guild 1 (green bar): n=50, Guild 2 (purple bar): n=91.

FIGS. 8A1, 8A2, 8A3, 8A4, and 8B collectively illustrate that a seesaw networked microbiome signature exists in other independent human cohorts and supports classification models for different diseases. (A) The microbiome signature supports classification models for the four different diseases. The area under the ROC curve (AUC) of the Random Forest classifier based on the 141 genomes in the microbiome signature to classify control and patients in each dataset. Leave-one-out cross validation was applied. Type 2 diabetes (T2DM): Control n=136, T2D n=136; Atherosclerotic cardiovascular disease (ACVD): Control n=171 and ACVD n=214; Liver cirrhosis (LC): control n=83 and LC n=84; Ankylosing spondylitis: Control n=83 and AS n=97. (B) The microbiome signature is associated with key T2D phenotypes.

FIG. 9 illustrates a flow diagram of participants in the trial described in Example 1.

FIGS. 10A, 10B, 10C, and 10D collectively illustrate violin plots of energy and macronutrient intake during the trial in W and U group. Friedman test followed by Nemenyi post-hoc test was used for comparison in the same group. Mann-Whitney test (two-sided) was used for comparison between W and U at the same time point. *P<0.05, **P<0.01 and ***P<0.001. Boxes show the medians and the interquartile ranges (IQRs), the whiskers denote the lowest and highest values that were within 1.5 times the IQR from the first and third quartiles, and outliers are shown as individual points.

FIGS. 11A, 11B, 11C, and 11D collectively illustrate violin plots of the change in alpha diversity of gut microbiomes during the trial in W and U group. (A) Shannon Index; (B) Simpson Index; (C) Observed Genomes; (D) Chao 1 Index. Friedman test followed by Nemenyi post-hoc test was used for comparison in the same group. Mann-Whitney test (two-sided) was used for comparison between W and U at the same time point. *P<0.05, **P<0.01 and ***P<0.001. Boxes show the medians and the interquartile ranges (IQRs), the whiskers denote the lowest and highest values that were within 1.5 times the IQR from the first and third quartiles, and outliers are shown as individual points.

FIGS. 12A, 12B and 12C collectively illustrate that co-abundance networks of the prevalent genomes were scale-free networks across the trial. Degree distribution were fitted well with power law model.

FIG. 13 illustrates that introduction and withdrawal of high fiber intervention significantly change the network degree distribution.

FIGS. 14A, 14B, 14C, 14D, 14E, and 14F collectively illustrate that the 141 genomes contribute most of the interactions in the network. (A) The stacked bar plot shows the distribution of positive and negative edges within the 141 genomes, between the 141 genomes and the other nodes, and within the other nodes. The 141 genomes had significantly higher degree (B), betweenness centrality (C), eigenvector centrality (D), closeness centrality (E) and stress centrality (F) than the rest of the nodes in the networks. Mann-Whitey test (two-sided) was performed. ***P<0.0001.

FIG. 15 illustrates that the 141 nodes were widely shared by the patients in the W group. The histogram shows the distribution of genomes shared by the 74 patients with various prevalence.

FIGS. 16A, 16B, and 16C collectively illustrate that a similar beta-diversity pattern was found based on the 141 genomes as compared with that based on all the 1845 genomes. (A) Global changes of the gut microbiome as shown by the principal coordinate analysis based on the Bray-Curtis distance with the abundance of the 141 genomes. (B) Average Bray-Curtis distance between the groups (B). PERMANOVA test (9,999 permutations) was performed to compare the groups. *P<0.05 and ***P<0.001. The color of the square showed the magnitude of average Bray-Curtis distance. (C) Procrustes analysis combing the principal coordinate analysis for 1845 genomes and 141 genomes based on Bray-Curtis distance.

FIGS. 17A, 17B and 17C collectively illustrate that the 141 nodes organized themselves into two clusters with robust co-occurrence behavior within each cluster and can be recognized as potential ecological guilds. (A-C) The stacked bar plot shows the number of positive and negative edges within or between the guilds. Red, within Guild 1; Blue: within Guild 2; Green, between the two guilds.

FIGS. 18A and 18B collectively illustrate that genomes in Guild 1 had much lower genetic capacity for pathogenicity and antibiotic resistance. (A) The bar plot shows the number of genes encoding virulence factors (VF) and classes of VFs. (B) The bar plot shows the number of ARGs and the corresponding antibiotic resistance types.

FIG. 19 illustrates a workflow for validating the microbiome signature in other datasets, in accordance with some embodiments of the disclosure.

FIG. 20 illustrates that the microbiome signature is associated with host phenotypes in a liver cirrhosis dataset (Qin 2014, et al.). Random Forest regression with leave-one-out cross-validation was used to explore the associations between the microbiome signature and the clinical parameters. The bar plot shows the Pearson's correlations coefficient between the predicted and measured values. The asterisk before the parameter's name shows the significance of the Pearson's correlations. P values were adjusted by Benjamini & Hochberg's method. ** adjusted P<0.01 and ***P<0.001. TB: total bilirubin, Crea: creatinine level, Alb: albumin level, BMI: Body mass index. N=167.

FIGS. 21A and 21B collectively illustrate receiver operating characteristic (ROC) curves for performance of random forest classifiers trained to predict human disease against genomic abundance values for 141 gut microorganisms in diseased and healthy subjects in studies of various diseases, as described in Example 1.

FIGS. 22A and 22B collectively illustrate clinical parameters during intervention in the W and U group.

FIG. 23 illustrates the characteristics of the co-abundance networks of the prevalent genomes in the W group at M0, M3 and M15 during the trial, denoted as GM0, GM3 and GM15.

FIG. 24 illustrates the design of a high fiber intervention clinical study in T2D patients in China. Type 2 diabetes patients were randomized to treatment group, the W group, receive WTP diet for 3 month, and One-year follow-up after withdrawal of WTP diet; or to the control group, the u group, receive usual care and one-year follow-up.

FIG. 25 illustrates the genome-resolved metagenomic analysis used in the high fiber intervention clinical study of T2D patients. Shotgun metagenomics was applied to explore the gut microbiome in this study. On average, each sample had 91.5 million raw reads, and 86.5 million high quality reads. In brief, after de novo assembly, binning, quality control and dereplication, 1845 high quality and non-redundant genomes were obtained for further analysis at genome level. These genomes accounted >70% of the total reads in our metagenomic dataset.

FIGS. 26A, 26B, 26C, 26D, 26E, 26F, 26G, 26H, 26I, 26J, 26K, 26L, and 26M collectively illustrate classification performance with different numbers of genomes selected by degree based backward selection for eight types of diseases.

FIGS. 27A, 27B, 27C, 27D, 27E, 27F, 27G, 27H, 27I, 27J, 27K, 27L, and 27M collectively illustrate random forest classification performance with different numbers of genomes selected randomly for eight types of diseases.

FIGS. 28A, 28B, 28C, 28D,28E,28F, 28G, and 28H collectively illustrate the classification capacity of the two competing guilds identified from QD and various types of diseases. Microbiome signature comprising the genomes of two competing guilds are obtained from various disease: T2D (FIG. 28A), LC (FIG. 28B), SCZ (FIG. 28C), IBD (FIG. 28D), AS (FIG. 28E), ACVD (FIG. 28F), CRC (FIG. 28G), and QD (FIG. 28H). The identified microbiome signature for each condition is utilized to classify control and patients in each dataset using Random Forest classifiers. FIGS. 28A-28H shows all microbiome signature have the capacity to classify case and control across different studies.

FIGS. 29A and 29B collectively illustrate the rank of the classification of the microbiome signature from QD and other types of diseases. The eight sets of microbiome signature obtained from QD and from various diseases cases: T2D, LC, SCZ, IBD, AS, ACVD, CRC are ranked according to their performance in classifying case and control across 11 datasets. The ranking number for the best performer with the highest AUC value for each dataset is the lowest, whereas the ranking number for worst performer with the lowest AUC value is the highest. All the ranking numbers assigned to each set of signature microbiome is plotted FIG. 29A. FIG. 29B shows the sum of the ranking numbers for each set of microbiome signatures. The microbiome signature obtained from QD has the best performance to classify the healthy subjects vs. patients across different datasets.

FIGS. 30A and 30B collectively illustrate the capacity of the combined pool to classify case and control across different studies. The eight sets of signature microbiome obtained from QD and various diseases cases: T2D, LC, SCZ, IBD, AS, ACVD, CRC were pooled together as a combined microbiome signature. FIG. 30A shows the comparison of classification performance of the combined pool with each of the individual signature microbiome based on AUC values. FIG. 30B shows the significance of intra-group comparison. Friedman test followed by Dunn's post hoc was performed for the analysis (#BH adjusted P<0.1, * BH adjusted P<0.05).

FIGS. 31A, 31B and 31C collectively illustrate the rank of the classification performance of the microbiome signature. The nine sets of microbiome signature obtained from combined pool, QD or various diseases cases: T2D, LC, SCZ, IBD, AS, ACVD, CRC were ranked according to their performance in classifying case and control across 11 datasets. All the ranking numbers assigned to each set of signature microbiome are plotted FIG. 31A. FIG. 31B shows the significance of intra-group comparison. FIG. 31C shows the sum of the ranks for each set of microbiome signatures. Kruskal-Wallis test followed by Dunn's post hoc was performed for the analysis (#BH adjusted P<0.1, * BH adjusted P<0.05). The microbiome signature obtained from the combined pool has the best performance to classify the healthy subjects vs. patients across different datasets.

FIG. 32 illustrates the selection of the combined core pool. Random Forest classification based on a combined 788 genomes are performed for each dataset. Each of the 788 genome was ranked based on its importance. A summed rank was obtained by adding up the value of ranks across 11 datasets all 788 genomes are ranked again based on the summed value. The most important genome across 11 dataset gets the lowest summed rank value. Starting from the least important genome, every genome one by one was removed from each dataset based on order of importance. The classification performance (AUCs) was calculated for the remaining numbers of genomes after each removal by Random Forest model and all the genome numbers are ranked based on AUC values. The rank values for each genome number across 11 datasets was summed. The sum of ranks for each genome number across 11 datasets was plotted. 302 genomes achieved lowest summed AUC ranks. After removing 18 genomes which exhibit inconsistent C1A and C1B assignment, 284 genomes remained as the combined core pool.

FIGS. 33A, 33B, 33C, 33D, 33E, 33F, 33G, 33H, 33I, and 33J collectively illustrate the classification capacity of the two competing guilds identified from QD, various types of diseases, combined pool, and combined core pool. Microbiome signature comprising the genomes of two competing guilds were obtained from various disease: T2D (FIG. 33A), LC (FIG. 33B), AS (FIG. 33C), CRC (FIG. 33D), IBD (FIG. 33E), QD (FIG. 33F), AVCD (FIG. 33G), SCZ (FIG. 33H), combined pool (FIG. 33I), and combined core pool (FIG. 33J). The identified microbiome signature for each condition was utilized to classify control and patients in each dataset using Random Forest classifiers. FIG. 31 shows all microbiome signature have the capacity to classify case and control across different studies.

FIGS. 34A and 34B collectively illustrate the capacity of the combined core pool to classify case and control across different studies. FIG. 34A shows the comparison of classification performance based on AUC of the combined core pool with signature microbiome obtained from combined pool, QD and various diseases cases: T2D, LC, SCZ, IBD, AS, ACVD, CRC. FIG. 34B shows the significance of intra-group comparison. Friedman test followed by Dunn's post hoc was performed for the analysis (#BH adjusted P<0.1, * BH adjusted P<0.05, **BH adjusted P<0.01).

FIGS. 35A, 35B, and 35C collectively illustrate the rank of the classification performance of the microbiome signature. Ten sets of microbiome signature obtained from combined core pool, combined pool, QD or various diseases cases: T2D, LC, SCZ, IBD, AS, ACVD, CRC were ranked according to their performance in classifying case and control across 11 datasets. All the rank values assigned to each set of signature microbiome were plotted FIG. 35A. FIG. 35B shows the significance of intra-group comparison. FIG. 35C shows the sum of the ranks for each set of microbiome signatures. Kruskal-Wallis test followed by Dunn's post hoc was performed for the analysis (#BH adjusted P<0.1, * BH adjusted P<0.05, **BH adjusted P<0.01). The microbiome signature obtained from the combined core pool has the best performance to classify the healthy subjects vs. patients across different datasets.

FIG. 36 illustrates the flow of identifying microbiome signature from a case cohort and a control cohort.

FIG. 37 illustrates combined case and control samples from the 25 datasets that corresponded to 15 various diseases (type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC).

FIGS. 38A1, 38A2, 38A3, 38B1, 38B2, and 38B3 collectively illustrate the Universal Random Forest classification model for case vs control based on the abundance of the 284 core genomes. A: 80% training: Control, n=1285; Case, n=1424, 10-fold CV (A1: The area under the ROC curve (AUC) of the Random Forest classifier; A2: Score density for case and control; A3: Probability score for case and control); B: 20% testing: Control, n=319; Case, n=356 (B1: The area under the ROC curve (AUC) of the Random Forest classifier; B2: Score density for case and control; B3: probability score for case and control).

FIGS. 39A and 39B collectively illustrate the repeated training of Universal Random Forest classification model for case vs control with randomly selected number of genomes. (A) Each data point represents average AUC for a Random Forest model trained ten times using a different set of randomly selected genomes at a total number of X (as indicated by the X-axis) determined against the training set. (B) Each data point represents average AUC for a Random Forest model trained ten times using a different set of randomly selected genomes at a total number of X (as indicated by the X-axis) determined against a testing set.

FIGS. 40A and 40B collectively illustrate genome pairwise ANI comparison. FIG. 40A depicts all genome pairwise ANI comparison among the 788 combined pool of genomes. FIG. 40B depicts the pairwise ANI comparison between Guild 1 genomes and Guild 2 genomes.

FIGS. 41A, 41B, 41C, 41D, 41E, 41F, 41G, 41H, and 41I collectively illustrate the corresponding contigs, referenced by SEQ IDs, obtained for each of the 788 genomes.

FIGS. 42A, 42B, 42C, 42D, 42E, 42F, 42G, 42H, 42I, 42J, 42K, 42L, 42M, 42N, 420, 42P, 42Q, 42R, 42S, 42T, 42U, 42V, 42W, 42X, 42Y, 42Z, 42AA, 42BB, 42CC, 42DD, 42EE, 42FF, 42GG, 42HH, 4211, 42JJ, 42KK, 42LL, 42MM, 42NN, 4200, 42PP, 42QQ, 42RR, 42SS, 42TT, 42UU, 42VV, 42WW and 42XX collectively illustrate the Taxonomy Assignment of 788 combined microbiome.

Like reference numerals refer to corresponding parts throughout the several views of the drawings.

### DETAILED DESCRIPTION

The methods and systems described herein facilitate determination of a disease state, in a plurality of disease states, of a subject based on the constitution of the subject's microbiome.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

As used herein, the term “genomic abundance value” refers to an absolute or relative amount of a microorganism's genome in a biological sample from the gut of a subject. A genomic abundance value can be expressed different units, including copy number, molarity, mass (e.g., normalized against the size of the genome), unique sequence reads (e.g., normalized against the size of the genome), a percentage of any of the former metrics relative to the total amount of the metric across all genomes in the sample, a percentage of any of the former metrics relative to the total amount of the metric across a plurality of genomes in the sample, etc. In some embodiments, a genomic abundance value is normalized against a total genomic abundance in the sample. In some embodiments, a genomic abundance value is normalized against a genomic abundance value for a control genome in the sample. In some embodiments, the values for a plurality of genomic abundance values in a sample are standardized, normalized, and/or scaled. Examples of methods for normalizing genomic abundance values are described, for example, in Lin, H., Peddada, S. D., Analysis of microbial compositions: a review of normalization and differential abundance analysis, Biofilms Microbiomes, 6(60) (2020) and Lutz K. C., et al., A Survey of Statistical Methods for Microbiome Data Analysis, Frontiers in Applied Mathematics and Statistics, 8 (2022) the contents of which are incorporated herein by reference in their entireties. Methods for measuring genomic abundance values are known in the art. For example, metagenomic sequencing can be used to largely reconstruct microbial genomes from next generation sequencing of genomic DNA in biological samples, such as biological samples from the gut of a subject. For a review of metagenomic sequence see, for example, Quince C, et al., Shotgun metagenomics, from sampling to analysis, Nat Biotechnol, 35(9):833-44 (2017), the content of which is incorporated herein by reference in its entirety. Genomic abundance may also be determined by quantification of the copy number of a ribosomal gene, for example the 16S rRNA gene. Examples of rRNA quantification are described in Manzari C., et al., Accurate quantification of bacterial abundance in metagenomic DNAs accounting for variable DNA integrity levels, Microb Genom., 6(10):mgen000417 (2020) and Barlow, J. T., et al., A quantitative sequencing framework for absolute abundance measurements of mucosal and lumenal microbial communities, Nat Commun., 11:2590 (2020), the contents of which are incorporated herein by reference in their entireties.

As used herein, the term “relative abundance” refers to a ratio of a first amount of a compound measured in a sample, e.g., a genome for a first microorganism, to a second amount of a compound measured in a second sample. In some embodiments, relative abundance refers to a ratio of an amount of a compound, e.g., a genome for a first microorganism, to a total amount of compounds, e.g., the total amount of microorganism genomes or the total amount of a plurality of genomes, in the same sample. In other embodiments, relative abundance refers to a ratio of an amount of a compound, e.g., a genome for a first microorganism, in a first sample to an amount of the compound of the compound in a second sample. For instance, a ratio of a normalized amount of a genome for a first microorganism in a first sample to a normalized amount of the genome for the first microorganism in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” refers to any form of nucleotide sequence read including the raw sequence reads obtained directly from a nucleic acid sequencing technique or from a sequence derived therefrom, e.g., an aligned sequence read, a collapsed sequence read, or a stitched sequence read.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a microorganism that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a microorganism that are sequenced in a particular sequencing reaction. For example, in some embodiments, sequencing depth refers to the average depth of every locus across a targeted sequencing panel, an exome, or an entire genome for the microorganism. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “sequencing breadth” refers to what fraction of a particular microorganism genome has been sequenced. Sequencing breadth can be expressed as a fraction, a decimal, or a percentage, and is generally calculated as (the number of loci analyzed/the total number of loci in the genome). The denominator of the fraction can be a repeat-masked genome, and thus 100% can correspond to all of the reference genome minus the masked parts. A repeat-masked genome can refer to a genome in which sequence repeats are masked (e.g., sequence reads align to unmasked portions of the genome). In some embodiments, any part of a genome can be masked and, thus, sequencing breadth can be evaluated for any desired portion of a genome.

As used herein, the terms “sequence ratio” and “coverage ratio” interchangeably refer to any measurement of a number of units of a genomic sequence in a first one or more biological samples (e.g., a test and/or tumor sample) compared to the number of units of the respective genomic sequence in a second one or more biological samples (e.g., a reference and/or control sample). In some embodiments, a sequence ratio is a copy ratio, a log 2-transformed copy ratio (e.g., log 2 copy ratio), a coverage ratio, a base fraction, an allele fraction (e.g., a variant allele fraction), and/or a tumor ploidy. In some embodiments sequence ratio is a log N-transformed copy ratio, where N is any real number greater than 1.

As used herein, the term “sequencing probe” refers to a molecule that binds to a nucleic acid with affinity that is based on the expected nucleotide sequence of the RNA or DNA present at that locus.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest in a genome.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having a particular biological characteristic.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having a particular biological characteristic.

As used interchangeably herein, the term “classifier” or “model” refers to a machine learning model or algorithm.

In some embodiments, a model includes an unsupervised learning algorithm. One example of an unsupervised learning algorithm is cluster analysis. In some embodiments, a model includes supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, diffusion models, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent or backward propagation method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 50 parameters, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of ‘kernels’, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x_{(O)}∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in McLachlan et al., Bioinformatics 18(3):413-422, 2002. In some embodiments, in particular, those embodiments including a temporal component, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

As used herein, the term “instruction” refers to an order given to a computer processor by a computer program. On a digital computer, each instruction is a sequence of Os and is that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Example System Embodiments.

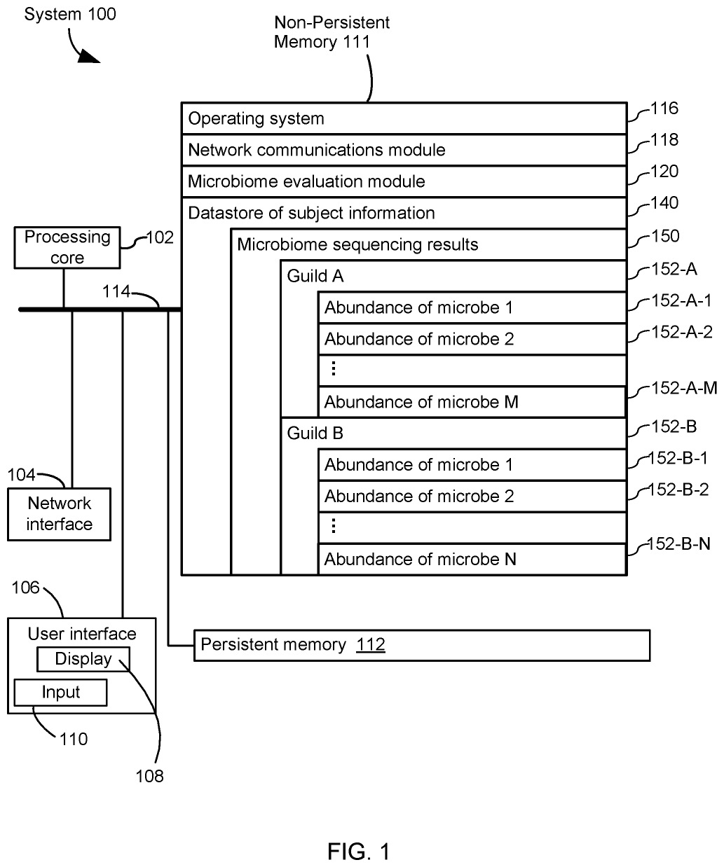

Now that an overview of some aspects of the present disclosure and some definitions used in the present disclosure have been provided, details of an exemplary system are described in conjunction with FIG. 1. FIG. 1 is a block diagram illustrating a system 100 in accordance with some implementations. The system 100 in some implementations includes one or more processing units CPU(s) 102 (also referred to as processors), one or more network interfaces 104, a user interface 106 including (optionally) a display 108 and an input system 110, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 104;
a microbiome evaluation module 140 for determining a disease state, in a plurality of disease states, of a subject based on the constitution of the subject's microbiome; and
a datastore of subject information 140 based on microbiome sequencing results 150, including abundance values 152 for microbes in each of guilds 152-A and 152-B as described herein.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of visualization system 100, that is addressable by visualization system 100 so that visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules instead may be stored in persistent memory 112.

### 1. Methods of Identifying a Set of Gut Microorganisms

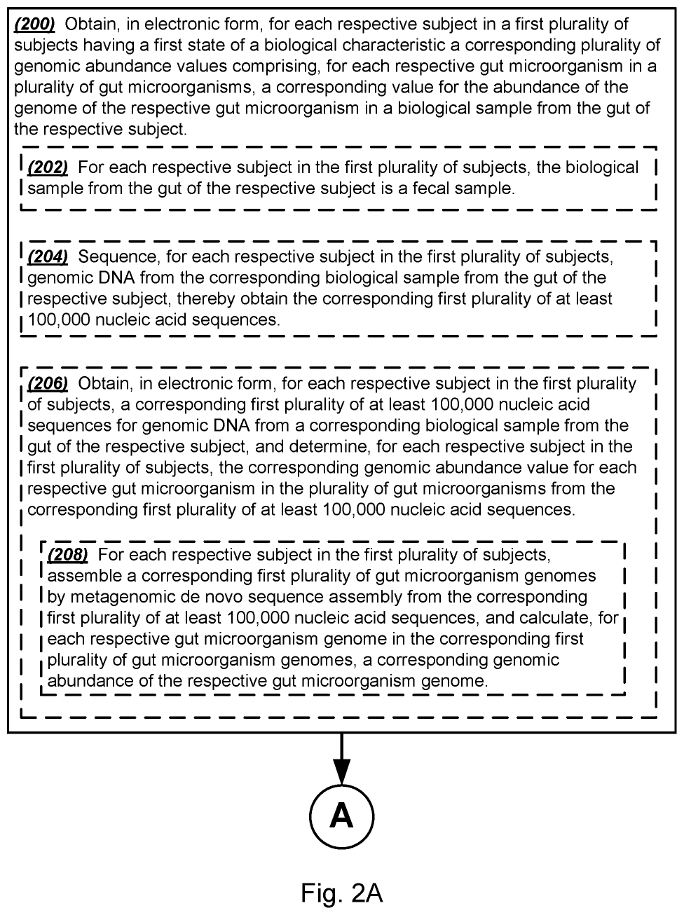

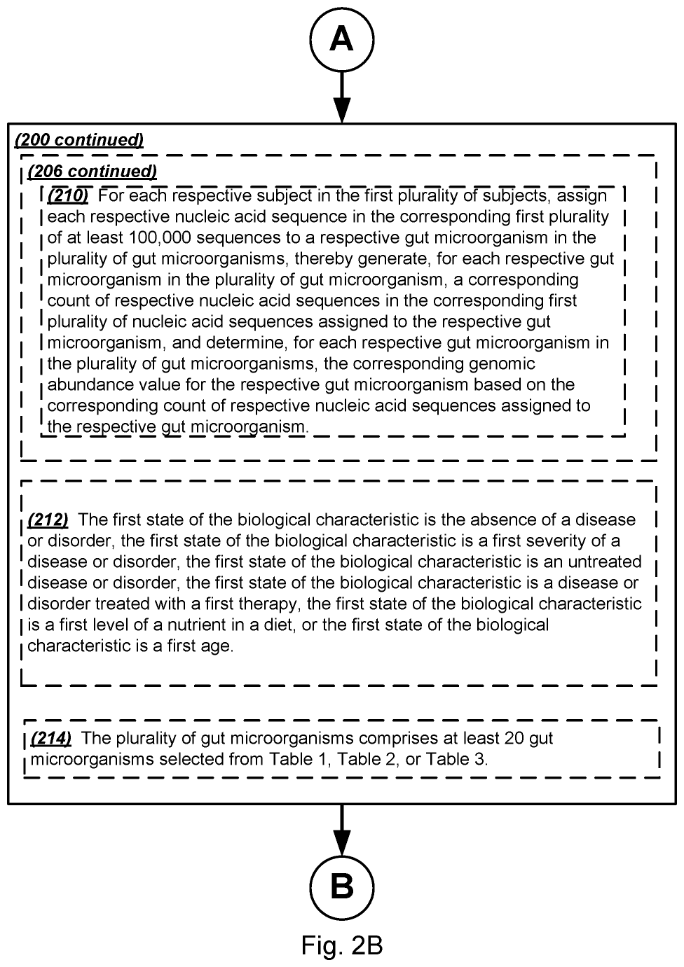

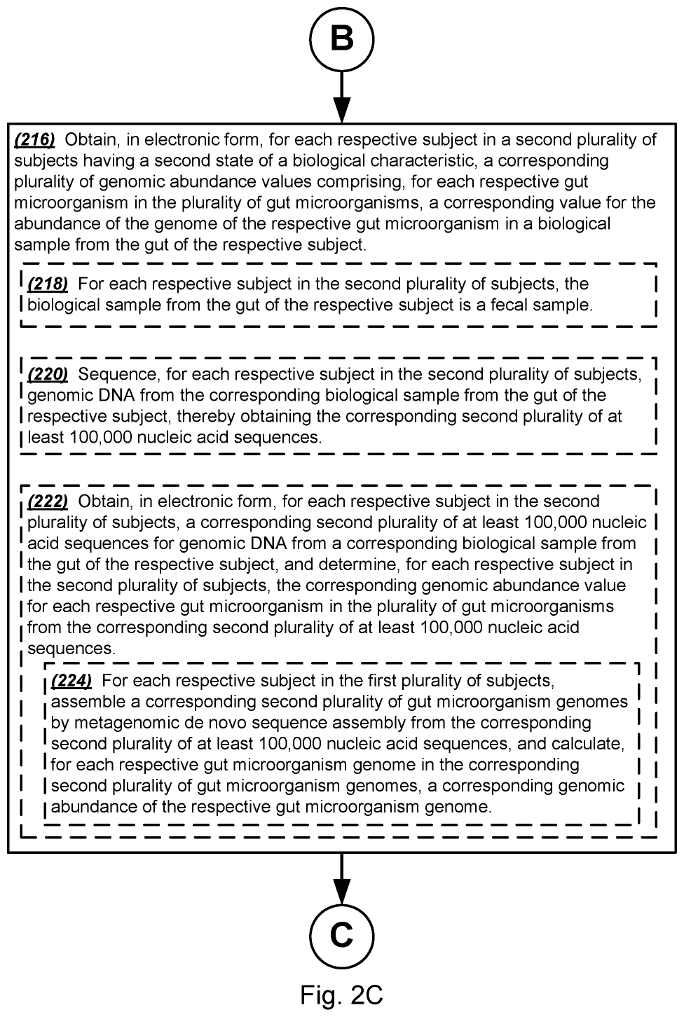

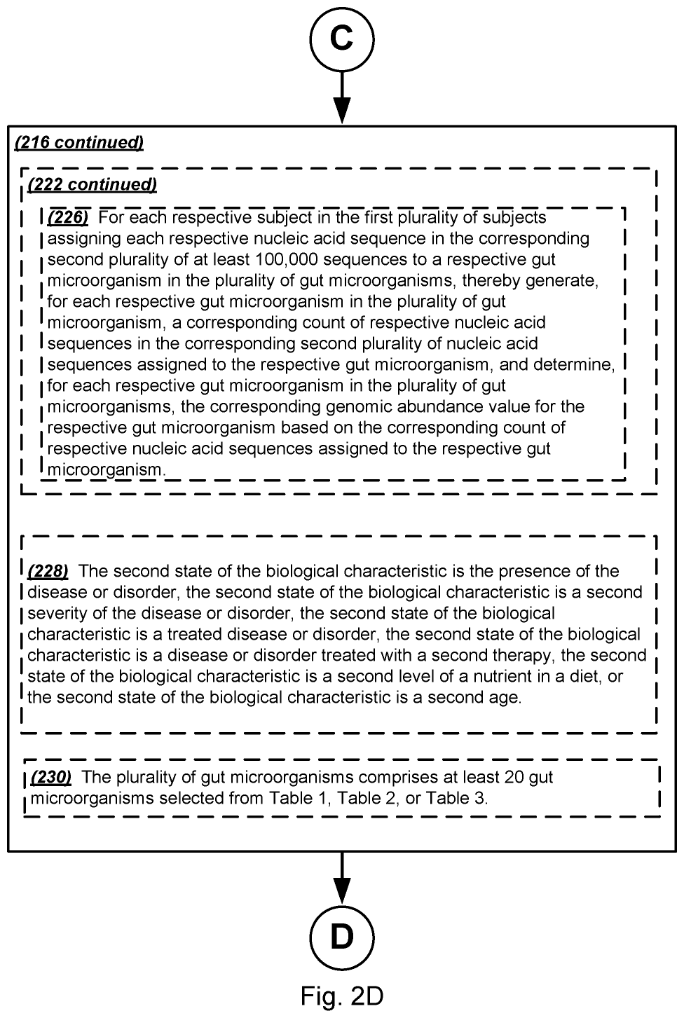

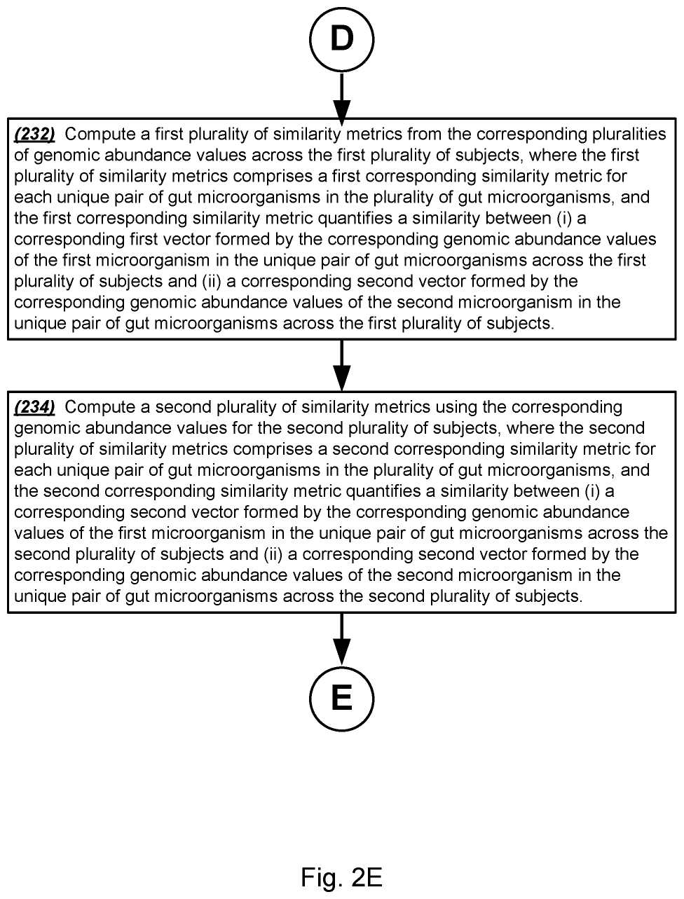

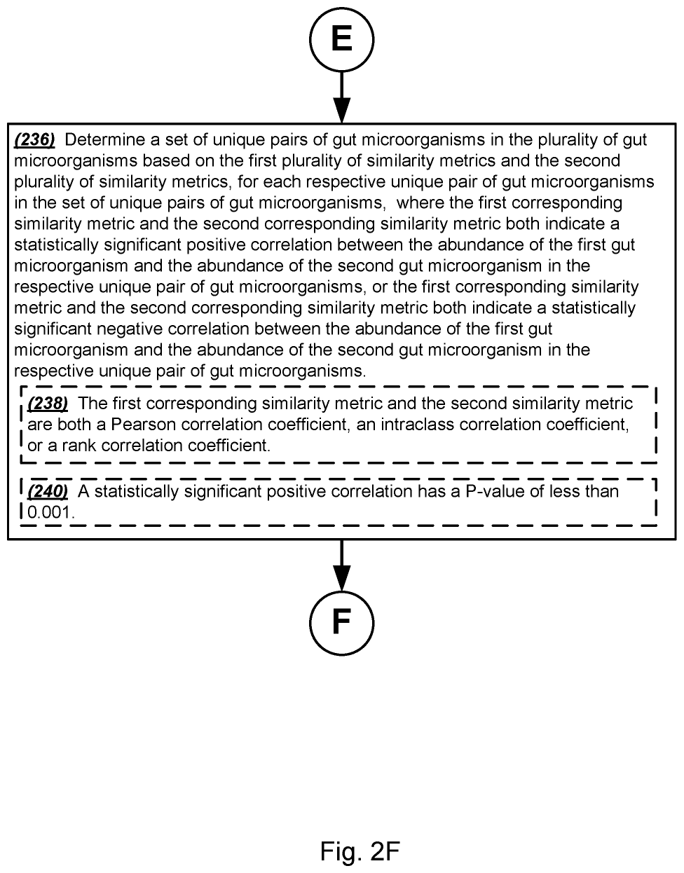

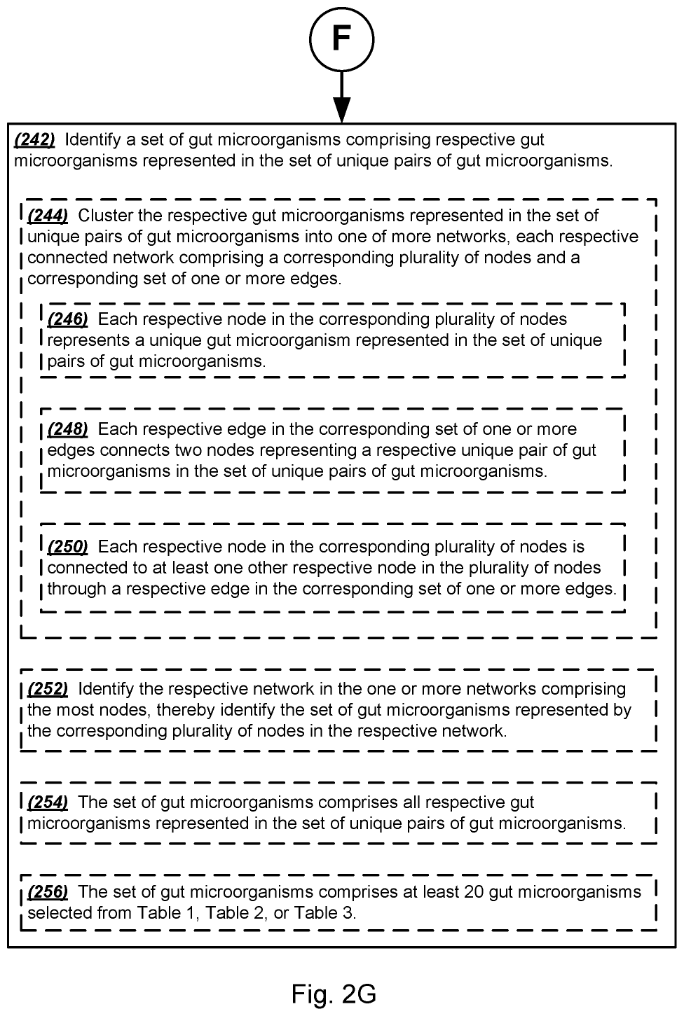

FIG. 2 is a schematic diagram of a method for identifying a set of gut microorganisms as discussed below. The method may be implemented using a computer system (e.g., the computer system 100 shown and described above in reference to FIG. 1).

Referring to block 200, in some embodiments, the method includes obtaining, in electronic form, for each respective subject in a first plurality of subjects having a first state of a biological characteristic a corresponding plurality of genomic abundance values comprising, for each respective gut microorganism in a plurality of gut microorganisms, a corresponding value for the abundance of the genome of the respective gut microorganism in a biological sample from the gut of the respective subject. In some embodiments, the first plurality of subjects comprises at least 50, at least 100, at least 200, at least 500, at least 1000, at least 5000, at least 10,000, at least 20,000, at least 50,000, at least 100,000, at least 500,000, or at least 1,000,000 subjects. In some embodiments, the first plurality of subjects comprises no more than 1,000,000, no more than 500,000, no more than 100,000, no more than 50,000, no more than 20,000, no more than 10,000, no more than 1000 subjects, no more than 500 subjects, no more than 100 subjects, or no more than 50 subjects. In some embodiments, the first plurality of subjects consists of from 50 to 100, from 50 to 200, from 50 to 500, from 100 to 500, from 200 to 500, from 200 to 1000, from 500 to 1000, from 200 to 5,000, from 1000 to 10,000, from 5000 from 200,00, from 10,000 to 50,000, from 20,000 to 100,000, or from 500,000 to 1,000,000. In some embodiments, the first plurality of subjects falls within another range starting no lower than 50 subjects and ending no higher than 10,000,000 subjects. In some embodiments, the first plurality of subjects share similar demographic characteristics (such as age, gender, ethnicity). In some embodiments, the first plurality of subjects share similar physical characteristics (such as weight, height, BMI value). In some embodiments, the first plurality of subjects share similar health status (such as physical or mental conditions, medical history, gene carrier, or medication use). In some embodiments, the first plurality of subjects share or similar behavior and lifestyle preferences (such as diet, physical exercise, or substance use).

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g., 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 70, at least 80, at least 90, at least 100, at least 110, at least 120, at least 130, at least 150, at least 200, at least 300, at least 400, at least 500, at least 750, at least 1000, or more. In some embodiments, shotgun metagenomic sequencing is employed to provide sequence reads for genomes in a sample, e.g., as described in U.S. Pat. No. 11,028,449, the content of which is incorporated herein by reference in its entirety.

Referring to block 202, in some embodiments, for each respective subject in the first plurality of subjects, the biological sample from the gut of the respective subject is a fecal sample. In some embodiments, the sample is a tissue biopsy, an intestinal, or mucosal sample. See, for example, Tang Q, J et al., Current Sampling Methods for Gut Microbiota: A Call for More Precise Devices, Front Cell Infect Microbiol., 10:151 (2020), the content of which is incorporated herein by reference in its entirety.

Referring to block 204, in some embodiments, the method includes sequencing, for each respective subject in the first plurality of subjects, genomic DNA from the corresponding biological sample from the gut of the respective subject, thereby obtaining the corresponding first plurality of (e.g., at least 100,000) nucleic acid sequences. In some embodiments, the first plurality of nucleic acid sequences comprises at least 100,000, at least 250,000, at least 500,000, at least 1,000,000, at least 2,500,000, at least 5,000,000, at least 10,000,000 or at least 50,000,000 nucleic acid sequences. In some embodiments, the first plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the first plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the first plurality of nucleic acid sequences falls within another range starting no lower than 100,000 nucleic acid sequences and ending no higher than 250,000,000 nucleic acid sequences.

In some embodiments, the first plurality of (e.g., at least 100,000) nucleic acid sequences are obtained through metagenomic sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

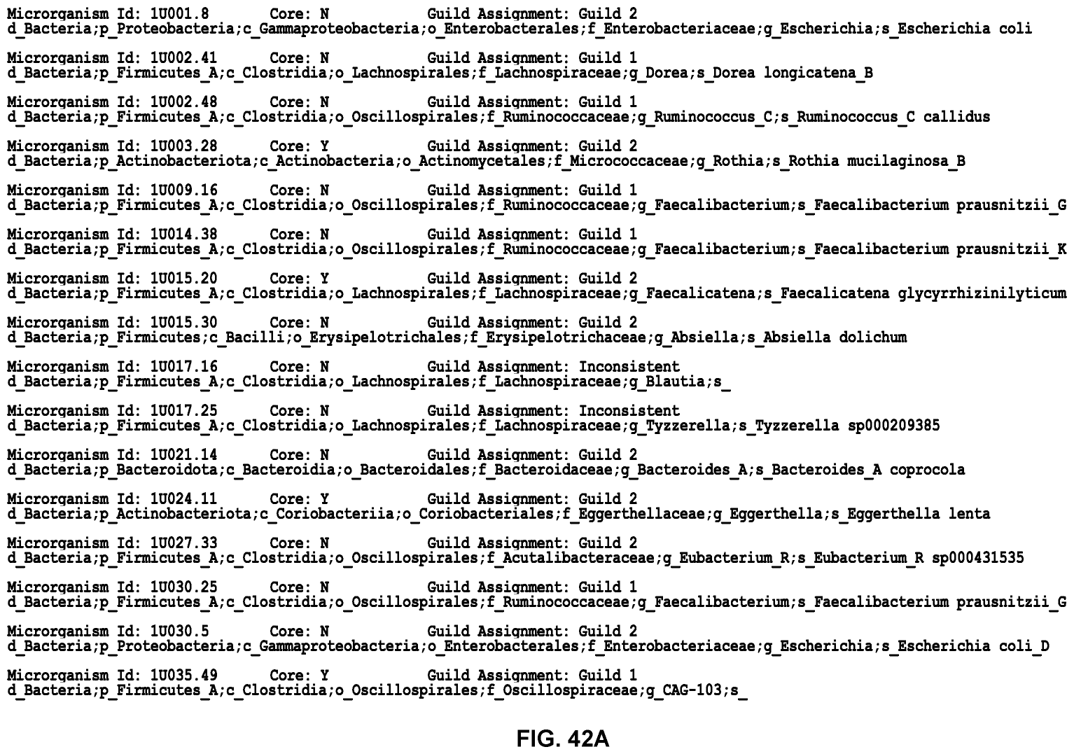

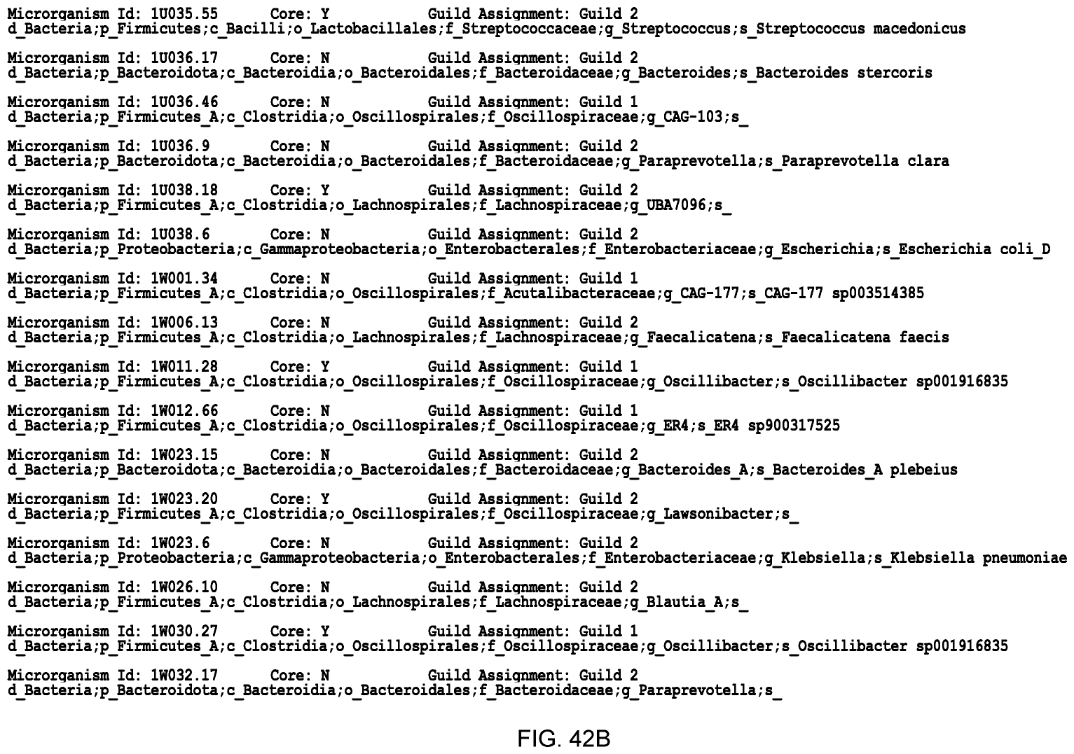

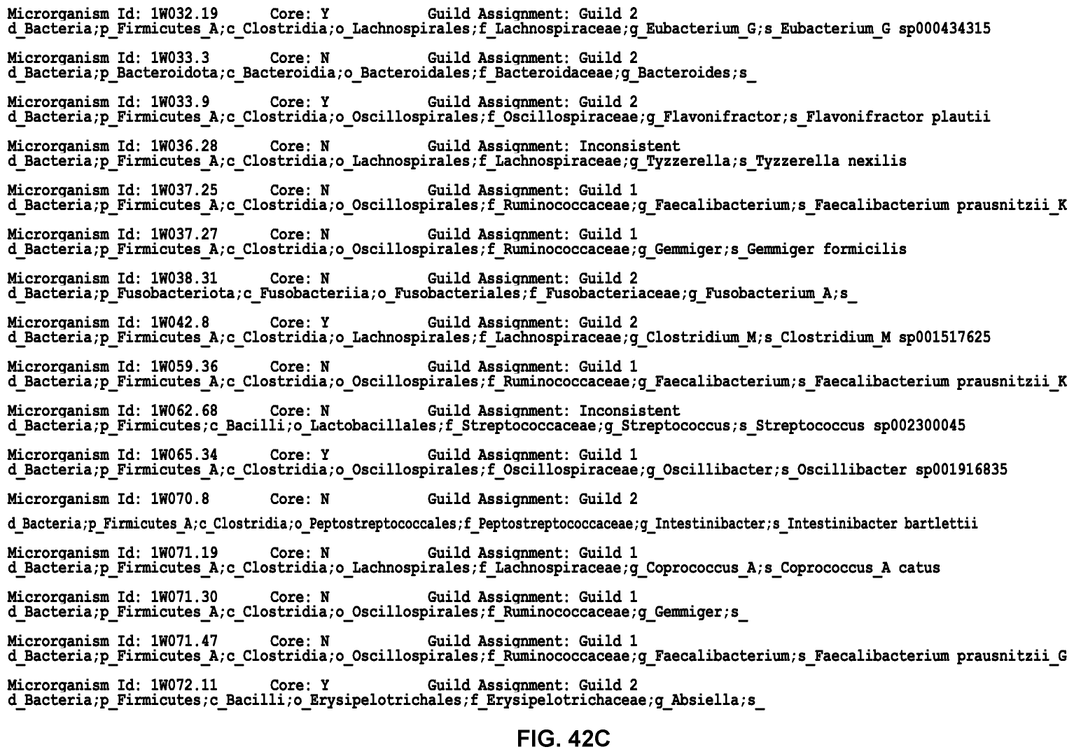

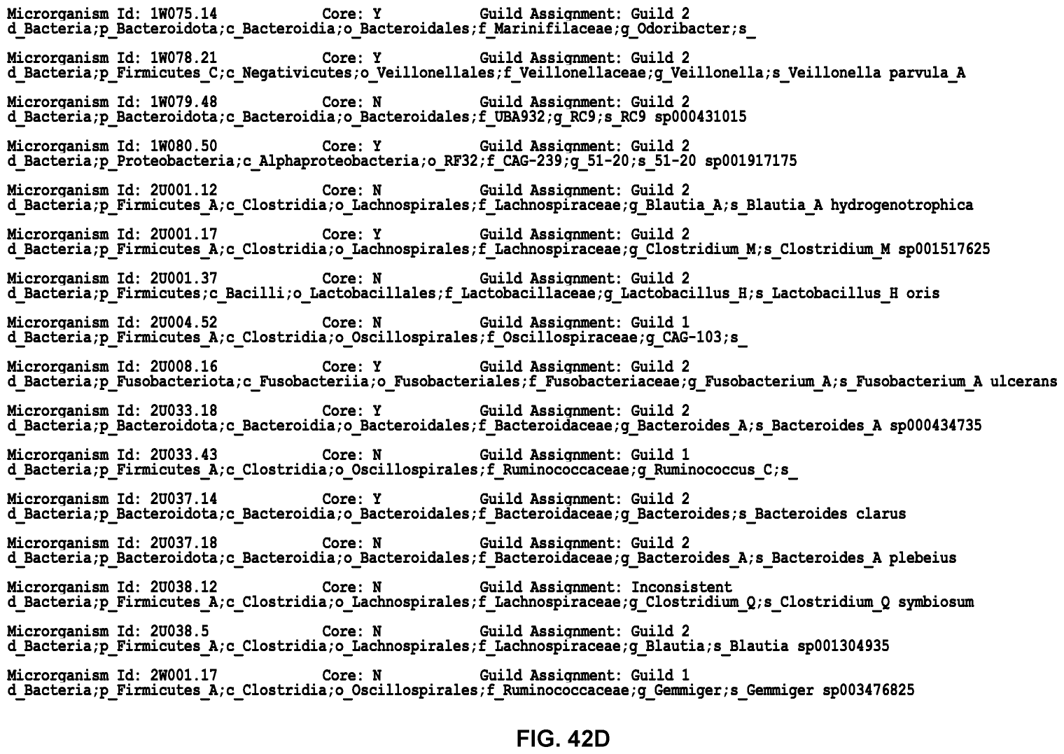

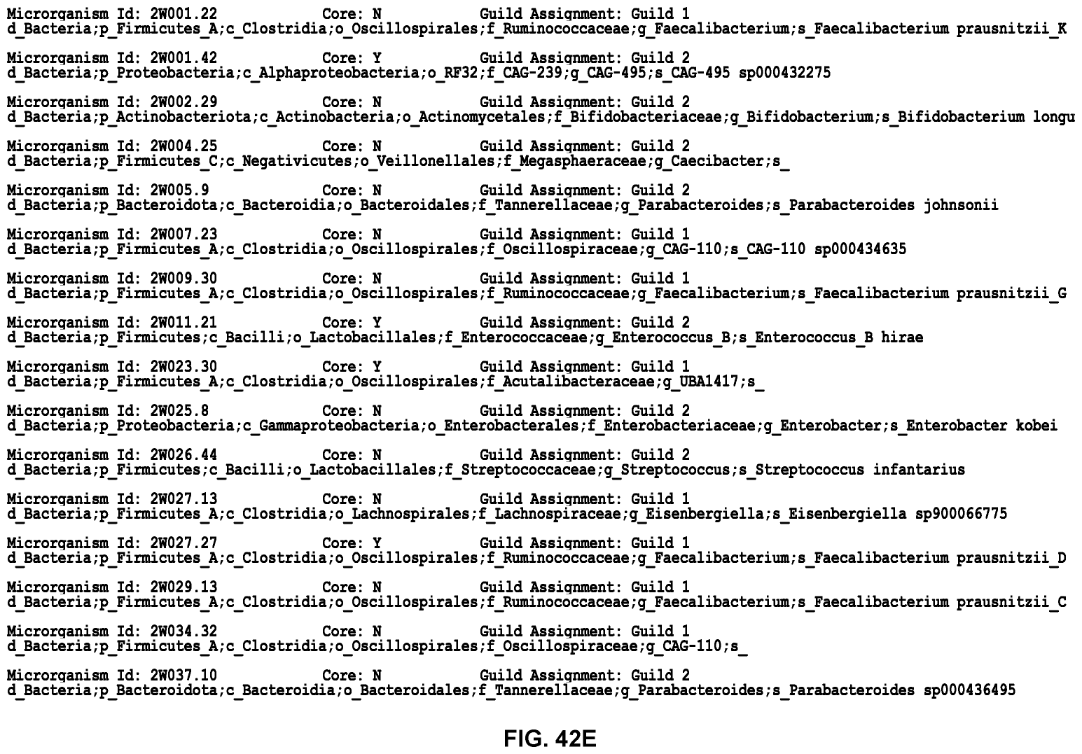

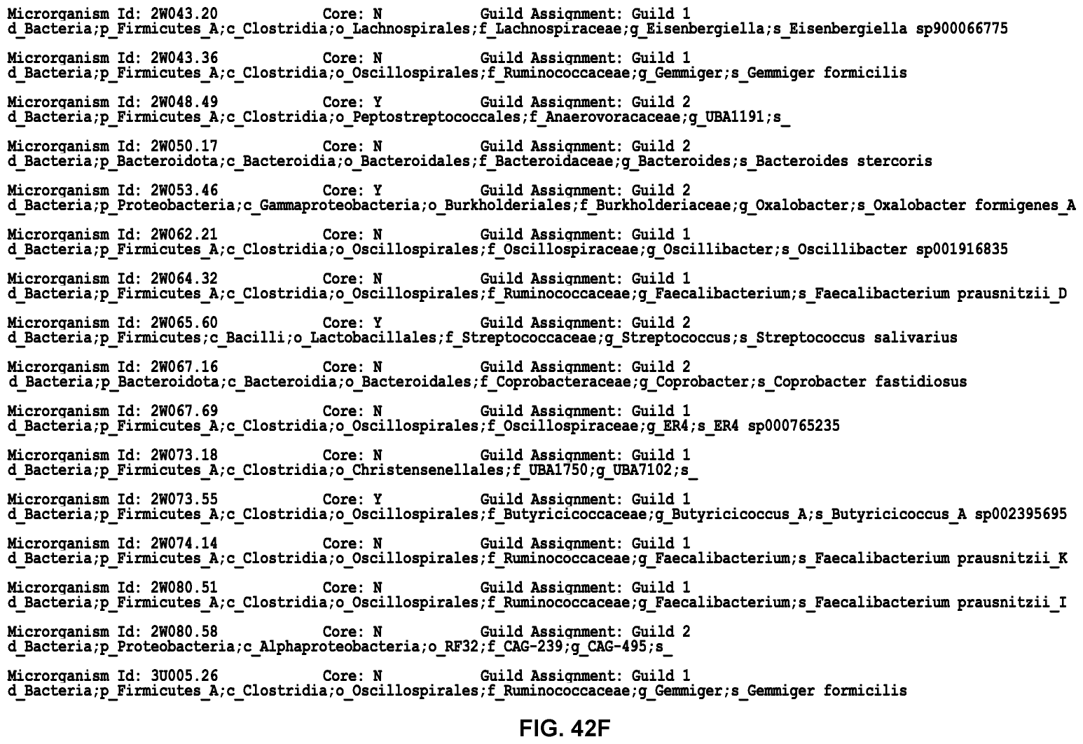

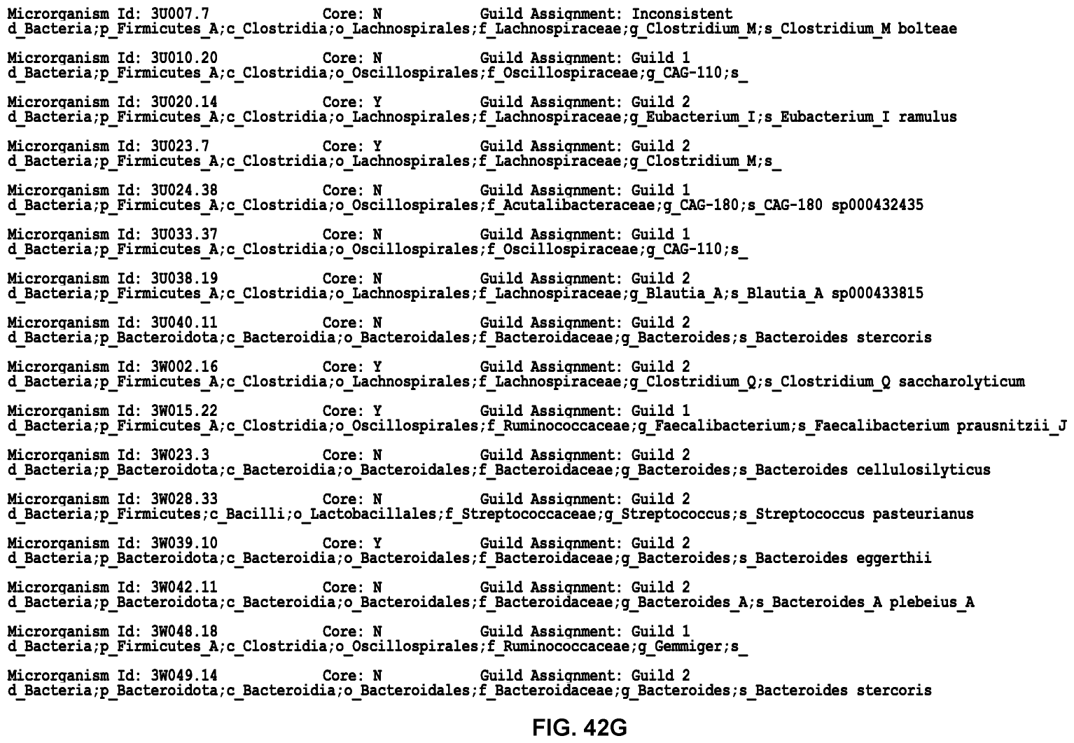

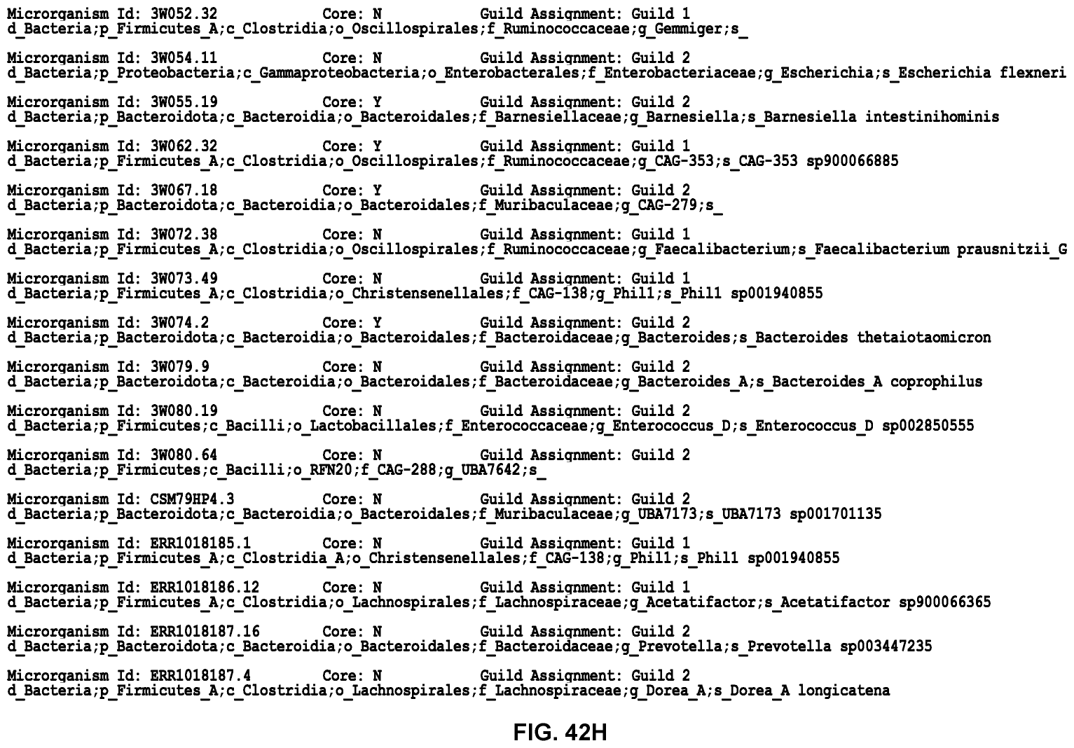

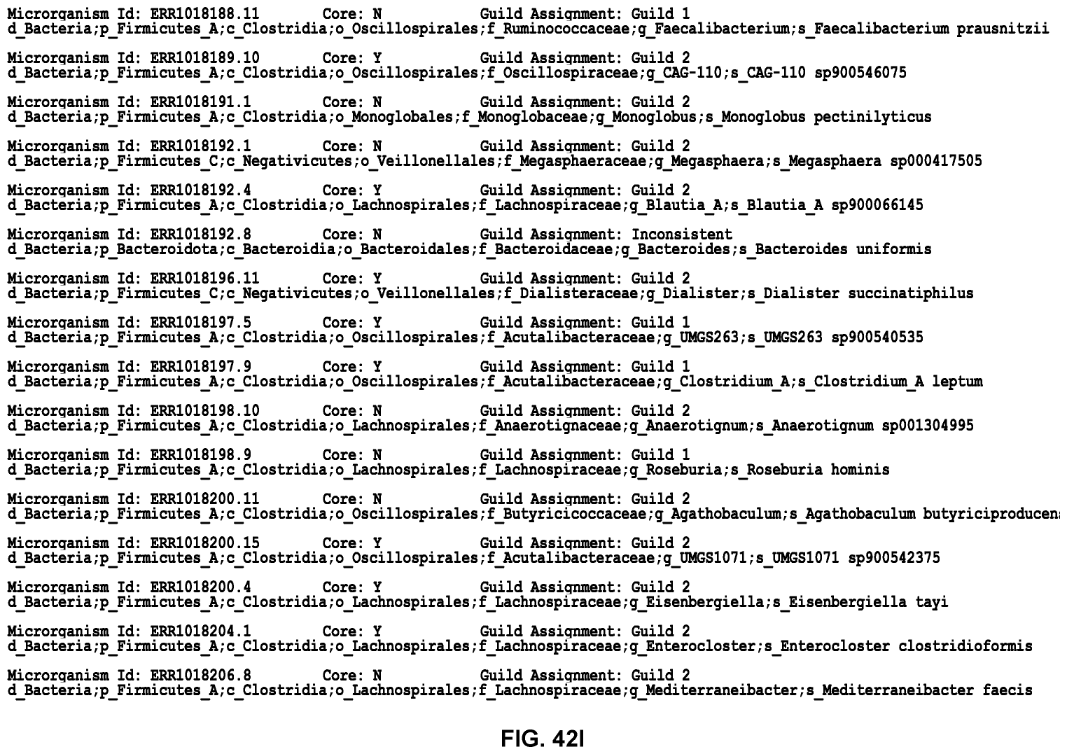

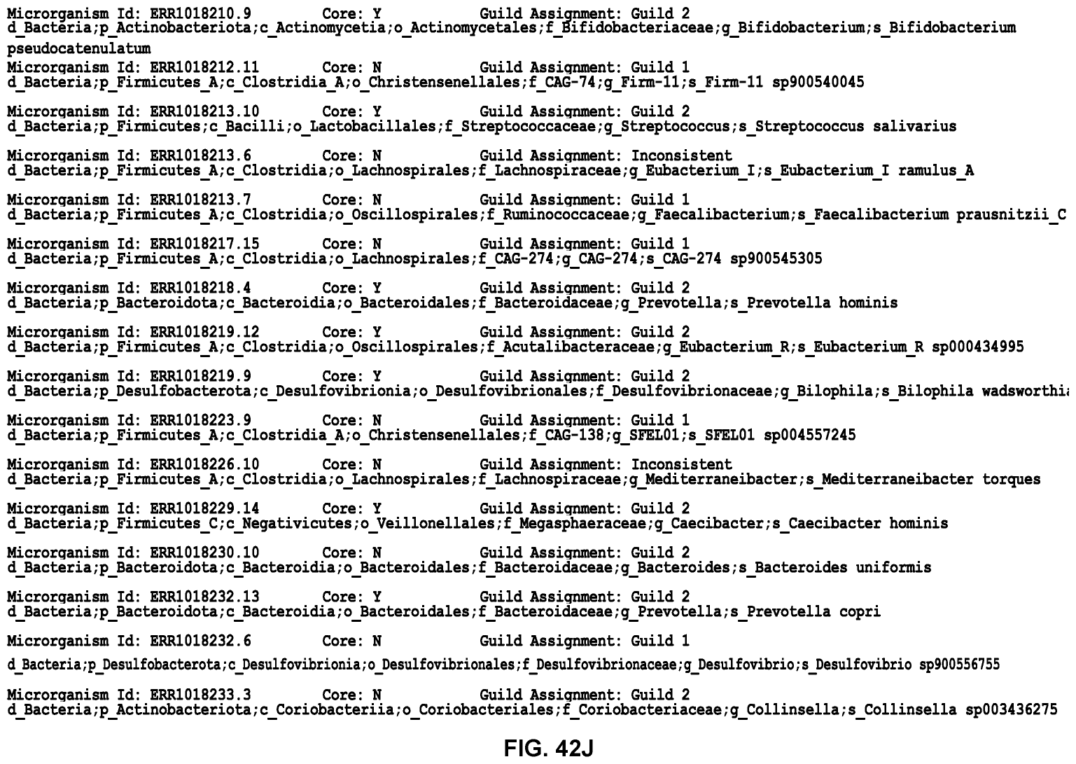

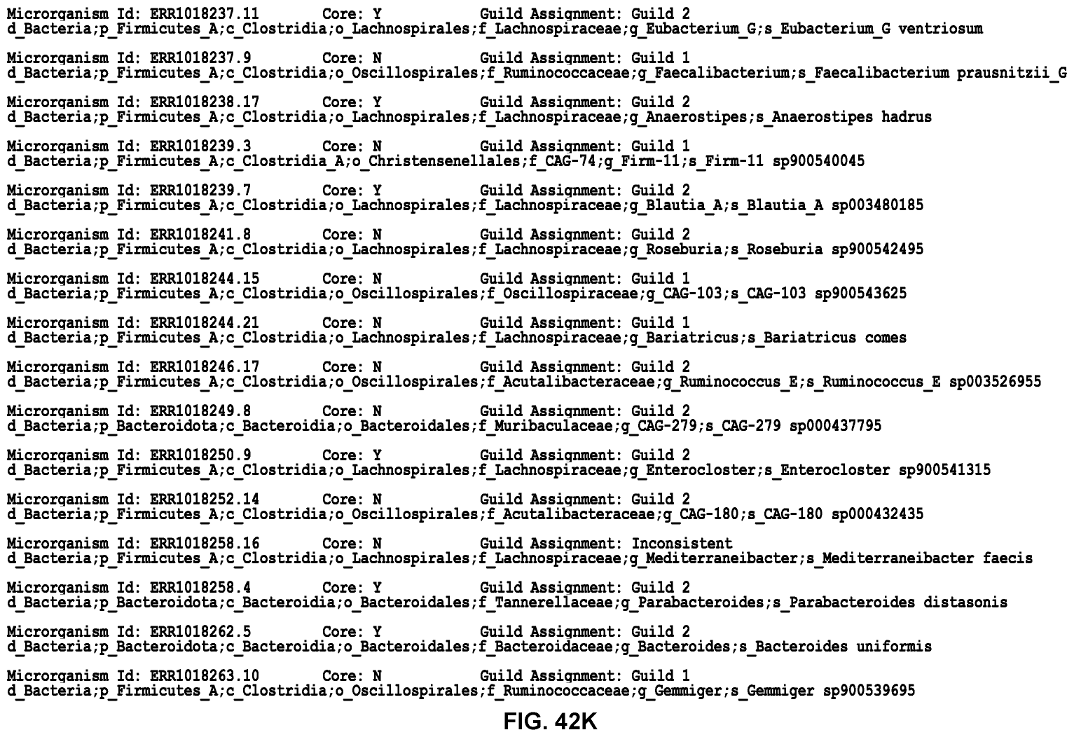

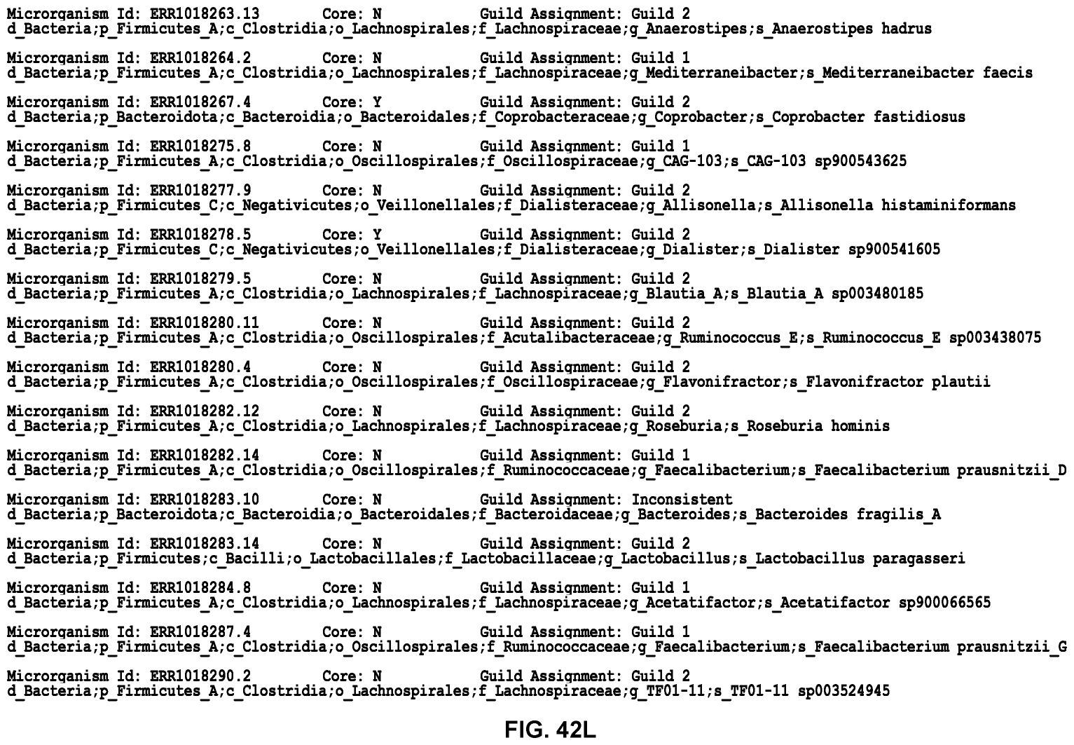

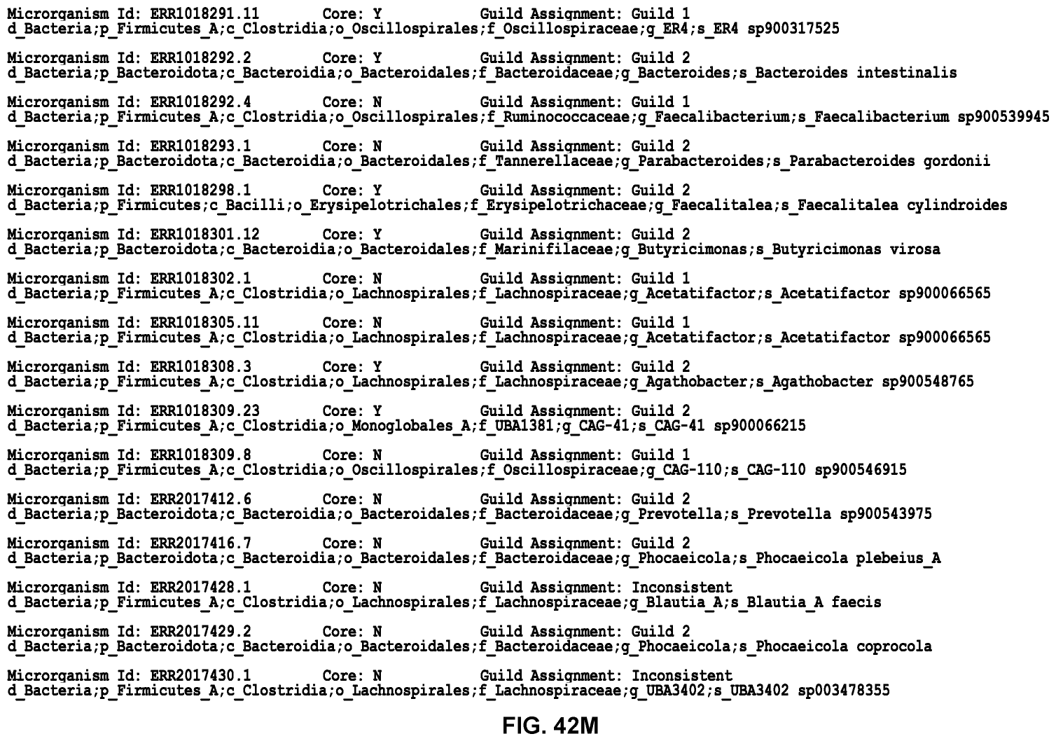

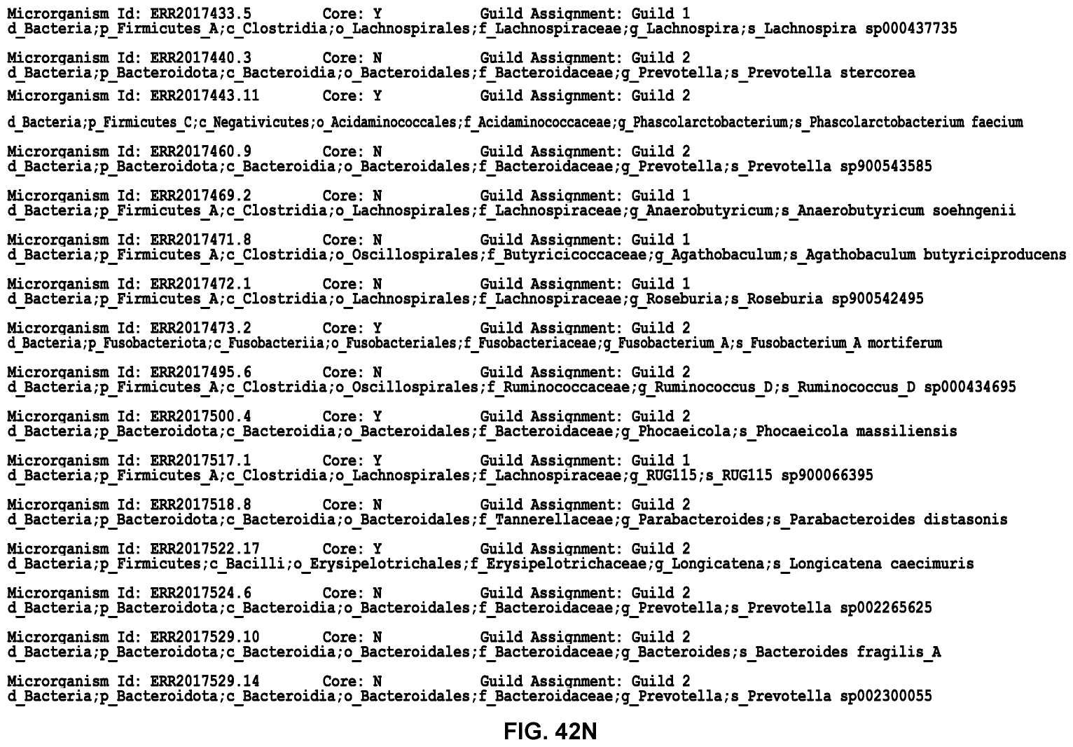

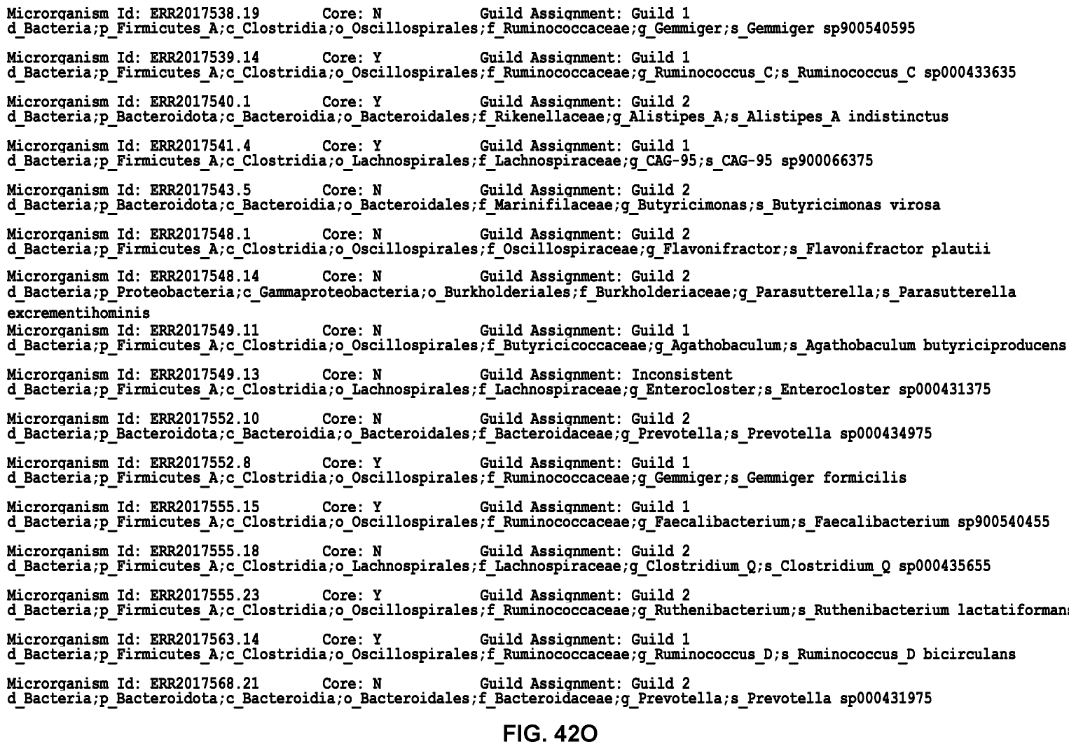

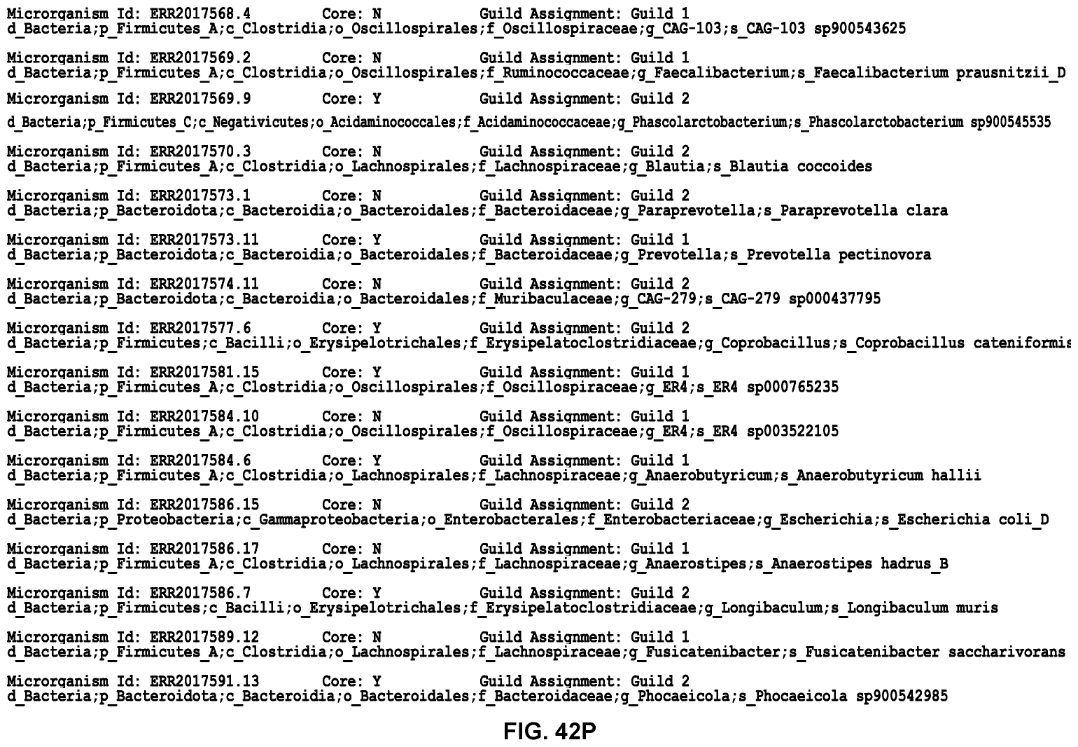

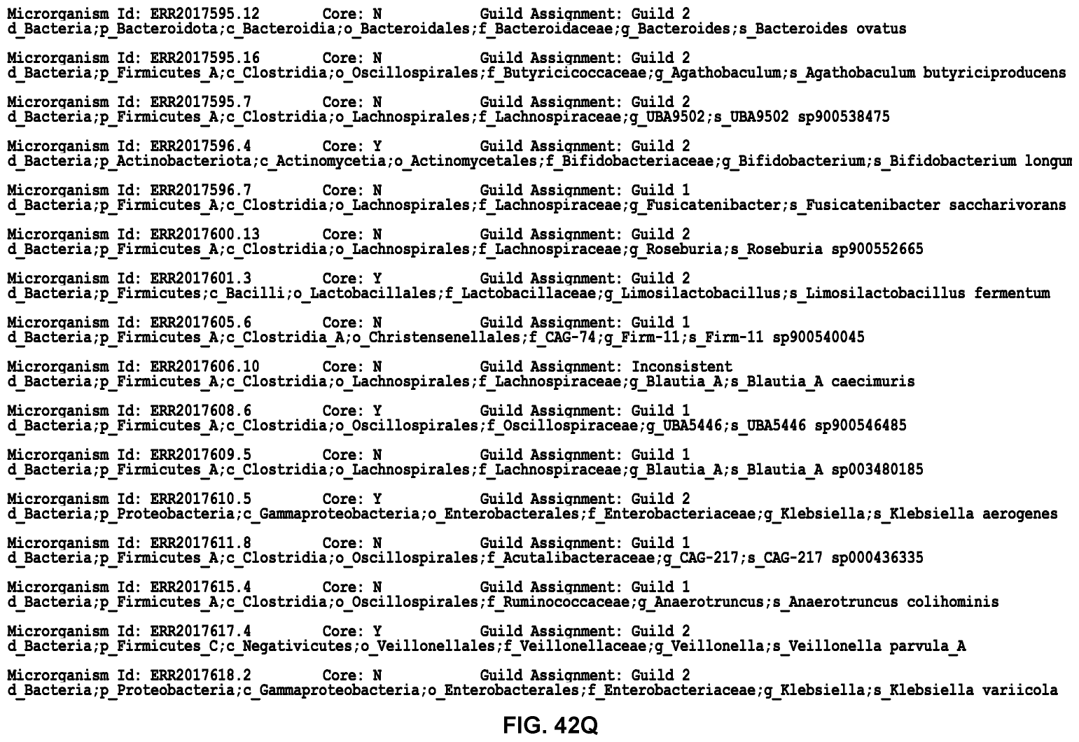

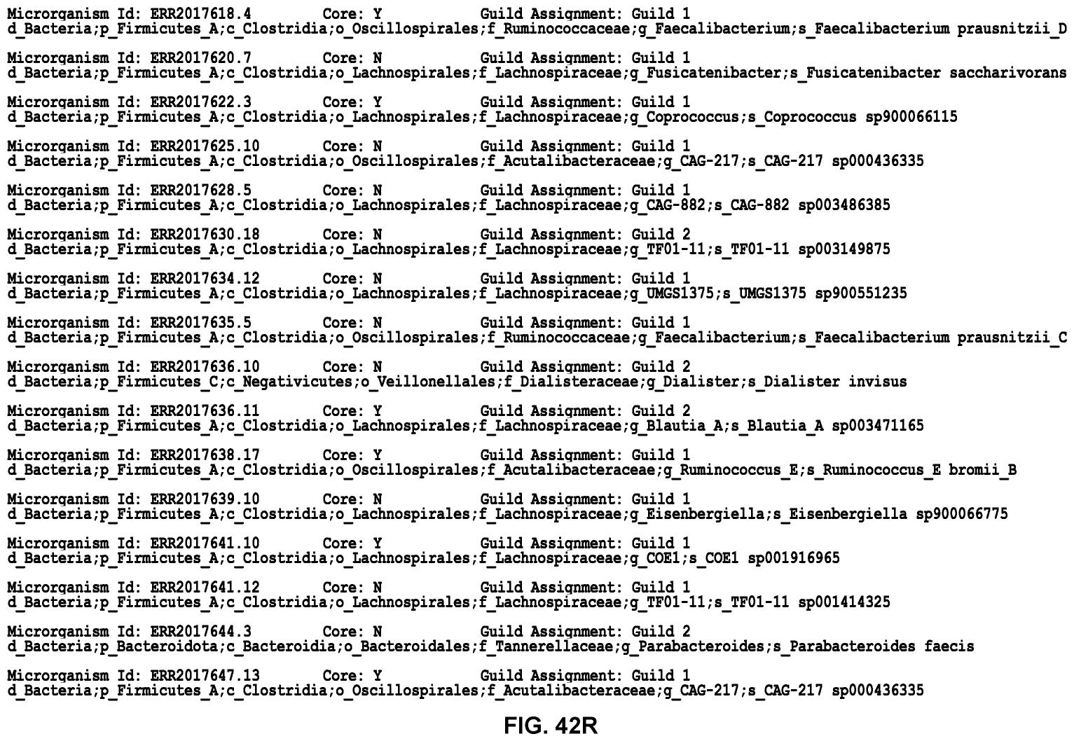

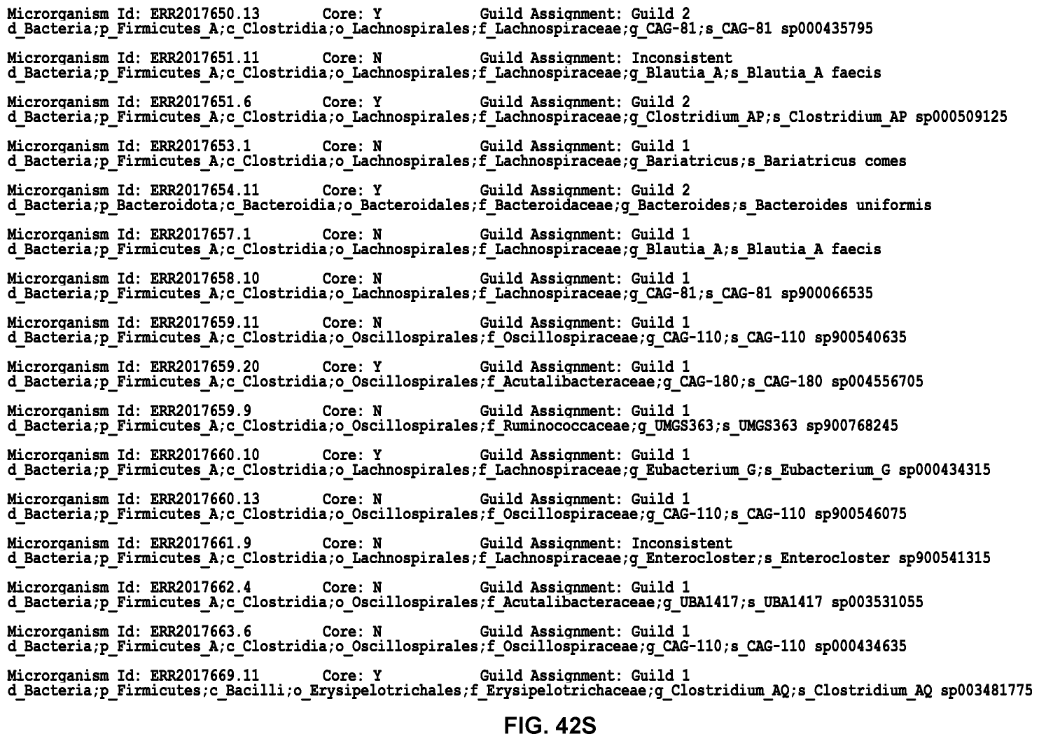

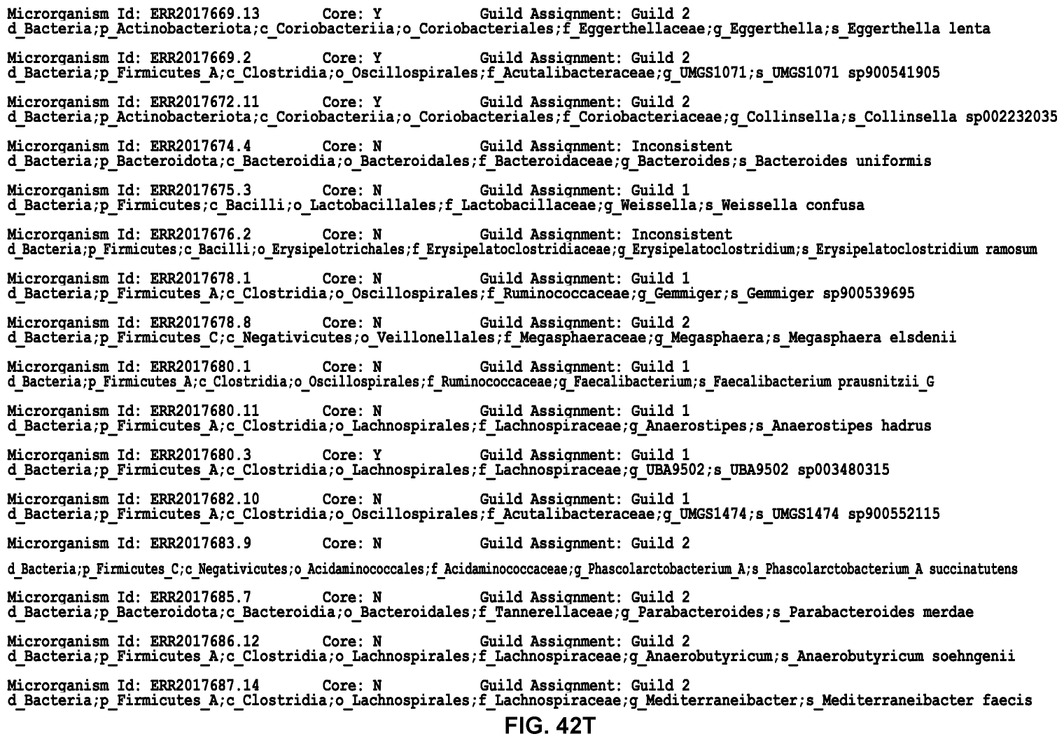

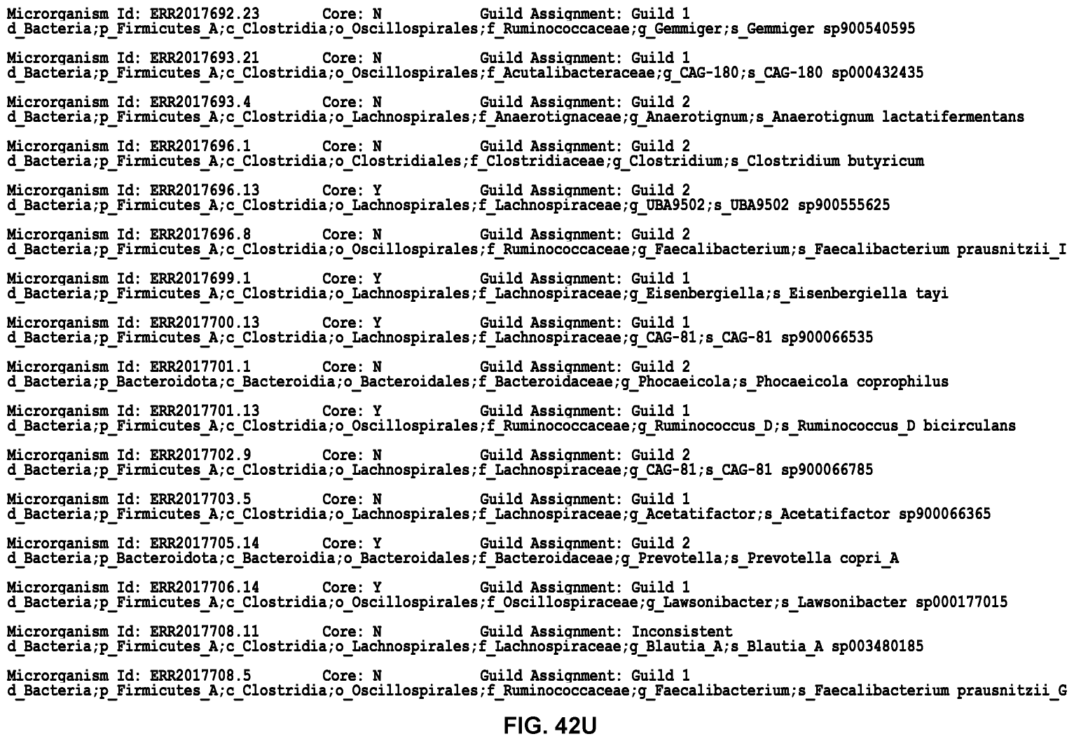

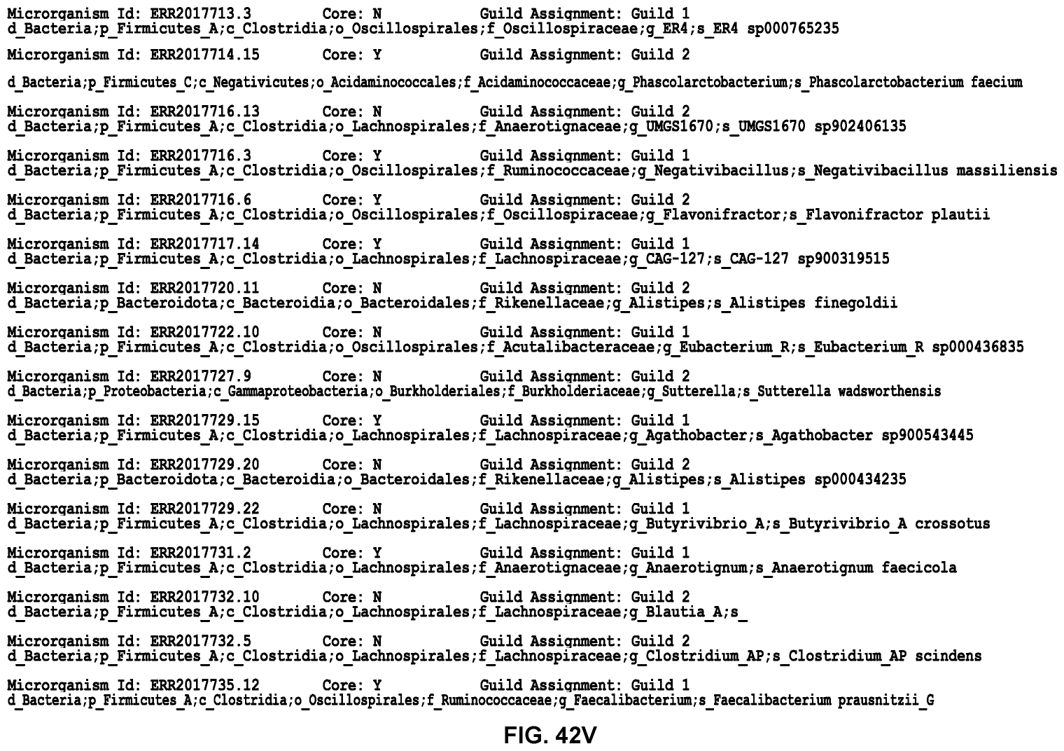

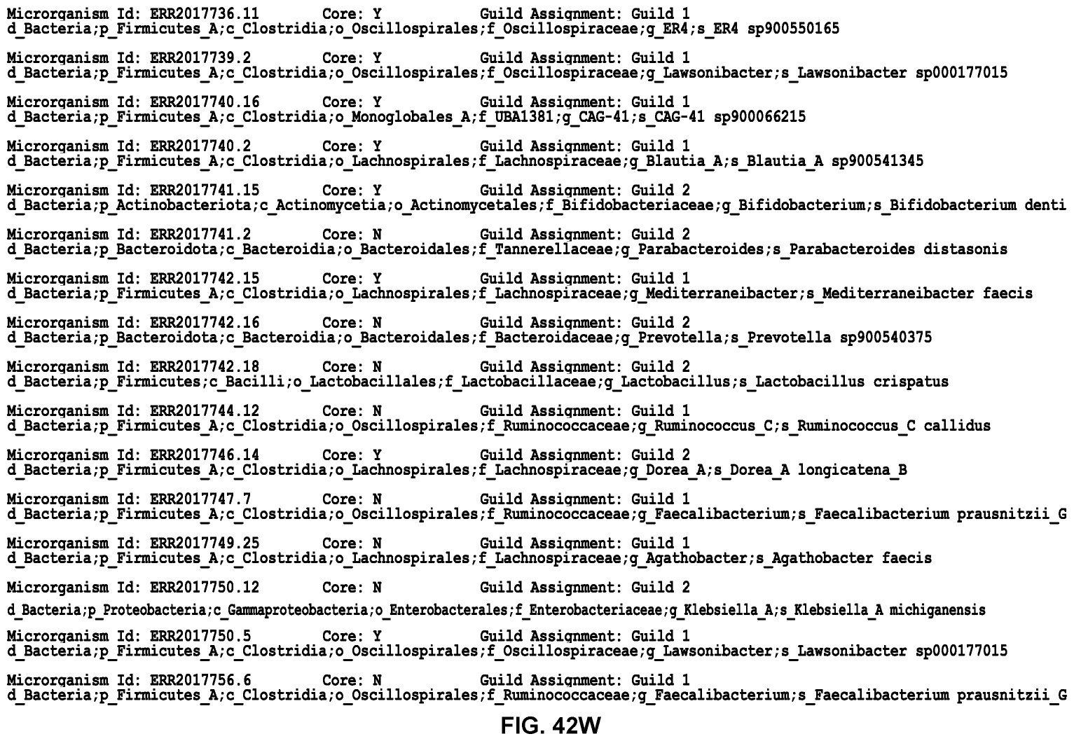

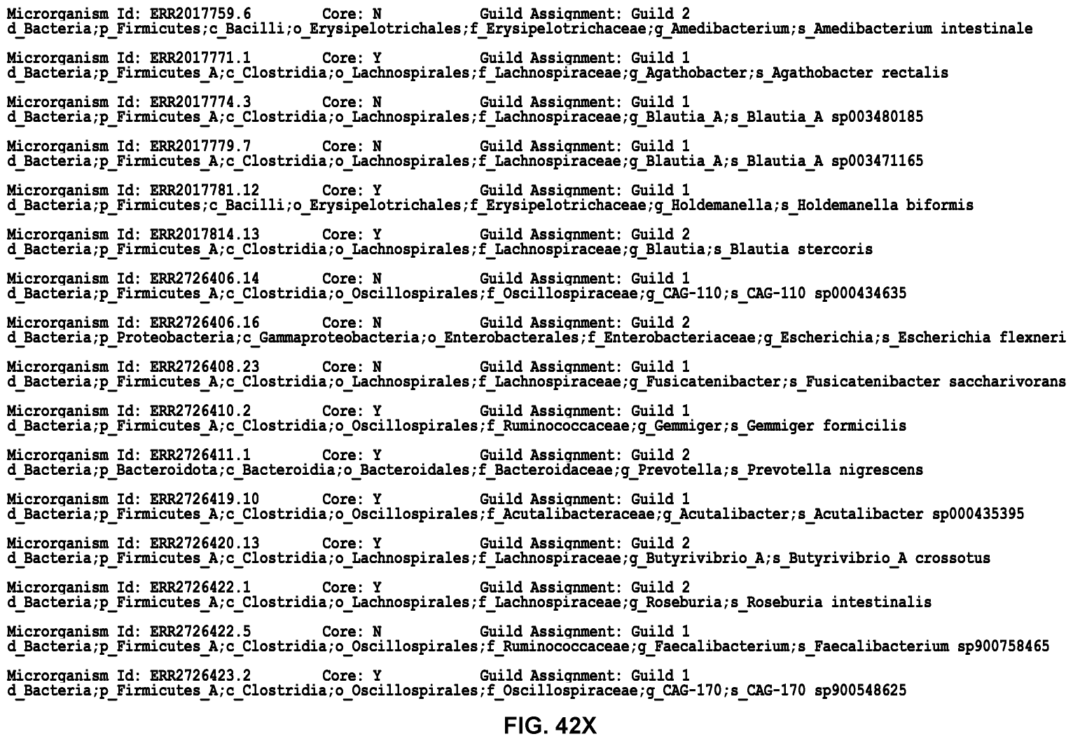

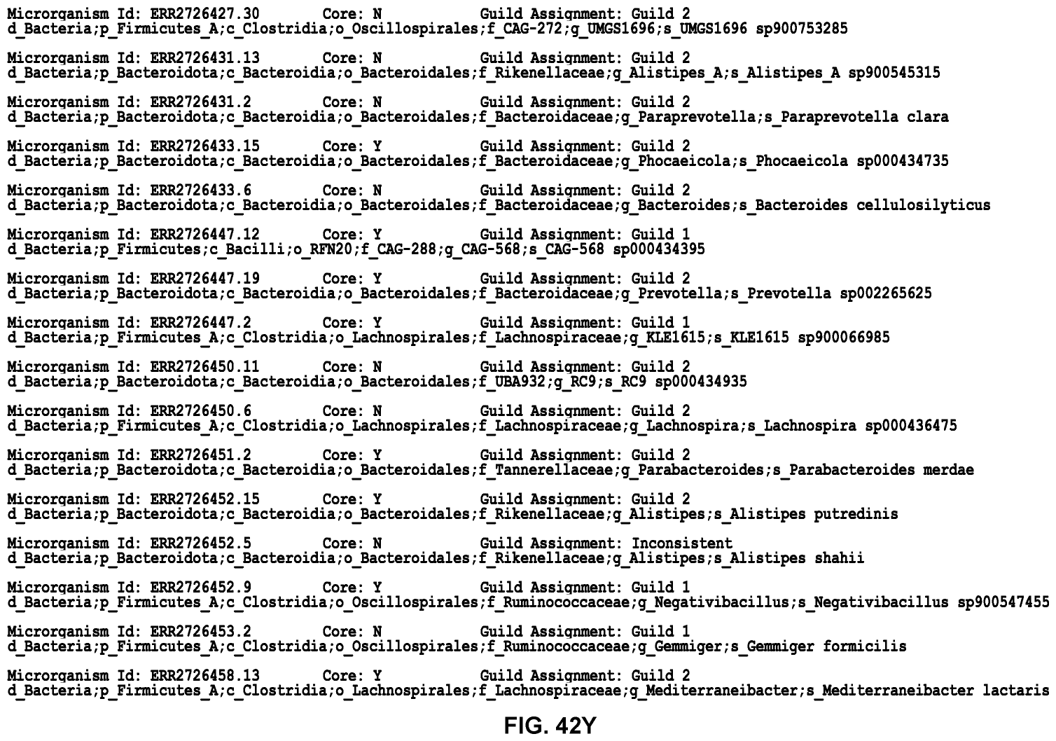

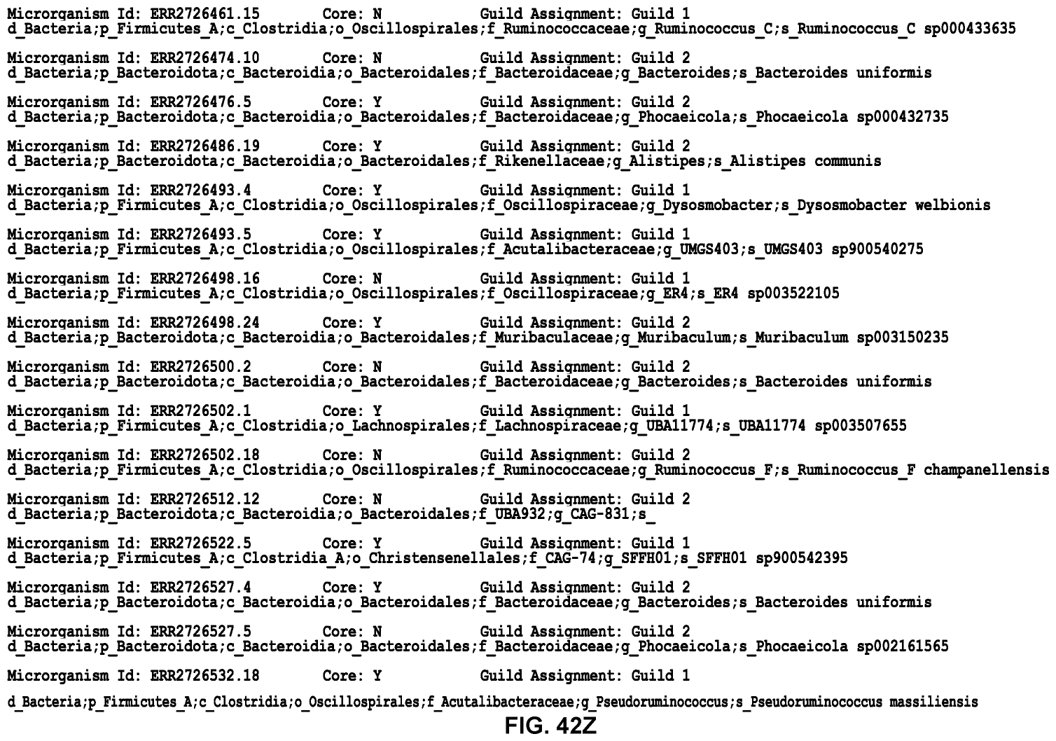

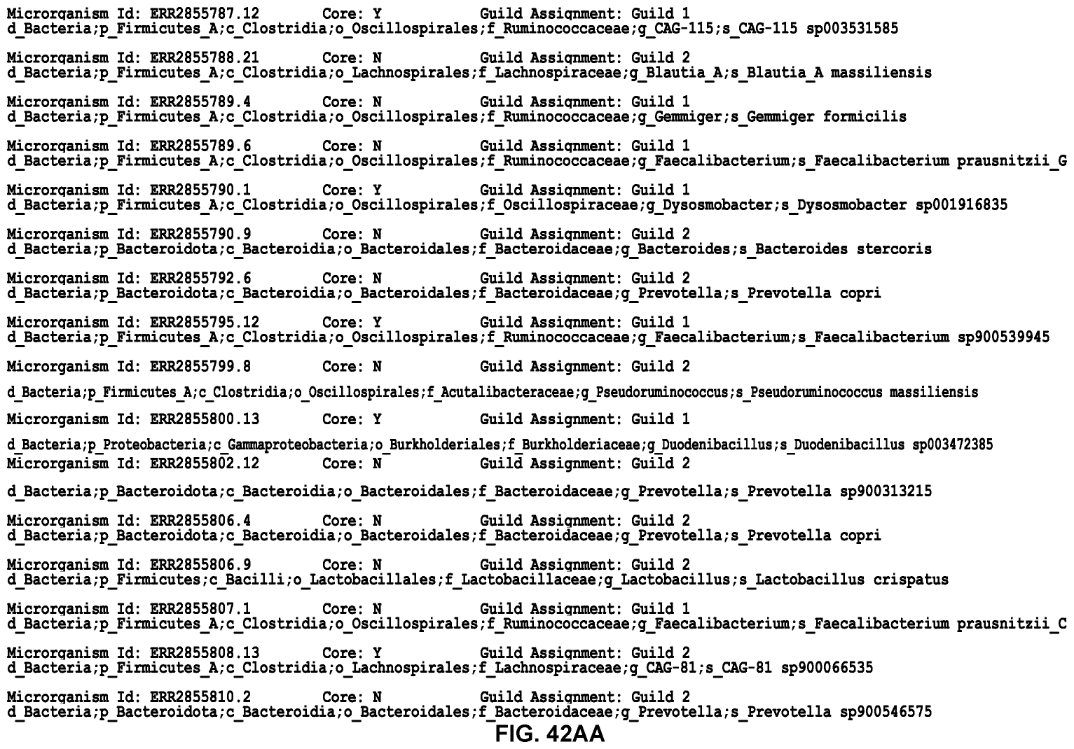

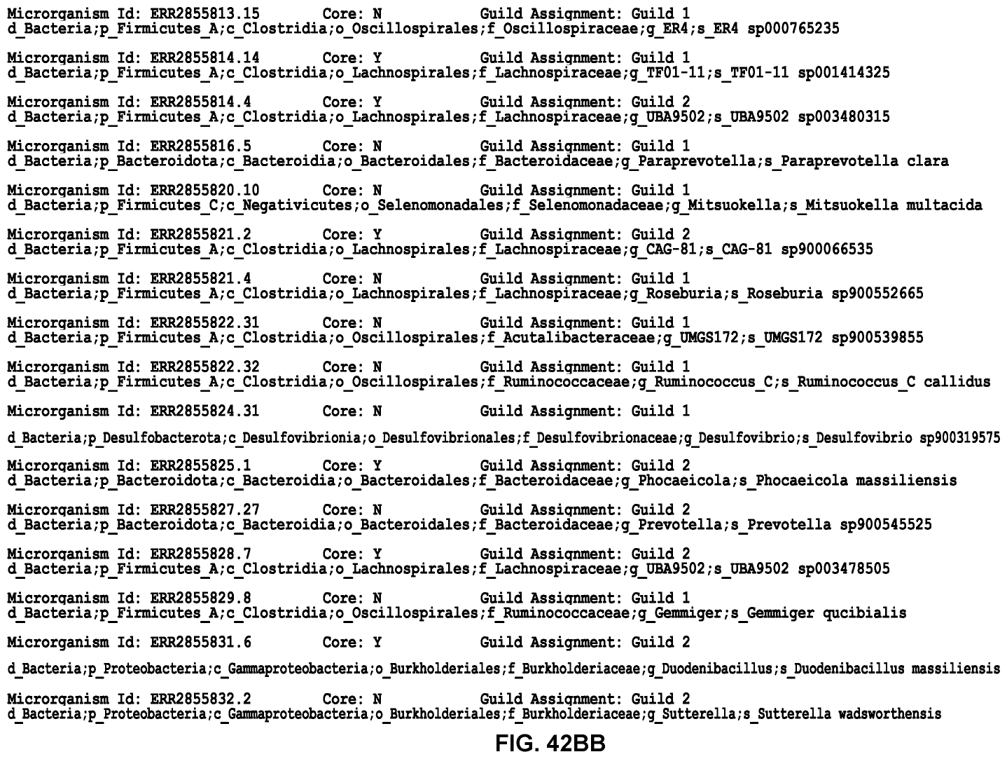

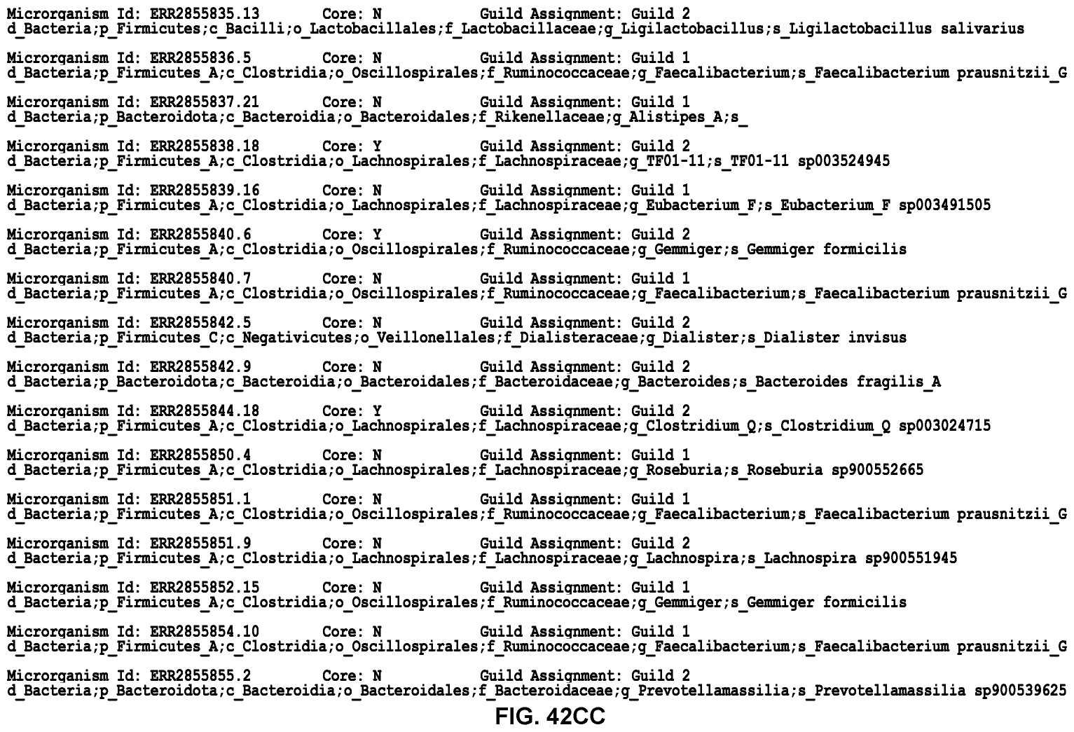

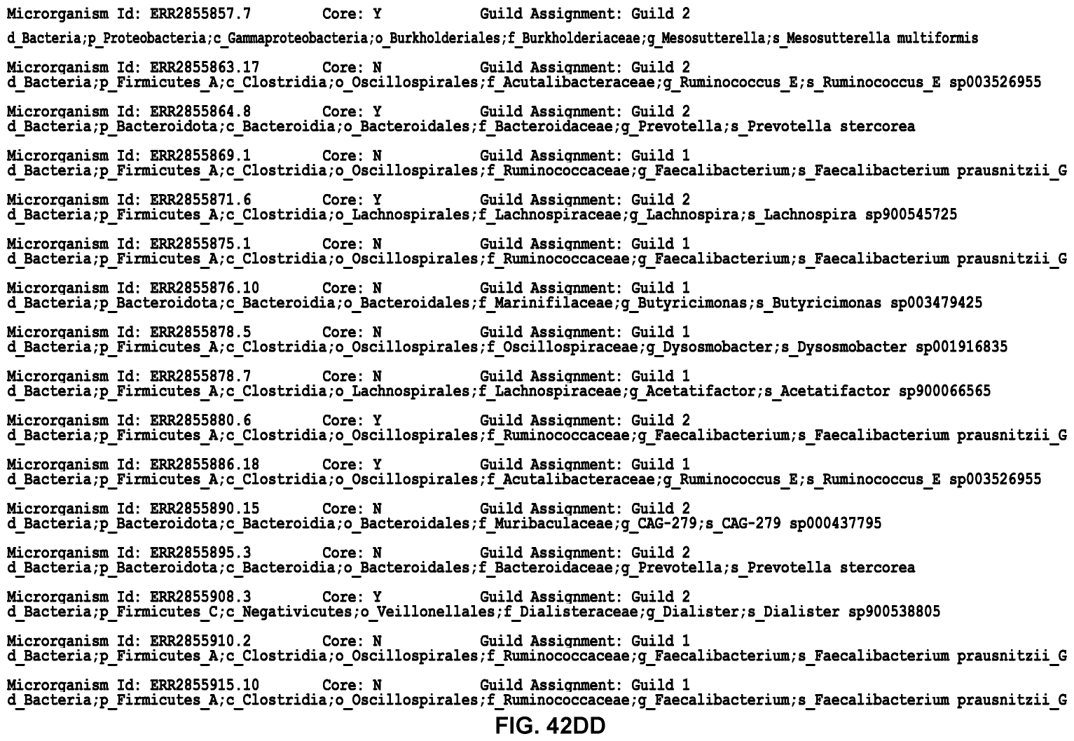

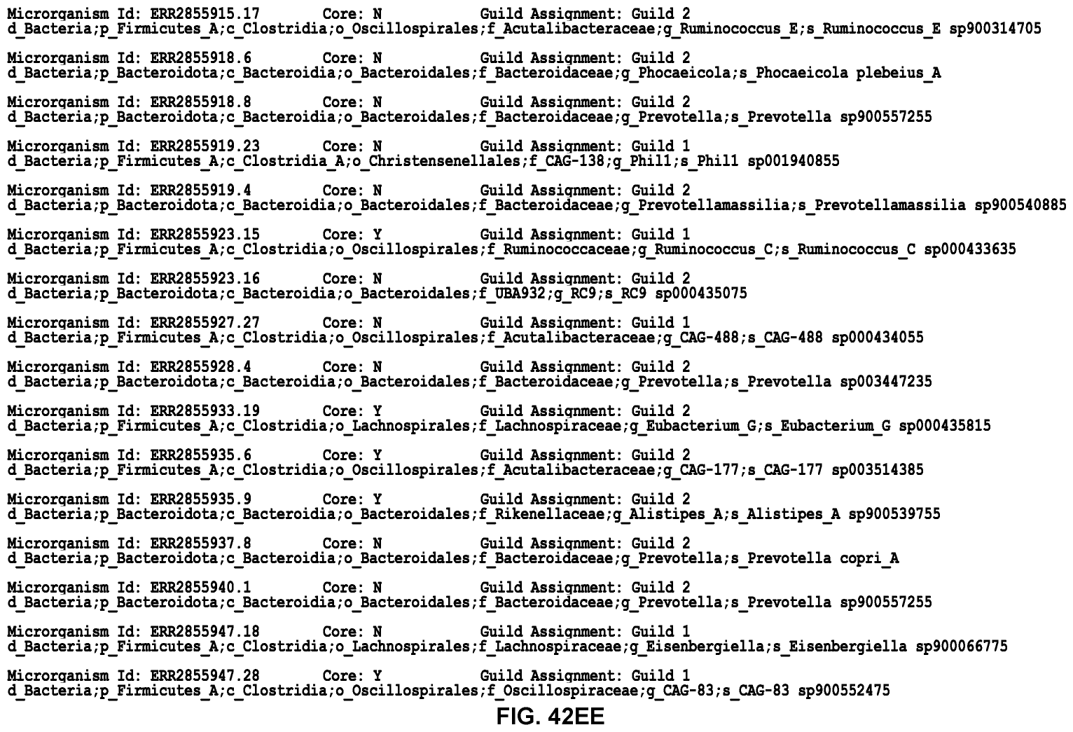

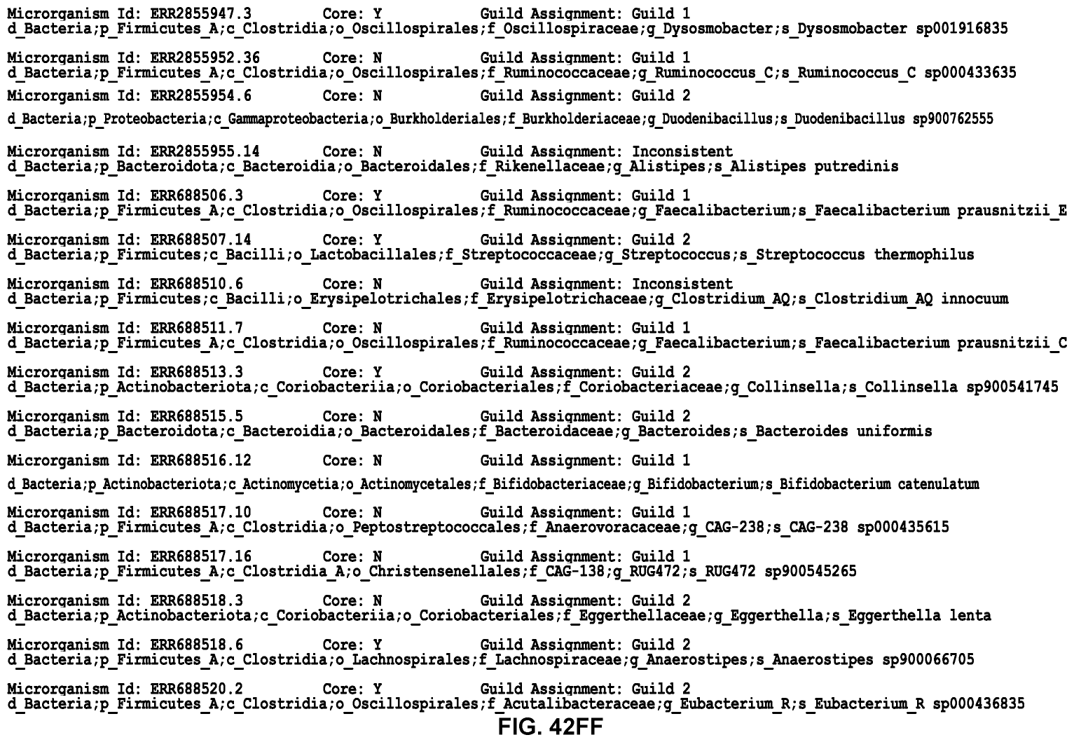

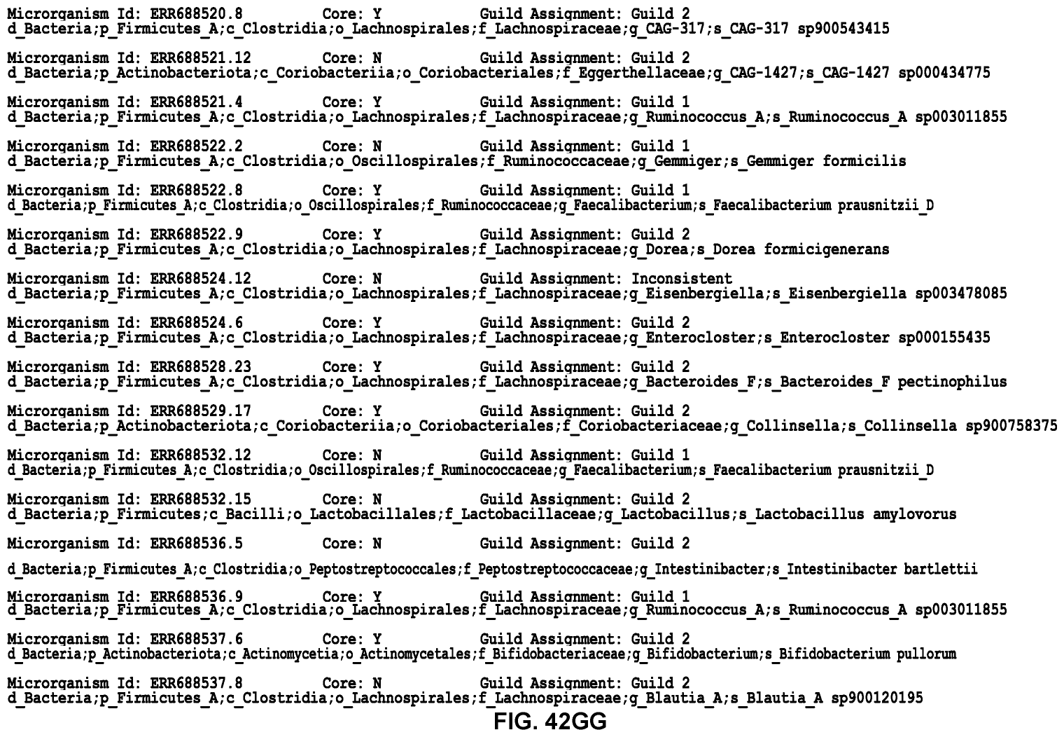

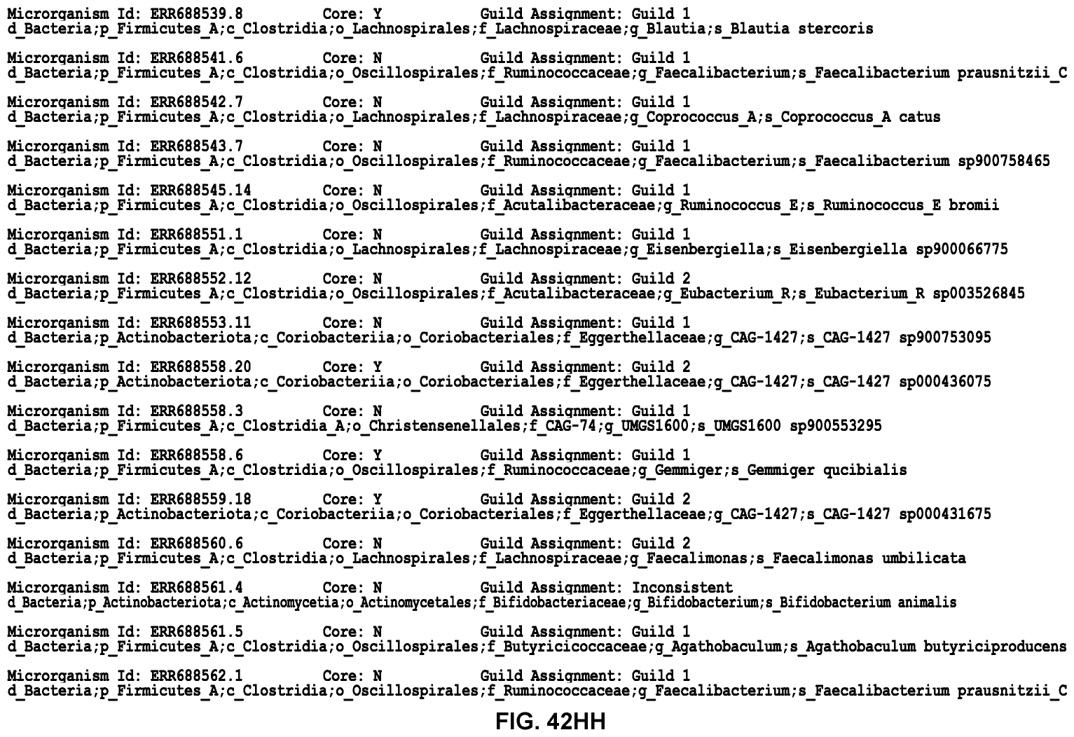

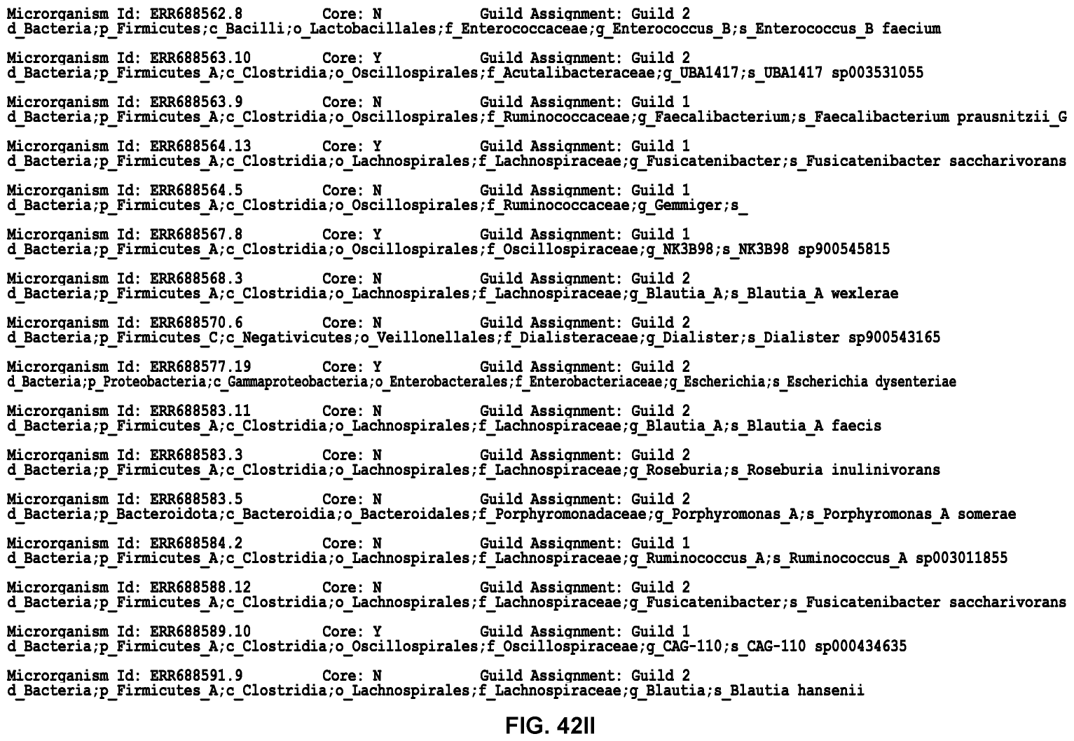

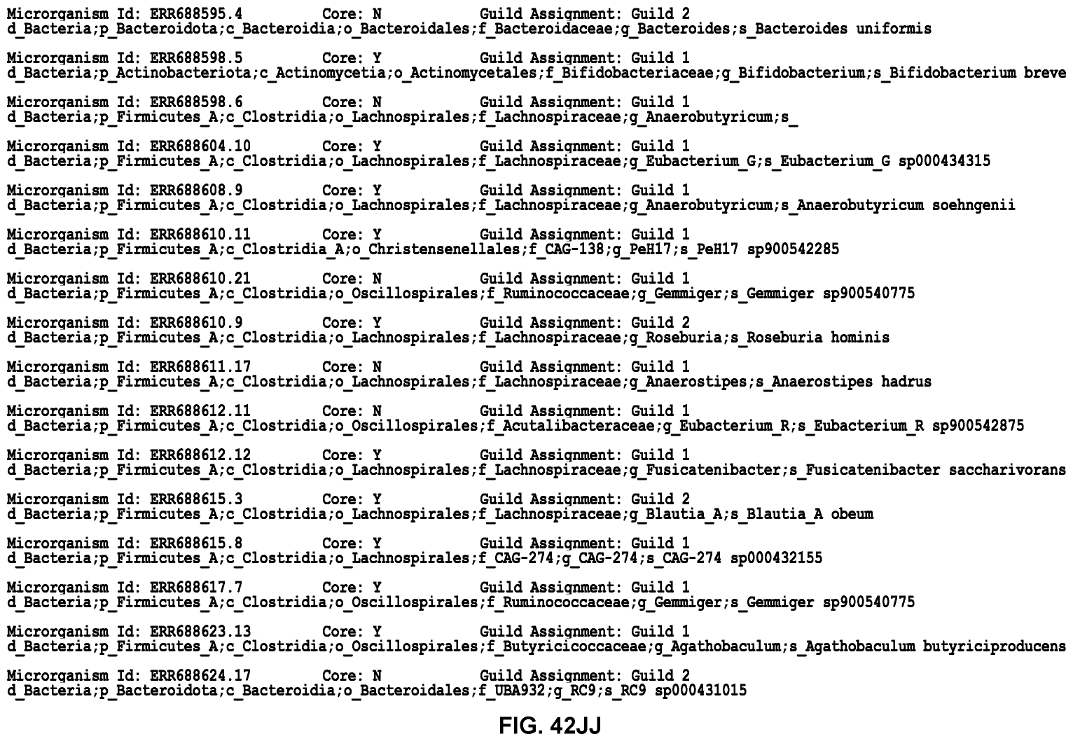

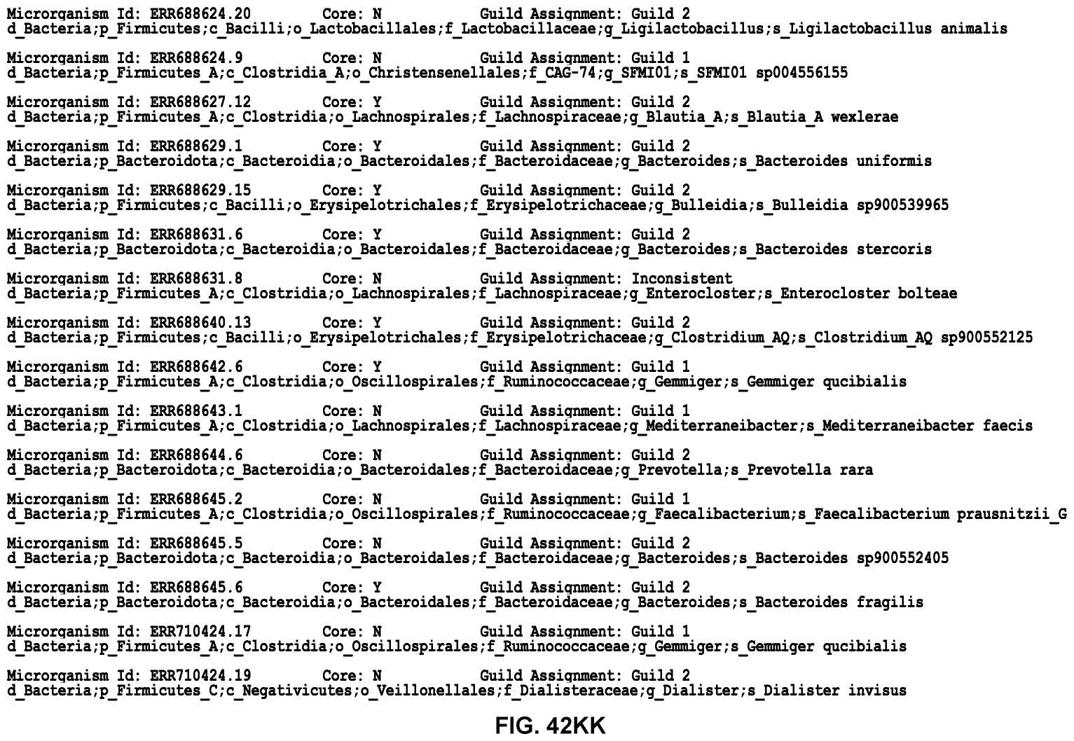

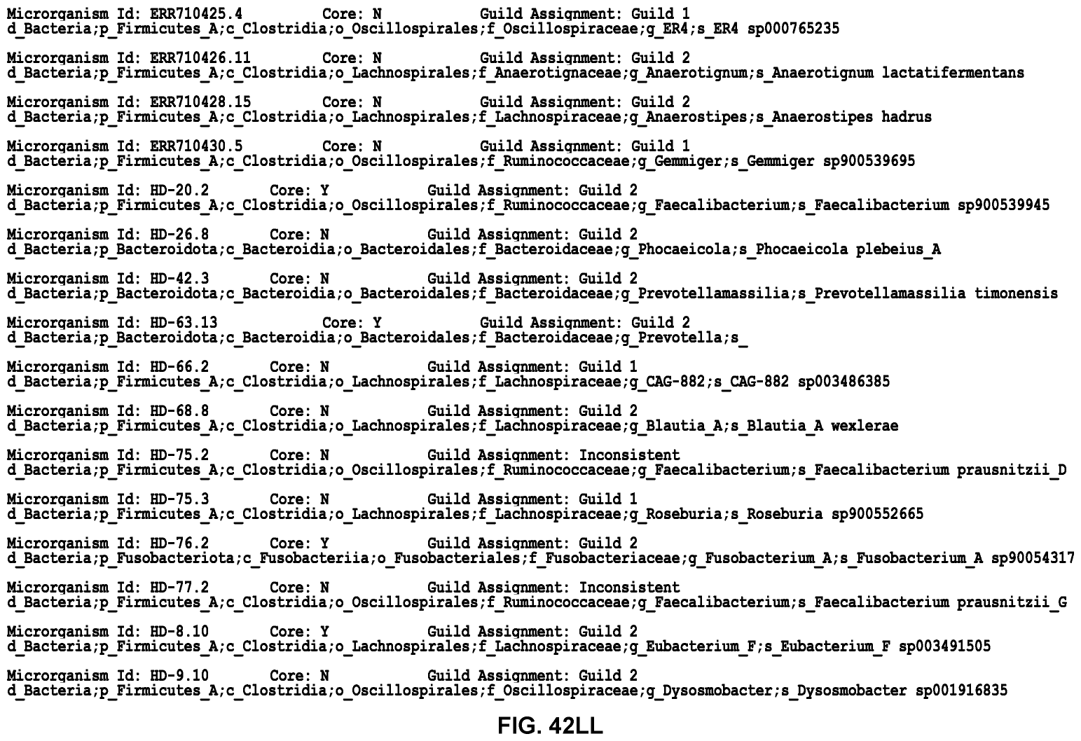

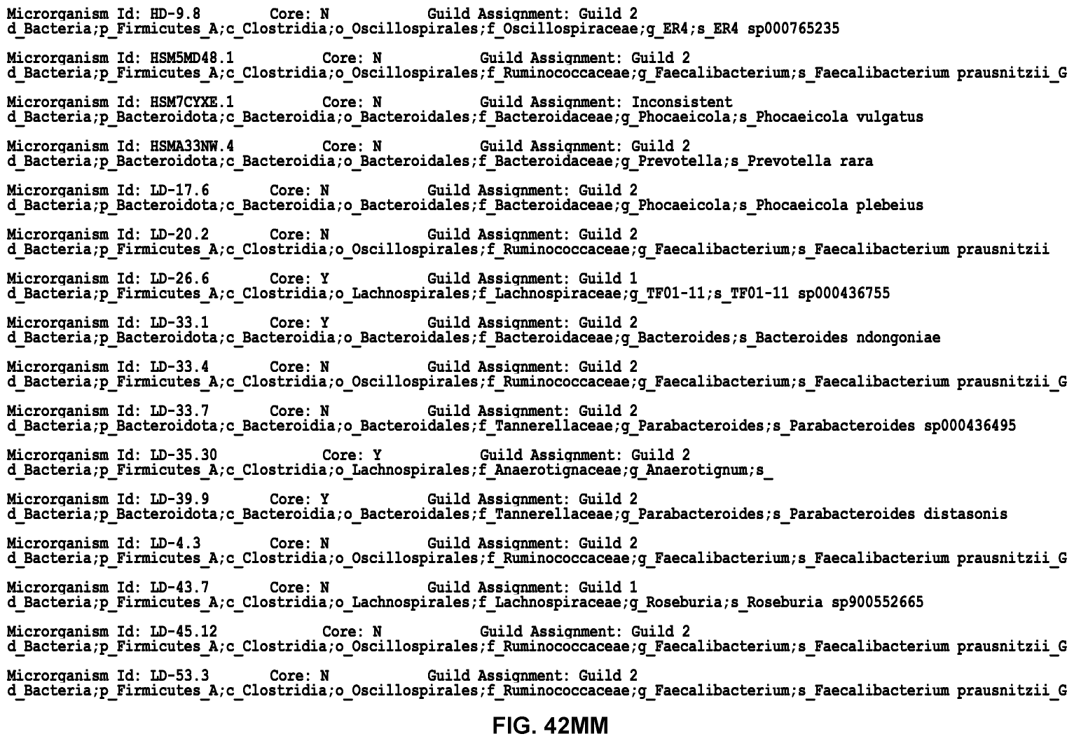

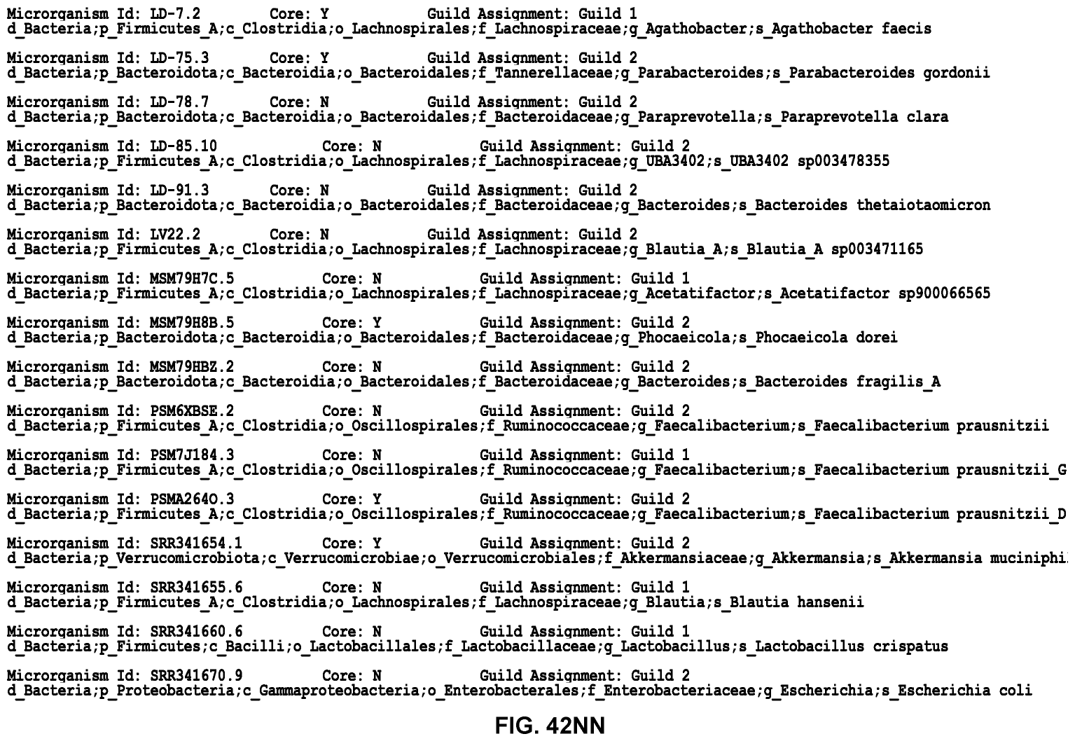

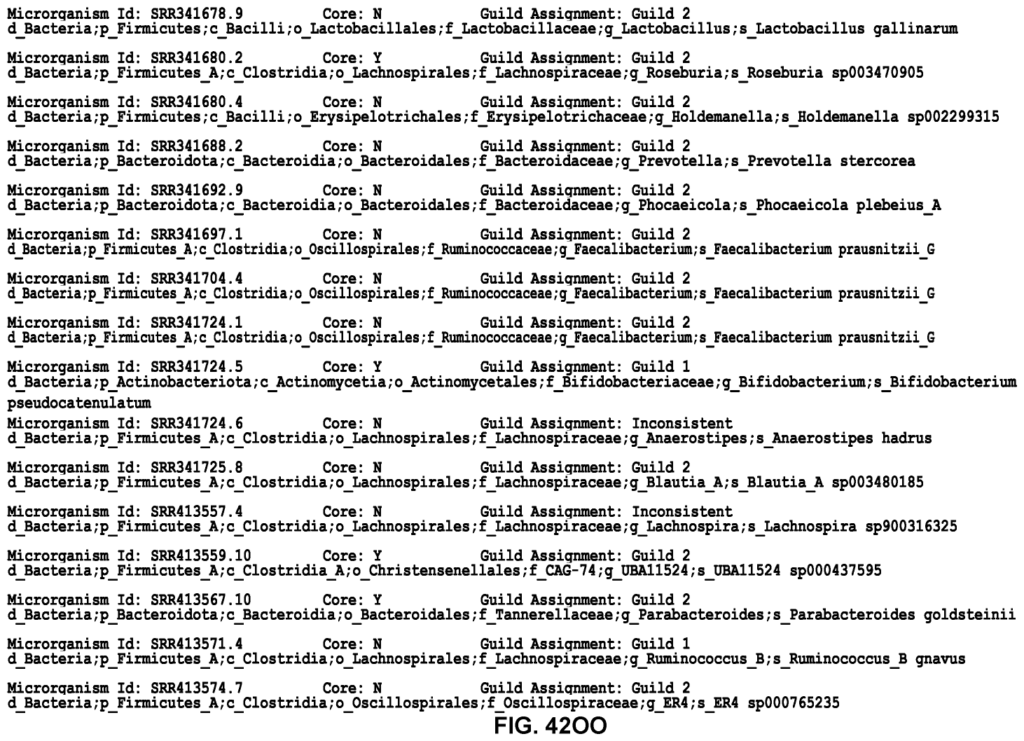

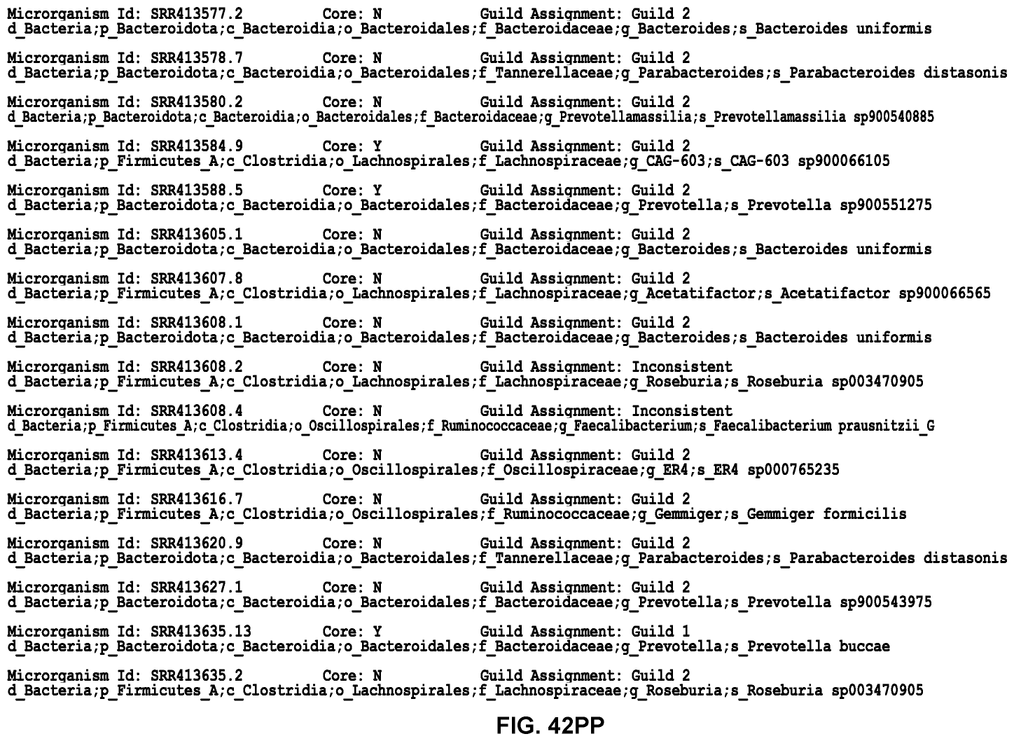

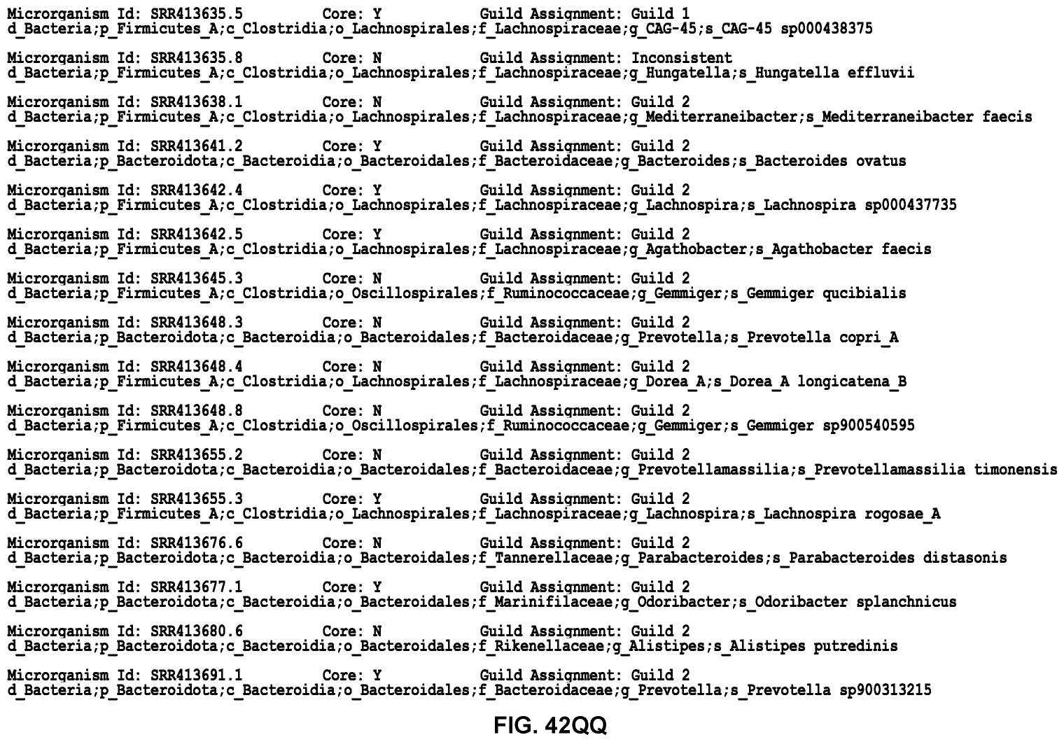

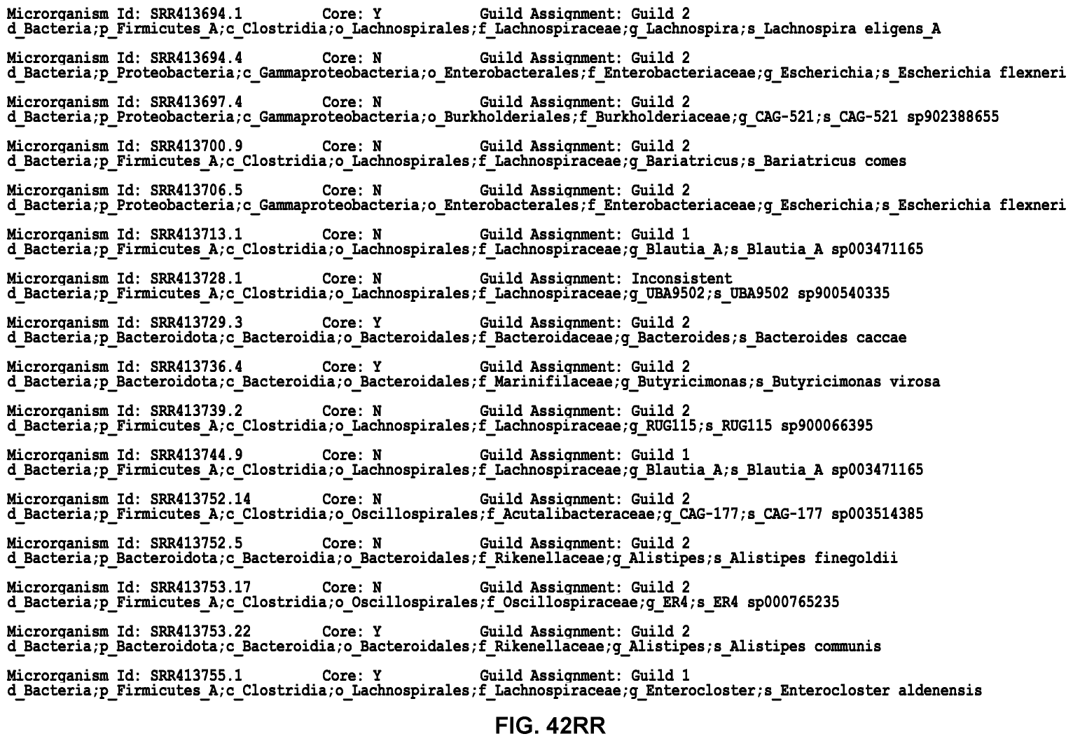

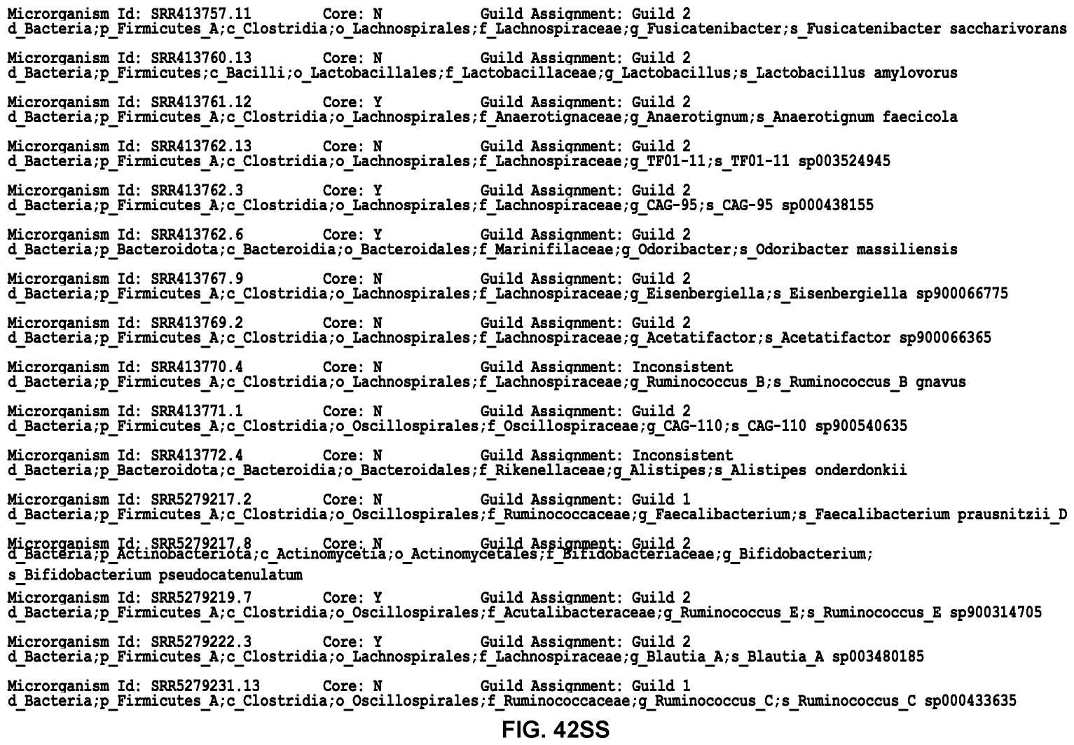

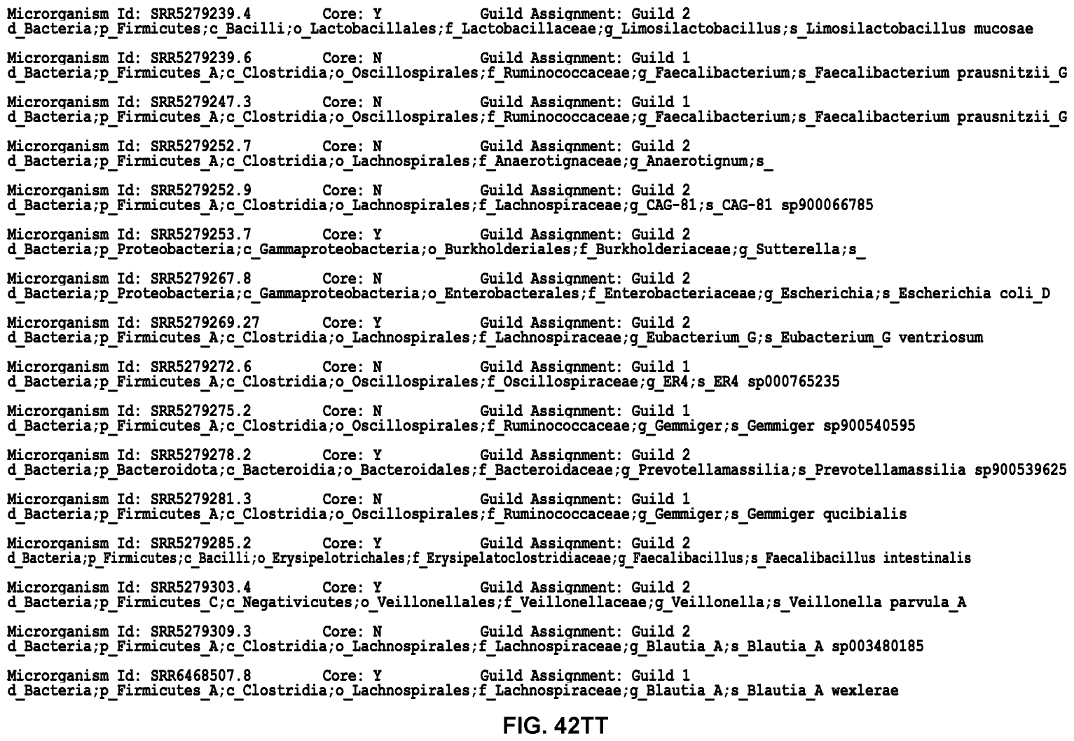

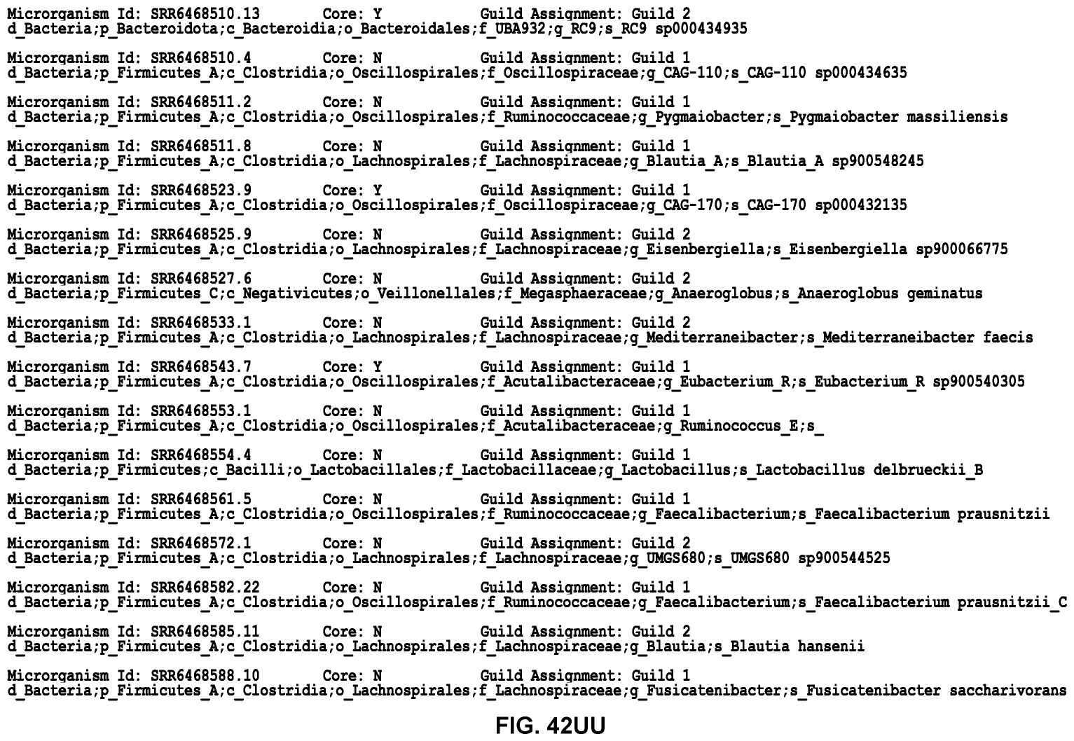

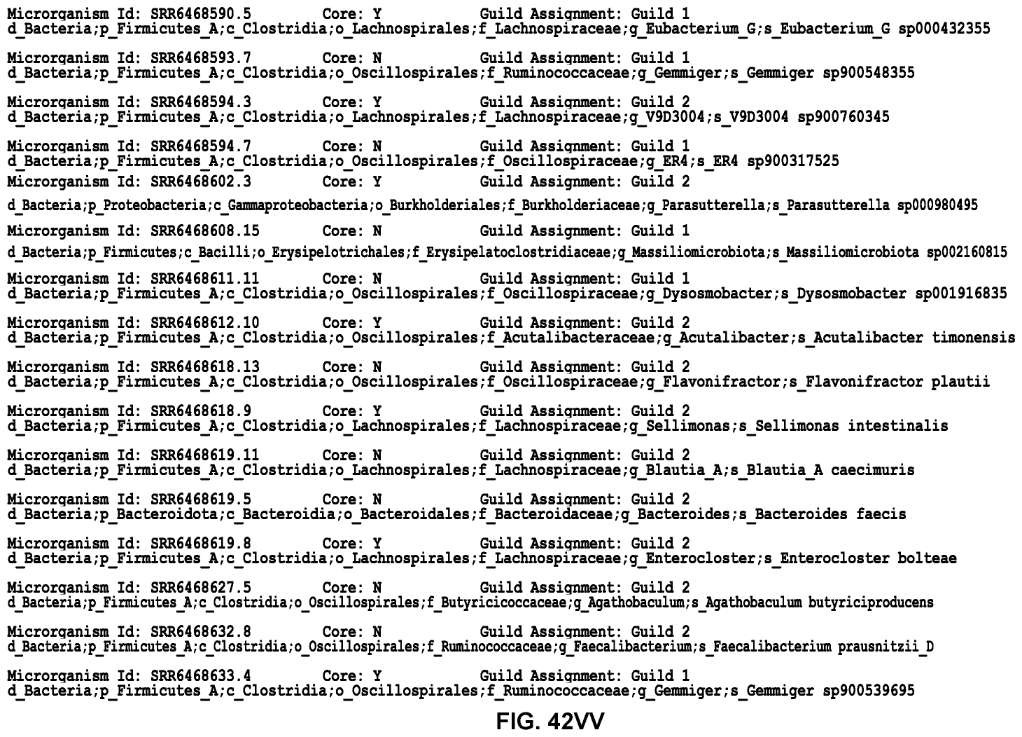

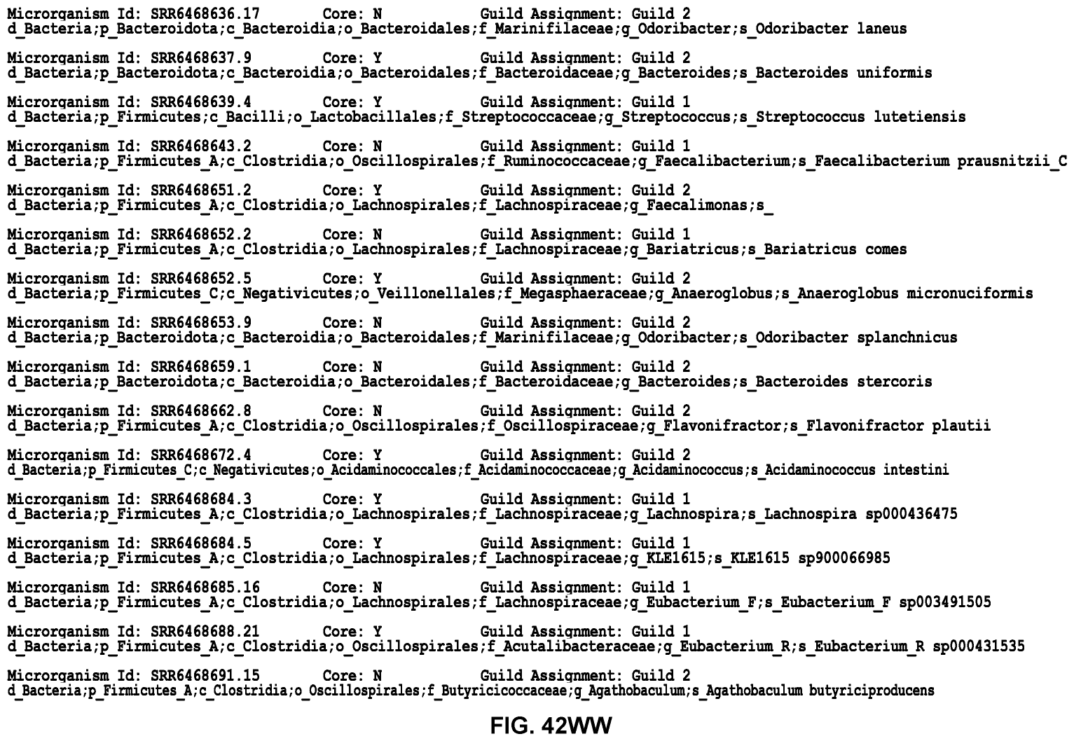

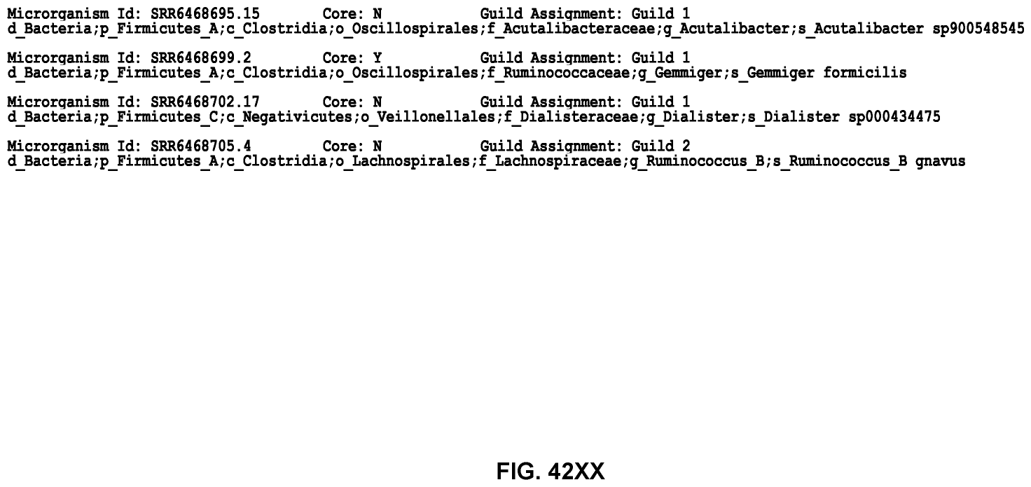

In some embodiments, the first plurality of (e.g., at least 100,000) nucleic acid sequences are obtained through targeted panel sequencing, e.g., as described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, e.g., each of a plurality of the microorganisms listed in Table 1, Table 2, and/or FIGS. 42A-42XX, prior to sequencing recovered nucleic acids. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample comprises a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLiD sequencing systems from Life TechnologiesT™, the 454 GS FLX+ and GS Junior sequencing systems from Roche, the MinION™ system from Oxford Nanopore, or any other sequencing platform of interest.

In some embodiments, the plurality of genomic abundance values is determined using a microarray comprising a probe sequence capable of detecting a unique genomic sequence of each respective genome for the plurality of gut microorganisms. In some embodiments, the panel of probes on a microarray includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

Referring to block 206, in some embodiments, the method includes obtaining, in electronic form, for each respective subject in the first plurality of subjects, a corresponding first plurality of nucleic acid sequences (e.g., at least 100,000 nucleic acid sequences) for genomic DNA from a corresponding biological sample from the gut of the respective subject, and determining, for each respective subject in the first plurality of subjects, the corresponding genomic abundance value for each respective gut microorganism in the plurality of gut microorganisms from the corresponding first plurality of (e.g., at least 100,000) nucleic acid sequences. In some embodiments, the genomic abundance values determined for each respective subject in the first plurality of subjects comprise at least 20, at least 25, at least 50, at least 100, at least 250, at least 500, at least 1,000, at least 5,000 or at least 10,000 genome abundance values. In some embodiments, the genomic abundance values determined for each respective subject in the first plurality of subjects comprise no more than 250,000, no more than 100,000, no more than 50,000, no more than 25,000, no more than 10,000, no more than 5,000, no more than 1,000, no more than 100, no more than 50, no more than 30, or no more than 20 genome abundance values. In some embodiments, the genomic abundance values determined for each respective subject in the first plurality of subjects consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the genomic abundance values determined for each respective subject in the first plurality of subjects fall within another range starting no lower than 10 genome abundance values and ending no higher than 250,000 genome abundance values.

Referring to block 208, in some embodiments, the method includes, for each respective subject in the first plurality of subjects, assembling a corresponding first plurality of gut microorganism genomes by metagenomic de novo sequence assembly from the corresponding first plurality of (e.g., at least 100,000) nucleic acid sequences, and calculating, for each respective gut microorganism genome in the corresponding first plurality of gut microorganism genomes, a corresponding genomic abundance of the respective gut microorganism genome. In some embodiments, metagenomic de novo sequence assembly further comprise generating contigs based on the sequencing reads generated by a shotgun sequencing technique, e.g., as described in U.S. Pat. No. 10,529,443, the content of which is incorporated herein by reference in its entirety. In some embodiments, the first plurality of (e.g., at least 100,000) nucleic acid sequences can be assembled into full genomes of the plurality of gut microorganisms. In some embodiments, the first plurality of (e.g., at least 100,000) nucleic acid sequences can be assembled into partial genomes of the plurality of gut microorganisms.

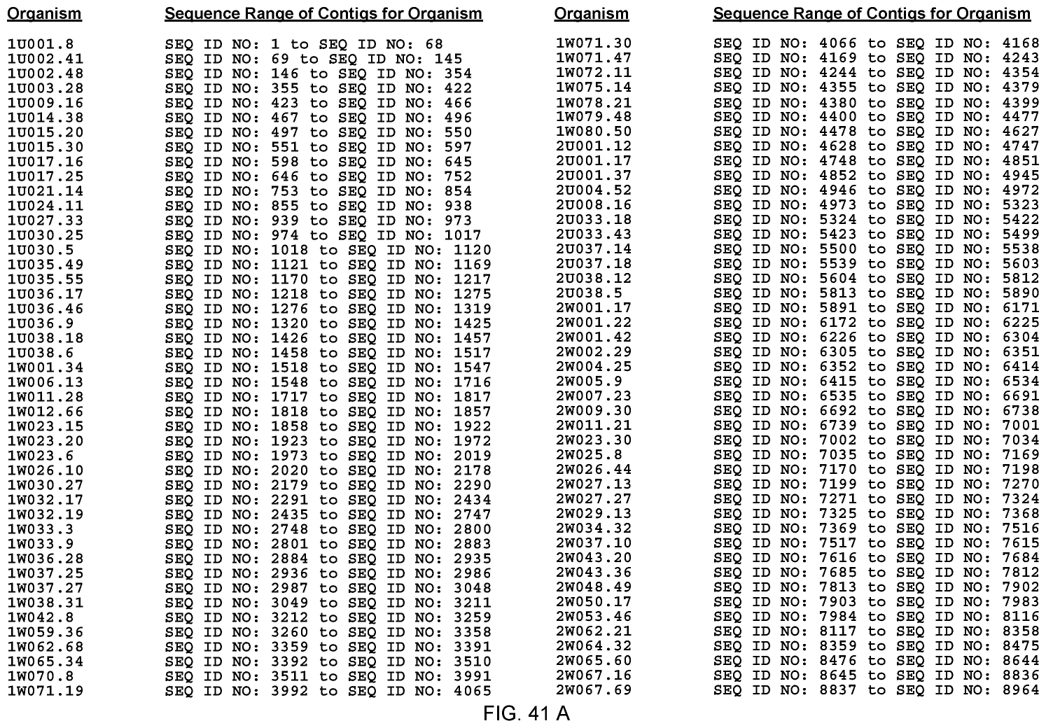

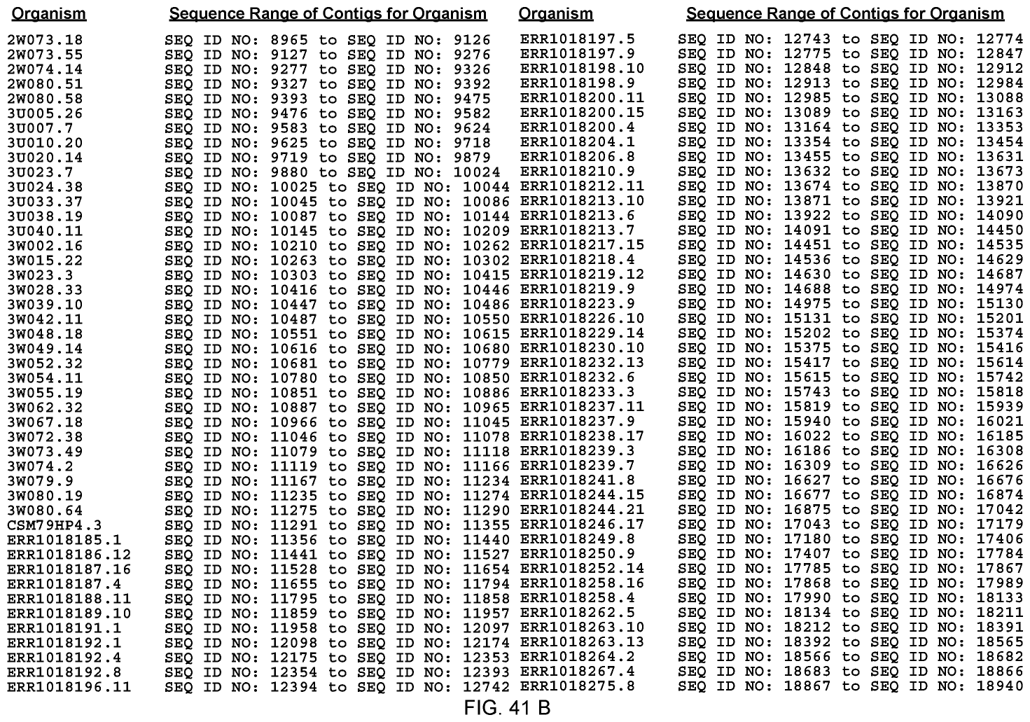

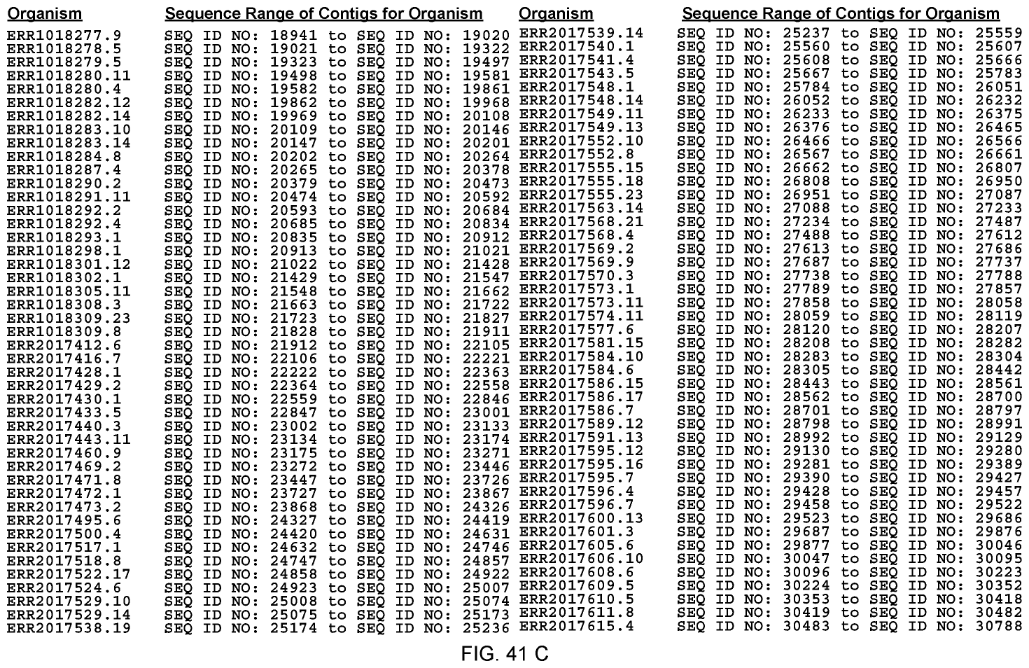

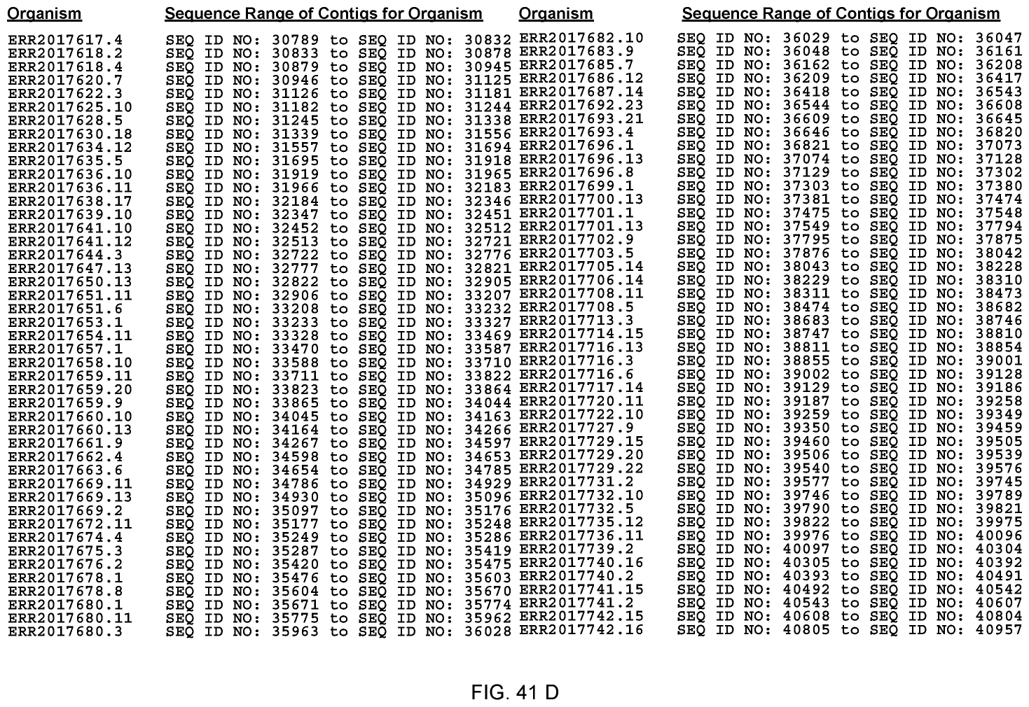

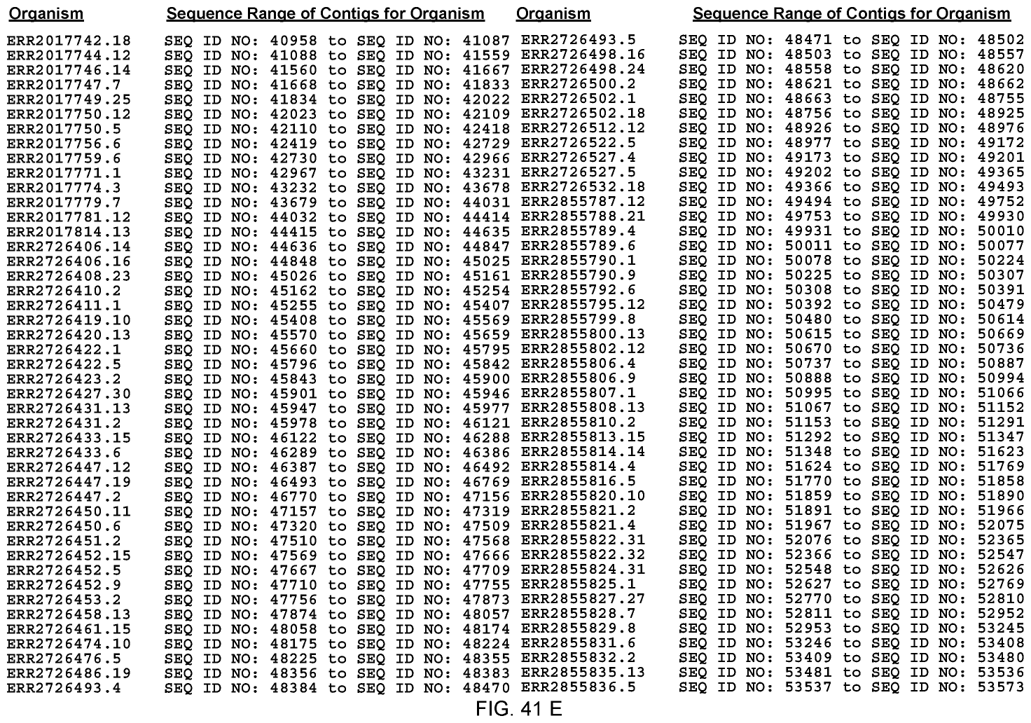

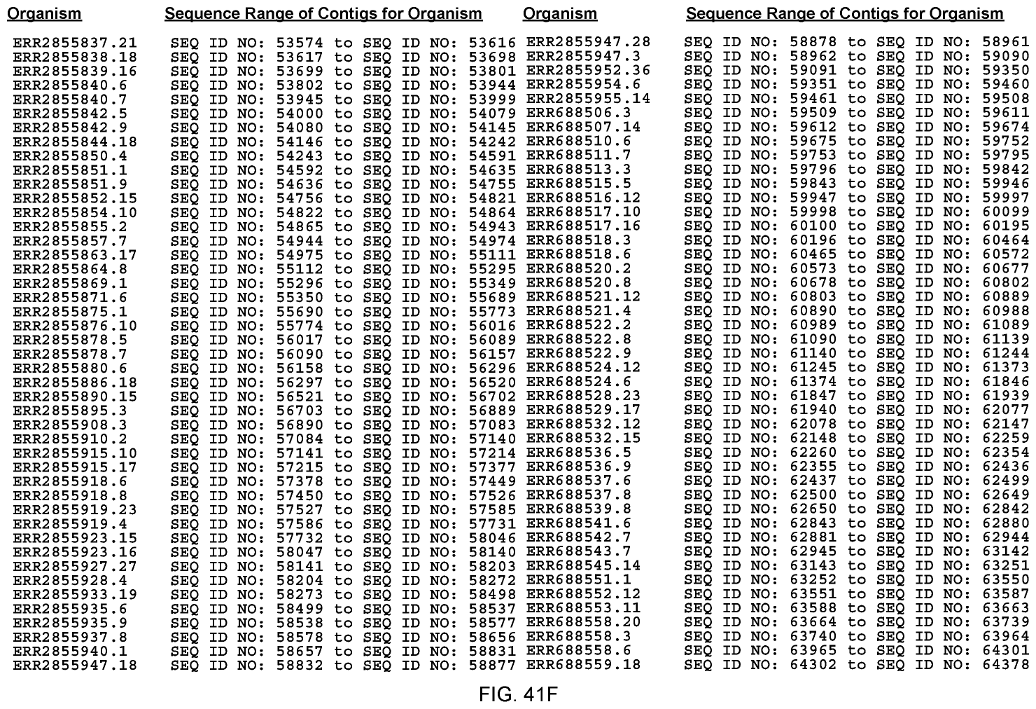

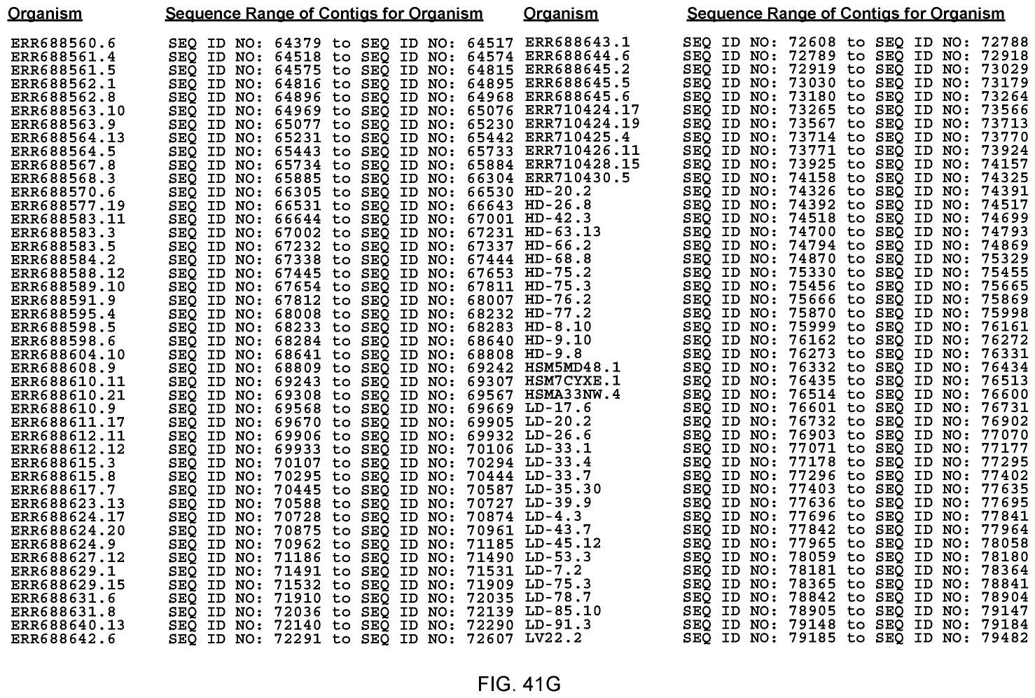

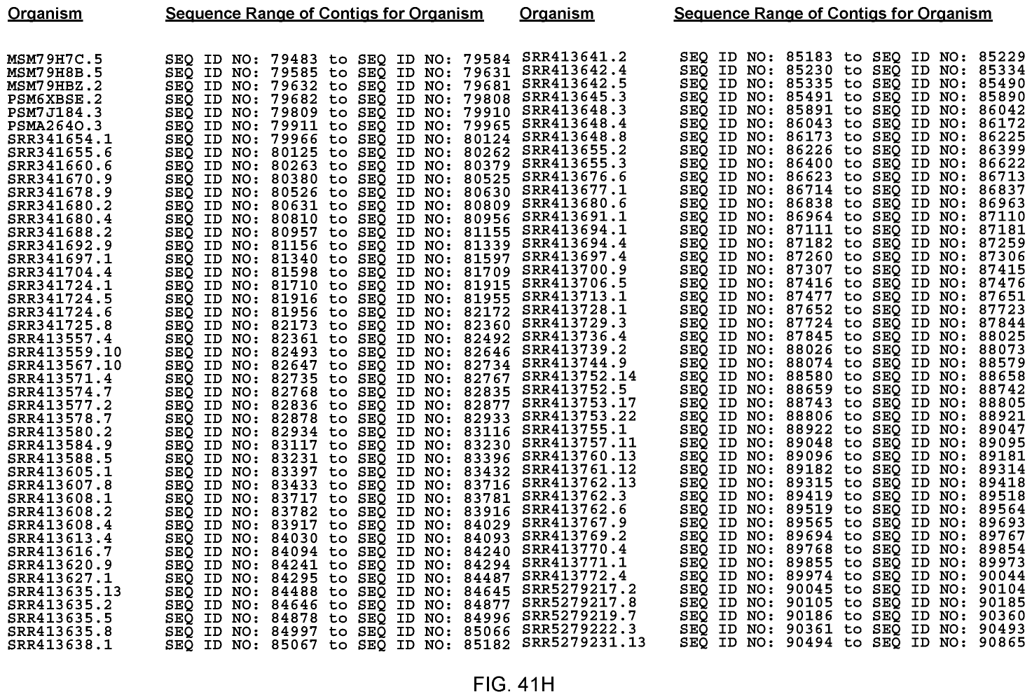

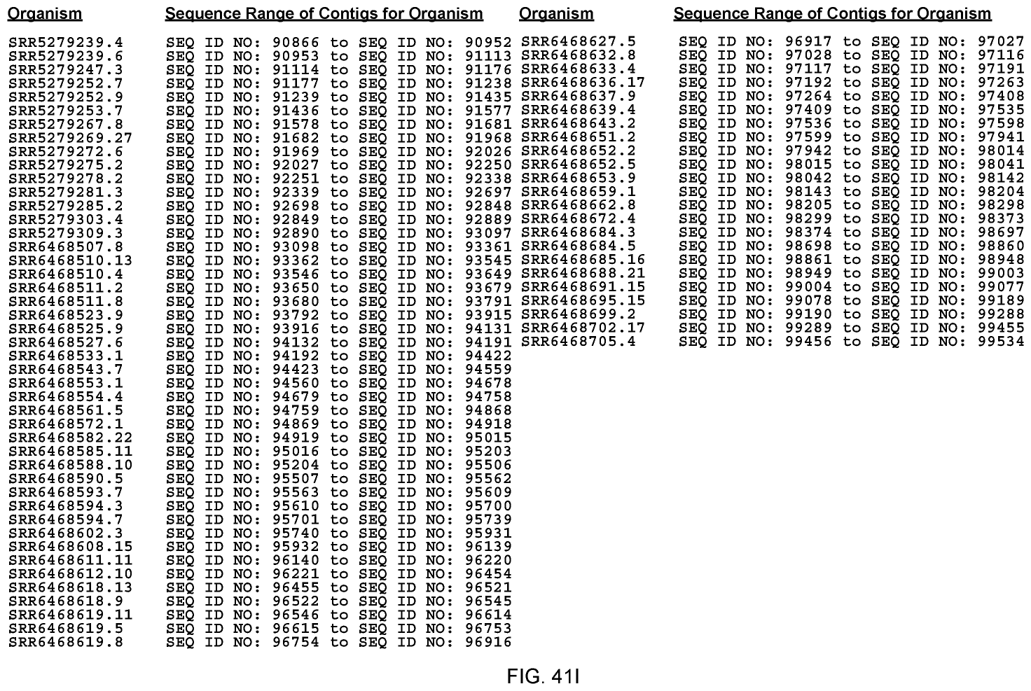

Referring to block 210, in some embodiments, the method includes, for each respective subject in the first plurality of subjects, assigning each respective nucleic acid sequence in the corresponding first plurality of (e.g., at least 100,000) sequences to a respective gut microorganism in the plurality of gut microorganisms, thereby generating, for each respective gut microorganism in the plurality of gut microorganism, a corresponding count of respective nucleic acid sequences in the corresponding first plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid, e.g., a contig listed in FIG. 41. In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity-based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

Referring to block 212, in some embodiments, the first state of the biological characteristic is the absence of a disease or disorder, e.g., type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), and Parkinson's disease (PD), Multiple Sclerosis (MS), Gaucher disease type II (GDII), COVID-19 (COV), Behcet's disease (BD), autism spectrum disorder (ASD), or pancreatic cancer (PC). In some embodiments, the disease or disorder is cancer, Alzheimer diseases, a cardiovascular disease, an autoimmune disease, a mental health disease, an infectious disease, or a genetic disorder.

In some embodiments, the first state of the biological characteristic is a first severity of a disease or disorder. In some embodiments, the severity of the diseases is categorized by type, frequency or intensity experienced by a subject. In some embodiments, the severity of the disease is categorized by the progression or prognosis of a disease or disorder, e.g., different stages of cancer. In some embodiments, the first state of the biological characteristic is an untreated disease or disorder. In some embodiments, the first state of the biological characteristic is a disease or disorder treated with a first therapy, e.g., surgery, radiation therapy, chemotherapy, targeted therapy, gene therapy, immunotherapy, medication, diet change, lifestyle modification.

In some embodiments, the first state of the biological characteristic is a first level of a nutrient in a diet, such as carbohydrate, proteins, fats, vitamins, fibers. In some embodiments the first state of the biological characteristic is a first age. In some embodiments, a threshold value is provided for determining the first state of biological characteristics, e.g., a level of biomarker, a diagnostic cut-off value, or a threshold nutrient intake level.

Referring to block 214, in some embodiments, the plurality of gut microorganisms comprises at least 20 gut microorganisms selected from Table 1, Table 2, or FIG. 42 A-42XX. In some embodiments, gut microorganisms of at least about 20, 25, 30, 35, 40, 45, 50, 55, 60, 65, 70, 75, 80, 85, 90, 95, 100 or greater are selected from Table 1, Table 2 or FIG. 42 A-42XX.

TABLE 1
Taxonomy Assignment of 141 non-redundant genomes
identified in two competing guilds

| | Taxonomy Assignment | |
|---|---|---|
| | (d = domain; p = phylum; | |
| | c = class; o = order; | |
| | f = family; g = genus; | |
| ID | s = species) | Guilds |
| 1U030.25 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 1U035.49 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-103; | |
| | s_{——} | |
| 1U036.46 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-103; | |
| | s_{——} | |
| 1W012.66 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp900317525 | |
| 1W030.27 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W037.25 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_K | |
| 1W059.36 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_K | |
| 1W065.34 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W071.30 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——} | |
| 1W071.47 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 2U004.52 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-103; | |
| | s_{——} | |
| 2U017.37 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 2U033.43 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_C; | |
| | s_{——} | |
| 2W001.22 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_K | |
| 2W007.23 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-110; | |
| | s_{——}CAG-110 sp000434635 | |
| 2W009.30 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 2W023.30 | d_{——}Bacteria; p_{——}Firmicutes_A; | Guild 1 |
| | c_{——}Clostridia; o_{——}Oscillospirales; | |
| | f_{——}Acutalibacteraceae; g_{——}UBA1417; | |
| | s_{——} | |
| 2W027.13 | d_{——}Bacteria; p_{——}Firmicutes_A; | Guild 1 |
| | c_{——}Clostridia; o_{——}Lachnospirales; | |
| | f_{——}Lachnospiraceae; g_{——}Eisenbergiella; | |
| | s_{——}Eisenbergiella sp900066775 | |
| 2W027.27 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| 2W029.13 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_C | |
| 2W034.32 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-110; | |
| | s_{——} | |
| 2W062.21 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 2W064.32 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| 2W067.69 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp000765235 | |
| 2W073.18 | d_{——}Bacteria; p_{——}Firmicutes_A; | Guild 1 |
| | c_{——}Clostridia; o_{——}Christensenellales; | |
| | f_{——}UBA1750; g_{——}UBA7102; | |
| | s_{——} | |
| 2W073.55 | d_{——}Bacteria; p_{——}Firmicutes_A; | Guild 1 |
| | c_{——}Clostridia; o_{——}Oscillospirales; | |
| | f_{——}Butyricicoccaceae; g_{——}Butyricicoccus_A; | |
| | s_{——}Butyricicoccus_A sp002395695 | |
| 2W074.14 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_K | |
| 2W080.51 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_I | |
| 3U005.26 | d_{——}Bacteria; p_{——}Firmicutes_A; | Guild 1 |
| | c_{——}Clostridia; o_{——}Oscillospirales; | |
| | f_{——}Fuminococcaceae; g_{——}Gemmiger; | |
| | s_{——}Gemmiger formicilis | |
| 3U010.20 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-110; | |
| | s_{——} | |
| 3U024.38 | d_{——}Bacteria; p_{——}Firmicutes_A; | Guild 1 |
| | c_{——}Clostridia; o_{——}Oscillospirales; | |
| | f_{——}Acutalibacteraceae; g_{——}CAG-180; | |
| | s_{——}CAG-180 sp000432435 | |
| 3U033.37 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-110; | |
| | s_{——} | |
| 3W015.22 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_J | |
| 3W048.18 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——} | |
| 3W052.32 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——} | |
| 3W062.32 | d_{——}Bacteria; p_{——}Firmicutes_A; | Guild 1 |
| | c_{——}Clostridia; o_{——}Oscillospirales; | |
| | f_{——}Ruminococcaceae; g_{——}CAG-353; | |
| | s_{——}CAG-353 sp900066885 | |
| 3W072.38 | Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 3W073.49 | d_{——}Bacteria; p_{——}Firmicutes_A; | Guild 1 |
| | c_{——}Clostridia; o_{——}Christensenellales; | |
| | f_{——}CAG-138; g_{——}Phil1; | |
| | s_{——}Phil1 sp001940855 | |
| 1U009.16 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| 1W011.28 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W001.34 | d_{——}Bacteria; p_{——}Firmicutes_A; | Guild 1 |
| | c_{——}Clostridia; o_{——}Oscillospirales; | |
| | f_{——}Acutalibacteraceae; g_{——}CAG-177; | |
| | s_{——}CAG-177 sp003514385 | |
| 1U014.38 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_K | |
| 1W037.27 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger formicilis | |
| 1U002.48 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_C; | |
| | s_{——}Ruminococcus_C callidus | |
| 2W066.63 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp900317525 | |
| 2W043.36 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger formicilis | |
| 2W043.20 | d_{——}Bacteria; p_{——}Firmicutes_A; | Guild 1 |
| | c_{——}Clostridia; o_{——}Lachnospirales; | |
| | f_{——}Lachnospiraceae; g_{——}Eisenbergiella; | |
| | s_{——}Eisenbergiella sp900066775 | |
| 2W001.17 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger sp003476825 | |
| 1U002.41 | d_{——}Bacteria; p_{——}Firmicutes_A; | Guild 1 |
| | c_{——}Clostridia; o_{——}Lachnospirales; | |
| | f_{——}Lachnospiraceae; g_{——}Dorea; | |
| | s_{——}Dorea longicatena_B | |
| 1W071.19 | d_{——}Bacteria; p_{——}Firmicutes_A; | Guild 1 |
| | c_{——}Clostridia; o_{——}Lachnospirales; | |
| | f_{——}Lachnospiraceae; g_{——}Coprococcus_A; | |
| | s_{——}Coprococcus_A catus | |
| 1U017.16 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia; | |
| | s_{——} | |
| 1U017.25 | d_{——}Bacteria; p_{——}Firmicutes_A; | Guild 2 |
| | c_{——}Clostridia; o_{——}Lachnospirales; | |
| | f_{——}Lachnospiraceae; g_{——}Tyzzerella; | |
| | s_{——}Tyzzerella sp000209385 | |
| 1U028.21 | d_{——}Bacteria; p_{——}Firmicutes_A; | Guild 2 |
| | c_{——}Clostridia; o_{——}Lachnospirales; | |
| | f_{——}Lachnospiraceae; g_{——}Dorea; | |
| | s_{——}Dorea scindens | |
| 1U030.5 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_{——}Escherichia coli_D | |
| 1U035.55 | d_{——}Bacteria; p_{——}Firmicutes; | Guild 2 |
| | c_{——}Bacilli; o_{——}Lactobacillus; | |
| | f_{——}Streptococcaceae; g_{——}Streptococcus; | |
| | s_{——}Streptococcus macedonicus | |
| 1U038.18 | d_{——}Bacteria; p_{——}Firmicutes_A; | Guild 2 |
| | c_{——}Clostridia; o_{——}Lachnospirales; | |
| | f_{——}Lachnospiraceae; g_{——}UBA7096; | |
| | s_{——} | |
| 1U038.6 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_{——}Escherichia coli_D | |
| 1W006.13 | d_{——}Bacteria; p_{——}Firmicutes_A; | Guild 2 |
| | c_{——}Clostridia; o_{——}Lachnospirales; | |
| | f_{——}Lachnospiraceae; g_{——}Faecalicatena; | |
| | s_{——}Faecalicatena faecis | |
| 1W032.17 | d_{——}Bacteria; p_{——}Bacteroidota; | Guild 2 |
| | c_{——}Bacteroidia; o_{——}Bacteroidales; | |
| | f_{——}Bacteroidaceae; g_{——}Paraprevotella; | |
| | s_{——} | |
| 1W032.19 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_G; | |
| | s_{——}Eubacterium_G sp000434315 | |
| 1W033.3 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——} | |
| 1W033.9 | d_{——}Bacteria; p_{——}Firmicutes_A; | Guild 2 |
| | c_{——}Clostridia; o_{——}Oscillospirales; | |
| | f_{——}Oscillospiraceae; g_{——}Flavonifractor; | |
| | s_{——}Flavonifractor plautii | |
| 1W036.28 | d_{——}Bacteria; p_{——}Firmicutes_A; | Guild 2 |
| | c_{——}Clostridia; o_{——}Lachnospirales; | |
| | f_{——}Lachnospiraceae; g_{——}Tyzzerella; | |
| | s_{——}Tyzzerella nexilis | |
| 1W038.31 | d_{——}Bacteria; p_{——}Fusobacteriota; | Guild 2 |
| | c_{——}Fusobacteriia; o_{——}Fusobacteriales; | |
| | f_{——}Fusobacteriaceae; g_{——}Fusobacterium_A; | |
| | s_{——} | |
| 1W042.8 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M sp001517625 | |
| 1W047.42 | d_{——}Bacteria; p_{——}Firmicutes_C; | Guild 2 |
| | c_{——}Negativicutes; o_{——}Acidaminococcales; | |
| | f_{——}Acidaminococcaceae; g_{——}Acidaminococcus; | |
| | s_{——}Acidaminococcus intestini | |
| 1W062.68 | d_{——}Bacteria; p_{——}Firmicutes; | Guild 2 |
| | c_{——}Bacilli; o_{——}Lactobacillales; | |
| | f_{——}Streptococcaceae; g_{——}Streptococcus; | |
| | s_{——}Streptococcus sp002300045 | |
| 1W070.8 | d_{——}Bacteria; p_{——}Firmicutes_A; | Guild 2 |
| | c_{——}Clostridia; o_{——}Peptostreptococcales; | |
| | f_{——}Peptostreptococcaceae; g_{——}Intestinibacter; | |
| | s_{——}Intestinibacter bartlettii | |
| 1W072.11 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Absiella; | |
| | s_{——} | |
| 1W075.10 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A wexlerae | |
| 1W075.14 | d_{——}Bacteria; p_{——}Bacteroidota; | Guild 2 |
| | c_{——}Bacteroidia; o_{——}Bacteroidales; | |
| | f_{——}Marinifilaceae; g_{——}Odoribacter; | |
| | s_{——} | |
| 1W078.21 | d_{——}Bacteria; p_{——}Firmicutes_C; | Guild 2 |
| | c_{——}Negativicutes; o_{——}Veillonellales; | |
| | f_{——}Veillonellaceae; g_{——}Veillonella; | |
| | s_{——}Veillonella parvula_A | |
| 1W080.50 | d_{——}Bacteria; p_{——}Proteobacteria; | Guild 2 |
| | c_{——}Alphaproteobacteria; o_{——}RF32; | |
| | f_{——}CAG-239; g_{——}51-20; | |
| | s_{——}51-20 sp001917175 | |
| 2U001.12 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A hydrogenotrophica | |
| 2U001.17 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M sp001517625 | |
| 2U001.37 | d_{——}Bacteria; p_{——}Firmicutes; | Guild 2 |
| | c_{——}Bacilli; o_{——}Lactobacillales; | |
| | f_Lactobacillaceae; g_{——}Lactobacillus_H; | |
| | s_{——}Lactobacillus_H oris | |
| 2U008.16 | d_{——}Bacteria; p_{——}Fusobacteriota; | Guild 2 |
| | c_{——}Fusobacteriia; o_{——}Fusobacteriales; | |
| | f_{——}Fusobacteriaceae; g_{——}Fusobacterium_A; | |
| | s_{——}Fusobacterium_A ulcerans | |
| 2U017.6 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Klebsiella; | |
| | s_{——}Klebsiella variicola | |
| 2U033.18 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A sp000434735 | |
| 2U037.14 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides clarus | |
| 2U037.18 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A plebeius | |
| 2U037.6 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_{——}Escherichia flexneri | |
| 2U038.12 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_Q; | |
| | s_{——}Clostridium_Q symbiosum | |
| 2U038.5 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia; | |
| | s_{——}Blautia sp001304935 | |
| 2W001.42 | d_{——}Bacteria; p_{——}Proteobacteria; | Guild 2 |
| | c_{——}Alphaproteobacteria; o_{——}RF32; | |
| | f_{——}CAG-239; g_{——}CAG-495; | |
| | s_{——}CAG-495 sp000432275 | |
| 2W002.29 | d_{——}Bacteria; p_{——}Actinobacteriota; | Guild 2 |
| | c_{——}Actinobacteria; o_{——}Actinomycetales; | |
| | f_{——}Bifidobacteriaceae; g_{——}Bifidobacterium; | |
| | s_{——}Bifidobacterium longum | |
| 2W003.7 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_B; | |
| | s_{——}Bacteroides_B vulgatus | |
| 2W004.25 | d_{——}Bacteria; p_{——}Firmicutes_C; | Guild 2 |
| | c_{——}Negativicutes; o_{——}Veillonellales; | |
| | f_{——}Megasphaeraceae; g_{——}Caecibacter; | |
| | s_{——} | |
| 2W005.9 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides johnsonii | |
| 2W011.21 | d_{——}Bacteria; p_{——}Firmicutes; | Guild 2 |
| | c_{——}Bacilli; o_{——}Lactobacillales; | |
| | f_Enterococcaceae; g_{——}Enterococcus_B; | |
| | s_{——}Enterococcus_B hirae | |
| 2W012.75 | d_{——}Bacteria; p_{——}Firmicutes; | Guild 2 |
| | c_{——}Bacilli; o_{——}Lactobacillales; | |
| | f_Streptococcaceae; g_{——}Streptococcus; | |
| | s_{——}Streptococcus thermophilus | |
| 2W025.8 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Enterobacter; | |
| | s_{——}Enterobacter kobei | |
| 2W026.44 | d_{——}Bacteria; p_{——}Firmicutes; | Guild 2 |
| | c_{——}Bacilli; o_{——}Lactobacillus; | |
| | f_{——}Streptococcaceae; g_{——}Streptococcus; | |
| | s_{——}Streptococcus infantarius | |
| 2W037.10 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides sp000436495 | |
| 2W048.49 | d_{——}Bacteria; p_{——}Firmicutes_A; | Guild 2 |
| | c_{——}Clostridia; o_{——}Peptostreptococcales; | |
| | f_{——}Anaerovoracaceae; g_{——}UBA1191; | |
| | s_{——} | |
| 2W050.17 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides stercoris | |
| 2W053.46 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Oxalobacter; | |
| | s_{——}Oxalobacter formigenes_A | |
| 2W054.20 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp900066165 | |
| 2W065.60 | d_{——}Bacteria; p_{——}Firmicutes; | Guild 2 |
| | c_{——}Bacilli; o_{——}Lactobacillus | |
| | f_Streptococcaceae; g_{——}Streptococcus; | |
| | s_{——}Streptococcus salivarius | |
| 2W067.20 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A coprocola | |
| 2W075.14 | d_{——}Bacteria; p_{——}Firmicutes_A; | Guild 2 |
| | c_{——}Clostridia; o_{——}Lachnospirales; | |
| | f_{——}Lachnospiraceae; g_{——}Faecalicatena; | |
| | s_{——}Faecalicatena gnavus | |
| 2W080.58 | d_{——}Bacteria; p_{——}Proteobacteria; | Guild 2 |
| | c_{——}Alphaproteobacteria; o_{——}RF32; | |
| | f_{——}CAG-239; g_{——}CAG-495; | |
| | s_{——} | |
| 3U007.7 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M bolteae | |
| 3U020.14 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_I; | |
| | s_{——}Eubacterium_I ramulus | |
| 3U023.7 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——} | |
| 3U038.19 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp000433815 | |
| 3U040.11 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides stercoris | |
| 3W002.16 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_Q; | |
| | s_{——}Clostridium_Q saccharolyticum | |
| 3W023.3 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides cellulosilyticus | |
| 3W028.33 | d_{——}Bacteria; p_{——}Firmicutes; | Guild 2 |
| | c_{——}Bacilli; o_{——}Lactobacilles; | |
| | f_{——}Streptococcaceae; g_{——}Streptococcus; | |
| | s_{——}Streptococcus pasteurianus | |
| 3W036.5 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_B; | |
| | s_{——}Bacteroides_B dorei | |
| 3W036.6 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides fragilis | |
| 3W039.10 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides eggerthii | |
| 3W042.11 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A plebeius_A | |
| 3W042.9 | d_{——}Bacteria; p_{——}Firmicutes; | Guild 2 |
| | c_{——}Bacilli; o_{——}Erysipelotrichales; | |
| | f_{——}Erysipelatoclostridiaceae; g_{——}Erysipelatoclostridium; | |
| | s_{——}Erysipelatoclostridium ramosum | |
| 3W049.14 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides stercoris | |
| 3W054.11 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_Escherichia flexneri | |
| 3W055.19 | d_{——}Bacteria; p_{——}Bacteroidota; | Guild 2 |
| | c_{——}Bacteroidia; o_{——}Bacteroidales; | |
| | f_{——}Barnesiellaceae; g_{——}Barnesiella; | |
| | s_{——}Barnesiella intestinihominis | |
| 3W067.18 | d_{——}Bacteria; p_{——}Bacteroidota; | Guild 2 |
| | c_{——}Bacteroidia; o_{——}Bacteroidales; | |
| | f_{——}Muribaculaceae; g_{——}CAG-279; | |
| | s_{——} | |
| 3W074.2 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides thetaiotaomicron | |
| 3W079.9 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A coprophilus | |
| 3W080.19 | d_{——}Bacteria; p_{——}Firmicutes; | Guild 2 |
| | c_{——}Bacilli; o_{——}Lactobacillales; | |
| | f_Enterococcaceae; g_{——}Enterococcus_D; | |
| | s_{——}Enterococcus_D sp002850555 | |
| 3W080.64 | d_{——}Bacteria; p_{——}Firmicutes; | Guild 2 |
| | c_{——}Bacilli; o_{——}RFN20; | |
| | f_{——}CAG-288; g_{——}UBA7642; | |
| | s_{——} | |
| 1U003.28 | d_{——}Bacteria; p_{——}Actinobacteriota; | Guild 2 |
| | c_{——}Actinobacteria; o_{——}Actinomycetales; | |
| | f_{——}Micrococcaceae; g_{——}Rothia; | |
| | s_{——}Rothia mucilaginosa_B | |
| 1U017.15 | d_{——}Bacteria; p_{——}Firmicutes_A; | Guild 2 |
| | c_{——}Clostridia; o_{——}Lachnospirales; | |
| | f_{——}Lachnospiraceae; g_{——}Sellimonas; | |
| | s_{——}Sellimonas intestinalis | |
| 1U001.8 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_{——}Escherichia coli | |
| 1U017.10 | d_{——}Bacteria; p_{——}Firmicutes_A; | Guild 2 |
| | c_{——}Clostridia; o_{——}Lachnospirales; | |
| | f_{——}Lachnospiraceae; g_{——}Dorea; | |
| | s_{——}Dorea sp000509125 | |
| 1U027.33 | d_{——}Bacteria; p_{——}Firmicutes_A; | Guild 2 |
| | c_{——}Clostridia; o_{——}Oscillospirales; | |
| | f_{——}Acutalibacteraceae; g_{——}Eubacterium_R; | |
| | s_{——}Eubacterium_R sp000431535 | |
| 1U015.30 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Absiella; | |
| | s_{——}Absiella dolichum | |
| 1U036.9 | d_{——}Bacteria; p_{——}Bacteroidota; | Guild 2 |
| | c_{——}Bacteroidia; o_{——}Bacteroidales; | |
| | f_{——}Bacteroidaceae; g_{——}Paraprevotella; | |
| | s_{——}Paraprevotella clara | |
| 1U015.20 | d_{——}Bacteria; p_{——}Firmicutes_A; | Guild 2 |
| | c_{——}Clostridia; o_{——}Lachnospirales; | |
| | f_{——}Lachnospiraceae; g_{——}Faecalicatena; | |
| | s_{——}Faecalicatena glycyrrhizinilyticum | |
| 1W023.6 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Klebsiella; | |
| | s_{——}Klebsiella pneumoniae | |
| 1W023.15 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A plebeius | |
| 1U024.11 | d_{——}Bacteria; p_{——}Actinobacteriota; | Guild 2 |
| | c_{——}Coriobacteriia; o_{——}Coriobacteriales; | |
| | f_{——}Eggerthellaceae; g_{——}Eggerthella; | |
| | s_{——}Eggerthella lenta | |
| 1W023.20 | d_{——}Bacteria; p_{——}Firmicutes_A; | Guild 2 |
| | c_{——}Clostridia; o_{——}Oscillospirales; | |
| | f_{——}Oscillospiraceae; g_{——}Lawsonibacter; | |
| | s_{——} | |
| 1U036.17 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides stercoris | |
| 1U021.14 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A coprocola | |
| 1W026.10 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——} | |
| 1U014.18 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M sp000431375 | |
| 2W067.16 | d_{——}Bacteria; p_{——}Bacteroidota; | Guild 2 |
| | c_{——}Bacteroidia; o_{——}Bacteroidales; | |
| | f_{——}Coprobacteraceae; g_{——}Coprobacter; | |
| | s_{——}Coprobacter fastidiosus | |
| 1W079.48 | d_{——}Bacteria; p_{——}Bacteroidota; | Guild 2 |
| | c_{——}Bacteroidia; o_{——}Bacteroidales; | |
| | f_{——}UBA932; g_{——}RC9; | |
| | s_{——}RC9 sp000431015 | |

TABLE 2
Taxonomy Assignment of 284 core microbiome

| | Taxonomy Assignment | |
|---|---|---|
| | (d = domain; p = phylum; | |
| | c = class; o = order; | |
| | f = family; g = genus; | |
| ID | s = species) | Assignment |
| 1U003.28 | d_{——}Bacteria; p_{——}Actinobacteriota; | Guild 2 |
| | c_{——}Actinobacteria; o_{——}Actinomycetales; | |
| | f_{——}Micrococcaceae; g_{——}Rothia; | |
| | s_{——}Rothia mucilaginosa_B | |
| 1U015.20 | d_{——}Bacteria; p_{——}Firmicutes_A; | Guild 2 |
| | c_{——}Clostridia; o_{——}Lachnospirales; | |
| | f_{——}Lachnospiraceae; g_{——}Faecalicatena; | |
| | s_{——}Faecalicatena glycyrrhizinilyticum | |
| 1U024.11 | d_{——}Bacteria; p_{——}Actinobacteriota; | Guild 2 |
| | c_{——}Coriobacteriia; o_{——}Coriobacteriales; | |
| | f_{——}Eggerthellaceae; g_{——}Eggerthella; | |
| | s_{——}Eggerthella lenta | |
| 1U035.49 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-103; s | |
| 1U035.55 | d_{——}Bacteria; p_{——}Firmicutes; | Guild 2 |
| | c_{——}Bacilli; o_{——}Lactobacillales; | |
| | f_{——}Streptococcaceae; g_{——}Streptococcus; | |
| | s_{——}Streptococcus macedonicus | |
| 1U038.18 | d_{——}Bacteria; p_{——}Firmicutes_A; | Guild 2 |
| | c_{——}Clostridia; o_{——}Lachnospirales; | |
| | f_{——}Lachnospiraceae; g_{——}UBA7096; | |
| | s_{——} | |
| 1W011.28 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W023.20 | d_{——}Bacteria; p_{——}Firmicutes_A; | Guild 2 |
| | c_{——}Clostridia; o_{——}Oscillospirales; | |
| | f_{——}Oscillospiraceae; g_{——}Lawsonibacter; | |
| | s_{——} | |
| 1W030.27 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W032.19 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_G; | |
| | s_{——}Eubacterium_G sp000434315 | |
| 1W033.9 | d_{——}Bacteria; p_{——}Firmicutes_A; | Guild 2 |
| | c_{——}Clostridia; o_{——}Oscillospirales; | |
| | f_{——}Oscillospiraceae; g_{——}Flavonifractor; | |
| | s_{——}Flavonifractor plautii | |
| 1W042.8 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M sp001517625 | |
| 1W065.34 | d_{——}Bacteria; p_{——}Firmicutes_A; | Guild 1 |
| | c_{——}Clostridia; o_{——}Oscillospirales; | |
| | f_{——}Oscillospiraceae; g_{——}Oscillibacter; | |
| | s_{——}Oscillibacter sp001916835 | |
| 1W072.11 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Absiella; | |
| | s_{——} | |
| 1W075.14 | d_{——}Bacteria; p_{——}Bacteroidota; | Guild 2 |
| | c_{——}Bacteroidia; o_{——}Bacteroidales; | |
| | f_{——}Marinifilaceae; g_{——}Odoribacter; | |
| | s_{——} | |
| 1W078.21 | d_{——}Bacteria; p_{——}Firmicutes_C; | Guild 2 |
| | c_{——}Negativicutes; o_{——}Veillonellales; | |
| | f_{——}Veillonellaceae; g_{——}Veillonella; | |
| | s_{——}Veillonella parvula_A | |
| 1W080.50 | d_{——}Bacteria; p_{——}Proteobacteria; | Guild 2 |
| | c_{——}Alphaproteobacteria; o_{——}RF32; | |
| | f_{——}CAG-239; g_{——}51-20; | |
| | s_{——}_{_{—}}51-20 sp001917175 | |
| 2U001.17 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——}Clostridium_M sp001517625 | |
| 2U008.16 | d_{——}Bacteria; p_{——}Fusobacteriota; | Guild 2 |
| | c_{——}Fusobacteriia; o_{——}Fusobacteriales; | |
| | f_{——}Fusobacteriaceae; g_{——}Fusobacterium_A; | |
| | s_{——}Fusobacterium_A ulcerans | |
| 2U033.18 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides_A; | |
| | s_{——}Bacteroides_A sp000434735 | |
| 2U037.14 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides clarus | |
| 2W001.42 | d_{——}Bacteria; p_{——}Proteobacteria; | Guild 2 |
| | c_{——}Alphaproteobacteria; o_{——}RF32; | |
| | f_{——}CAG-239; g_{——}CAG-495; | |
| | s_{——}CAG-495 sp000432275 | |
| 2W011.21 | d_{——}Bacteria; p_{——}Firmicutes; | Guild 2 |
| | c_{——}Bacilli; o_{——}Lactobacilles; | |
| | f_{——}Enterococcaceae; g_{——}Enterococcus_B; | |
| | s_{——}Enterococcus_B hirae | |
| 2W023.30 | d_{——}Bacteria; p_{——}Firmicutes_A; | Guild 1 |
| | c_{——}Clostridia; o_{——}Oscillospirales; | |
| | f_{——}Acutalibacteraceae; g_{——}UBA1417; | |
| | s_{——} | |
| 2W027.27 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| 2W048.49 | d_{——}Bacteria; p_{——}Firmicutes_A; | Guild 2 |
| | c_{——}Clostridia; o_{——}Peptostreptococcales; | |
| | f_{——}Anaerovoracaceae; g_{——}UBA1191; | |
| | s_{——} | |
| 2W053.46 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Oxalobacter; | |
| | s_{——}Oxalobacter formigenes_A | |
| 2W065.60 | d_{——}Bacteria; p_{——}Firmicutes; | Guild 2 |
| | c_{——}Bacilli; o_{——}Lactobacilles; | |
| | f_{——}Streptococcaceae; g_{——}Streptococcus; | |
| | s_{——}Streptococcus salivarius | |
| 2W073.55 | d_{——}Bacteria; p_{——}Firmicutes_A; | Guild 1 |
| | c_{——}Clostridia; o_{——}Oscillospirales; | |
| | f_{——}Butyricicoccaceae; g_{——}Butyricicoccus_A; | |
| | s_{——}Butyricicoccus_A sp002395695 | |
| 3U020.14 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_I; | |
| | s_{——}Eubacterium_I ramulus | |
| 3U023.7 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_M; | |
| | s_{——} | |
| 3W002.16 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_Q; | |
| | s_{——}Clostridium_Q saccharolyticum | |
| 3W015.22 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_J | |
| 3W039.10 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides eggerthii | |
| 3W055.19 | d_{——}Bacteria; p_{——}Bacteroidota; | Guild 2 |
| | c_{——}Bacteroidia; o_{——}Bacteroidales; | |
| | f_{——}Barnesiellaceae; g_{——}Barnesiella; | |
| | s_{——}Barnesiella intestinihominis | |
| 3W062.32 | d_{——}Bacteria; p_{——}Firmicutes_A; | Guild 1 |
| | c_Clostridia; o_{——}Oscillospirales; | |
| | f_{——}Ruminococcaceae; g_{——}CAG-353; | |
| | s_{——}CAG-353 sp900066885 | |
| 3W067.18 | d_{——}Bacteria; p_{——}Bacteroidota; | Guild 2 |
| | c_{——}Bacteroidia; o_{——}Bacteroidales; | |
| | f_{——}Muribaculaceae; g_{——}CAG-279; | |
| | s_{——} | |
| 3W074.2 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides thetaiotaomicron | |
| ERR1018189.10 | d_{——}Bacteria; p_{——}Firmicutes_A; | Guild 2 |
| | c_{——}Clostridia; o_{——}Oscillospirales; | |
| | f_{——}Oscillospiraceae; g_{——}CAG-110; | |
| | s_{——}CAG-110 sp900546075 | |
| ERR1018192.4 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp900066145 | |
| ERR1018196.11 | d_{——}Bacteria; p_{——}Firmicutes_C; | Guild 2 |
| | c_{——}Negativicutes; o_{——}Veillonellales; | |
| | f_{——}Dialisteraceae; g_{——}Dialister; | |
| | s_{——}Dialister succinatiphilus | |
| ERR1018197.5 | d_{——}Bacteria; p_{——}Firmicutes_A; | Guild 1 |
| | c_{——}Clostridia; o_{——}Oscillospirales; | |
| | f_{——}Acutalibacteraceae; g_{——}UMGS263; | |
| | s_{——}UMGS263 sp900540535 | |
| ERR1018197.9 | d_{——}Bacteria; p_{——}Firmicutes_A; | Guild 1 |
| | c_{——}Clostridia; o_{——}Oscillospirales; | |
| | f_{——}Acutalibacteraceae; g_{——}Clostridium_A; | |
| | s_{——}Clostridium_A leptum | |
| ERR1018200.15 | d_{——}Bacteria; p_{——}Firmicutes_A; | Guild 2 |
| | c_{——}Clostridia; o_{——}Oscillospirales; | |
| | f_{——}Acutalibacteraceae; g_{——}UMGS1071; | |
| | s_{——}UMGS1071 sp900542375 | |
| ERR1018200.4 | d_{——}Bacteria; p_{——}Firmicutes_A; | Guild 2 |
| | c_{——}Clostridia; o_{——}Lachnospirales; | |
| | f_{——}Lachnospiraceae; g_{——}Eisenbergiella; | |
| | s_{——}Eisenbergiella tayi | |
| ERR1018204.1 | d_{——}Bacteria; p_{——}Firmicutes_A; | Guild 2 |
| | c_{——}Clostridia; o_{——}Lachnospirales; | |
| | f_{——}Lachnospiraceae; g_{——}Enterocloster; | |
| | s_{——}Enterocloster clostridioformis | |
| ERR1018210.9 | d_{——}Bacteria; p_{——}Actinobacteriota; | Guild 2 |
| | c_{——}Actinomycetia; o_{——}Actinomycetales; | |
| | f_{——}Bifidobacteriaceae; g_{——}Bifidobacterium; | |
| | s_{——}Bifidobacterium pseudocatenulatum | |
| ERR1018213.1 | d_{——}Bacteria; p_{——}Firmicutes; | Guild 2 |
| | c_{——}Bacilli; o_{——}Lactobacillales; | |
| | f_{——}Streptococcaceae; g_{——}Streptococcus; | |
| | s_{——}Streptococcus salivarius | |
| ERR1018218.4 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella hominis | |
| ERR1018219.12 | d_{——}Bacteria; p_{——}Firmicutes_A; | Guild 2 |
| | c_{——}Clostridia; o_{——}Oscillospirales; | |
| | f_{——}Acutalibacteraceae; g_{——}Eubacterium_R; | |
| | s_{——}Eubacterium_R sp000434995 | |
| ERR1018219.9 | d_{——}Bacteria; p_{——}Desulfobacterota; | Guild 2 |
| | c_{——}Desulfovibrionia; o_{——}Desulfovibrionales; | |
| | f_{——}Desulfovibrionaceae; g_{——}Bilophila; | |
| | s_{——}Bilophila wadsworthia | |
| ERR1018229.14 | d_{——}Bacteria; p_{——}Firmicutes_C; | Guild 2 |
| | c_{——}Negativicutes; o_{——}Veillonellales; | |
| | f_{——}Megasphaeraceae; g_{——}Caecibacter; | |
| | s_{——}Caecibacter hominis | |
| ERR1018232.13 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella copri | |
| ERR1018237.11 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_G; | |
| | s_{——}Eubacterium_G ventriosum | |
| ERR1018238.17 | d_{——}Bacteria; p_{——}Firmicutes_A; | Guild 2 |
| | c_{——}Clostridia; o_{——}Lachnospirales; | |
| | f_{——}Lachnospiraceae; g_{——}Anaerostipes; | |
| | s_{——}Anaerostipes hadrus | |
| ERR1018239.7 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp003480185 | |
| ERR1018250.9 | d_{——}Bacteria; p_{——}Firmicutes_A; | Guild 2 |
| | c_{——}Clostridia; o_{——}Lachnospirales; | |
| | f_{——}Lachnospiraceae; g_{——}Enterocloster; | |
| | s_{——}Enterocloster sp900541315 | |
| ERR1018258.4 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides distasonis | |
| ERR1018262.5 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides uniformis | |
| ERR1018267.4 | d_{——}Bacteria; p_{——}Bacteroidota; | Guild 2 |
| | c_{——}Bacteroidia; o_{——}Bacteroidales; | |
| | f_{——}Coprobacteraceae; g_{——}Coprobacter; | |
| | s_{——}Coprobacter fastidiosus | |
| ERR1018278.5 | d_{——}Bacteria; p_{——}Firmicutes_C; | Guild 2 |
| | c_{——}Negativicutes; o_{——}Veillonellales; | |
| | f_{——}Dialisteraceae; g_{——}Dialister; | |
| | s_{——}Dialister sp900541605 | |
| ERR1018291.11 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp900317525 | |
| ERR1018292.2 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides intestinalis | |
| ERR1018298.1 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Faecalitalea; | |
| | s_{——}Faecalitalea cylindroides | |
| ERR1018301.12 | d_{——}Bacteria; p_{——}Bacteroidota; | Guild 2 |
| | c_{——}Bacteroidia; o_{——}Bacteroidales; | |
| | f_{——}Marinifilaceae; g_{——}Butyricimonas; | |
| | s_{——}Butyricimonas virosa | |
| ERR1018308.3 | d_{——}Bacteria; p_{——}Firmicutes_A; | Guild 2 |
| | c_{——}Clostridia; o_{——}Lachnospirales; | |
| | f_{——}Lachnospiraceae; g_{——}Agathobacter; | |
| | s_{——}Agathobacter sp900548765 | |
| ERR1018309.23 | d_{——}Bacteria; p_{——}Firmicutes_A; | Guild 2 |
| | c_{——}Clostridia; o_{——}Monoglobales_A; | |
| | f_{——}UBA1381; g_{——}CAG-41; | |
| | s_{——}CAG-41 sp900066215 | |
| ERR2017433.5 | d_{——}Bacteria; p_{——}Firmicutes_A; | Guild 1 |
| | c_{——}Clostridia; o_{——}Lachnospirales; | |
| | f_{——}Lachnospiraceae; g_{——}Lachnospira; | |
| | s_{——}Lachnospira sp000437735 | |
| ERR2017443.11 | d_{——}Bacteria; p_{——}Firmicutes_C; | Guild 2 |
| | c_{——}Negativicutes; o_{——}Acidaminococcales; | |
| | f_{——}Acidaminococcaceae; g_{——}Phascolarctobacterium; | |
| | s_{——}Phascolarctobacterium faecium | |
| ERR2017473.2 | d_{——}Bacteria; p_{——}Fusobacteriota; | Guild 2 |
| | c_{——}Fusobacteriia; o_{——}Fusobacteriales; | |
| | f_{——}Fusobacteriaceae; g_{——}Fusobacterium_A; | |
| | s_{——}Fusobacterium_A mortiferum | |
| ERR2017500.4 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola massiliensis | |
| ERR2017517.1 | d_{——}Bacteria; p_{——}Firmicutes_A; | Guild 1 |
| | c_{——}Clostridia; o_{——}Lachnospirales; | |
| | f_{——}Lachnospiraceae; g_{——}RUG115; | |
| | s_{——}RUG115 sp900066395 | |
| ERR2017522.17 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Longicatena; | |
| | s_{——}Longicatena caecimuris | |
| ERR2017539.14 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_C; | |
| | s_{——}Ruminococcus_C sp000433635 | |
| ERR2017540.1 | d_{——}Bacteria; p_{——}Bacteroidota; | Guild 2 |
| | c_{——}Bacteroidia; o_{——}Bacteroidales; | |
| | f_{——}Rikenellaceae; g_{——}Alistipes_A; | |
| | s_{——}Alistipes_A indistinctus | |
| ERR2017541.4 | d_{——}Bacteria; p_{——}Firmicutes_A; | Guild 1 |
| | c_{——}Clostridia; o_{——}Lachnospirales; | |
| | f_{——}Lachnospiraceae; g_{——}CAG-95; | |
| | s_{——}CAG-95 sp900066375 | |
| ERR2017552.8 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger formicilis | |
| ERR2017555.15 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium sp900540455 | |
| ERR2017555.23 | d_{——}Bacteria; p_{——}Firmicutes_A; | Guild 2 |
| | c_{——}Clostridia; o_{——}Oscillospirales; | |
| | f_{——}Ruminococcaceae; g_{——}Ruthenibacterium; | |
| | s_{——}Ruthenibacterium lactatiformans | |
| ERR2017563.14 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_D; | |
| | s_{——}Ruminococcus_D bicirculans | |
| ERR2017569.9 | d_{——}Bacteria; p_{——}Firmicutes_C; | Guild 2 |
| | c_{——}Negativicutes; o_{——}Acidaminococcales; | |
| | f_{——}Acidaminococcaceae; g_{——}Phascolarctobacterium; | |
| | s_{——}Phascolarctobacterium sp900545535 | |
| ERR2017573.11 | d_{——}Bacteria; p_{——}Bacteroidota; | Guild 1 |
| | c_{——}Bacteroidia; o_{——}Bacteroidales; | |
| | f_{——}Bacteroidaceae; g_{——}Prevotella; | |
| | s_{——}Prevotella pectinovora | |
| ERR2017577.6 | d_{——}Bacteria; p_{——}Firmicutes; | Guild 2 |
| | c_{——}Bacilli; o_{——}Erysipelotrichales; | |
| | f_{——}Erysipelatoclostridiaceae; g_{——}Coprobacillus; | |
| | s_{——}Coprobacillus cateniformis | |
| ERR2017581.15 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp000765235 | |
| ERR2017584.6 | d_{——}Bacteria; p_{——}Firmicutes_A; | Guild 1 |
| | c_{——}Clostridia; o_{——}Lachnospirales; | |
| | f_{——}Lachnospiraceae; g_{——}Anaerobutyricum; | |
| | s_{——}Anaerobutyricum hallii | |
| ERR2017586.7 | d_{——}Bacteria; p_{——}Firmicutes; | Guild 2 |
| | c_{——}Bacilli; o_{——}Erysipelotrichales; | |
| | f_{——}Erysipelatoclostridiaceae; g_{——}Longibaculum; | |
| | s_{——}Longibaculum muris | |
| ERR2017591.13 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola sp900542985 | |
| ERR2017596.4 | d_{——}Bacteria; p_{——}Actinobacteriota; | Guild 2 |
| | c_{——}Actinomycetia; o_{——}Actinomycetales; | |
| | f_{——}Bifidobacteriaceae; g_{——}Bifidobacterium; | |
| | s_{——}Bifidobacterium longum | |
| ERR2017601.3 | d_{——}Bacteria; p_{——}Firmicutes; | Guild 2 |
| | c_{——}Bacilli; o_{——}Lactobacillales; | |
| | f_{——}Lactobacillaceae; g_{——}Limosilactobacillus; | |
| | s_{——}Limosilactobacillus fermentum | |
| ERR2017608.6 | d_{——}Bacteria; p_{——}Firmicutes_A; | Guild 1 |
| | c_{——}Clostridia; o_{——}Oscillospirales; | |
| | f_{——}Oscillospiraceae; g_{——}UBA5446; | |
| | s_{——}UBA5446 sp900546485 | |
| ERR2017610.5 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Klebsiella; | |
| | s_{——}Klebsiella aerogenes | |
| ERR2017617.4 | d_{——}Bacteria; p_{——}Firmicutes_C; | Guild 2 |
| | c_{——}Negativicutes; o_{——}Veillonellales; | |
| | f_{——}Veillonellaceae; g_{——}Veillonella; | |
| | s_{——}Veillonella parvula_A | |
| ERR2017618.4 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| ERR2017622.3 | d_{——}Bacteria; p_{——}Firmicutes_A; | Guild 1 |
| | c_{——}Clostridia; o_{——}Lachnospirales; | |
| | f_{——}Lachnospiraceae; g_{——}Coprococcus; | |
| | s_{——}Coprococcus sp900066115 | |
| ERR2017636.11 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp003471165 | |
| ERR2017638.17 | d_{——}Bacteria; p_{——}Firmicutes_A; | Guild 1 |
| | c_{——}Clostridia; o_{——}Oscillospirales; | |
| | f_{——}Acutalibacteraceae; g_{——}Ruminococcus_E; | |
| | s_{——}Ruminococcus_E bromii_B | |
| ERR2017641.10 | d_{——}Bacteria; p_{——}Firmicutes_A; | Guild 1 |
| | c_{——}Clostridia; o_{——}Lachnospirales; | |
| | f_{——}Lachnospiraceae; g_{——}COE1; | |
| | s_{——}COE1 sp001916965 | |
| ERR2017647.13 | d_{——}Bacteria; p_{——}Firmicutes_A; | Guild 1 |
| | c_{——}Clostridia; o_{——}Oscillospirales; | |
| | f_{——}Acutalibacteraceae; g_{——}CAG-217; | |
| | s_{——}CAG-217 sp000436335 | |
| ERR2017650.13 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-81; | |
| | s_{——}CAG-81 sp000435795 | |
| ERR2017651.6 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_AP; | |
| | s_{——}Clostridium_AP sp000509125 | |
| ERR2017654.11 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides | |
| | uniformis | |
| ERR2017659.2 | d_{——}Bacteria; p_{——}Firmicutes_A; | Guild 1 |
| | c_{——}Clostridia; o_{——}Oscillospirales; | |
| | f_{——}Acutalibacteraceae; g_{——}CAG-180; | |
| | s_{——}CAG-180 sp004556705 | |
| ERR2017660.10 | d_{——}Bacteria; p_{——}Firmicutes_A; | Guild 1 |
| | c_{——}Clostridia; o_{——}Lachnospirales; | |
| | f_{——}Lachnospiraceae; g_{——}Eubacterium_G; | |
| | s_{——}Eubacterium_G sp000434315 | |
| ERR2017669.11 | d_{——}Bacteria; p_{——}Firmicutes; | Guild 1 |
| | c_{——}Bacilli; o_{——}Erysipelotrichales; | |
| | f_{——}Erysipelotrichaceae; g_{——}Clostridium_AQ; | |
| | s_{——}Clostridium_AQ sp003481775 | |
| ERR2017669.13 | d_{——}Bacteria; p_{——}Actinobacteriota; | Guild 2 |
| | c_{——}Coriobacteriia; o_{——}Coriobacteriales; | |
| | f_{——}Eggerthellaceae; g_{——}Eggerthella; | |
| | s_{——}Eggerthella lenta | |
| ERR2017669.2 | d_{——}Bacteria; p_{——}Firmicutes_A; | Guild 2 |
| | c_{——}Clostridia; o_{——}Oscillospirales; | |
| | f_{——}Acutalibacteraceae; g_{——}UMGS1071; | |
| | s_{——}UMGS1071 sp900541905 | |
| ERR2017672.11 | d_{——}Bacteria; p_{——}Actinobacteriota; | Guild 2 |
| | c_{——}Coriobacteriia; o_{——}Coriobacteriales; | |
| | f_{——}Coriobacteriaceae; g_{——}Collinsella; | |
| | s_{——}Collinsella sp002232035 | |
| ERR2017680.3 | d_{——}Bacteria; p_{——}Firmicutes_A; | Guild 1 |
| | c_{——}Clostridia; o_{——}Lachnospirales; | |
| | f_{——}Lachnospiraceae; g_{——}UBA9502; | |
| | s_{——}UBA9502 sp003480315 | |
| ERR2017696.13 | d_{——}Bacteria; p_{——}Firmicutes_A; | Guild 2 |
| | c_{——}Clostridia; o_{——}Lachnospirales; | |
| | f_{——}Lachnospiraceae; g_{——}UBA9502; | |
| | s_{——}UBA9502 sp900555625 | |
| ERR2017699.1 | d_{——}Bacteria; p_{——}Firmicutes_A; | Guild 1 |
| | c_{——}Clostridia; o_{——}Lachnospirales; | |
| | f_{——}Lachnospiraceae; g_{——}Eisenbergiella; | |
| | s_{——}Eisenbergiella tayi | |
| ERR2017700.13 | d_{——}Bacteria; p_{——}Firmicutes_A; | Guild 1 |
| | c_{——}Clostridia; o_{——}Lachnospirales; | |
| | f_{——}Lachnospiraceae; g_{——}CAG-81; | |
| | s_{——}CAG-81 sp900066535 | |
| ERR2017701.13 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_D; | |
| | s_{——}Ruminococcus_D bicirculans | |
| ERR2017705.14 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella copri_A | |
| ERR2017706.14 | d_{——}Bacteria; p_{——}Firmicutes_A; | Guild 1 |
| | c_{——}Clostridia; o_{——}Oscillospirales; | |
| | f_{——}Oscillospiraceae; g_{——}Lawsonibacter; | |
| | s_{——}Lawsonibacter sp000177015 | |
| ERR2017714.15 | d_{——}Bacteria; p_{——}Firmicutes_C; | Guild 2 |
| | c_{——}Negativicutes; o_{——}Acidaminococcales; | |
| | f_{——}Acidaminococcaceae; g_{——}Phascolarctobacterium; | |
| | s_{——}Phascolarctobacterium faecium | |
| ERR2017716.3 | d_{——}Bacteria; p_{——}Firmicutes_A; | Guild 1 |
| | c_{——}Clostridia; o_{——}Oscillospirales; | |
| | f_{——}Ruminococcaceae; g_{——}Negativibacillus; | |
| | s_{——}Negativibacillus massiliensis | |
| ERR2017716.6 | d_{——}Bacteria; p_{——}Firmicutes_A; | Guild 2 |
| | c_{——}Clostridia; o_{——}Oscillospirales; | |
| | f_{——}Oscillospiraceae; g_{——}Flavonifractor; | |
| | s_{——}Flavonifractor plautii | |
| ERR2017717.14 | d_{——}Bacteria; p_{——}Firmicutes_A; | Guild 1 |
| | c_{——}Clostridia; o_{——}Lachnospirales; | |
| | f_{——}Lachnospiraceae; g_{——}CAG-127; | |
| | s_{——}CAG-127 sp900319515 | |
| ERR2017729.15 | d_{——}Bacteria; p_{——}Firmicutes_A; | Guild 1 |
| | c_{——}Clostridia; o_{——}Lachnospirales; | |
| | f_{——}Lachnospiraceae; g_{——}Agathobacter; | |
| | s_{——}Agathobacter sp900543445 | |
| ERR2017731.2 | d_{——}Bacteria; p_{——}Firmicutes_A; | Guild 1 |
| | c_{——}Clostridia; o_{——}Lachnospirales; | |
| | f_{——}Anaerotignaceae; g_{——}Anaerotignum; | |
| | s_{——}Anaerotignum faecicola | |
| ERR2017735.12 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| ERR2017736.11 | d_{——}Bacteria; p_{——}Firmicutes_A; | Guild 1 |
| | c_{——}Clostridia; o_{——}Oscillospirales; | |
| | f_{——}Oscillospiraceae; g_{——}ER4; | |
| | s_{——}ER4 sp900550165 | |
| ERR2017739.2 | d_{——}Bacteria; p_{——}Firmicutes_A; | Guild 1 |
| | c_{——}Clostridia; o_{——}Oscillospirales; | |
| | f_{——}Oscillospiraceae; g_{——}Lawsonibacter; | |
| | s_{——}Lawsonibacter sp000177015 | |
| ERR2017740.16 | d_{——}Bacteria; p_{——}Firmicutes_A; | Guild 1 |
| | c_{——}Clostridia; o_{——}Monoglobales_A; | |
| | f_{——}UBA1381; g_{——}CAG-41; | |
| | s_{——}CAG-41 sp900066215 | |
| ERR2017740.2 | d_{——}Bacteria; p_{——}Firmicutes_A; | Guild 1 |
| | c_{——}Clostridia; o_{——}Lachnospirales; | |
| | f_{——}Lachnospiraceae; g_{——}Blautia_A; | |
| | s_{——}Blautia_A sp900541345 | |
| ERR2017741.15 | d_{——}Bacteria; p_{——}Actinobacteriota; | Guild 2 |
| | c_{——}Actinomycetia; o_{——}Actinomycetales; | |
| | f_{——}Bifidobacteriaceae; g_{——}Bifidobacterium; | |
| | s_{——}Bifidobacterium dentium | |
| ERR2017742.15 | d_{——}Bacteria; p_{——}Firmicutes_A; | Guild 1 |
| | c_{——}Clostridia; o_{——}Lachnospirales; | |
| | f_{——}Lachnospiraceae; g_{——}Mediterraneibacter; | |
| | s_{——}Mediterraneibacter faecis | |
| ERR2017746.14 | d_{——}Bacteria; p_{——}Firmicutes_A; | Guild 2 |
| | c_{——}Clostridia; o_{——}Lachnospirales; | |
| | f_{——}Lachnospiraceae; g_{——}Dorea_A; | |
| | s_{——}Dorea_A longicatena_B | |
| ERR2017750.5 | d_{——}Bacteria; p_{——}Firmicutes_A; | Guild 1 |
| | c_{——}Clostridia; o_{——}Oscillospirales; | |
| | f_{——}Oscillospiraceae; g_{——}Lawsonibacter; | |
| | s_{——}Lawsonibacter sp000177015 | |
| ERR2017771.1 | d_{——}Bacteria; p_{——}Firmicutes_A; | Guild 1 |
| | c_{——}Clostridia; o_{——}Lachnospirales; | |
| | f_{——}Lachnospiraceae; g_{——}Agathobacter; | |
| | s_{——}Agathobacter rectalis | |
| ERR2017781.12 | d_{——}Bacteria; p_{——}Firmicutes; | Guild 1 |
| | c_{——}Bacilli; o_{——}Erysipelotrichales; | |
| | f_{——}Erysipelotrichaceae; g_{——}Holdemanella; | |
| | s_{——}Holdemanella biformis | |
| ERR2017814.13 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia; | |
| | s_{——}Blautia stercoris | |
| ERR2726410.2 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger formicilis | |
| ERR2726411.1 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella nigrescens | |
| ERR2726419.1 | d_{——}Bacteria; p_{——}Firmicutes_A; | Guild 1 |
| | c_{——}Clostridia; o_{——}Oscillospirales; | |
| | f_{——}Acutalibacteraceae; g_{——}Acutalibacter; | |
| | s_{——}Acutalibacter sp000435395 | |
| ERR2726420.13 | d_{——}Bacteria; p_{——}Firmicutes_A; | Guild 2 |
| | c_{——}Clostridia; o_{——}Lachnospirales; | |
| | f_{——}Lachnospiraceae; g_{——}Butyrivibrio_A; | |
| | s_{——}Butyrivibrio_A crossotus | |
| ERR2726422.1 | d_{——}Bacteria; p_{——}Firmicutes_A; | Guild 2 |
| | c_{——}Clostridia; o_{——}Lachnospirales; | |
| | f_{——}Lachnospiraceae; g_{——}Roseburia; | |
| | s_{——}Roseburia intestinalis | |
| ERR2726423.2 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-170; | |
| | s_{——}CAG-170 sp900548625 | |
| ERR2726433.15 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola sp000434735 | |
| ERR2726447.12 | d_{——}Bacteria; p_{——}Firmicutes; | Guild 1 |
| | c_{——}Bacilli; o_{——}RFN20; | |
| | f_{——}CAG-288; g_{——}CAG-568; | |
| | s_{——}CAG-568 sp000434395 | |
| ERR2726447.19 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella sp002265625 | |
| ERR2726447.2 | d_{——}Bacteria; p_{——}Firmicutes_A; | Guild 1 |
| | c_{——}Clostridia; o_{——}Lachnospirales; | |
| | f_{——}Lachnospiraceae; g_{——}KLE1615; | |
| | s_{——}KLE1615 sp900066985 | |
| ERR2726451.2 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides merdae | |
| ERR2726452.15 | d_{——}Bacteria; p_{——}Bacteroidota; | Guild 2 |
| | c_{——}Bacteroidia; o_{——}Bacteroidales; | |
| | f_{——}Rikenellaceae; g_{——}Alistipes; | |
| | s_{——}Alistipes putredinis | |
| ERR2726452.9 | d_{——}Bacteria; p_{——}Firmicutes_A; | Guild 1 |
| | c_{——}Clostridia; o_{——}Oscillospirales; | |
| | f_{——}Ruminococcaceae; g_{——}Negativibacillus; | |
| | s_{——}Negativibacillus sp900547455 | |
| ERR2726458.13 | d_{——}Bacteria; p_{——}Firmicutes_A; | Guild 2 |
| | c_{——}Clostridia; o_{——}Lachnospirales; | |
| | f_{——}Lachnospiraceae; g_{——}Mediterraneibacter; | |
| | s_{——}Mediterraneibacter lactaris | |
| ERR2726476.5 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola sp000432735 | |
| ERR2726486.19 | d_{——}Bacteria; p_{——}Bacteroidota; | Guild 2 |
| | c_{——}Bacteroidia; o_{——}Bacteroidales; | |
| | f_{——}Rikenellaceae; g_{——}Alistipes; | |
| | s_{——}Alistipes communis | |
| ERR2726493.4 | d_{——}Bacteria; p_{——}Firmicutes_A; | Guild 1 |
| | c_{——}Clostridia; o_{——}Oscillospirales; | |
| | f_{——}Oscillospiraceae; g_{——}Dysosmobacter; | |
| | s_{——}Dysosmobacter welbionis | |
| ERR2726493.5 | d_{——}Bacteria; p_{——}Firmicutes_A; | Guild 1 |
| | c_{——}Clostridia; o_{——}Oscillospirales; | |
| | f_{——}Acutalibacteraceae; g_{——}UMGS403; | |
| | s_{——}UMGS403 sp900540275 | |
| ERR2726498.24 | d_{——}Bacteria; p_{——}Bacteroidota; | Guild 2 |
| | c_{——}Bacteroidia; o_{——}Bacteroidales; | |
| | f_{——}Muribaculaceae; g_{——}Muribaculum; | |
| | s_{——}Muribaculum sp003150235 | |
| ERR2726502.1 | d_{——}Bacteria; p_{——}Firmicutes_A; | Guild 1 |
| | c_{——}Clostridia; o_{——}Lachnospirales; | |
| | f_{——}Lachnospiraceae; g_{——}UBA11774; | |
| | s_{——}UBA11774 sp003507655 | |
| ERR2726522.5 | d_{——}Bacteria; p_{——}Firmicutes_A; | Guild 1 |
| | c_{——}Clostridia_A; o_{——}Christensenellales; | |
| | f_{——}CAG-74; g_{——}SFFH01; | |
| | s_{——}SFFH01 sp900542395 | |
| ERR2726527.4 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides uniformis | |
| ERR2726532.18 | d_{——}Bacteria; p_{——}Firmicutes_A; | Guild 1 |
| | c_{——}Clostridia; o_{——}Oscillospirales; | |
| | f_{——}Acutalibacteraceae; g_{——}Pseudoruminococcus; | |
| | s_{——}Pseudoruminococcus massiliensis | |
| ERR2855787.12 | d_{——}Bacteria; p_{——}Firmicutes_A; | Guild 1 |
| | c_{——}Clostridia; o_{——}Oscillospirales; | |
| | f_{——}Ruminococcaceae; g_{——}CAG-115; | |
| | s_{——}CAG-115 sp003531585 | |
| ERR2855790.1 | d_{——}Bacteria; p_{——}Firmicutes_A; | Guild 1 |
| | c_{——}Clostridia; o_{——}Oscillospirales; | |
| | f_{——}Oscillospiraceae; g_{——}Dysosmobacter; | |
| | s_{——}Dysosmobacter sp001916835 | |
| ERR2855795.12 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium sp900539945 | |
| ERR2855800.13 | d_{——}Bacteria; p_{——}Proteobacteria; | Guild 1 |
| | c_{——}Gammaproteobacteria; o_{——}Burkholderiales; | |
| | f_{——}Burkholderiaceae; g_{——}Duodenibacillus; | |
| | s_{——}Duodenibacillus sp003472385 | |
| ERR2855808.13 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-81; | |
| | s_{——}CAG-81 sp900066535 | |
| ERR2855814.14 | d_{——}Bacteria; p_{——}Firmicutes_A; | Guild 1 |
| | c_{——}Clostridia; o_{——}Lachnospirales; | |
| | f_{——}Lachnospiraceae; g_{——}TF01-11; | |
| | s_{——}TF01-11 sp001414325 | |
| ERR2855814.4 | d_{——}Bacteria; p_{——}Firmicutes_A; | Guild 2 |
| | c_{——}Clostridia; o_{——}Lachnospirales; | |
| | f_{——}Lachnospiraceae; g_{——}UBA9502; | |
| | s_{——}UBA9502 sp003480315 | |
| ERR2855821.2 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-81; | |
| | s_{——}CAG-81 sp900066535 | |
| ERR2855825.1 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola massiliensis | |
| ERR2855828.7 | d_{——}Bacteria; p_{——}Firmicutes_A; | Guild 2 |
| | c_{——}Clostridia; o_{——}Lachnospirales; | |
| | f_{——}Lachnospiraceae; g_{——}UBA9502; | |
| | s_{——}UBA9502 sp003478505 | |
| ERR2855831.6 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Duodenibacillus; | |
| | s_{——}Duodenibacillus massiliensis | |
| ERR2855838.18 | d_{——}Bacteria; p_{——}Firmicutes_A; | Guild 2 |
| | c_{——}Clostridia; o_{——}Lachnospirales; | |
| | f_{——}Lachnospiraceae; g_{——}TF01-11; | |
| | s_{——}TF01-11 sp003524945 | |
| ERR2855840.6 | d_{——}Bacteria; p_{——}Firmicutes_A; | Guild 2 |
| | c_{——}Clostridia; o_{——}Oscillospirales; | |
| | f_{——}Ruminococcaceae; g_{——}Gemmiger; | |
| | s_{——}Gemmiger formicilis | |
| ERR2855844.18 | d_{——}Bacteria; p_{——}Firmicutes_A; | Guild 2 |
| | c_{——}Clostridia; o_{——}Lachnospirales; | |
| | f_{——}Lachnospiraceae; g_{——}Clostridium_Q; | |
| | s_{——}Clostridium_Q sp003024715 | |
| ERR2855857.7 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Mesosutterella; | |
| | s_{——}Mesosutterella multiformis | |
| ERR2855864.8 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella_stercorea | |
| ERR2855871.6 | d_{——}Bacteria; p_{——}Firmicutes_A; | Guild 2 |
| | c_{——}Clostridia; o_{——}Lachnospirales; | |
| | f_{——}Lachnospiraceae; g_{——}Lachnospira; | |
| | s_{——}Lachnospira sp900545725 | |
| ERR2855880.6 | d_{——}Bacteria; p_{——}Firmicutes_A; | Guild 2 |
| | c_{——}Clostridia; o_{——}Oscillospirales; | |
| | f_{——}Ruminococcaceae; g_{——}Faecalibacterium; | |
| | s_{——}Faecalibacterium prausnitzii_G | |
| ERR2855886.18 | d_{——}Bacteria; p_{——}Firmicutes_A; | Guild 1 |
| | c_{——}Clostridia; o_{——}Oscillospirales; | |
| | f_{——}Acutalibacteraceae; g_{——}Ruminococcus_E; | |
| | s_{——}Ruminococcus_E sp003526955 | |
| ERR2855908.3 | d_{——}Bacteria; p_{——}Firmicutes_C; | Guild 2 |
| | c_{——}Negativicutes; o_{——}Veillonellales; | |
| | f_{——}Dialisteraceae; g_{——}Dialister; | |
| | s_{——}Dialister sp900538805 | |
| ERR2855923.15 | d_{——}Bacteria; p_{——}Firmicutes_A; | Guild 1 |
| | c_{——}Clostridia; o_{——}Oscillospirales; | |
| | f_{——}Ruminococcaceae; g_{——}Ruminococcus_C; | |
| | s_{——}Ruminococcus_C sp000433635 | |
| ERR2855933.19 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_G; | |
| | s_{——}Eubacterium_G sp000435815 | |
| ERR2855935.6 | d_{——}Bacteria; p_{——}Firmicutes_A; | Guild 2 |
| | c_{——}Clostridia; o_{——}Oscillospirales; | |
| | f_{——}Acutalibacteraceae; g_{——}CAG-177; | |
| | s_{——}CAG-177 sp003514385 | |
| ERR2855935.9 | d_{——}Bacteria; p_{——}Bacteroidota; | Guild 2 |
| | c_{——}Bacteroidia; o_{——}Bacteroidales; | |
| | f_{——}Rikenellaceae; g_{——}Alistipes_A; | |
| | s_{——}Alistipes_A sp900539755 | |
| ERR2855947.28 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-83; | |
| | s_{——}CAG-83 sp900552475 | |
| ERR2855947.3 | d_{——}Bacteria; p_{——}Firmicutes_A; | Guild 1 |
| | c_{——}Clostridia; o_{——}Oscillospirales; | |
| | f_{——}Oscillospiraceae; g_{——}Dysosmobacter; | |
| | s_{——}Dysosmobacter sp001916835 | |
| ERR68856.3 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_E | |
| ERR688507.14 | d_{——}Bacteria; p_{——}Firmicutes; | Guild 2 |
| | c_{——}Bacilli; o_{——}Lactobacilles; | |
| | f_{——}Streptococcaceae; g_{——}Streptococcus; | |
| | s_{——}Streptococcus thermophilus | |
| ERR688513.3 | d_{——}Bacteria; p_{——}Actinobacteriota; | Guild 2 |
| | c_{——}Coriobacteriia; o_{——}Coriobacteriales; | |
| | f_{——}Coriobacteriaceae; g_{——}Collinsella; | |
| | s_{——}Collinsella sp900541745 | |
| ERR688518.6 | d_{——}Bacteria; p_{——}Firmicutes_A; | Guild 2 |
| | c_{——}Clostridia; o_{——}Lachnospirales; | |
| | f_{——}Lachnospiraceae; g_{——}Anaerostipes; | |
| | s_{——}Anaerostipes sp900066705 | |
| ERR688520.2 | d_{——}Bacteria; p_{——}Firmicutes_A; | Guild 2 |
| | c_{——}Clostridia; o_{——}Oscillospirales; | |
| | f_{——}Acutalibacteraceae; g_{——}Eubacterium_R; | |
| | s_{——}Eubacterium_R sp000436835 | |
| ERR688520.8 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-317; | |
| | s_{——}CAG-317 sp900543415 | |
| ERR688521.4 | d_{——}Bacteria; p_{——}Firmicutes_A; | Guild 1 |
| | c_{——}Clostridia; o_{——}Lachnospirales; | |
| | f_{——}Lachnospiraceae; g_{——}Ruminococcus_A; | |
| | s_{——}Ruminococcus_A sp003011855 | |
| ERR688522.8 | d_{——}Bacteria; p_{——}Firmicutes_A; | Guild 1 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| ERR688522.9 | d_{——}Bacteria; p_{——}Firmicutes_A; | Guild 2 |
| | c_{——}Clostridia; o_{——}Lachnospirales; | |
| | f_{——}Lachnospiraceae; g_{——}Dorea; | |
| | s_{——}Dorea formicigenerans | |
| ERR688524.6 | d_{——}Bacteria; p_{——}Firmicutes_A; | Guild 2 |
| | c_{——}Clostridia; o_{——}Lachnospirales; | |
| | f_{——}Lachnospiraceae; g_{——}Enterocloster; | |
| | s_{——}Enterocloster sp000155435 | |
| ERR688528.23 | d_{——}Bacteria; p_{——}Firmicutes_A; | Guild 2 |
| | c_{——}Clostridia; o_{——}Lachnospirales; | |
| | f_{——}Lachnospiraceae; g_{——}Bacteroides_F; | |
| | s_{——}Bacteroides_F pectinophilus | |
| ERR688529.17 | d_{——}Bacteria; p_{——}Actinobacteriota; | Guild 2 |
| | c_{——}Coriobacteriia; o_{——}Coriobacteriales; | |
| | f_{——}Coriobacteriaceae; g_{——}Collinsella; | |
| | s_{——}Collinsella sp900758375 | |
| ERR688536.9 | d_{——}Bacteria; p_{——}Firmicutes_A; | Guild 1 |
| | c_{——}Clostridia; o_{——}Lachnospirales; | |
| | f_{——}Lachnospiraceae; g_{——}Ruminococcus_A; | |
| | s_{——}Ruminococcus_A sp003011855 | |
| ERR688537.6 | d_{——}Bacteria; p_{——}Actinobacteriota; | Guild 2 |
| | c_{——}Actinomycetia; o_{——}Actinomycetales; | |
| | f_{——}Bifidobacteriaceae; g_{——}Bifidobacterium; | |
| | s_{——}Bifidobacterium pullorum | |
| ERR688539.8 | d_{——}Bacteria; p_{——}Firmicutes_A; | Guild 1 |
| | c_{——}Clostridia; o_{——}Lachnospirales; | |
| | f_{——}Lachnospiraceae; g_{——}Blautia; | |
| | s_{——}Blautia stercoris | |
| ERR688558.20 | d_{——}Bacteria; p_{——}Actinobacteriota; | Guild 2 |
| | c_{——}Coriobacteriia; o_{——}Coriobacteriales; | |
| | f_{——}Eggerthellaceae; g_{——}CAG-1427; | |
| | s_{——}CAG-1427 sp000436075 | |
| ERR688558.6 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger qucibialis | |
| ERR688559.18 | d_{——}Bacteria; p_{——}Actinobacteriota; | Guild 2 |
| | c_{——}Coriobacteriia; o_{——}Coriobacteriales; | |
| | f_{——}Eggerthellaceae; g_{——}CAG-1427; | |
| | s_{——}CAG-1427 sp000431675 | |
| ERR688563.10 | d_{——}Bacteria; p_{——}Firmicutes_A; | Guild 2 |
| | c_{——}Clostridia; o_{——}Oscillospirales; | |
| | f_{——}Acutalibacteraceae; g_{——}UBA1417; | |
| | s_{——}UBA1417 sp003531055 | |
| ERR688564.13 | d_{——}Bacteria; p_{——}Firmicutes_A; | Guild 1 |
| | c_{——}Clostridia; o_{——}Lachnospirales; | |
| | f_{——}Lachnospiraceae; g_{——}Fusicatenibacter; | |
| | s_{——}Fusicatenibacter saccharivorans | |
| ERR688567.8 | d_{——}Bacteria; p_{——}Firmicutes_A; | Guild 1 |
| | c_{——}Clostridia; o_{——}Oscillospirales; | |
| | f_{——}Oscillospiraceae; g_{——}NK3B98; | |
| | s_{——}NK3B98 sp900545815 | |
| ERR688577.19 | d_{——}Bacteria; p_{——}Proteobacteria; | Guild 2 |
| | c_{——}Gammaproteobacteria; o_{——}Enterobacterales; | |
| | f_{——}Enterobacteriaceae; g_{——}Escherichia; | |
| | s_{——}Escherichia dysenteriae | |
| ERR688589.10 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-110; | |
| | s_{——}CAG-110 sp000434635 | |
| ERR688598.5 | d_{——}Bacteria; p_{——}Actinobacteriota; | Guild 1 |
| | c_{——}Actinomycetia; o_{——}Actinomycetales; | |
| | f_{——}Bifidobacteriaceae; g_{——}Bifidobacterium; | |
| | s_{——}Bifidobacterium breve | |
| ERR68864.10 | d_{——}Bacteria; p_{——}Firmicutes_A; | Guild 1 |
| | c_{——}Clostridia; o_{——}Lachnospirales; | |
| | f_{——}Lachnospiraceae; g_{——}Eubacterium_G; | |
| | s_{——}Eubacterium_G sp000434315 | |
| ERR68868.9 | d_{——}Bacteria; p_{——}Firmicutes_A; | Guild 1 |
| | c_{——}Clostridia; o_{——}Lachnospirales; | |
| | f_{——}Lachnospiraceae; g_{——}Anaerobutyricum; | |
| | s_{——}Anaerobutyricum soehngenii | |
| ERR688610.11 | d_{——}Bacteria; p_{——}Firmicutes_A; | Guild 1 |
| | c_{——}Clostridia_A; o_{——}Christensenellales; | |
| | f_{——}CAG-138; g_{——}PeH17; | |
| | s_{——}PeH17 sp900542285 | |
| ERR688610.9 | d_{——}Bacteria; p_{——}Firmicutes_A; | Guild 2 |
| | c_{——}Clostridia; o_{——}Lachnospirales; | |
| | f_{——}Lachnospiraceae; g_{——}Roseburia; | |
| | s_{——}Roseburia hominis | |
| ERR688612.12 | d_{——}Bacteria; p_{——}Firmicutes_A; | Guild 1 |
| | c_{——}Clostridia; o_{——}Lachnospirales; | |
| | f_{——}Lachnospiraceae; g_{——}Fusicatenibacter; | |
| | s_{——}Fusicatenibacter saccharivorans | |
| ERR688615.3 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A obeum | |
| ERR688615.8 | d_{——}Bacteria; p_{——}Firmicutes_A; | Guild 1 |
| | c_{——}Clostridia; o_{——}Lachnospirales; | |
| | f_{——}CAG-274; g_{——}CAG-274; | |
| | s_{——}CAG-274 sp000432155 | |
| ERR688617.7 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger sp900540775 | |
| ERR688623.13 | d_{——}Bacteria; p_{——}Firmicutes_A; | Guild 1 |
| | c_Clostridia; o_{——}Oscillospirales; | |
| | f_{——}Butyricicoccaceae; g_{——}Agathobaculum; | |
| | s_{——}Agathobaculum butyriciproducens | |
| ERR688627.12 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A wexlerae | |
| ERR688629.1 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides uniformis | |
| ERR688629.15 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Bulleidia; | |
| | s_{——}Bulleidia sp900539965 | |
| ERR688631.6 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides stercoris | |
| ERR688640.13 | d_{——}Bacteria; p_{——}Firmicutes; | Guild 2 |
| | c_{——}Bacilli; o_{——}Erysipelotrichales; | |
| | f_{——}Erysipelotrichaceae; g_{——}Clostridium_AQ; | |
| | s_{——}Clostridium_AQ sp900552125 | |
| ERR688642.6 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger qucibialis | |
| ERR688645.6 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides fragilis | |
| HD-20.2 | d_{——}Bacteria; p_{——}Firmicutes_A; | Guild 2 |
| | c_{——}Clostridia; o_{——}Oscillospirales; | |
| | f_{——}Ruminococcaceae; g_{——}Faecalibacterium; | |
| | s_{——}Faecalibacterium sp900539945 | |
| HD-63.13 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——} | |
| HD-76.2 | d_{——}Bacteria; p_{——}Fusobacteriota; | Guild 2 |
| | c_{——}Fusobacteriia; o_{——}Fusobacteriales; | |
| | f_{——}Fusobacteriaceae; g_{——}Fusobacterium_A; | |
| | s_{——}Fusobacterium_A sp900543175 | |
| HD-8.10 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_F; | |
| | s_{——}Eubacterium_F sp003491505 | |
| LD-26.6 | d_{——}Bacteria; p_{——}Firmicutes_A; | Guild 1 |
| | c_{——}Clostridia; o_{——}Lachnospirales; | |
| | f_{——}Lachnospiraceae; g_{——}TF01-11; | |
| | s_{——}TF01-11 sp000436755 | |
| LD-33.1 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides ndongoniae | |
| LD-35.30 | d_{——}Bacteria; p_{——}Firmicutes_A; | Guild 2 |
| | c_{——}Clostridia; o_{——}Lachnospirales; | |
| | f_{——}Anaerotignaceae; g_{——}Anaerotignum; | |
| | s_{——} | |
| LD-39.9 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides distasonis | |
| LD-7.2 | d_{——}Bacteria; p_{——}Firmicutes_A; | Guild 1 |
| | c_{——}Clostridia; o_{——}Lachnospirales; | |
| | f_{——}Lachnospiraceae; g_{——}Agathobacter; | |
| | s_{——}Agathobacter faecis | |
| LD-75.3 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides gordonii | |
| MSM79H8B.5 | d_{——}Bacteria; p_{——}Bacteroidota; | Guild 2 |
| | c_{——}Bacteroidia; o_{——}Bacteroidales; | |
| | f_{——}Bacteroidaceae; g_{——}Phocaeicola; | |
| | s_{——}Phocaeicola dorei | |
| PSMA264O.3 | d_{——}Bacteria; p_{——}Firmicutes_A; | Guild 2 |
| | c_{——}Clostridia; o_{——}Oscillospirales; | |
| | f_{——}Ruminococcaceae; g_{——}Faecalibacterium; | |
| | s_{——}Faecalibacterium prausnitzii_D | |
| SRR341654.1 | d_{——}Bacteria; p_{——}Verrucomicrobiota; | Guild 2 |
| | c_{——}Verrucomicrobiae; o_{——}Verrucomicrobiales; | |
| | f_{——}Akkermansiaceae; g_{——}Akkermansia; | |
| | s_{——}Akkermansia muciniphila | |
| SRR341680.2 | d_{——}Bacteria; p_{——}Firmicutes_A; | Guild 2 |
| | c_{——}Clostridia; o_{——}Lachnospirales; | |
| | f_{——}Lachnospiraceae; g_{——}Roseburia; | |
| | s_{——}Roseburia sp003470905 | |
| SRR341724.5 | d_{——}Bacteria; p_{——}Actinobacteriota; | Guild 1 |
| | c_{——}Actinomycetia; o_{——}Actinomycetales; | |
| | f_{——}Bifidobacteriaceae; g_{——}Bifidobacterium; | |
| | s_{——}Bifidobacterium pseudocatenulatum | |
| SRR413559.10 | d_{——}Bacteria; p_{——}Firmicutes_A; | Guild 2 |
| | c_{——}Clostridia_A; o_{——}Christensenellales; | |
| | f_{——}CAG-74; g_{——}UBA11524; | |
| | s_{——}UBA11524 sp000437595 | |
| SRR413567.10 | d_{——}Bacteria; p_{——}Bacteroidota; | Guild 2 |
| | c_{——}Bacteroidia; o_{——}Bacteroidales; | |
| | f_{——}Tannerellaceae; g_{——}Parabacteroides; | |
| | s_{——}Parabacteroides goldsteinii | |
| SRR413584.9 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-603; | |
| | s_{——}CAG-603 sp900066105 | |
| SRR413588.5 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella sp900551275 | |
| SRR413635.13 | d_{——}Bacteria; p_{——}Bacteroidota; | Guild 1 |
| | c_{——}Bacteroidia; o_{——}Bacteroidales; | |
| | f_{——}Bacteroidaceae; g_{——}Prevotella; | |
| | s_{——}Prevotella buccae | |
| SRR413635.5 | d_{——}Bacteria; p_{——}Firmicutes_A; | Guild 1 |
| | c_{——}Clostridia; o_{——}Lachnospirales; | |
| | f_{——}Lachnospiraceae; g_{——}CAG-45; | |
| | s_{——}CAG-45 sp000438375 | |
| SRR413641.2 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides ovatus | |
| SRR413642.4 | d_{——}Bacteria; p_{——}Firmicutes_A; | Guild 2 |
| | c_{——}Clostridia; o_{——}Lachnospirales; | |
| | f_{——}Lachnospiraceae; g_{——}Lachnospira; | |
| | s_{——}Lachnospira sp000437735 | |
| SRR413642.5 | d_{——}Bacteria; p_{——}Firmicutes_A; | Guild 2 |
| | c_{——}Clostridia; o_{——}Lachnospirales; | |
| | f_{——}Lachnospiraceae; g_{——}Agathobacter; | |
| | s_{——}Agathobacter faecis | |
| SRR413655.3 | d_{——}Bacteria; p_{——}Firmicutes_A; | Guild 2 |
| | c_{——}Clostridia; o_{——}Lachnospirales; | |
| | f_{——}Lachnospiraceae; g_{——}Lachnospira; | |
| | s_{——}Lachnospira rogosae_A | |
| SRR413677.1 | d_{——}Bacteria; p_{——}Bacteroidota; | Guild 2 |
| | c_{——}Bacteroidia; o_{——}Bacteroidales; | |
| | f_{——}Marinifilaceae; g_{——}Odoribacter; | |
| | s_{——}Odoribacter splanchnicus | |
| SRR413691.1 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotella; | |
| | s_{——}Prevotella sp900313215 | |
| SRR413694.1 | d_{——}Bacteria; p_{——}Firmicutes_A; | Guild 2 |
| | c_{——}Clostridia; o_{——}Lachnospirales; | |
| | f_{——}Lachnospiraceae; g_{——}Lachnospira; | |
| | s_{——}Lachnospira eligens_A | |
| SRR413729.3 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides caccae | |
| SRR413736.4 | d_{——}Bacteria; p_{——}Bacteroidota; | Guild 2 |
| | c_{——}Bacteroidia; o_{——}Bacteroidales; | |
| | f_{——}Marinifilaceae; g_{——}Butyricimonas; | |
| | s_{——}Butyricimonas virosa | |
| SRR413753.22 | d_{——}Bacteria; p_{——}Bacteroidota; | Guild 2 |
| | c_{——}Bacteroidia; o_{——}Bacteroidales; | |
| | f_{——}Rikenellaceae; g_{——}Alistipes; | |
| | s_{——}Alistipes communis | |
| SRR413755.1 | d_{——}Bacteria; p_{——}Firmicutes_A; | Guild 1 |
| | c_{——}Clostridia; o_{——}Lachnospirales; | |
| | f_{——}Lachnospiraceae; g_{——}Enterocloster; | |
| | s_{——}Enterocloster aldenensis | |
| SRR413761.12 | d_{——}Bacteria; p_{——}Firmicutes_A; | Guild 2 |
| | c_{——}Clostridia; o_{——}Lachnospirales; | |
| | f_{——}Anaerotignaceae; g_{——}Anaerotignum; | |
| | s_{——}Anaerotignum faecicola | |
| SRR413762.3 | d_{——}Bacteria; p_{——}Firmicutes_A; | Guild 2 |
| | c_{——}Clostridia; o_{——}Lachnospirales; | |
| | f_{——}Lachnospiraceae; g_{——}CAG-95; | |
| | s_{——}CAG-95 sp000438155 | |
| SRR413762.6 | d_{——}Bacteria; p_{——}Bacteroidota; | Guild 2 |
| | c_{——}Bacteroidia; o_{——}Bacteroidales; | |
| | f_{——}Marinifilaceae; g_{——}Odoribacter; | |
| | s_{——}Odoribacter massiliensis | |
| SRR5279219.7 | d_{——}Bacteria; p_{——}Firmicutes_A; | Guild 2 |
| | c_{——}Clostridia; o_{——}Oscillospirales; | |
| | f_{——}Acutalibacteraceae; g_{——}Ruminococcus_E; | |
| | s_{——}Ruminococcus_E sp900314705 | |
| SRR5279222.3 | d_{——}Bacteria; p_{——}Firmicutes_A; | Guild 2 |
| | c_{——}Clostridia; o_{——}Lachnospirales; | |
| | f_{——}Lachnospiraceae; g_{——}Blautia_A; | |
| | s_{——}Blautia_A sp003480185 | |
| SRR5279239.4 | d_{——}Bacteria; p_{——}Firmicutes; | Guild 2 |
| | c_{——}Bacilli; o_{——}Lactobacillus; | |
| | f_{——}Lactobacillaceae; g_{——}Limosilactobacillus; | |
| | s_{——}Limosilactobacillus mucosae | |
| SRR5279253.7 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Sutterella; | |
| | s_{——} | |
| SRR5279269.27 | d_{——}Bacteria; p_{——}Firmicutes_A; | Guild 2 |
| | c_{——}Clostridia; o_{——}Lachnospirales; | |
| | f_{——}Lachnospiraceae; g_{——}Eubacterium_G; | |
| | s_{——}Eubacterium_G ventriosum | |
| SRR5279278.2 | d_{——}Bacteria; p_{——}Bacteroidota; | Guild 2 |
| | c_{——}Bacteroidia; o_{——}Bacteroidales; | |
| | f_{——}Bacteroidaceae; g_{——}Prevotellamassilia; | |
| | s_{——}Prevotellamassilia sp900539625 | |
| SRR5279285.2 | d_{——}Bacteria; p_{——}Firmicutes; | Guild 2 |
| | c_{——}Bacilli; o_{——}Erysipelotrichales; | |
| | f_{——}Erysipelatoclostridiaceae; g_{——}Faecalibacillus; | |
| | s_{——}Faecalibacillus intestinalis | |
| SRR5279303.4 | d_{——}Bacteria; p_{——}Firmicutes_C; | Guild 2 |
| | c_{——}Negativicutes; o_{——}Veillonellales; | |
| | f_{——}Veillonellaceae; g_{——}Veillonella; | |
| | s_{——}Veillonella parvula_A | |
| SRR6468507.8 | d_{——}Bacteria; p_{——}Firmicutes_A; | Guild 1 |
| | c_{——}Clostridia; o_{——}Lachnospirales; | |
| | f_{——}Lachnospiraceae; g_{——}Blautia_A; | |
| | s_{——}Blautia_A wexlerae | |
| SRR6468510.13 | d_{——}Bacteria; p_{——}Bacteroidota; | Guild 2 |
| | c_{——}Bacteroidia; o_{——}Bacteroidales; | |
| | f_{——}UBA932; g_{——}RC9; | |
| | s_{——}RC9 sp000434935 | |
| SRR6468523.9 | d_{——}Bacteria; p_{——}Firmicutes_A; | Guild 1 |
| | c_{——}Clostridia; o_{——}Oscillospirales; | |
| | f_{——}Oscillospiraceae; g_{——}CAG-170; | |
| | s_{——}CAG-170 sp000432135 | |
| SRR6468543.7 | d_{——}Bacteria; p_{——}Firmicutes_A; | Guild 1 |
| | c_{——}Clostridia; o_{——}Oscillospirales; | |
| | f_{——}Acutalibacteraceae; g_{——}Eubacterium_R; | |
| | s_{——}Eubacterium_R sp900540305 | |
| SRR6468590.5 | d_{——}Bacteria; p_{——}Firmicutes_A; | Guild 1 |
| | c_{——}Clostridia; o_{——}Lachnospirales; | |
| | f_{——}Lachnospiraceae; g_{——}Eubacterium_G; | |
| | s_{——}Eubacterium_G sp000432355 | |
| SRR6468594.3 | d_{——}Bacteria; p_{——}Firmicutes_A; | Guild 2 |
| | c_{——}Clostridia; o_{——}Lachnospirales; | |
| | f_{——}Lachnospiraceae; g_{——}V9D3004; | |
| | s_{——}V9D3004 sp900760345 | |
| SRR6468602.3 | d_{——}Bacteria; p_{——}Proteobacteria; | Guild 2 |
| | c_{——}Gammaproteobacteria; o_{——}Burkholderiales; | |
| | f_{——}Burkholderiaceae; g_{——}Parasutterella; | |
| | s_{——}Parasutterella sp000980495 | |
| SRR6468612.10 | d_{——}Bacteria; p_{——}Firmicutes_A; | Guild 2 |
| | c_{——}Clostridia; o_{——}Oscillospirales; | |
| | f_{——}Acutalibacteraceae; g_{——}Acutalibacter; | |
| | s_{——}Acutalibacter timonensis | |
| SRR6468618.9 | d_{——}Bacteria; p_{——}Firmicutes_A; | Guild 2 |
| | c_{——}Clostridia; o_{——}Lachnospirales; | |
| | f_{——}Lachnospiraceae; g_{——}Sellimonas; | |
| | s_{——}Sellimonas intestinalis | |
| SRR6468619.8 | d_{——}Bacteria; p_{——}Firmicutes_A; | Guild 2 |
| | c_{——}Clostridia; o_{——}Lachnospirales; | |
| | f_{——}Lachnospiraceae; g_{——}Enterocloster; | |
| | s_{——}Enterocloster bolteae | |
| SRR6468633.4 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger sp900539695 | |
| SRR6468637.9 | d_{——}Bacteria; p_{——}Bacteroidota; | Guild 2 |
| | c_{——}Bacteroidia; o_{——}Bacteroidales; | |
| | f_{——}Bacteroidaceae; g_{——}Bacteroides; | |
| | s_{——}Bacteroides uniformis | |
| SRR6468639.4 | d_{——}Bacteria; p_{——}Firmicutes; | Guild 1 |
| | c_{——}Bacilli; o_{——}Lactobacillus; | |
| | f_{——}Streptococcaceae; g_{——}Streptococcus; | |
| | s_{——}Streptococcus lutetiensis | |
| SRR6468651.2 | d_{——}Bacteria; p_{——}Firmicutes_A; | Guild 2 |
| | c_{——}Clostridia; o_{——}Lachnospirales; | |
| | f_{——}Lachnospiraceae; g_{——}Faecalimonas; | |
| | s_{——} | |
| SRR6468652.5 | d_{——}Bacteria; p_{——}Firmicutes_C; | Guild 2 |
| | c_{——}Negativicutes; o_{——}Veillonellales; | |
| | f_{——}Megasphaeraceae; g_{——}Anaeroglobus; | |
| | s_{——}Anaeroglobus micronuciformis | |
| SRR6468672.4 | d_{——}Bacteria; p_{——}Firmicutes_C; | Guild 2 |
| | c_{——}Negativicutes; o_{——}Acidaminococcales; | |
| | f_{——}Acidaminococcaceae; g_{——}Acidaminococcus; | |
| | s_{——}Acidaminococcus intestini | |
| SRR6468684.3 | d_{——}Bacteria; p_{——}Firmicutes_A; | Guild 1 |
| | c_{——}Clostridia; o_{——}Lachnospirales; | |
| | f_{——}Lachnospiraceae; g_{——}Lachnospira; | |
| | s_{——}Lachnospira sp000436475 | |
| SRR6468684.5 | d_{——}Bacteria; p_{——}Firmicutes_A; | Guild 1 |
| | c_{——}Clostridia; o_{——}Lachnospirales; | |
| | f_{——}Lachnospiraceae; g_{——}KLE1615; | |
| | s_{——}KLE1615 sp900066985 | |
| SRR6468688.21 | d_{——}Bacteria; p_{——}Firmicutes_A; | Guild 1 |
| | c_{——}Clostridia; o_{——}Oscillospirales; | |
| | f_{——}Acutalibacteraceae; g_{——}Eubacterium_R; | |
| | s_{——}Eubacterium_R sp000431535 | |
| SRR6468699.2 | d_{——}Bacteria; p_{——}Firmicutes_A; | Guild 1 |
| | c_{——}Clostridia; o_{——}Oscillospirales; | |
| | f_{——}Ruminococcaceae; g_{——}Gemmiger; | |
| | s_{——}Gemmiger formicilis | |

The bacterial species listed in Table 1, Table 2, and FIGS. 42A-42XX were identified by metagenomic sequencing of genomic DNA isolated from human fecal samples and determined to be part of two competing microbiota guilds relative to at least one biological characteristic, as described in the Examples. Briefly, genomic DNA was isolated from each fecal sample was sequenced by next generation sequencing and contigs for microorganism genome sequences were constructed de novo. Generally, the contigs identified for each microorganism are predicted to represent greater than 95% of the entire genome for the microorganism. Genomic constructs having less than 1% sequence divergence from each other were combined and defined to be from the same microorganism. Genomic contigs for each microorganism listed in Table 1, Table 2, and FIGS. 42A-42XX are provided in the sequence listing filed with the application. The taxonomic assignment of each microorganism is given in Table 1, Table 2, or FIGS. 42A-42XX. Correspondence between the sequence identifier assigned to each contig and the microorganism to which it belongs is provided in FIG. 41. For example, the contigs provided as SEQ ID NOS:1-68 correspond to the genomic sequence of microorganism 1U001.8 (as indicated in FIG. 41A), which is a microorganism classified as domain Bacteria, phylum Proteobacteria, class Gammaproteobacteria, order Enterobacterales, family Enterobacteria, genus Escherichia, and species Escherichia coli and is in Guild 2 of the 141 core microorganisms identified in Table 1.

Accordingly, in some embodiments of the methods described herein, a genome identified in a metagenomic analysis is classified as corresponding to a microorganism listed in Table 1, Table 2, and/or FIGS. 42A-42XX if the identified genomic constructs have at least 97% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 98% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99.5% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 999%, or more sequence identity when compared to the contigs for the microorganism provided in the sequence listing, as denoted in FIG. 41.

Referring to block 216, in some embodiments, the method includes obtaining, in electronic form, for each respective subject in a second plurality of subjects having a second state of a biological characteristic, a corresponding plurality of genomic abundance values comprising, for each respective gut microorganism in the plurality of gut microorganisms, a corresponding value for the abundance of the genome of the respective gut microorganism in a biological sample from the gut of the respective subject. In some embodiments, the second plurality of subjects comprises at least 50, at least 100, at least 200, at least 500, at least 1000, at least 5000, at least 10,000, at least 20,000, at least 50,000, at least 100,000, at least 500,000, or at least 1,000,000 subjects. In some embodiments, the second plurality of subjects comprises no more than 1,000,000, no more than 500,000, no more than 100,000, no more than 50,000, no more than 20,000, no more than 10,000, no more than 1000 subjects, no more than 500 subjects, no more than 100 subjects, or no more than 50 subjects. In some embodiments, the second plurality of subjects consists of from 50 to 100, from 50 to 200, from 50 to 500, from 100 to 500, from 200 to 500, from 200 to 1000, from 500 to 1000, from 200 to 5,000, from 1000 to 10,000, from 5000 from 200,00, from 10,000 to 50,000, from 20,000 to 100,000, or from 500,000 to 1,000,000. In some embodiments, the second plurality of subjects falls within another range starting no lower than 50 subjects and ending no higher than 10,000,000 subjects. In some embodiments, the second plurality of subjects share similar demographic characteristics (such as age, gender, ethnicity). In some embodiments, the second plurality of subjects share similar physical characteristics (such as weight, height, BMI value). In some embodiments, the second plurality of subjects share similar health status (such as physical or mental conditions, medical history, gene carrier, or medication use). In some embodiments, the second plurality of subjects share or similar behavior and lifestyle preferences (such as diet, physical exercise, or substance use).

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g. 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 150, 200, 300, 500, 500, 700, 1000, or more. In some embodiments, shotgun metagenomic sequencing is employed to provide sequence reads for genomes in a sample, e.g., as described in U.S. Pat. No. 11,028,449, the content of which is incorporated herein by reference in its entirety.

Referring to block 218, in some embodiments, for each respective subject in the second plurality of subjects, the biological sample from the gut of the respective subject is a fecal sample. In some embodiments, the biological sample is selected from a tissue biopsy, an intestinal, or mucosal sample.

Referring to block 220, in some embodiments, the method includes sequencing, for each respective subject in the second plurality of subjects, genomic DNA from the corresponding biological sample from the gut of the respective subject, thereby obtaining the corresponding second plurality of (e.g., at least 100,000) nucleic acid sequences. In some embodiments, the second plurality of nucleic acid sequences comprises at least 100,000, at least 250,000, at least 500,000, at least 1,000,000, at least 2,500,000, at least 5,000,000, at least 10,000,000 or at least 50,000,000 nucleic acid sequences. In some embodiments, the second plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the second plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the second plurality of nucleic acid sequences falls within another range starting no lower than 100,000 nucleic acid sequences and ending no higher than 250,000,000 nucleic acid sequences.

In some embodiments, the second plurality of (e.g., at least 100,000) nucleic acid sequences are obtained through metagenomes sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

In some embodiments, the second plurality of (e.g., at least 100,000) nucleic acid sequences are obtained through targeted panel sequencing, e.g., as described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, e.g., each of a plurality of the microorganisms listed in Table 1, Table 2, and/or FIGS. 42A-42XX, prior to sequencing recovered nucleic acids. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample may comprise a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLiD sequencing systems from Life TechnologiesT™, the 454 GS FLX+ and GS Junior sequencing systems from Roche, the MinION™ system from Oxford Nanopore, or any other sequencing platform of interest.

Referring to block 222, in some embodiments, the method includes obtaining, in electronic form, for each respective subject in the second plurality of subjects, a corresponding second plurality of (e.g., at least 100,000) nucleic acid sequences for genomic DNA from a corresponding biological sample from the gut of the respective subject, and determining, for each respective subject in the second plurality of subjects, the corresponding genomic abundance value for each respective gut microorganism in the plurality of gut microorganisms from the corresponding second plurality of (e.g., at least 100,000) nucleic acid sequences. In some embodiments, the genomic abundance values determined for each respective subject in the second plurality of subjects comprise at least 20, at least 25, at least 50, at least 100, at least 250, at least 500, at least 1,000, at least 5,000 or at least 10,000 genome abundance values. In some embodiments, the genomic abundance values determined for each respective subject in the second plurality of subjects comprise no more than 250,000, no more than 100,000, no more than 50,000, no more than 25,000, no more than 10,000, no more than 5,000, no more than 1,000, no more than 100, no more than 50, no more than 30, or no more than 20 genome abundance values. In some embodiments, the genomic abundance values determined for each respective subject in the second plurality of subjects consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the genomic abundance values determined for each respective subject in the second plurality of subjects fall within another range starting no lower than 20 genome abundance values and ending no higher than 250,000 genome abundance values.

Referring to block 224, in some embodiments, the method includes for each respective subject in the second plurality of subjects, assembling a corresponding second plurality of gut microorganism genomes by metagenomic de novo sequence assembly from the corresponding second plurality of (e.g., at least 100,000) nucleic acid sequences, and calculating, for each respective gut microorganism genome in the corresponding second plurality of gut microorganism genomes, a corresponding genomic abundance of the respective gut microorganism genome. In some embodiments, metagenomic de novo sequence assembly further comprise generating contigs based on the sequencing reads generated by a shotgun sequencing technique, e.g., as described in U.S. Pat. No. 10,529,443. In some embodiments, the second plurality of (e.g., at least 100,000) nucleic acid sequences can be assembled into full genomes of the plurality of gut microorganisms. In some embodiments, the second plurality of (e.g., at least 100,000) nucleic acid sequences can be assembled into partial genomes of the plurality of gut microorganisms.

Referring to block 226, in some embodiments, the method includes, for each respective subject in the second plurality of subjects, assigning each respective nucleic acid sequence in the corresponding second plurality of (e.g., at least 100,000) sequences to a respective gut microorganism in the plurality of gut microorganisms, thereby generating, for each respective gut microorganism in the plurality of gut microorganism, a corresponding count of respective nucleic acid sequences in the corresponding second plurality of nucleic acid sequences assigned to the respective gut microorganism, and determine, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid, e.g., a contig listed in FIG. 41. In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity-based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

Referring to block 228, in some embodiments, the second state of the biological characteristic is the presence of the disease or disorder, e.g., type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), and Parkinson's disease (PD), Multiple Sclerosis (MS), Gaucher disease type II (GDII), COVID-19 (COV), Behcet's disease (BD), autism spectrum disorder (ASD), or pancreatic cancer (PC). In some embodiments, the disease or disorder is cancer, Alzheimer diseases, a cardiovascular disease, an autoimmune disease, a mental health disease, an infectious disease, or a genetic disorder.

In some embodiments, the second state of the biological characteristic is a second severity of the disease or disorder. In some embodiments, the severity of the diseases is categorized by type, frequency or intensity experienced by a subject. In some embodiments, the severity of the disease is categorized by the progression or prognosis of a disease or disorder, e.g., different stages of cancer. In some embodiments, the second state of the biological characteristic is a treated disease or disorder, the second state of the biological characteristic is a disease or disorder treated with a second therapy, e.g., surgery, radiation therapy, chemotherapy, targeted therapy, gene therapy, immunotherapy, medication, diet change, lifestyle modification. In some embodiments, the second state of the biological characteristic is a second level of a nutrient in a diet, such as carbohydrate, proteins, fats, vitamins, fibers. In some embodiments, the second state of the biological characteristic is a second age. In some embodiments, a threshold value is provided for determining the second state of biological characteristics, e.g., a level of biomarker, a diagnostic cut-off value, or a threshold nutrient intake level.

Referring to block 230, in some embodiments, the plurality of gut microorganisms comprises at least 20 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 30 gut microorganisms selected from Table 1, Table 2, or FIGS. 42A-42XX. In some embodiments, the plurality of gut microorganisms comprises at least 40 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 42A-42XX.

Referring to block 232, in some embodiments, the method includes computing a first plurality of similarity metrics from the corresponding pluralities of genomic abundance values across the first plurality of subjects, where the first plurality of similarity metrics comprises a first corresponding similarity metric for each unique pair of gut microorganisms in the plurality of gut microorganisms, and the first corresponding similarity metric quantifies a similarity between (i) a corresponding first vector formed by the corresponding genomic abundance values of the first microorganism in the unique pair of gut microorganisms across the first plurality of subjects and (ii) a corresponding second vector formed by the corresponding genomic abundance values of the second microorganism in the unique pair of gut microorganisms across the first plurality of subjects. In some embodiments, a corresponding first vector is a set of values where each value represents a genome abundance value of the first microorganism for one subject in the first plurality of subjects. In some embodiments, a corresponding second vector is a set of values where each value represents a genome abundance value of the second microorganism for one subject in the first plurality of subjects. In some embodiments, the unique genome pairs are formed by any two genomes detected across the first plurality of subjects. In some embodiments, the number of unique pairs for a total number of N genomes can be calculated by N(N−1)/2, wherein N represents the non-repetitive number of genomes detected across the first plurality of subjects. As the number gut microorganisms in the set increases, the number of calculations required to determine the set of all similarity metrics increases as a second order function of the number of microorganisms.

Referring to block 234, in some embodiments, the method includes computing a second plurality of similarity metrics using the corresponding genomic abundance values for the second plurality of subjects, where the second plurality of similarity metrics comprises a second corresponding similarity metric for each unique pair of gut microorganisms in the plurality of gut microorganisms, and the second corresponding similarity metric quantifies a similarity between (i) a corresponding second vector formed by the corresponding genomic abundance values of the first microorganism in the unique pair of gut microorganisms across the second plurality of subjects and (ii) a corresponding second vector formed by the corresponding genomic abundance values of the second microorganism in the unique pair of gut microorganisms across the second plurality of subjects. In some embodiments, a corresponding second vector is a set of values where each value represents a genome abundance value of the second microorganism for one subject in the second plurality of subjects. In some embodiments, a corresponding second vector is a set of values where each value represents a genome abundance value of the second microorganism for one subject in the second plurality of subjects. In some embodiments, the unique genome pairs are formed by any two genomes detected across the second plurality of subjects. In some embodiments, the number of unique pairs for a total number of N genomes can be calculated by N(N-1)/2, wherein N represents the non-repetitive number of genomes detected across the first plurality of subjects. As the number gut microorganisms in the set increases, the number of calculations required to determine the set of all similarity metrics increases as a second order function of the number of microorganisms

Referring to block 236, in some embodiments, the method includes determining a set of unique pairs of gut microorganisms in the plurality of gut microorganisms based on the first plurality of similarity metrics and the second plurality of similarity metrics, for each respective unique pair of gut microorganisms in the set of unique pairs of gut microorganisms, where the first corresponding similarity metric and the second corresponding similarity metric both indicate a statistically significant positive correlation between the abundance of the first gut microorganism and the abundance of the second gut microorganism in the respective unique pair of gut microorganisms, or the first corresponding similarity metric and the second corresponding similarity metric both indicate a statistically significant negative correlation between the abundance of the first gut microorganism and the abundance of the second gut microorganism in the respective unique pair of gut microorganisms. In some embodiments, the similarity metrics is correlation coefficient. In some embodiments, a threshold or cut-off value for a statistically significant positive correlation is defined. In some embodiments, a threshold or cut-off value for a statistically significant negative correlation is defined.

Referring to block 238, in some embodiments, one or both of the first corresponding similarity metric and the second similarity metric may be a Pearson correlation coefficient, an intraclass correlation coefficient, or a rank correlation coefficient. In some embodiments, the similarity metrics is Spearman's correlation coefficient or maximal information coefficient (MIC). In some embodiments, the similarity metrics is Kendall tau rank correlation coefficient, also called Kendall's tau, which is used to measure association between two measures. In some embodiments, the similarity metrics is calculated by any Sparse Correlations for Compositional data (SparCC) based algorithm, or SParse InversE Covariance Estimation for Ecological ASsociation Inference (SPIEC-EASI) based algorithm. In some embodiments, the SparCC based algorithm is FastSpar.

Referring to block 240, in some embodiments, a statistically significant positive correlation has a P-value of less than 0.001. In some embodiments, a statistically significant positive correlation has a P-value of less than 0.05. In some embodiments, a statistically significant positive correlation has a P-value of less than 0.01. In some embodiments, a statistically significant positive correlation has a P-value of less than 0.001, less than 0.005, less than 0.01, less than 0.025, less than 0.05, or less than 0.075.

Referring to block 242, in some embodiments, the method includes identifying a set of gut microorganisms comprising respective gut microorganisms represented in the set of unique pairs of gut microorganisms. In some embodiments, microbiome network comprising respective gut microorganisms represented in the set of unique pairs of gut microorganisms are constructed. In some embodiments, the networks are visualized by a bioinformatic software, e.g., Cystoscape.

Referring to block 244, in some embodiments, the method includes clustering the respective gut microorganisms represented in the set of unique pairs of gut microorganisms into one of more networks, each respective connected network comprising a corresponding plurality of nodes and a corresponding set of one or more edges.

Referring to block 246, in some embodiments, each respective node in the corresponding plurality of nodes represents a unique gut microorganism represented in the set of unique pairs of gut microorganisms. In some embodiments, a node size represents the average abundance of the genome. In some embodiments, the links between the nodes are treated as metal springs attached to the pair of nodes. In some embodiments, the similarity metrics are used to determine the repulsion and attraction of the spring. In some embodiments, the values of the similarity metrics are used to determine the weight of links.

Referring to block 248, in some embodiments, each respective edge in the corresponding set of one or more edges connects two nodes representing a respective unique pair of gut microorganisms in the set of unique pairs of gut microorganisms. In some embodiments, the positively correlated unique pairs of genomes are differentiated from negatively correlated unique pairs of genomes.

Referring to block 250, in some embodiments, each respective node in the corresponding plurality of nodes is connected to at least one other respective node in the plurality of nodes through a respective edge in the corresponding set of one or more edges.

Referring to block 252, in some embodiments, the method includes identifying the respective network in the one or more networks comprising the most nodes, thereby identifying the set of gut microorganisms represented by the corresponding plurality of nodes in the respective network. In some embodiments, the nodes that are not connected to the one or more networks are removed.

Referring to block 254, in some embodiments, the set of identified gut microorganisms comprises all respective gut microorganisms represented in the set of unique pairs of gut microorganisms. In some embodiments, the set of identified gut microorganisms comprises all respective gut microorganisms represented by the nodes of the one or more microbiome networks.

Referring to block 256, in some embodiments, the set of identified gut microorganisms comprises at least 20 gut microorganisms from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 30 gut microorganisms selected from Table 1, Table 2, or FIGS. 42A-42XX. In some embodiments, the plurality of gut microorganisms comprises at least 40 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 42A-42XX.

In some embodiments, the set of identified gut microorganisms are selected from those microorganisms in Table 5 having a connectivity of at least 2, at least 3, at least 4, at least 5, at least 10, at least 15, at least 20, at least 25, or more. Referring to block 308, in some embodiments, the plurality of gut microorganisms comprises at least 20 microorganisms selected from those microorganisms listed in Table 5 as having a connectivity of at least 2. In some embodiments, the plurality of gut microorganisms comprises at least 20 microorganisms selected from those microorganisms listed in Table 5 as having a connectivity of at least 3, at least 4, at least 5, at least 10, at least 15, at least 20, at least 25, or more.

### 2. Methods of Training a Model for Evaluating Human Health

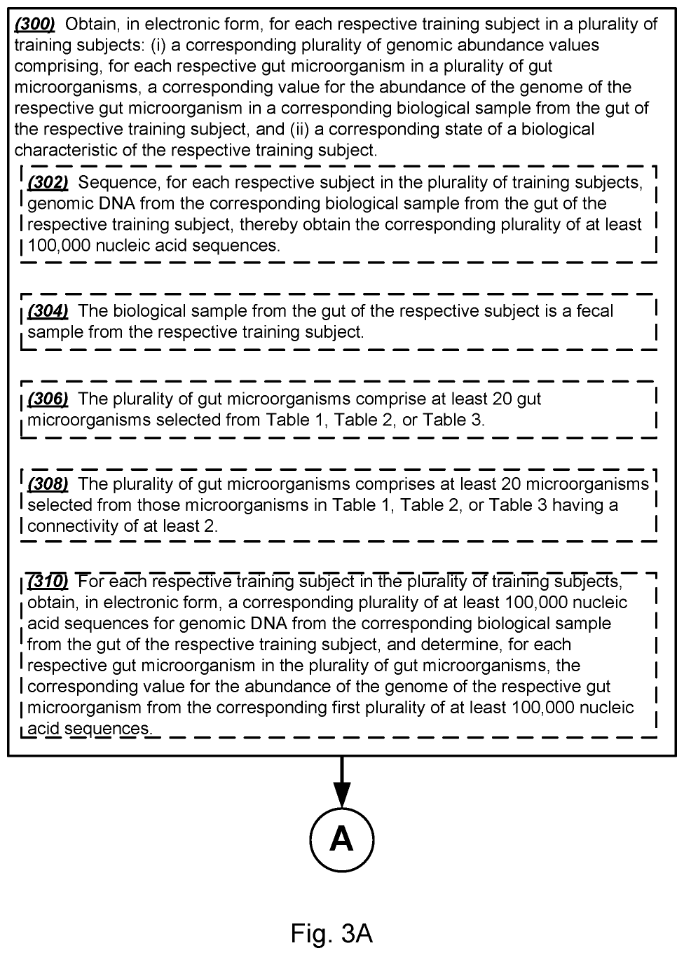

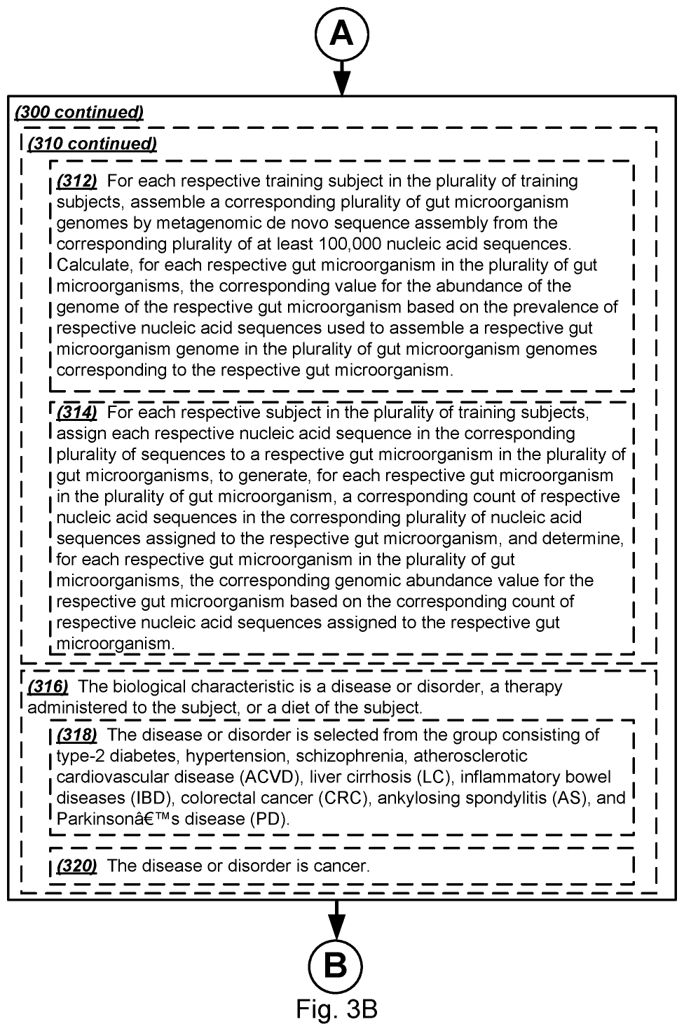

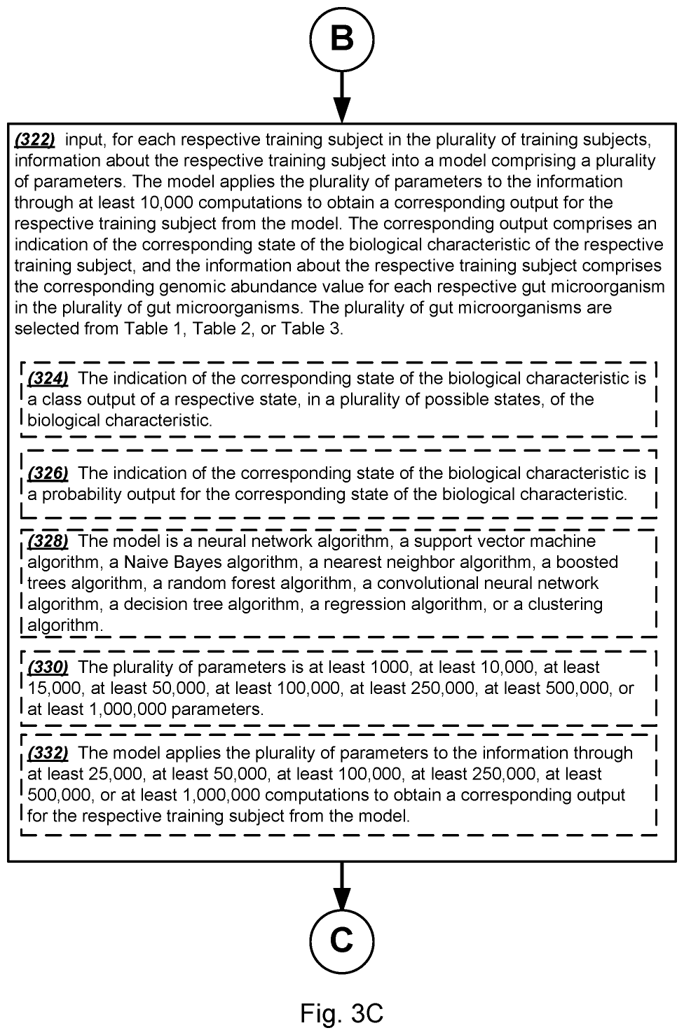

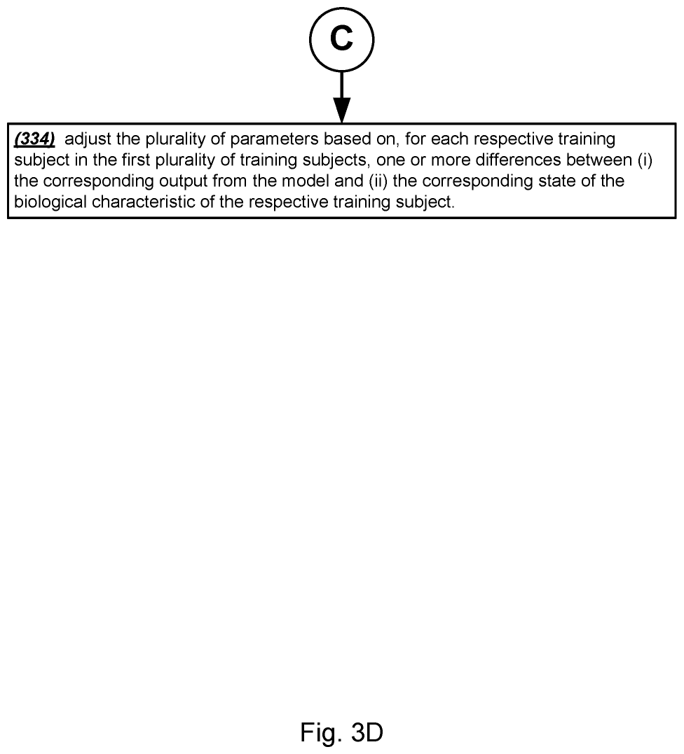

FIG. 3 is a schematic diagram of a method for training a model for evaluating human health as discussed below. The method 300 may be implemented using a computer system (e.g., the computer system 100 shown and described above in reference to FIG. 1).

Referring to block 300, in some embodiments, the method includes obtaining, in electronic form, for each respective training subject in a plurality of training subjects: (i) a corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and (ii) a corresponding state of a biological characteristic of the respective training subject.

In some embodiments, the plurality of training subjects comprises at least 50, at least 100, at least 200, at least 500, at least 1000, at least 5000, at least 10,000, at least 20,000, at least 50,000, at least 100,000, at least 500,000, or at least 1,000,000 subjects. In some embodiments, the plurality of subjects comprises no more than 1,000,000, no more than 500,000, no more than 100,000, no more than 50,000, no more than 20,000, no more than 10,000, no more than 1000 subjects, no more than 500 subjects, no more than 100 subjects, or no more than 50 subjects. In some embodiments, the plurality of training subjects consists of from 50 to 100, from 50 to 200, from 50 to 500, from 100 to 500, from 200 to 500, from 200 to 1000, from 500 to 1000, from 200 to 5,000, from 1000 to 10,000, from 5000 from 200,00, from 10,000 to 50,000, from 20,000 to 100,000, or from 500,000 to 1,000,000. In some embodiments, the plurality of training subjects falls within another range starting no lower than 50 subjects and ending no higher than 10,000,000 subjects. In some embodiments, the plurality of training subjects share similar demographic characteristics (such as age, gender, ethnicity). In some embodiments, the plurality of training subjects share similar physical characteristics (such as weight, height, BMI value). In some embodiments, the plurality of training subjects share similar health status (such as physical or mental conditions, medical history, gene carrier, or medication use). In some embodiments, the plurality of subjects share or similar behavior and lifestyle preferences (such as diet, physical exercise, or substance use).

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g. 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 150, 200, 300, 500, 500, 700, 1000, or more. In some embodiments, shotgun metagenomic sequencing is employed to provide sequence reads for genomes in a sample, e.g., as described in U.S. Pat. No. 11,028,449, the content of which is incorporated herein by reference in its entirety.

Referring to block 302, in some embodiments, the method includes sequencing, for each respective subject in the plurality of training subjects, genomic DNA from the corresponding biological sample from the gut of the respective training subject, thereby obtaining a corresponding plurality of (e.g., at least 100,000) nucleic acid sequences. In some embodiments, the plurality of nucleic acid sequences comprises at least 100,000, at least 250,000, at least 500,000, at least 1,000,000, at least 2,500,000, at least 5,000,000, at least 10,000,000 or at least 50,000,000 nucleic acid sequences. In some embodiments, the plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the plurality of nucleic acid sequences falls within another range starting no lower than 100,000 nucleic acid sequences and ending no higher than 250,000,000 nucleic acid sequences.

In some embodiments, the plurality of (e.g., at least 100,000) nucleic acid sequences are obtained through metagenomic sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

In some embodiments, the plurality of (e.g., at least 100,000) nucleic acid sequences are obtained through targeted panel sequencing, e.g., as described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, e.g., each of a plurality of the microorganisms listed in Table 1, Table 2, and/or FIGS. 42A-42XX, prior to sequencing recovered nucleic acids. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample comprise a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLiD sequencing systems from Life TechnologiesT™, the 454 GS FLX+ and GS Junior sequencing systems from Roche, the MinION™ system from Oxford Nanopore, or any other sequencing platform of interest.

Referring to block 304, in some embodiments, the biological sample from the gut of the respective subject is a fecal sample from the respective training subject. In some embodiments, the sample is a tissue biopsy, an intestinal, or mucosal sample. See, for example, Tang Q, J et al., Current Sampling Methods for Gut Microbiota: A Call for More Precise Devices, Front Cell Infect Microbiol., 10:151 (2020), the content of which is incorporated herein by reference in its entirety.

Referring to block 306, in some embodiments, the plurality of gut microorganisms comprises at least 20 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 30 gut microorganisms selected from Table 1, Table 2, or FIGS. 42A-42XX. In some embodiments, the plurality of gut microorganisms comprises at least 40 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 42A-42XX.

In some embodiments of the methods described herein, a genome identified in a metagenomic analysis is classified as corresponding to a microorganism listed in Table 1, Table 2, and/or FIGS. 42A-42XX if the identified genomic constructs have at least 97% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 98% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99.5% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9%, or more sequence identity when compared to the contigs for the microorganism provided in the sequence listing, as denoted in FIG. 41.

In some embodiments, the plurality of gut microorganisms is selected from those microorganisms in Table 5 having a connectivity of at least 2, at least 3, at least 4, at least 5, at least 10, at least 15, at least 20, at least 25, or more. Referring to block 308, in some embodiments, the plurality of gut microorganisms comprises at least 20 microorganisms selected from those microorganisms listed in Table 5 as having a connectivity of at least 2. In some embodiments, the plurality of gut microorganisms comprises at least 20 microorganisms selected from those microorganisms listed in Table 5 as having a connectivity of at least 3, at least 4, at least 5, at least 10, at least 15, at least 20, at least 25, or more.

Referring to block 310, in some embodiments, the method includes, for each respective training subject in the plurality of training subjects, obtaining, in electronic form, a corresponding plurality of (e.g., at least 100,000) nucleic acid sequences for genomic DNA from the corresponding biological sample from the gut of the respective training subject, and determining, for each respective gut microorganism in the plurality of gut microorganisms, the corresponding value for the abundance of the genome of the respective gut microorganism from the corresponding first plurality of (e.g., at least 100,000) nucleic acid sequences. In some embodiments, the genomic abundance values determined for each respective subject in the plurality of training subjects comprise at least 20, at least 25, at least 50, at least 100, at least 250, at least 500, at least 1,000, at least 5,000 or at least 10,000 genome abundance values. In some embodiments, the genomic abundance values determined for each respective subject in the plurality of training subjects comprise no more than 250,000, no more than 100,000, no more than 50,000, no more than 25,000, no more than 10,000, no more than 5,000, no more than 1,000, no more than 100, no more than 50, no more than 30, or no more than 20 genome abundance values. In some embodiments, the genomic abundance values determined for each respective subject in the plurality of training subjects consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the genomic abundance values determined for each respective subject in the plurality of training subjects fall within another range starting no lower than 20 genome abundance values and ending no higher than 250,000 genome abundance values.

Referring to block 312, in some embodiments, the method includes, for each respective training subject in the plurality of training subjects, assembling, in electronic form, a corresponding plurality of gut microorganism genomes by metagenomic de novo sequence assembly from the corresponding plurality of (e.g., at least 100,000) nucleic acid sequences, and calculating, for each respective gut microorganism in the plurality of gut microorganisms, the corresponding value for the abundance of the genome of the respective gut microorganism based on the prevalence of respective nucleic acid sequences, in the plurality of (e.g., at least 100,000) nucleic acid sequences, used to assemble a respective gut microorganism genome in the plurality of gut microorganism genomes corresponding to the respective gut microorganism. In some embodiments, metagenomic de novo sequence assembly further comprise generating contigs based on the sequencing reads generated by a shotgun sequencing technique, as described in U.S. Pat. No. 10,529,443, the content of which is incorporated herein by reference in its entirety. In some embodiments, the first plurality of (e.g., at least 100,000) nucleic acid sequences can be assembled into full genomes of the plurality of gut microorganisms. In some embodiments, the first plurality of (e.g., at least 100,000) nucleic acid sequences can be assembled into partial genomes of the plurality of gut microorganisms.

Referring to block 314, in some embodiments, the method includes, for each respective subject in the plurality of training subjects, assigning each respective nucleic acid sequence in the corresponding plurality of (e.g., at least 100,000) sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and determining, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid, e.g., a contig listed in FIG. 41. In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity-based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

Referring to block 316, in some embodiments, the biological characteristic is a disease or disorder, a therapy administered to the subject, e.g., surgery, radiation therapy, chemotherapy, targeted therapy, gene therapy, immunotherapy, medication, diet change, lifestyle modification, or a diet of the subject, such as a diet rich or poor in carbohydrate, proteins, fats, vitamins, or fibers.

Referring to block 318, in some embodiments, the disease or disorder is selected from the group consisting of type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), and Parkinson's disease (PD), Multiple Sclerosis (MS), Gaucher disease type II (GDII), COVID-19 (COV), Behcet's disease (BD), autism spectrum disorder (ASD), or pancreatic cancer (PC). In some embodiments, the disease or disorder is cancer, Alzheimer diseases, a cardiovascular disease, an autoimmune disease, a mental health disease, an infectious disease, or a genetic disorder.

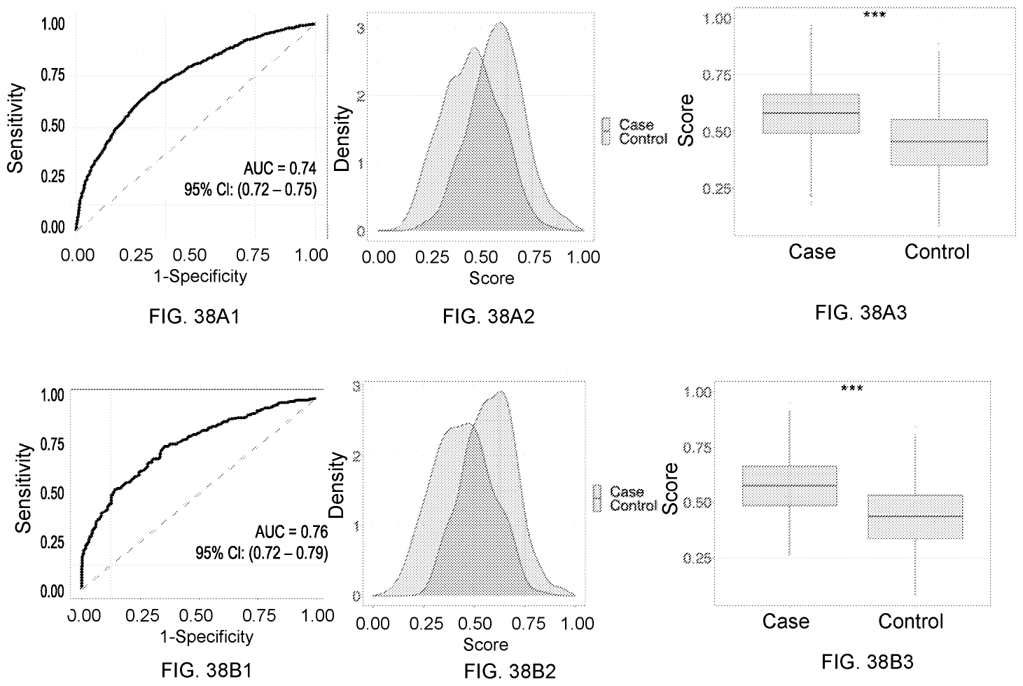

In some embodiments, the model is trained against datasets collected across a plurality of disorders and the model is trained to distinguish between a healthy state and an unhealthy state. For example, as described in Example 5, a random forest classifier was trained against datasets from 26 different studies collectively looking at microbiomes in 15 different disorders. As shown in FIG. 38, the resulting model was powered to predict healthy or unhealthy disorder states regardless of the disorder. Accordingly, in some embodiments, the biological characteristic is any one of a plurality of diseases and/or disorders, where the first state is the presence of any one of the diseases or disorders and the second state is the absence of any of the diseases or disorders.

Referring to block 320, in some embodiments, the disease or disorder is cancer.

Referring to block 322, in some embodiments, the method includes inputting, for each respective training subject in the plurality of training subjects, information about the respective training subject into a model comprising a plurality of parameters. The model applies the plurality of parameters to the information through at least 10,000 computations to obtain a corresponding output for the respective training subject from the model. The corresponding output comprises an indication of the corresponding state of the biological characteristic of the respective training subject. The information about the respective training subject comprises the corresponding genomic abundance value for each respective gut microorganism in the plurality of gut microorganisms, and the plurality of gut microorganisms are selected from Table 1, Table 2, or FIGS. 42A-42XX.

Referring to block 322, in some embodiments, the indication of the corresponding state of the biological characteristic is a class output of a respective state, in a plurality of possible states, of the biological characteristic. In some embodiments, the possible state is a state from a healthy subject. In some embodiments, the possible state is a state from a patient. In some embodiments, the state from a patient is categorized by type, frequency or intensity experienced by a patient. In some embodiments, the state from a patient is categorized by the progression or prognosis of a disease or disorder, e.g., different stages of cancer. In some embodiments, a threshold value is provided for determining the state of a healthy subject or a patient, such as a level of biomarker, a diagnostic cut-off value, or a threshold nutrient intake level.

Referring to block 324, in some embodiments, the indication of the corresponding state of the biological characteristic is a probability output for the corresponding state of the biological characteristic. In some embodiments, the corresponding state is a state from a healthy subject. In some embodiments, the corresponding state is a state from a patient. In some embodiments, the state from a patient is categorized by type, frequency or intensity experienced by a patient. In some embodiments, the state from a patient is categorized by the progression or prognosis of a disease or disorder, e.g., different stages of cancer. In some embodiments, a threshold value is provided for determining the state of a healthy subject or a patient, such as a level of biomarker, a diagnostic cut-off value, or a threshold nutrient intake level.

Referring to block 326, in some embodiments, the model is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

Referring to block 328, in some embodiments, the plurality of parameters is at least 1000, at least 10,000, at least 15,000, at least 50,000, at least 100,000, at least 250,000, at least 500,000, at least 1,000,000 parameters, at least 2,500,000 parameters, at least 5,000,000 parameters, at least 10,000,000 parameters, or more.

Referring to block 330, in some embodiments, the model applies the plurality of parameters to the information through at least 1000 computation, at least 5000 computations, at least 10,000 computations, at least 25,000 computations, at least 50,000 computations, at least 100,000 computations, at least 250,000 computations, at least 500,000 computations, at least 1,000,000 computations, at least 2,500,000 computations, at least 5,000,000 computations, at least 10,000,000 computations, or more to obtain a corresponding output for the respective training subject from the model.

Referring to block 330, in some embodiments, the method includes adjusting the plurality of parameters based on, for each respective training subject in the first plurality of training subjects, one or more differences between (i) the corresponding output from the model and (ii) the corresponding state of the biological characteristic of the respective training subject.

In some embodiments where deep learning techniques utilize a neural network as described above, the training of the neural network to improve the accuracy of its prediction involves modifying one or more parameters, including, but not limited to, weights in the filters in convolutional layers as well as biases in network layers. In some embodiments, the weights and biases are further constrained with various forms of regularization such as L1, L2, weight decay, and dropout.

For instance, in some embodiments, the neural network or any of the models disclosed herein optionally, where training data is labeled (e.g., with an indication of the state of the biological characteristic), have their parameters (e.g., weights) tuned (adjusted to potentially minimize the error between the system's predicted indications and the training data's measured indications). Various methods used to minimize error function, such as gradient descent methods, include, but are not limited to, log-loss, sum of squares error, hinge-loss methods. In some embodiments, these methods further include second-order methods or approximations such as momentum, Hessian-free estimation, Nesterov's accelerated gradient, adagrad, etc. In some embodiments, the methods also combine unlabeled generative pretraining and labeled discriminative training.

Accordingly, in some embodiments, the training of the neural network comprises adjusting one or more parameters in the plurality of parameters by back-propagation through a loss function. In some embodiments, the loss function is a regression task and/or a classification task. Non-limiting examples of loss functions suitable for the regression task include, but are not limited to, a mean squared error loss function, a mean absolute error loss function, a Huber loss function, a Log-Cosh loss function, or a quantile loss function. See, Wang et al., 2020, “A Comprehensive Survey of Loss Functions in Machine Learning,” Annals of Data Science, doi.org/10.1007/s40745-020-00253-5, last accessed Sep. 15, 2021, which is hereby incorporated by reference in its entirety. Non-limiting examples of loss functions suitable for the classification task include, but are not limited to, a binary cross entropy loss function, a hinge loss function, or a squared hinged loss function. In some embodiments, the loss function is any suitable regression task loss function or classification task loss function.

Other suitable methods for training the neural network that are contemplated for use in the present disclosure are further described herein (see, e.g., Definitions: Untrained model, above).

In some embodiments, the parameters of the neural network are randomly initialized prior to training.

In some embodiments, the neural network comprises a dropout regularization parameter. For example, in some embodiments, a regularization is performed by adding a penalty to the loss function, where the penalty is proportional to the values of the parameters in the trained or untrained model. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty.

In some embodiments, the training the neural network comprises an optimizer. In some embodiments, the optimizer may employ the loss function to update the parameters of the neural network or other model via back-propagation. In some embodiments, the training the neural network comprises a learning rate.

In some embodiments, the learning rate is at least 0.0001, at least 0.0005, at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1. In some embodiments, the learning rate is no more than 1, no more than 0.9, no more than 0.8, no more than 0.7, no more than 0.6, no more than 0.5, no more than 0.4, no more than 0.3, no more than 0.2, no more than 0.1 no more than 0.05, no more than 0.01, or less. In some embodiments, the learning rate is from 0.0001 to 0.01, from 0.001 to 0.5, from 0.001 to 0.01, from 0.005 to 0.8, or from 0.005 to 1. In some embodiments, the learning rate falls within another range starting no lower than 0.0001 and ending no higher than 1.

In some embodiments, the learning rate further comprises a learning rate decay (e.g., a reduction in the learning rate over one or more epochs). For example, a learning decay rate can be a reduction in the learning rate of 0.5 or 0.1. In some embodiments, the learning rate is a differential learning rate. In some embodiments, the training the neural network further uses a scheduler that conditionally applies the learning rate decay based on an evaluation of a performance metric over a threshold number of training epochs (e.g., the learning rate decay is applied when the performance metric fails to satisfy a threshold performance value for at least a threshold number of training epochs).

In some embodiments, the performance of the neural network is measured at one or more time points using a performance metric, including, but not limited to, a training loss metric, a validation loss metric, and/or a mean absolute error. In some embodiments, the performance metric is an area under receiving operating characteristic (AUROC) and/or an area under precision-recall curve (AUPRC).

For instance, in some embodiments, the performance of the neural network is measured by validating the model using a validation (e.g., development) dataset. In some such embodiments, the training the neural network forms a trained neural network when the neural network satisfies a minimum performance requirement based on a validation.

In some embodiments, any suitable method for validation can be used, including but not limited to K-fold cross-validation, advanced cross-validation, random cross-validation, grouped cross-validation (e.g., K-fold grouped cross-validation), bootstrap bias corrected cross-validation, random search, and/or Bayesian hyperparameter optimization.

In some embodiments, a method is provided for training a model comprising a plurality of parameters by a procedure comprising (i) inputting corresponding genomic abundance value for each respective gut microorganism in a plurality of gut microorganisms for each respective training subject in a plurality of training subjects, thereby obtaining as output from the model, for each respective training subject in the plurality of training subjects, a corresponding predicted state of a biological characteristic, and (ii) refining the plurality of model parameters based on a differential between the corresponding state of the biological characteristic for the respective training subject and the corresponding predicted state of the biological characteristic for each respective training subject in the plurality of training subjects.

### 3. Methods of Evaluating the Health of a Subject

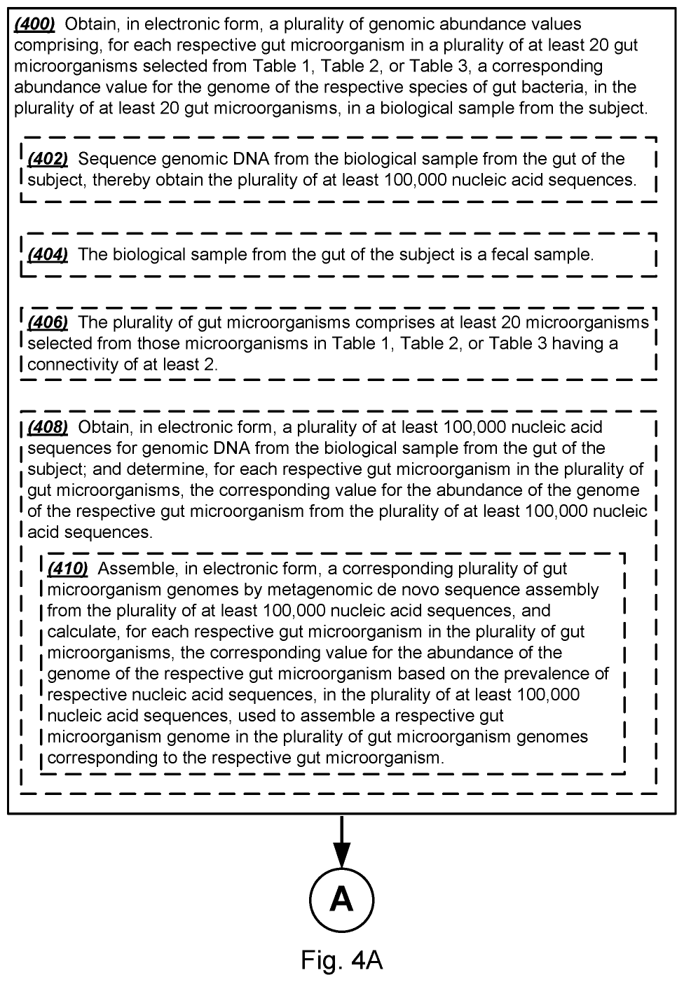

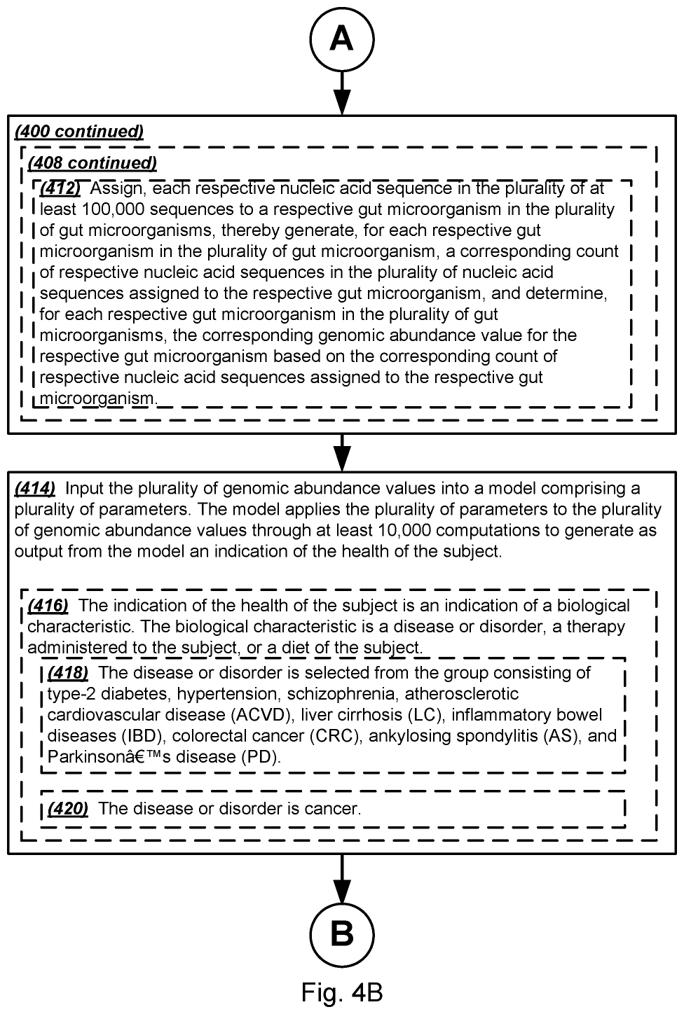

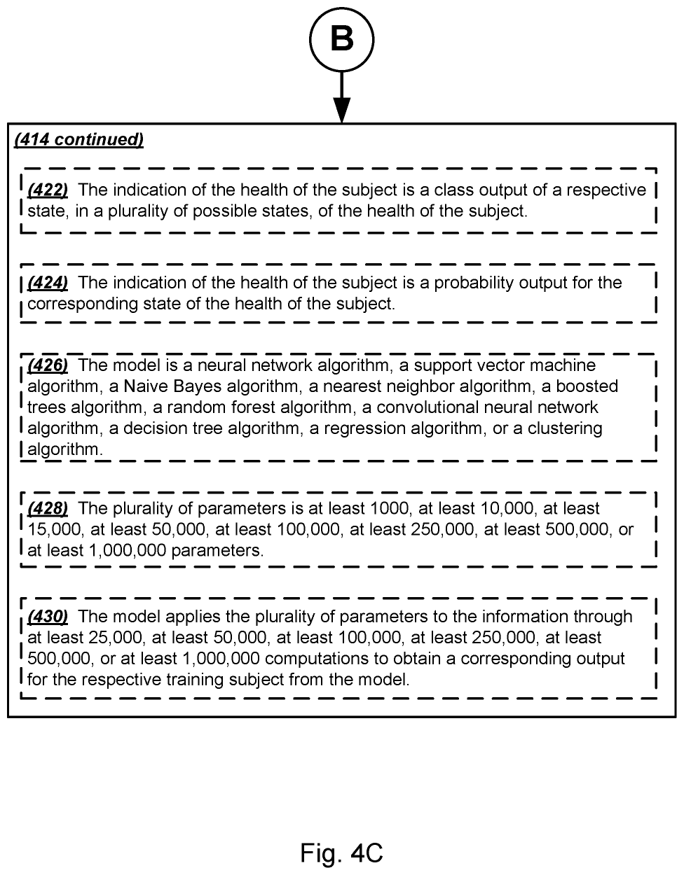

FIG. 4 is a schematic diagram of a method for training a model for evaluating human health as discussed below. The method may be implemented using a computer system (e.g., the computer system 100 shown and described above in reference to FIG. 1).

Referring to block 400, in some embodiments, obtain, in electronic form, a plurality of genomic abundance values comprising, for each respective gut microorganism in a plurality of (e.g., at least 20) gut microorganisms selected from Table 1, Table 2, or FIG. 42A-42XX, a corresponding abundance value for the genome of the respective species of gut bacteria, in the plurality of (e.g., at least 20) gut microorganisms, in a biological sample from the subject. In some embodiments, the plurality of gut microorganisms comprises at least 25 gut microorganisms selected from Table 1, Table 2, or FIGS. 42A-42XX. In some embodiments, the plurality of gut microorganisms comprises at least 30 gut microorganisms selected from Table 1, Table 2, or FIGS. 42A-42XX. In some embodiments, the plurality of gut microorganisms comprises at least 40 gut microorganisms selected from Table 1, Table 2, or FIGS. 42A-42XX. In some embodiments, the plurality of gut microorganisms comprises at least 25 gut microorganisms selected from Table 1, Table 2, or FIGS. 42A-42XX. In some embodiments, the plurality of gut microorganisms comprises at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 200, at least 250, at least 300, at least 400, at least 500, at least 600, at least 700, at least 800, or all of the gut microorganisms selected from Table 1, Table 2 or FIGS. 42A-42XX. In some embodiments, the plurality of gut microorganisms are all of the gut microorganisms listed in Table 1. In some embodiments, the plurality of gut microorganisms are all of the gut microorganisms listed in Table 2. In some embodiments, the plurality of gut microorganisms are all of the gut microorganisms listed in FIGS. 42A-42XX.

In some of the embodiments, the corresponding value for the abundance of the genome is a value representative of the absolute abundance of a microorganism genome. In some of the embodiments, the corresponding value for the abundance of the genome is a value representative of a normalized abundance value, or a relative abundance value (e.g., an abundance of one microorganism normalized against the abundance of total microbiome of interest). In some of the embodiments, corresponding value for the abundance of the genome is a value representative of an averaged abundance value (e.g., average of abundances obtained at different time points or from different biological samples from the patients, or average of abundances obtained using different probes, etc.), or a combination of any of above. The corresponding value for the abundance of the genome is measured by any technique known in the art. In some embodiments, the genomic abundance value for the genome is measured by quantitative PCR (qPCR), such as bacterial 16S rRNA qPCR, RT-PCR, or qRT-PCR, for quantifying the abundance of region of interests in the genome, e.g., as described in U.S. Pat. No. 11,427,865, the disclosure of which is hereby incorporated by reference in its entirety. In some embodiments, the genomic abundance value is measured by targeted sequencing (e.g., 16S rRNA sequencing, or any other suitable biomarker), partial genome sequencing or whole genome sequencing, thereby quantifying the number of reads of the targeted regions in a microorganism genome to determine the abundance of the genome, e.g., as disclosed in U.S. Patent Application Publication No. 2021/0403986 or U.S. Pat. No. 11,332,783, the disclosures of which are hereby incorporated by reference in their entireties. In some embodiments, deep sequencing is employed to determine the abundance of targeted sequences, e.g., as disclosed in U.S. Patent Application Publication No. 2018/0237863, the disclosure of which is incorporated herein by reference in its entirety. In some embodiments, the sequencing depth is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 70, at least 80, at least 90, at least 100, at least 110, at least 120, at least 130, at least 150, at least 200, at least 300, at least 400, at least 500, at least 750, at least 1000, or more. In some embodiments, shotgun metagenomic sequencing is employed to provide sequence reads for genomes in a sample, e.g., as described in U.S. Pat. No. 11,028,449, the content of which is incorporated herein by reference in its entirety.

In some embodiments of the methods described herein, a genome identified in a metagenomic analysis is classified as corresponding to a microorganism listed in Table 1, Table 2, and/or FIGS. 42A-42XX if the identified genomic constructs have at least 97% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 98% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 99.5% sequence identity when compared to the contigs for the microorganism provided in the sequence listing, as denoted in FIG. 41. In some embodiments, a genome identified in a metagenomic analysis is classified as corresponding to a microorganism listed in Table 1, Table 2, and/or FIGS. 42A-42XX if the identified genomic constructs have at least 97%, at least 97.5%, at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9%, or more sequence identity when compared to the contigs for the microorganism provided in the sequence listing, as denoted in FIG. 41.

Referring to block 402, in some embodiments, the method includes sequencing genomic DNA from the biological sample from the gut of the subject, thereby obtain the plurality of (e.g., at least 100,000) nucleic acid sequences. In some embodiments, the plurality of nucleic acid sequences comprises at least 100,000, at least 250,000, at least 500,000, at least 1,000,000, at least 2,500,000, at least 5,000,000, at least 10,000,000 or at least 50,000,000 nucleic acid sequences. In some embodiments, the plurality of nucleic acid sequences comprises no more than 250,000,000, no more than 100,000,000, no more than 50,000,000, no more than 25,000,000, no more than 10,000,000, no more than 5,000,000, no more than 1,000,000, no more than 100,000 nucleic acid sequences. In some embodiments, the plurality of nucleic acid sequences consists of from 100,000 to 1,000,000, from 200,000 to 5,000,000, from 500,000 from 10,000,000, from 1,000,000 to 20,000,000, from 5,000,000 to 50,000,000, from 10,000,000 to 100,000,000, or from 50,000,000 to 250,000,000 nucleic acid sequences. In some embodiments, the plurality of nucleic acid sequences falls within another range starting no lower than 100,000 nucleic acid sequences and ending no higher than 250,000,000 nucleic acid sequences.

In some embodiments, the plurality of (e.g., at least 100,000) nucleic acid sequences are obtained through metagenomic sequencing, e.g., as disclosed in U.S. Patent Application Publication No. 2016/0239602 or U.S. Pat. No. 11,495,326, the contents of which are incorporated herein by reference in their entireties. In some embodiments, metagenomes sequencing further comprise generating the plurality of metagenomic fragment reads. In some embodiments, metagenomic sequencing further comprise fragmenting microbial genomes into random fragments of targeted sizes. The resulting fragments can vary in size. In one embodiment, fragments of approximately 500 nucleotides can be obtained. In some embodiments, fragments of from 100-2000 nucleotides, e.g., 200-800, 100-900, 100-1000, 300-800, 400-900 nucleotides can be obtained. In some embodiments, the method may further comprise extracting the metagenomic fragments from the corresponding biological sample. In some embodiments, metagenomes sequencing further comprise sequencing the fragments using high throughput sequencing methods to generate a plurality of sequencing reads.

In some embodiments, the first plurality of (e.g., at least 100,000) nucleic acid sequences are obtained through targeted panel sequencing, e.g., as described in U.S. Patent Application Publication No. 2019/0316209. In some embodiments, the targeted panel sequencing comprises hybridizing genomic DNA isolated from a biological sample from the gut of a subject with a panel of probes that include one or more probes that hybridize to a unique sequence in the genome of each microorganism being quantified, e.g., each of a plurality of the microorganisms listed in Table 1, Table 2, and/or FIGS. 42A-42XX, prior to sequencing recovered nucleic acids. In some embodiments, a combination of semi-unique sequences (e.g., sequences found in a small number of the microorganism genomes) can be used to deconvolute genomic abundance values using an algorithm, e.g., a system of equations. In some embodiments, the panel of probes includes at least 1 probe that hybridizes to a sequence unique to each microorganism genome being detected. In some embodiments, the panel of probes includes at least 2, at least 3, at least 4, at least 5, at least 10, at least 25, at least 50, or more probes that hybridize to a different sequence unique to each microorganism genome being detected. In some embodiments, the panel of probes includes at least 20, at least 30, at least 40, at least 50, at least 75, at least 100, at least 125, at least 150, at least 200, at least 150, at least 300, at least 400, at least 500, at least 750, at least 1000, at least 1250, at least 1500, at least 2000, at least 2500, at least 3000, at least 4000, at least 5000, at least 7500, at least 10,000 or more unique probes.

In some embodiments, the sequencing genomic DNA from the corresponding biological sample comprise a partial or complete sequencing platform adapter sequence at their termini useful for sequencing using a sequencing platform of interest. Sequencing platforms of interest include, but are not limited to, the HiSeq™, MiSeq™ and Genome Analyzer™ sequencing systems from Illumina®; the Ion PGM™ and Ion Proton™ sequencing systems from Ion Torrent™; the PACBIO RS II Sequel system from Pacific Biosciences, the SOLiD sequencing systems from Life Technologies™, the 454 GS FLX+ and GS Junior sequencing systems from Roche, the MinION™ system from Oxford Nanopore, or any other sequencing platform of interest.

Referring to block 404, in some embodiments, the biological sample from the gut of the respective subject is a fecal sample. In some embodiments, the sample is a tissue biopsy, an intestinal, or mucosal sample. See, for example, Tang Q, J et al., Current Sampling Methods for Gut Microbiota: A Call for More Precise Devices, Front Cell Infect Microbiol., 10:151 (2020), the content of which is incorporated herein by reference in its entirety.

Referring to block 406, in some embodiments, the plurality of gut microorganisms is selected from those microorganisms in Table 5 having a connectivity of at least 2, at least 3, at least 4, at least 5, at least 10, at least 15, at least 20, at least 25, or more. Referring to block 308, in some embodiments, the plurality of gut microorganisms comprises at least 20 microorganisms selected from those microorganisms listed in Table 5 as having a connectivity of at least 2. In some embodiments, the plurality of gut microorganisms comprises at least 20 microorganisms selected from those microorganisms listed in Table 5 as having a connectivity of at least 3, at least 4, at least 5, at least 10, at least 15, at least 20, at least 25, or more.

Referring to block 408, in some embodiments, the method includes obtaining, in electronic form, a plurality of (e.g., at least 100,000) nucleic acid sequences for genomic DNA from the biological sample from the gut of the subject; and determining, for each respective gut microorganism in the plurality of gut microorganisms, the corresponding value for the abundance of the genome of the respective gut microorganism from the plurality of at least 100,000 nucleic acid sequences. In some embodiments, the genomic abundance values determined for the subject comprise no more than 250,000, no more than 100,000, no more than 50,000, no more than 25,000, no more than 10,000, no more than 5,000, no more than 1,000, no more than 100, no more than 50, no more than 30, or no more than 20 genome abundance values. In some embodiments, the genomic abundance values determined for the subject consist of from 10 to 40, from 20 to 50, from 30 to 80, from 40 to 100, from 50 to 150, from 60 to 200, from 80 to 300, from 90 to 500, from 100 to 1000, from 500 to 2,000, or from 1,000 to 5,000 genome abundance values. In some embodiments, the genomic abundance values determined for the subject fall within another range starting no lower than 20 genome abundance values and ending no higher than 250,000 genome abundance values.

Referring to block 410, in some embodiments, the method includes assembling, in electronic form, a corresponding plurality of gut microorganism genomes by metagenomic de novo sequence assembly from the plurality of (e.g., at least 100,000) nucleic acid sequences, and calculate, for each respective gut microorganism in the plurality of gut microorganisms, the corresponding value for the abundance of the genome of the respective gut microorganism based on the prevalence of respective nucleic acid sequences, in the plurality of (e.g., at least 100,000) nucleic acid sequences, used to assemble a respective gut microorganism genome in the plurality of gut microorganism genomes corresponding to the respective gut microorganism. In some embodiments, metagenomic de novo sequence assembly further comprise generating contigs based on the sequencing reads generated by a shotgun sequencing technique, as described in U.S. Pat. No. 10,529,443, the content of which is incorporated herein by reference in its entirety. In some embodiments, the plurality of (e.g., at least 100,000) nucleic acid sequences can be assembled into full genomes of the plurality of gut microorganisms. In some embodiments, the plurality of (e.g., at least 100,000) nucleic acid sequences can be assembled into partial genomes of the plurality of gut microorganisms.

Referring to block 412, in some embodiments, the methods includes assigning, each respective nucleic acid sequence in the plurality of (e.g., at least 100,000) sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and determine, for each respective gut microorganism in the plurality of gut microorganisms, the corresponding genomic abundance value for the respective gut microorganism based on the corresponding count of respective nucleic acid sequences assigned to the respective gut microorganism. In some embodiments, the assigning each respective nucleic acids to a respective gut microorganism includes mapping the nucleic acid to a reference nucleic acid. In some embodiments, the assigning each respective nucleic acids a respective gut microorganism includes annotating genome information based on existing databases. In some embodiments, nucleic acid sequences are analyzed, and annotations are to define taxonomic assignments using sequence similarity and phylogenetic placement methods or a combination of the two strategies.

Sequence similarity based methods include those familiar to individuals skilled in the art including, but not limited to BLAST, BLASTx, tBLASTn, tBLASTx, RDP-classifier, DNAclust, and various implementations of these algorithms such as Qiime or Mothur. These methods rely on mapping a sequence read to a reference database and selecting the match with the best score and e-value. In some embodiments, phylogenetic methods are used in combination with sequence similarity methods to improve the calling accuracy of an annotation or taxonomic assignment. Common databases include, but not limited to, GT-DBTK, National Center for Biotechnology Information (NCBI) Genbank, European Bioinformatics Institute-European Nucleotide Archive (European Bioinformatics Institute-European Nucleotide Archive; EBI-ENA), National Institute of Genetics, U.S. Department of ENERGY (USDOE) Integrated Microbial Genomes (Integrated Microbial Genomes) &Microbiomes; IMG/M) and other available databases in the art.

Referring to block 414, in some embodiments, the method includes inputting the plurality of genomic abundance values into a model comprising a plurality of parameters, wherein the model applies the plurality of parameters to the plurality of genomic abundance values through a plurality of (e.g., at least 10,000) computations to generate as output from the model an indication of the health of the subject.

Referring to block 416, in some embodiments, the indication of the health of the subject is an indication of a biological characteristic, wherein the biological characteristic is a disease or disorder, a therapy administered to the subject, e.g., surgery, radiation therapy, chemotherapy, targeted therapy, gene therapy, immunotherapy, medication, diet change, lifestyle modification, or a diet of the subject, such as a diet rich or poor in carbohydrate, proteins, fats, vitamins, fibers.

Referring to block 418, in some embodiments, the disease or disorder is selected from the group consisting of type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), and Parkinson's disease (PD), Multiple Sclerosis (MS), Gaucher disease type II (GDII), COVID-19 (COV), Behcet's disease (BD), autism spectrum disorder (ASD), or pancreatic cancer (PC). In some embodiments, the disease or disorder is cancer, Alzheimer diseases, a cardiovascular disease, an autoimmune disease, a mental health disease, an infectious disease, or a genetic disorder.

Referring to block 420, in some embodiments, the disease or disorder is cancer.

In some embodiments, the model has been trained against datasets collected across a plurality of disorders and the model is trained to distinguish between a healthy state and an unhealthy state. For example, as described in Example 5, a random forest classifier was trained against datasets from 26 different studies collectively looking at microbiomes in 15 different disorders. As shown in FIG. 38, the resulting model was powered to predict healthy or unhealthy disorder states regardless of the disorder. Accordingly, in some embodiments, the biological characteristic is any one of a plurality of diseases and/or disorders, where the first state is the presence of any one of the diseases or disorders and the second state is the absence of any of the diseases or disorders.

Referring to block 422, in some embodiments, the indication of the health of the subject is a class output of a respective state, in a plurality of possible states, of the health of the subject. In some embodiments, the respective state of the health of the subject is referenced by a severity of a disease or disorder. In some embodiments, severity of the diseases is categorized by the progression or prognosis of a disease or disorder, e.g., different stages of cancer. In some embodiments, a threshold value is provided for determining the state of the health of the subject, such as a level of biomarker, a diagnostic cut-off value, or a threshold nutrient intake level. In some embodiments, the respective state of the health of the subject is the absence or presence of a disease or disorder.

Referring to block 424, in some embodiments, the indication of the health of the subject is a probability output for the corresponding state of the health of the subject. In some embodiments, the corresponding state of the health of the subject is referenced by severity of a disease or disorder. In some embodiments, severity of the diseases is categorized by the progression or prognosis of a disease or disorder, e.g., different stages of cancer. In some embodiments, a threshold value is provided for determining the state of the subject, such as a level of biomarker, a diagnostic cut-off value, or a threshold nutrient intake level. In some embodiments, the corresponding state of the health of the subject is the absence or presence of a disease or disorder.

Referring to block 426, in some embodiments, the model is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

Referring to block 428, in some embodiments, the plurality of parameters is at least 1000, at least 10,000, at least 15,000, at least 50,000, at least 100,000, at least 250,000, at least 500,000, at least 1,000,000 parameters, at least 2,500,000 parameters, at least 5,000,000 parameters, at least 10,000,000 parameters, or more.

Referring to block 430, in some embodiments, the model applies the plurality of parameters to the information through at least 1000 computation, at least 5000 computations, at least 10,000 computations, at least 25,000 computations, at least 50,000 computations, at least 100,000 computations, at least 250,000 computations, at least 500,000 computations, at least 1,000,000 computations, at least 2,500,000 computations, at least 5,000,000 computations, at least 10,000,000 computations, or more to obtain a corresponding output for the respective training subject from the model.

### EXAMPLES

### Example 1—Seesaw-Networked Guilds as a Common Microbiome Signature for Human Diseases

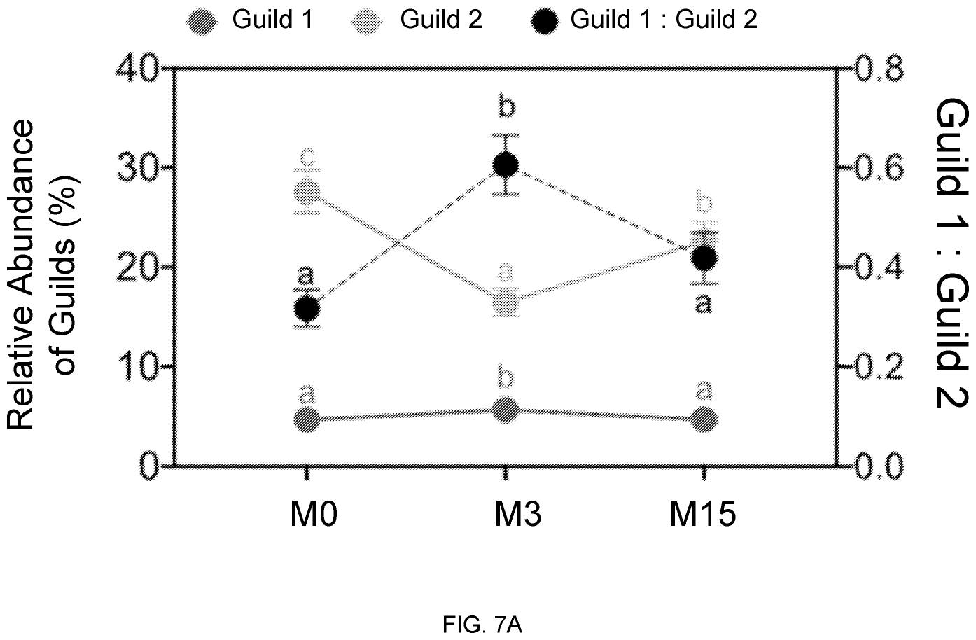

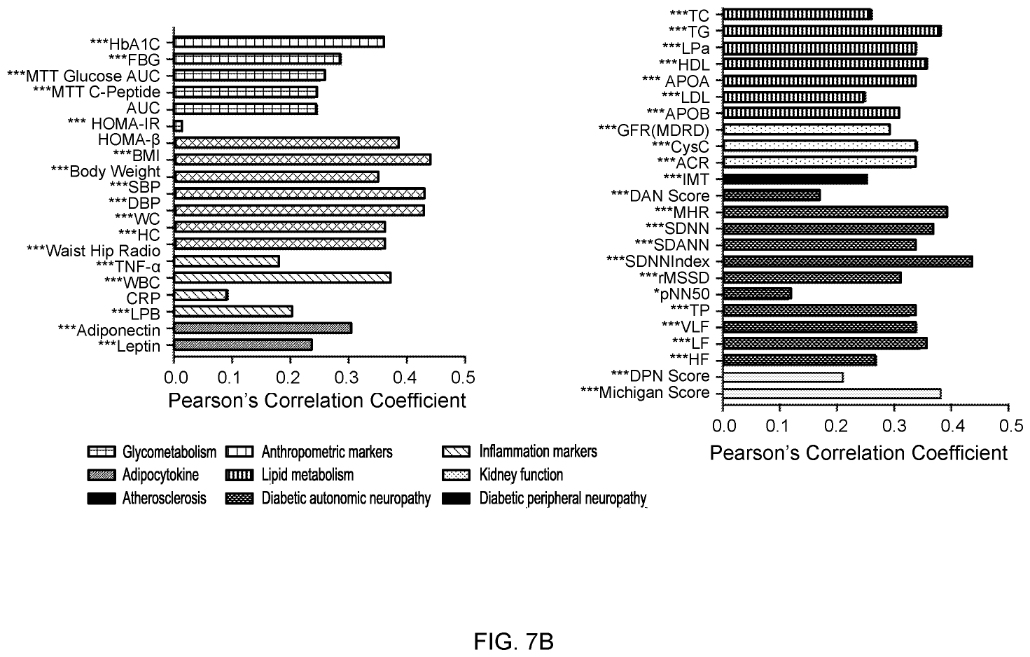

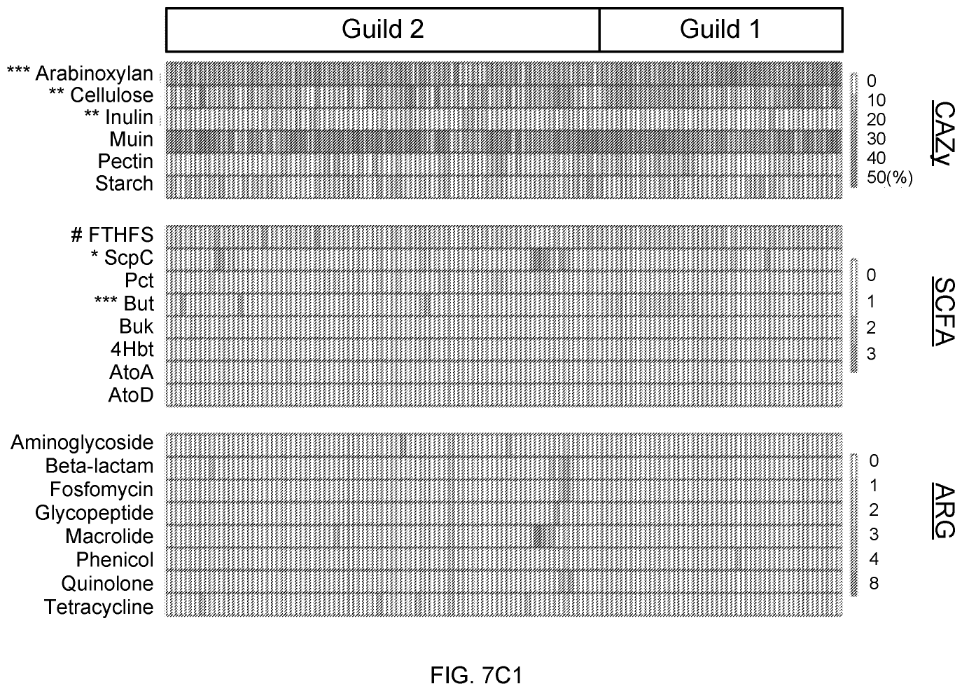

It was hypothesized that microbes that are required for providing essential health-relevant functions to the host [7] should maintain stable ecological interactions with each other for structural and functional stability [18,19]. To identify microbiome signatures that are based on stable interactions among MAGs, T2DM patients were randomized at baseline (M0) to receive either 3-month (M3) of high fiber intervention (W group; n=74) or standard care (U group; n=36) followed by a one-year follow-up (Ml 5) in an open label, controlled trial (FIG. 3A and FIG. 7). The high fiber intervention was used to exert a positive environmental perturbation to dramatically and reversibly change the abundance of members of the gut microbiome [16,17]. Co-abundance network analysis at each of the three time points enabled us to identify MAG pairs that can keep their correlations unchanged despite significant community-wide abundance changes by the perturbations. It was found that that these genome pairs were from 141 MAGs, and they formed two guilds, which were organized as the two ends of a robustly stable seesaw-like network. Together, these seesaw networked genomes supported machine learning models for predicting the response of a wide range of metabolic phenotypes to dietary intervention in the T2DM cohort, as well as for predictive classifications of case and control of 12 independent metagenomic datasets from 1,874 subjects across different cohorts and various chronic diseases including T2DM, atherosclerotic cardiovascular disease (ACVD), hypertension, liver cirrhosis (LC), inflammatory bowel diseases (IBD), colorectal cancer (CRC), ankylosing spondylitis (AS), schizophrenia, and Parkinson's disease (PD), suggesting identification of a common microbiome signature across different human diseases.

Reversible Changes in the Gut Microbiota Associate with Reversible Changes of Host Metabolic Phenotypes

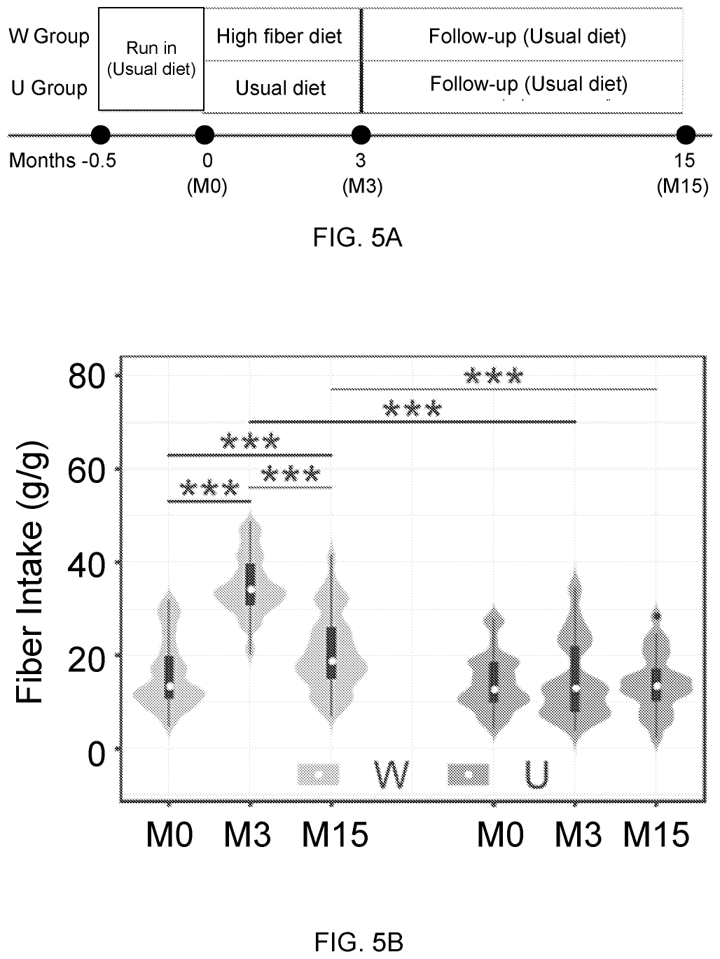

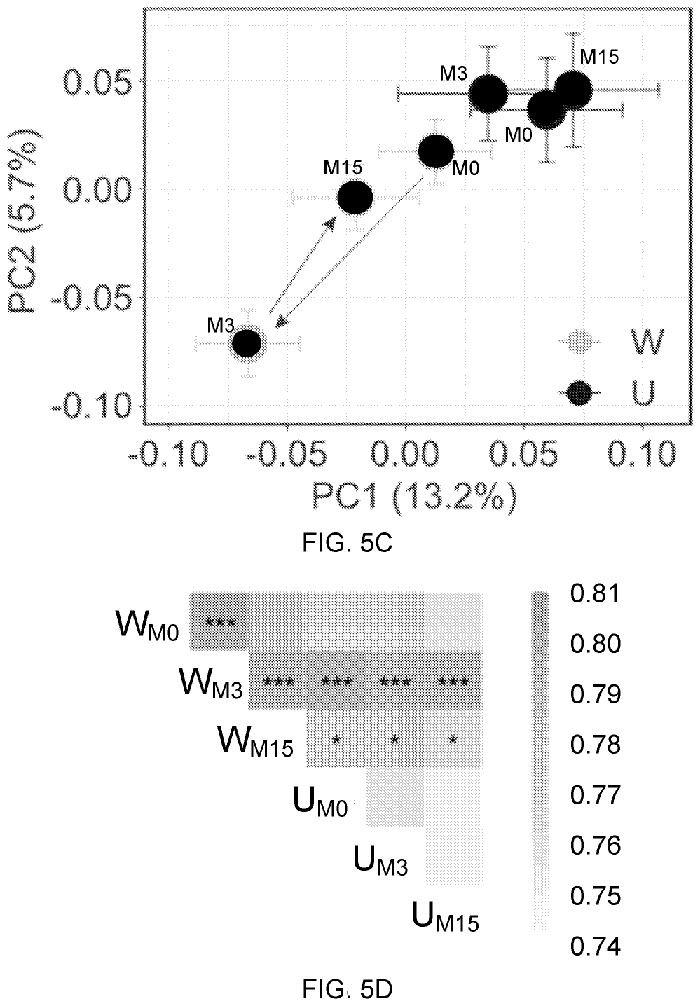

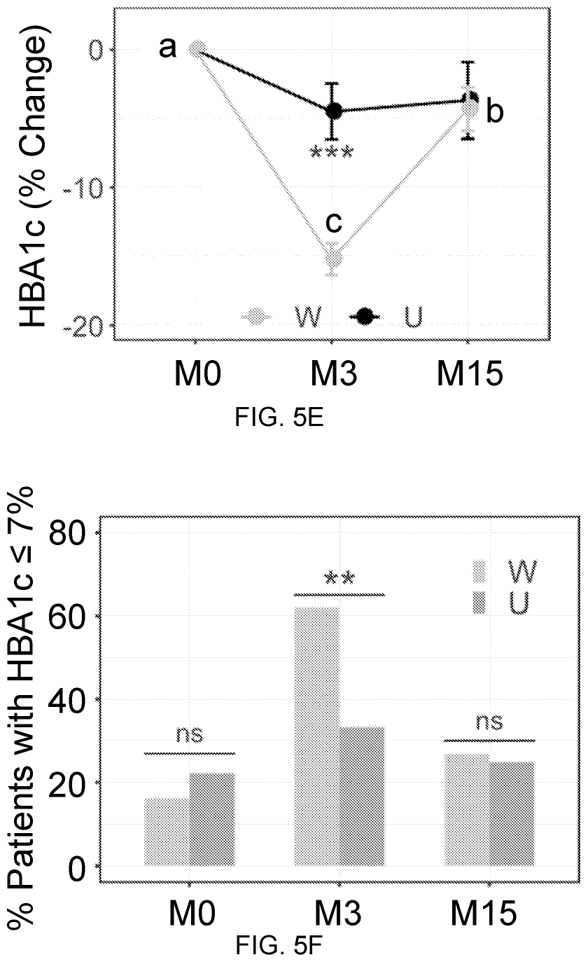

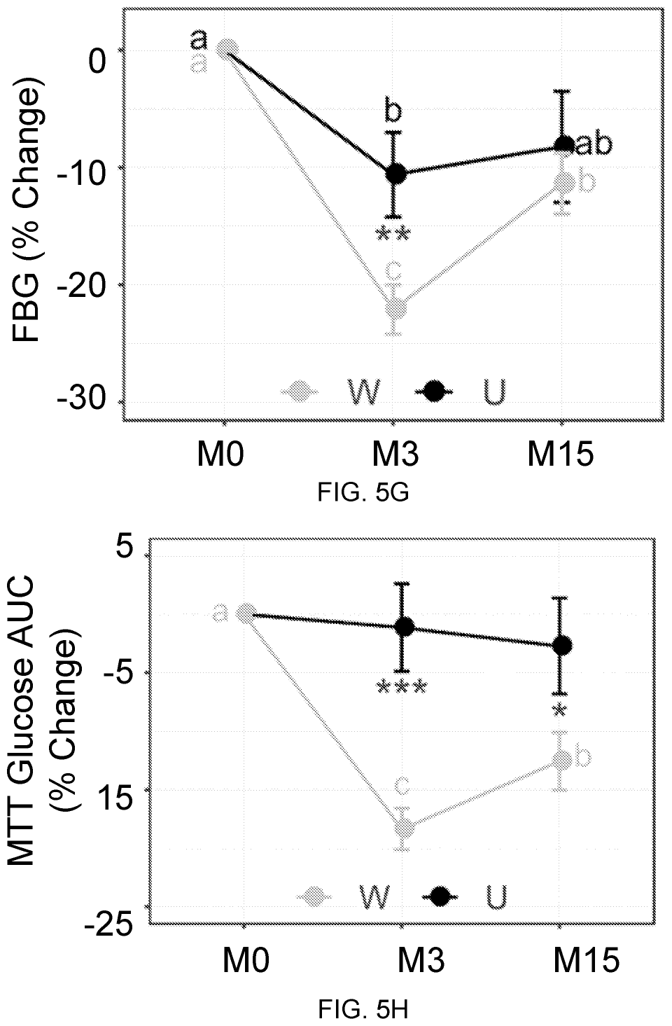

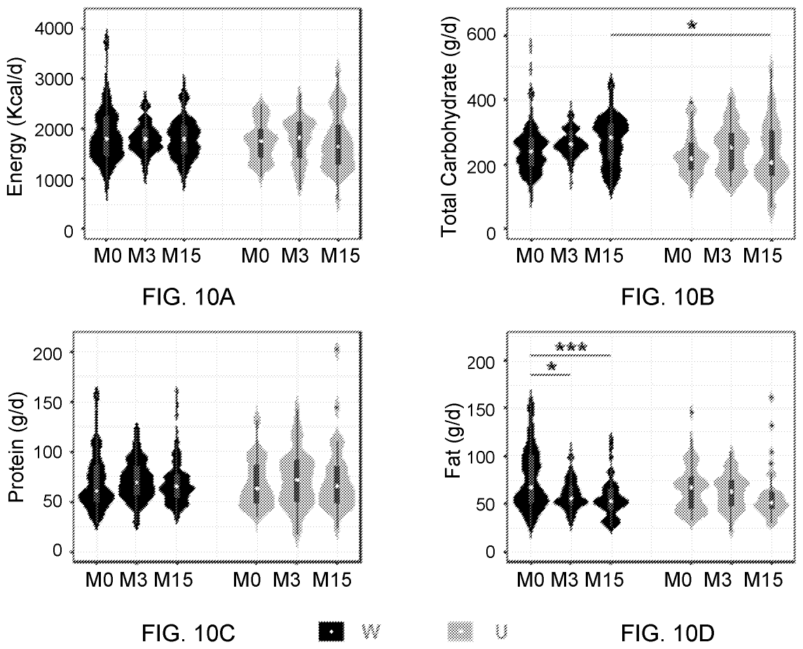

Dietary fiber intake in U group remained unchanged throughout the study, whereas W group had a significant increase in the intake of dietary fibers from M0 to M3 and a decrease from M3 to M15 but remained higher than M0 (FIG. 5B). Compared with U group, fiber intake was significantly higher in W group at M3 and M15, but energy and macronutrient consumption were similar (FIG. 10).

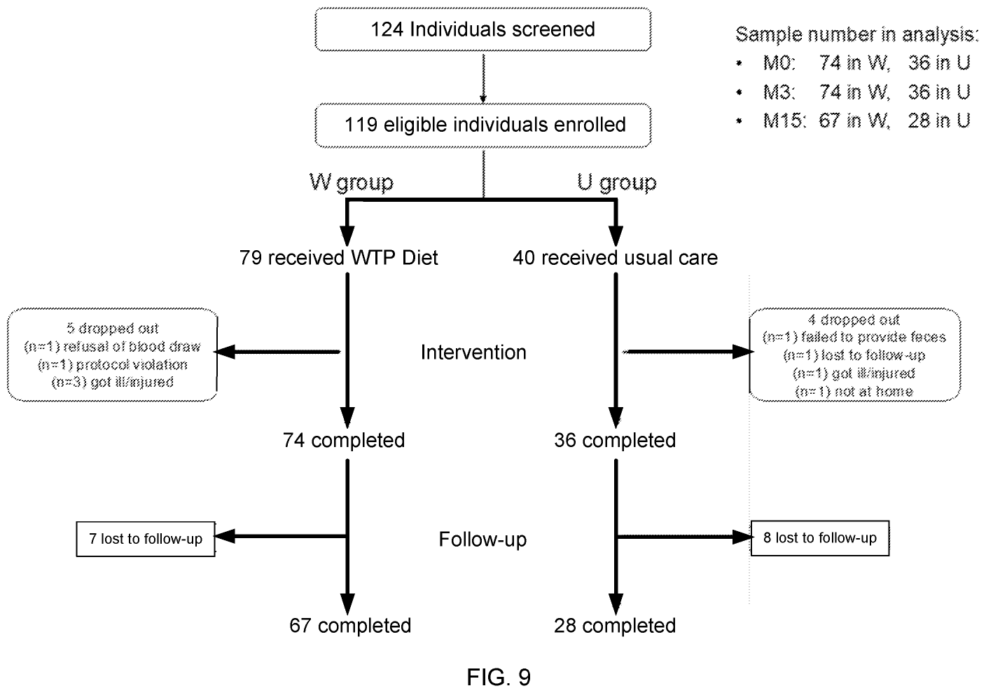

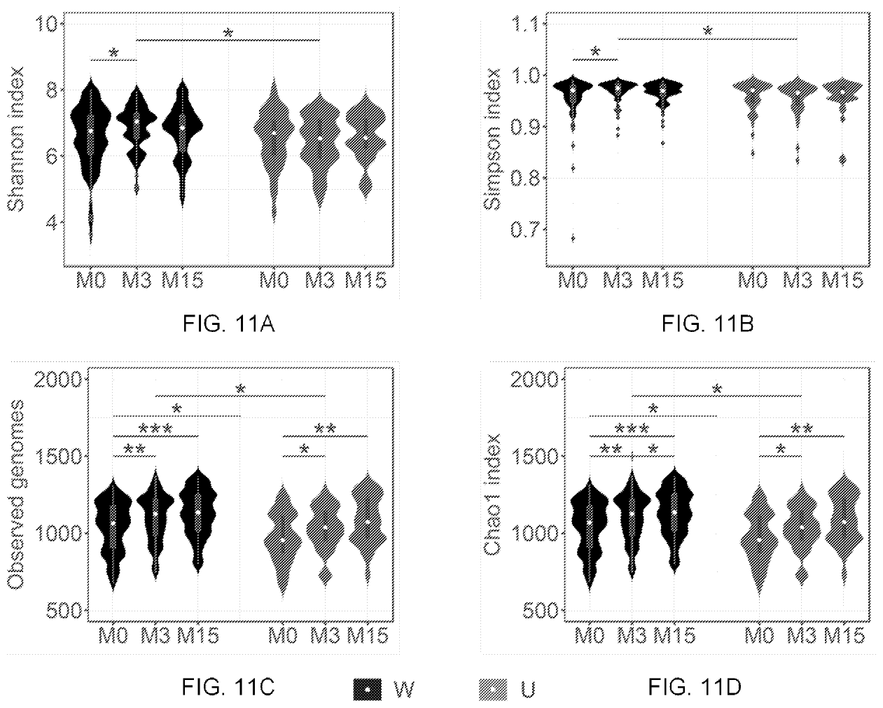

To investigate the structural changes of the gut microbiota in response to the introduction and withdrawal of the high fiber intervention, shotgun metagenomic sequencing was performed on 315 fecal samples collected from 110 patients of the W and U group, among whom 95 patients provided samples at all the 3 time points and 15 provided samples at M0 and M3 only (FIG. 9). To achieve strain and subspecies level resolution, 1,845 non-redundant high-quality draft genomes (two genomes were collapsed into one MAG if the average nucleotide identity, ANI, between them was >99%) were reconstructed from the metagenomic datasets and these MAGs accounted for more than 70% of the total reads. In the context of beta-diversity based on Bray-Curtis distance, the overall structure of the gut microbiota in the W group significantly changed from M0 to M3 (PERMANOVA test, P<0.001) and returned to that of M0 at M15, while there was no difference in the U group across the 3 timepoints (FIG. 5C, D). Similar changes in alpha-diversity based on Shannon and Simpson indices were also observed (FIG. 11). These results showed that high fiber intervention induced significant structural changes of the gut microbiota as previously reported [16], however the gut microbiota reverted to baseline after the intervention was withdrawn which indicates a high resilience in community structure.

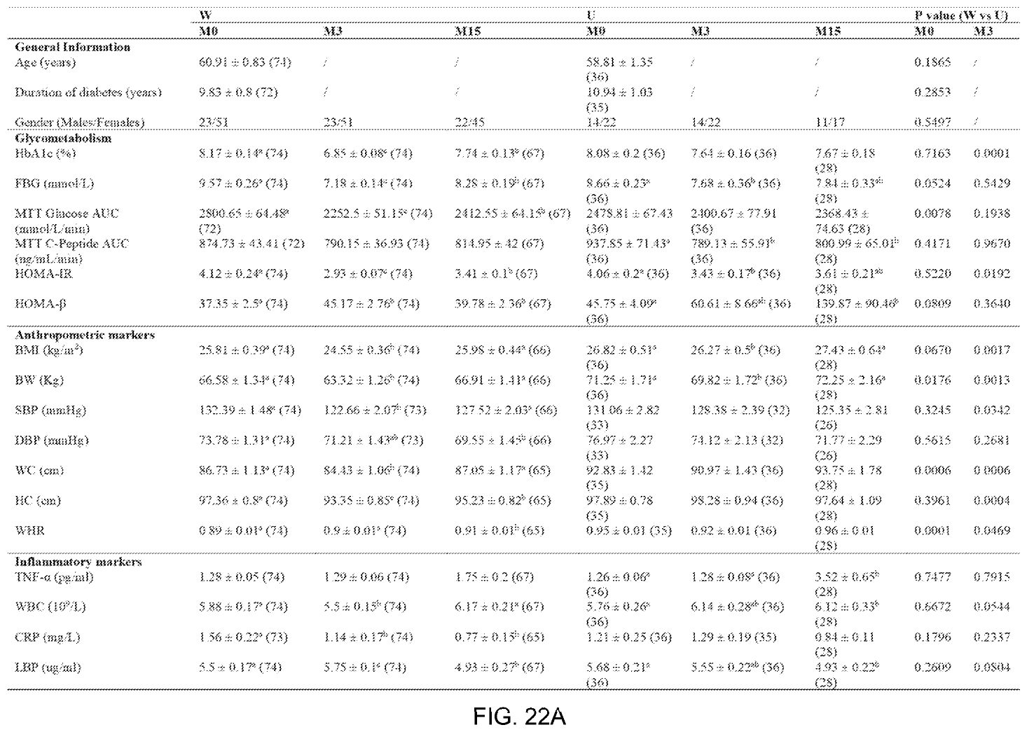

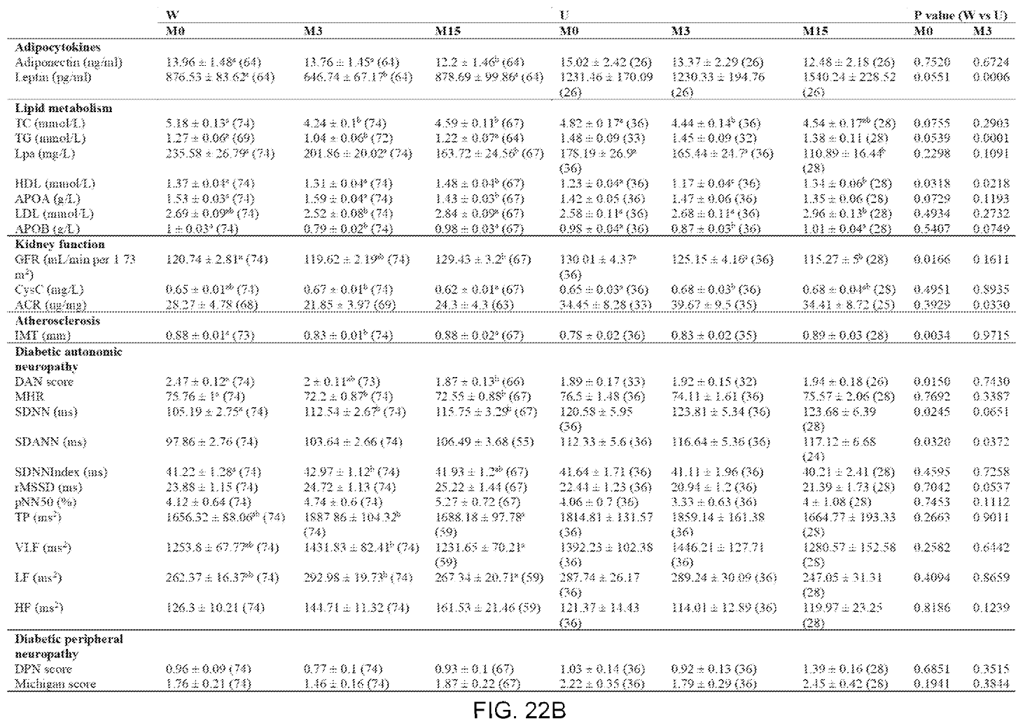

To determine if host metabolic phenotypes would also show similar reversible changes as the gut microbiota, 43 bio-clinical parameters were examined across the 3 time points. Hemoglobin A1c (HbA1c) in the U group showed no changes throughout the trial. The high fiber intervention reduced the level of HbA1c in the W group from M0 to M3 by 15.22%±9.82% (mean±s.d.) on average, and such reduction was significantly bigger than that in U group. At one-year follow-up, HbA1c was significantly increased from M3 but remained lower than M0 in the W group (FIG. 5E). The proportion of patients who achieved adequate glycemic control (HbA1c≤7%) was also significantly higher in the W group (61.6% versus 33.3% in the U group) at M3 but showed no difference at M15 between the two groups (FIG. 5F). The level of fasting blood glucose and postprandial glucose in meal tolerance test followed a similar trend as HbA1c (FIG. 5G, H). The W group also showed an alleviation of inflammation, hyperlipidemia, obesity, and T2DM complications from M0 to M3 but rebounded at one-year follow-up (FIG. 22). Mantel test with the Manhattan distance based on all the 43 bio-clinical parameters and the Bray-Curtis distance of the gut microbiota showed that the clinical outcomes were significantly correlated with the gut microbial structure (R2=0.09, P=2×10-4). These results indicate that changes of the host metabolic phenotypes were associated with the reversible changes of the gut microbiota in response to the presence/absence of the high fiber intervention.

Genome Pairs with Stable Interactions Form a Seesaw Network with Two Competing Guilds

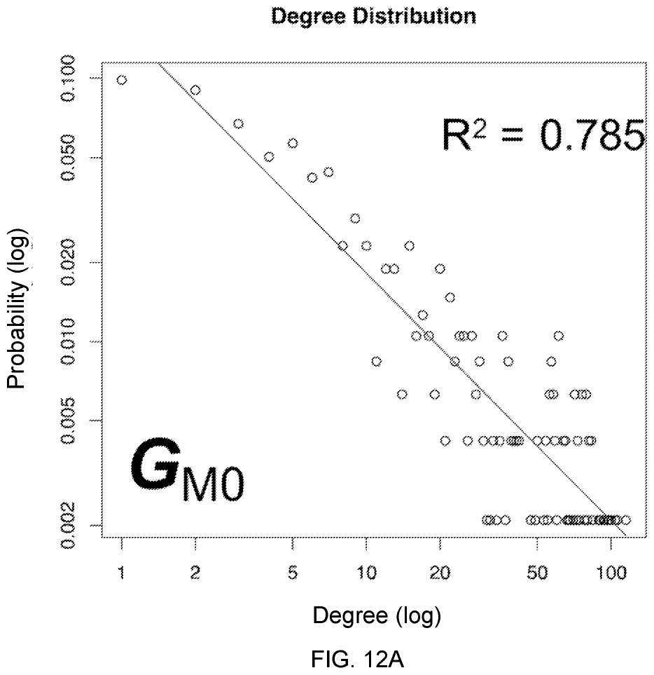

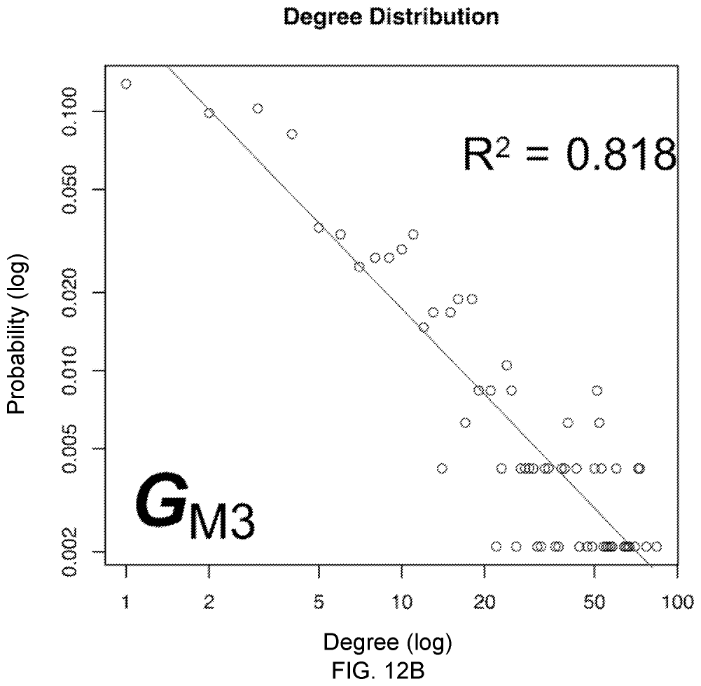

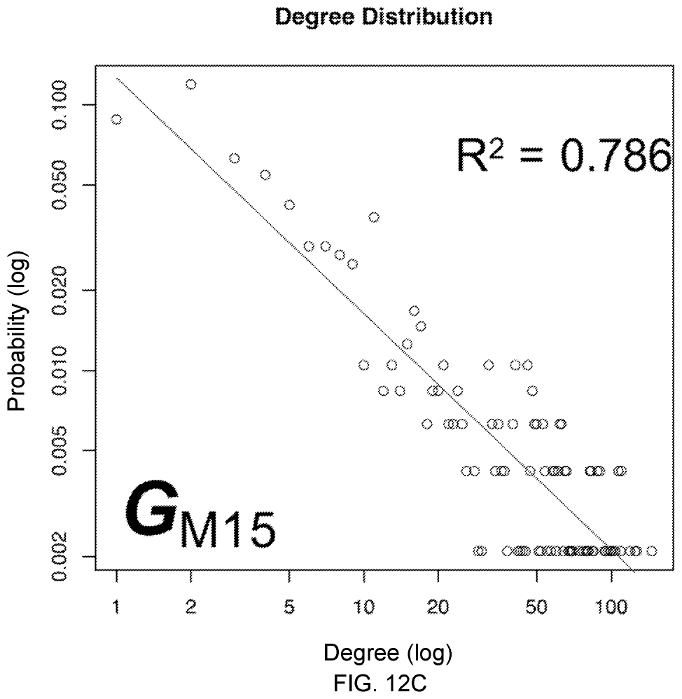

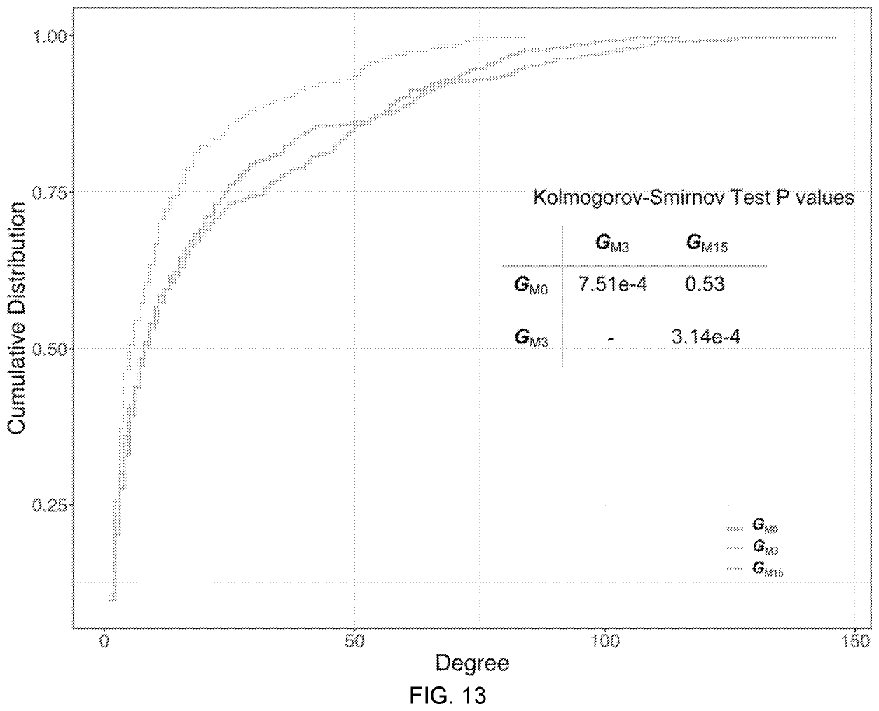

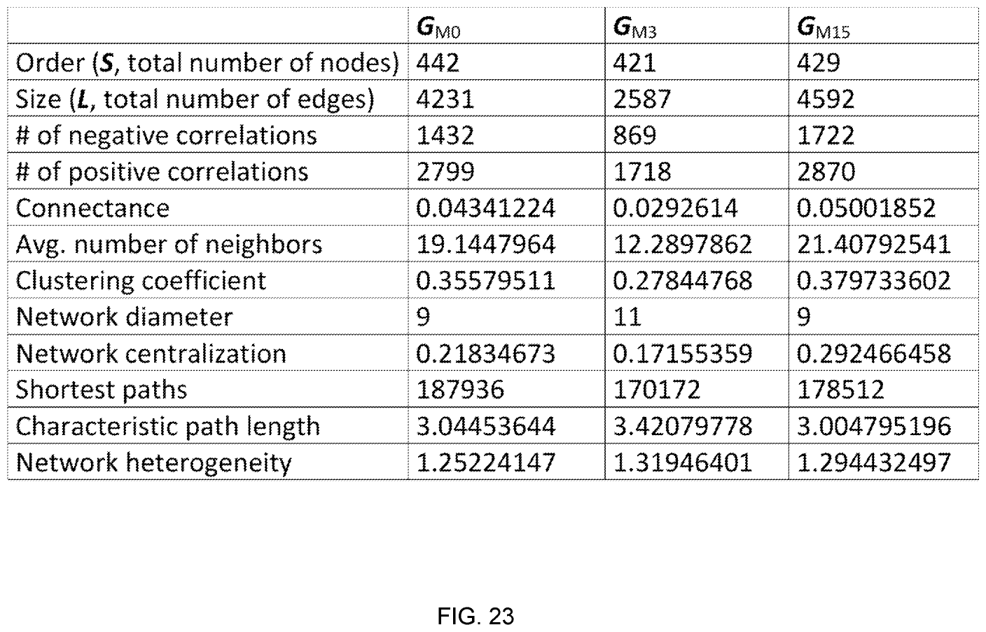

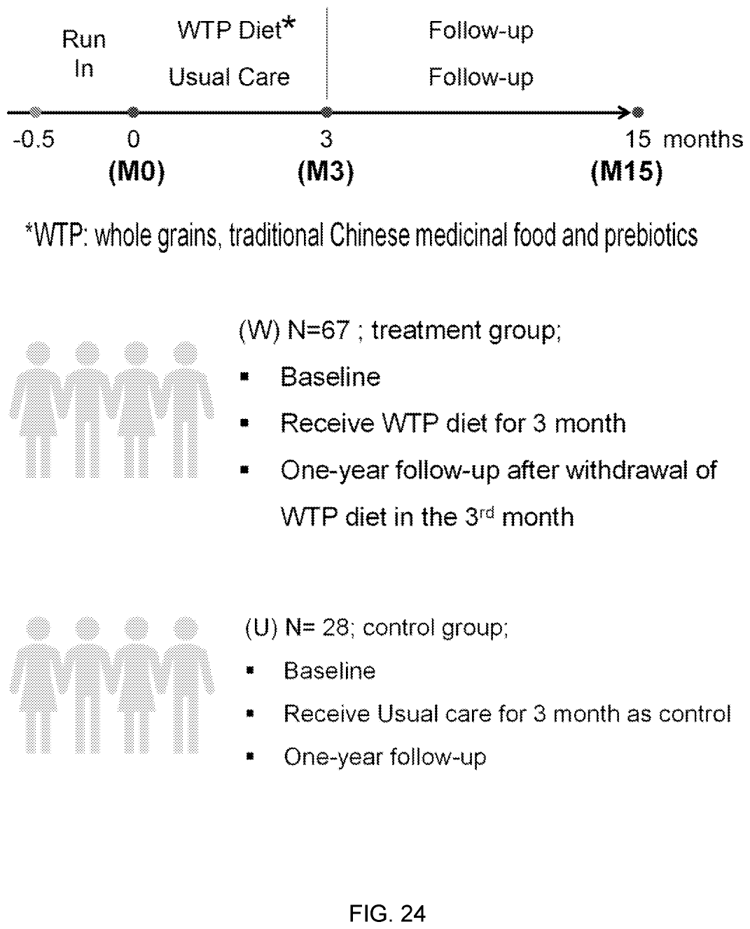

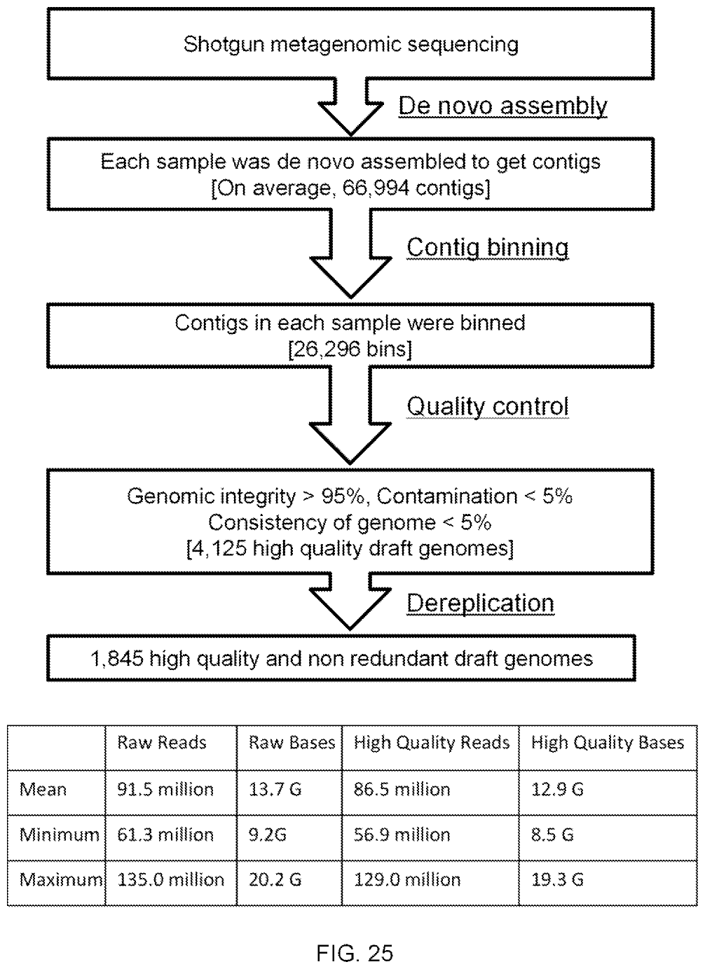

To facilitate the identification of genome pairs which can keep their ecological interactions stable during the trial, a co-abundance network was constructed for each time point based on the abundance matrix of the MAGs representing the prevalent microbes. A total of 477 MAGs were selected for network construction because they were detectable in more than 75% of the samples at each time point in the W group. They were also predominant because they accounted for ˜60% of the total abundance of the 1,845 MAGs. Pairwise correlations were calculated for all 113,526 possible genome pairs among these 477 prevalent MAGs and constructed 3 co-abundance networks, one for each time point (GM0, GM3 and GM15) (FIG. 23). Co-abundance networks of the prevalent genomes in the W group at M0, M3 and M15 during the trial are denoted as GM0(442; 4231), GM3(421; 2587) and GM15(429; 4592). Numbers in parenthesis are order and size of the network. The correlations between genomes were calculated using FastSpar, n=67 patients. All significant correlations with P≤0.001 were included. Co-abundance networks were visualized. Edges between nodes represent correlations. Red and blue colors indicate positive and negative correlations, respectively. Node size indicates the average abundance of the genomes. The layout of the nodes and edges was determined by Edge-weighted Spring Embedded Layout with correlation efficient as weight. The three networks had similar order S, i.e., the total number of nodes (MAGs), SM0(442), SM3(421), and SM15(429), but they varied considerably in their size L, i.e., the total number of edges (correlations), LM0(4231), LM3(2587) and LM15(4592). L at GM3 was decreased to 61.14% of that in GM0 and at GM15 it rebounded back to 108.53% of that in GM0. This was confirmed by changes in connectance, which is defined as the proportion of realized ecological interactions among the potential ones (in undirected network, connectance=L/(S(S−1)/2), the value is in the [0,1] interval)20. The connectance decreased from 0.043 in GM0 to 0.029 in GM3 and rebounded to 0.050 in GM15. High fiber intervention dramatically reduced the interactions among the prevalent genomes in the network. In addition, it was found that the distributions of degree, i.e., the number of edges a node has, were fitted well with power-law model (FIG. 12, R2 values from 0.79 to 0.82) indicating the presence of a small number of nodes with a high number of degree, a feature of scale-free networks which are resistant against random error or decay [21]. Here, hubs as nodes were defined as those that connect with more than one-fifth of the total nodes in the network (FIG. 13). Among the 24 hubs, 10 were in GM0 and 20 in GM15 but none in GM3. This is an indication that the overall structure of the gut microbiome may have undergone profound changes during the trial, particularly, high fiber intervention resulted in the loss of interactions between genome pairs.

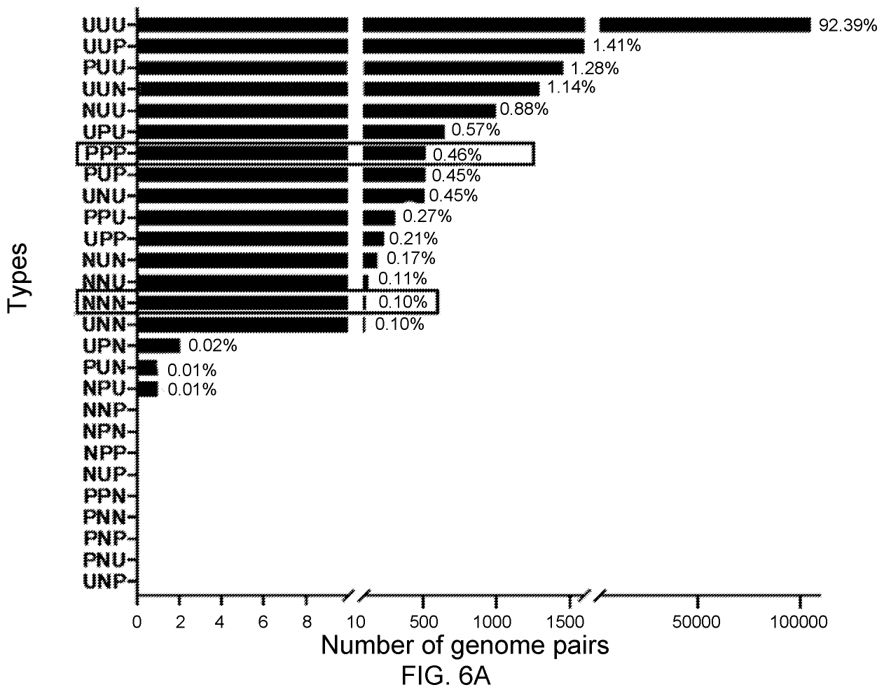

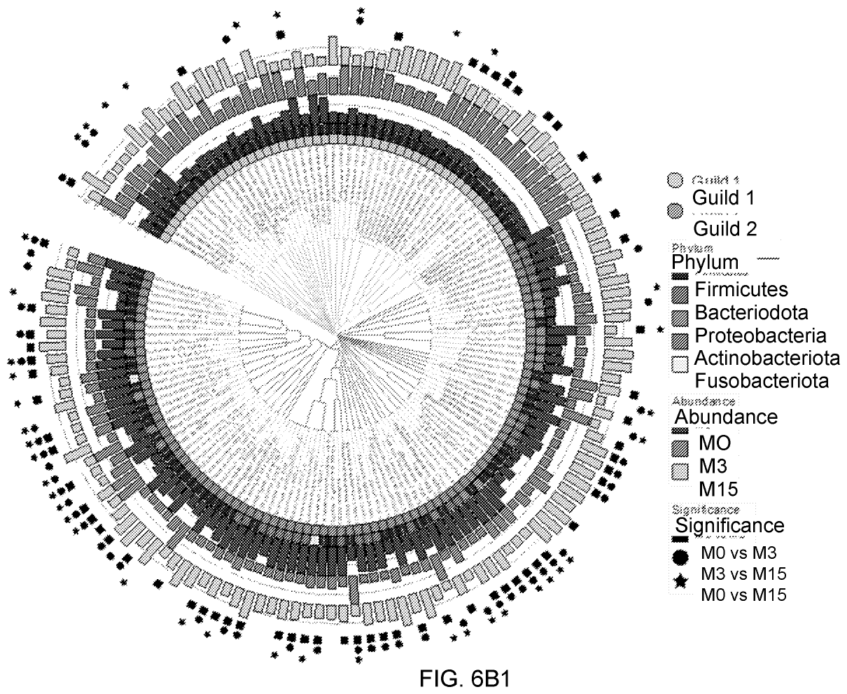

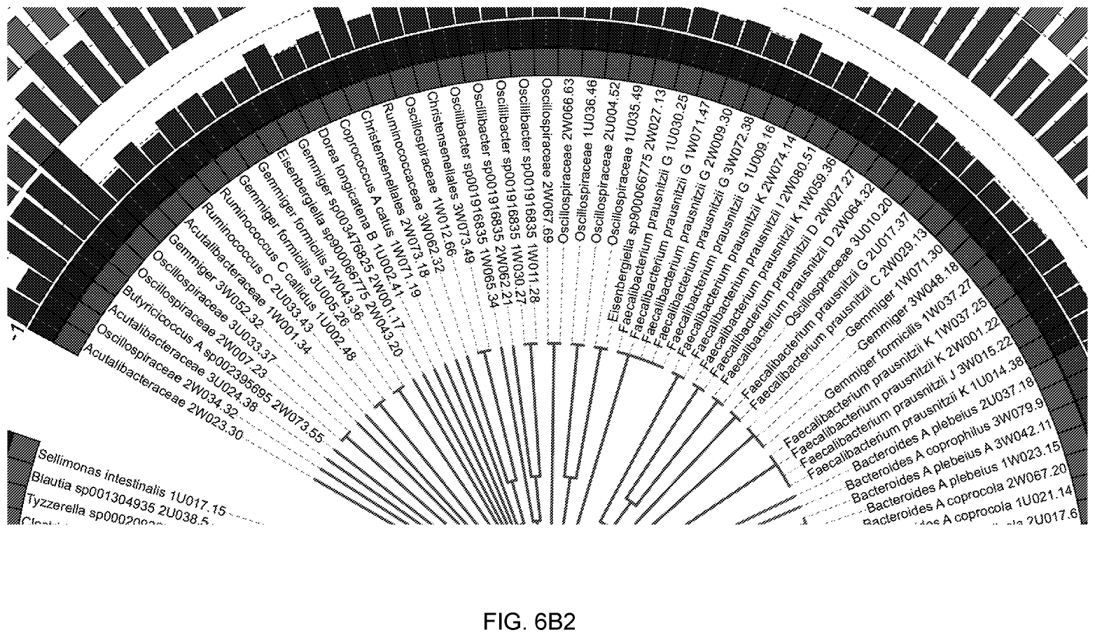

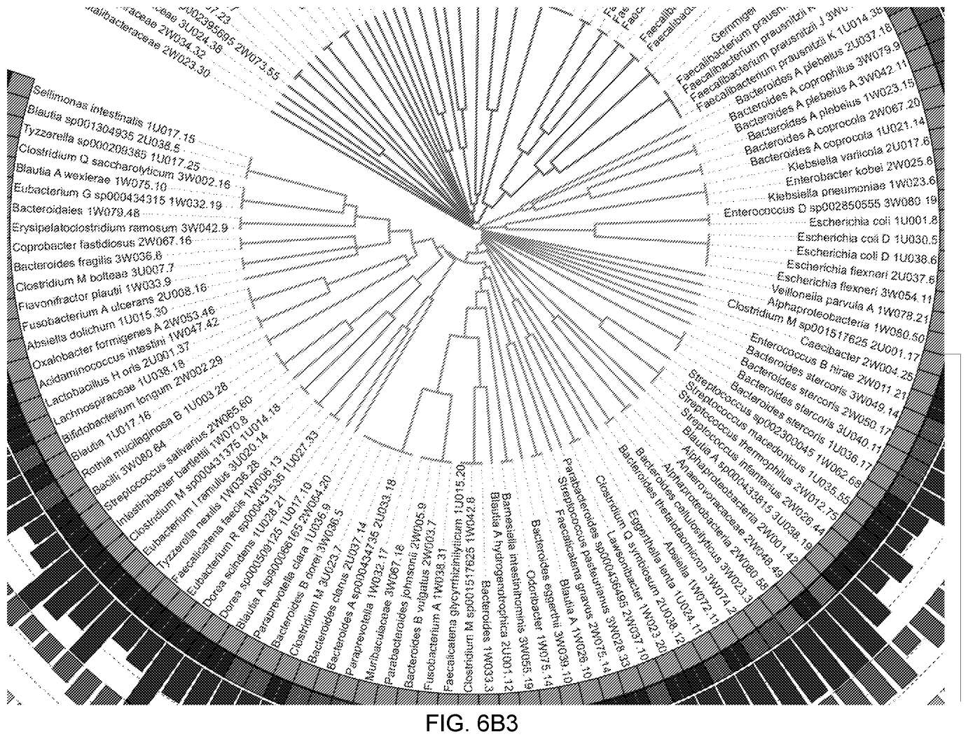

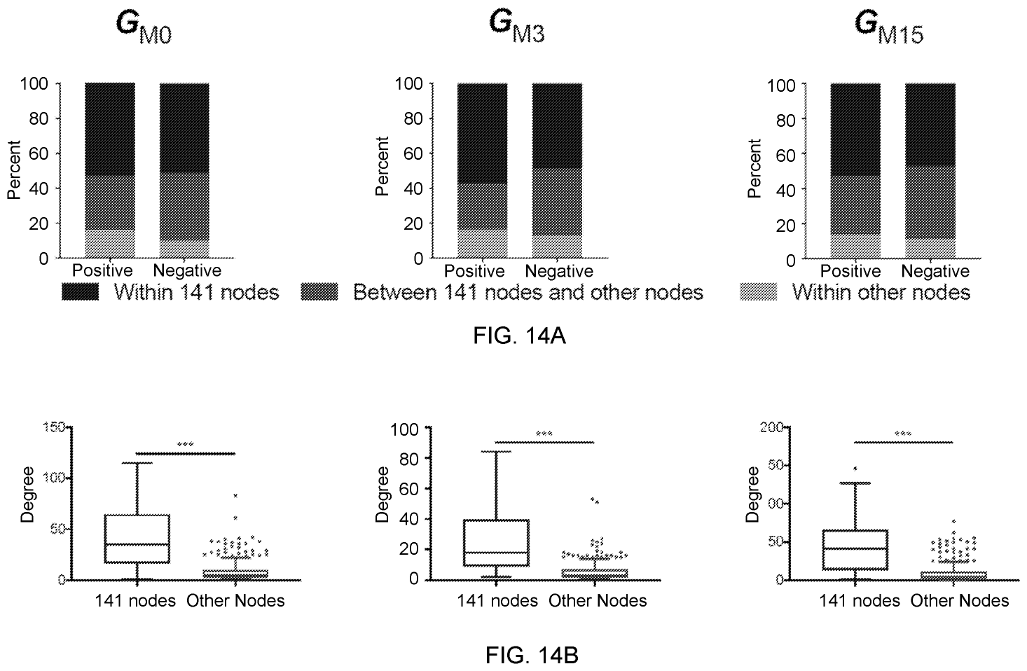

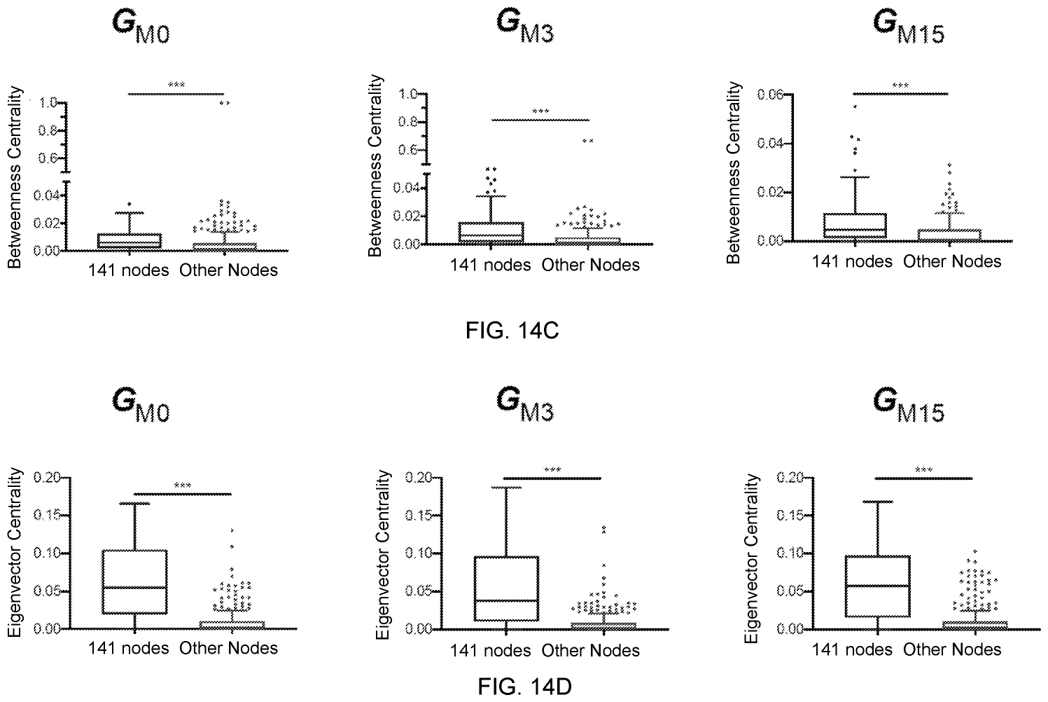

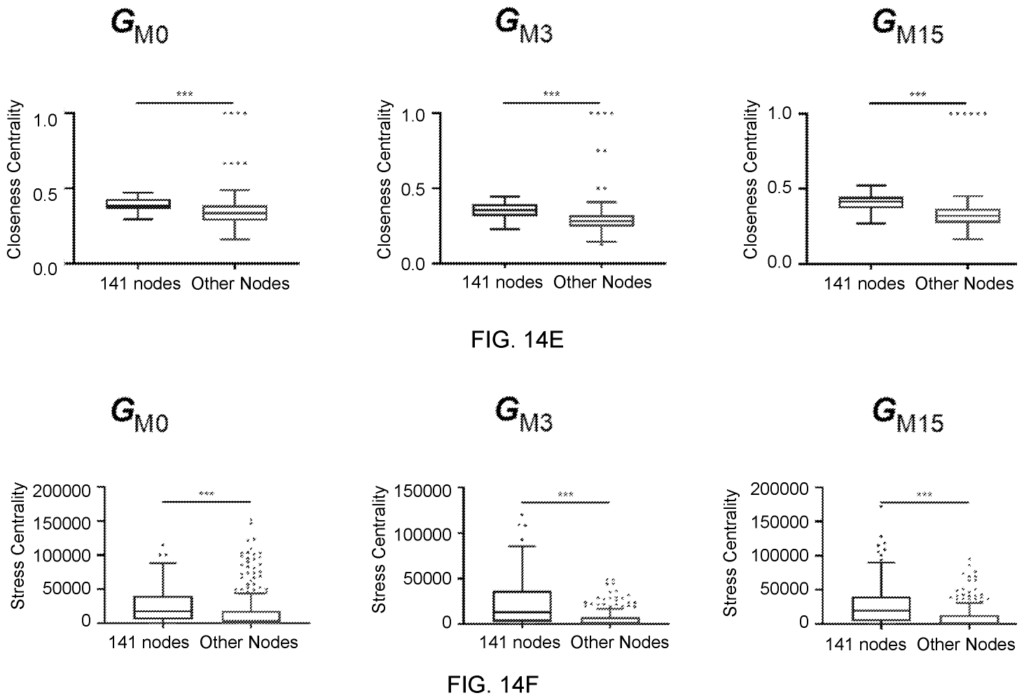

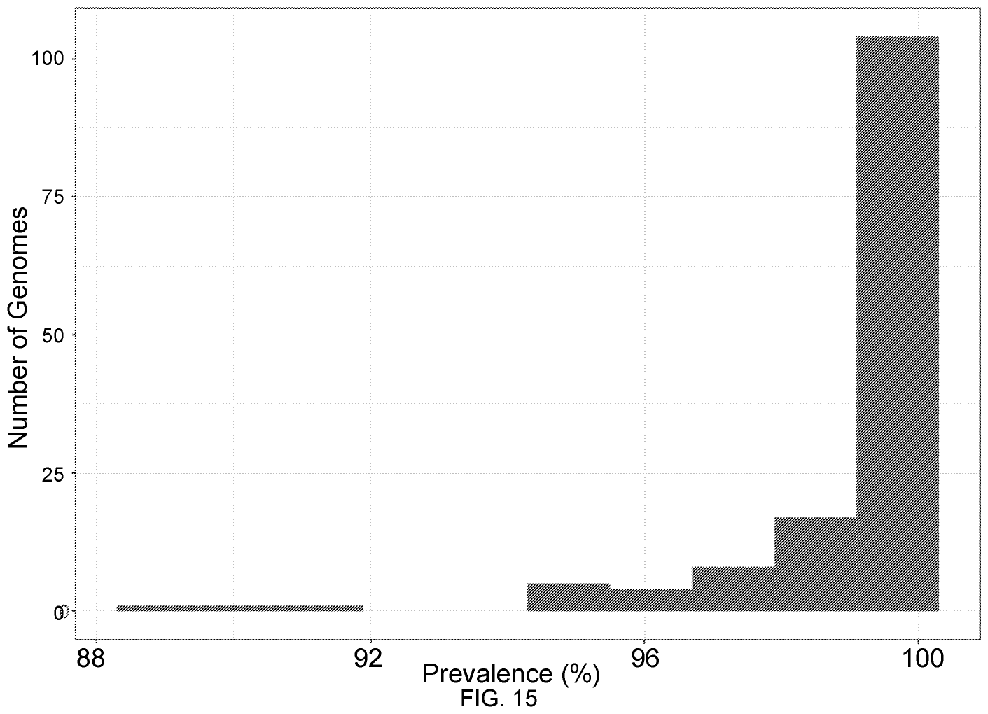

If a genome pair keeps the same ecological interaction across all three timepoints, their ecological relationship was considered robust and stable. Out of the 113,526 possible genome pairs, 92.39% had no correlations at any of the three time points, indicating that it is a rare event for two genomes to establish an ecological relationship (FIG. 6A). Out of the 477 prevalent genomes, 184 had 517 positive correlations and 118 negative correlations within themselves at all three time points. The co-abundance network of 184 genomes with unchanged correlations in GM0, GM3 and GM15 are visualized. The correlations between the genomes were calculated using FastSpar. All significant correlations with P<0.001 were included. Node size indicates the average abundance of the genomes. Lines between nodes represent correlations, and red and blue colors indicate positive and negative correlations, respectively. Edge-weighted Spring Embedded Layout with correlation efficient as weight (−1 or 1) was applied to layout the network. Among these 184 genomes, 43 were excluded from subsequent analysis because they had no interactions with the remaining 141 nodes. The remaining 141 genomes, which included 586 genome pairs with stable correlations throughout the trial, became candidates for the microbiome signature with robust interactions. It was then explored how these 141 genomes were connected with each other and with the rest of the nodes. (FIG. 14A). The 141 genomes had significantly higher degree, betweenness centrality, eigenvector centrality, closeness centrality and stress centrality than the rest of the genomes in the networks (FIG. 14B-F). These indicated that the 141 genomes exerted a relatively large amount of control over the interaction of other nodes (reflected by betweenness centrality and eigenvector centrality) and the information flow in the network (reflected by closeness centrality and stress centrality). Removing these 141 nodes would lead to the collapse of the networks as on average 86.08% of the total edges would have been lost. These suggest that the 141 genomes can be considered as the core nodes of the networks as they were highly connected not only within themselves but also with other nodes.

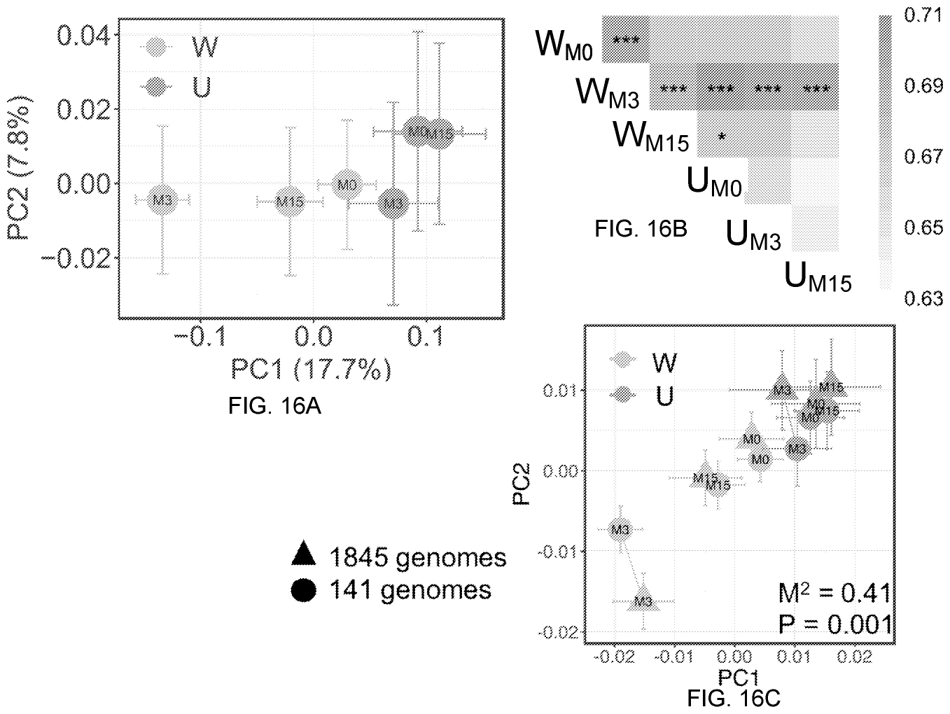

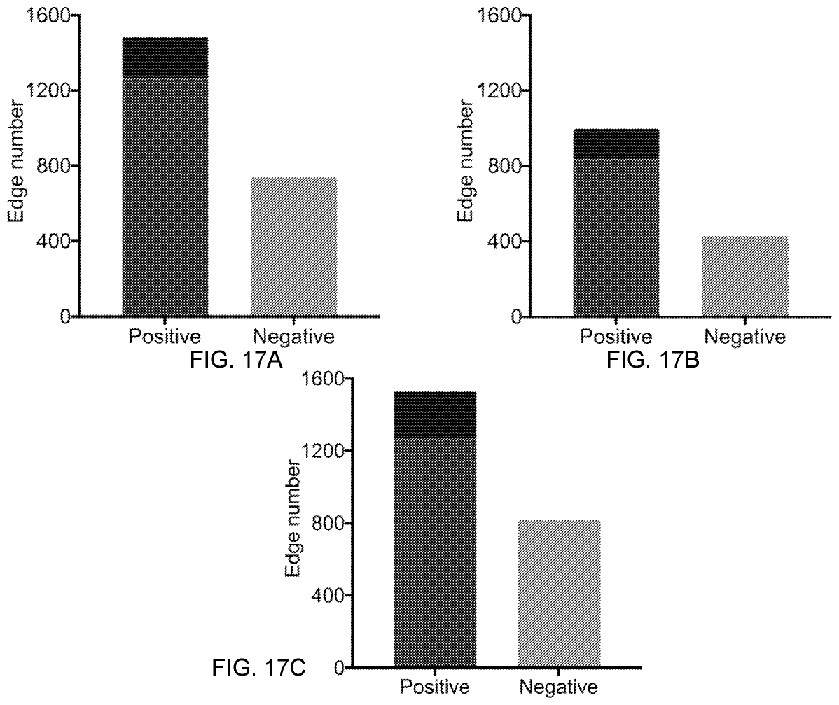

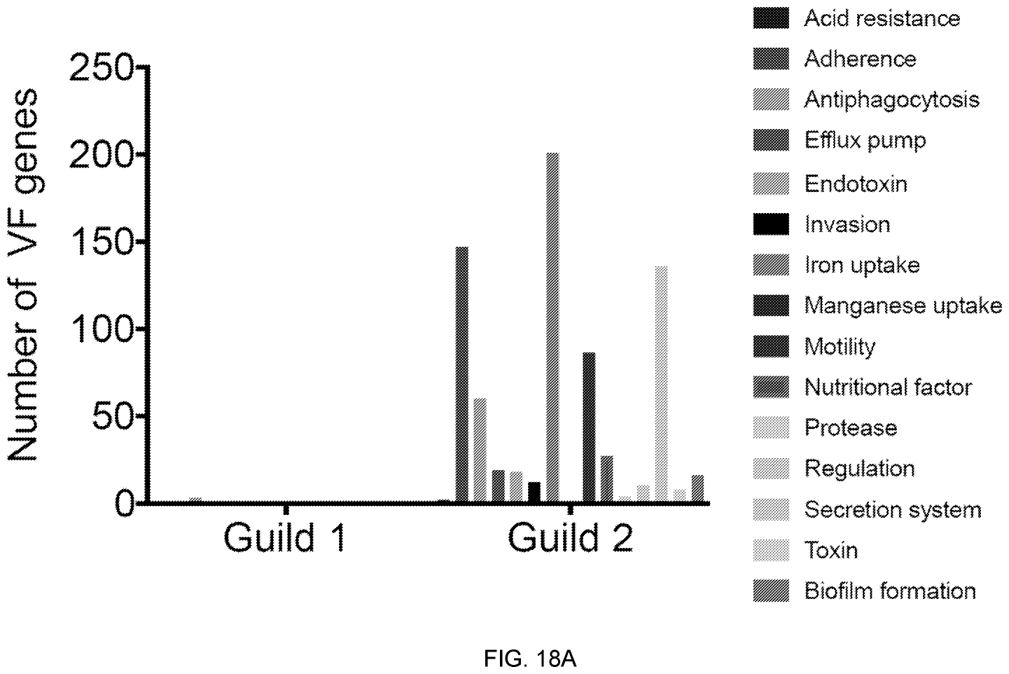

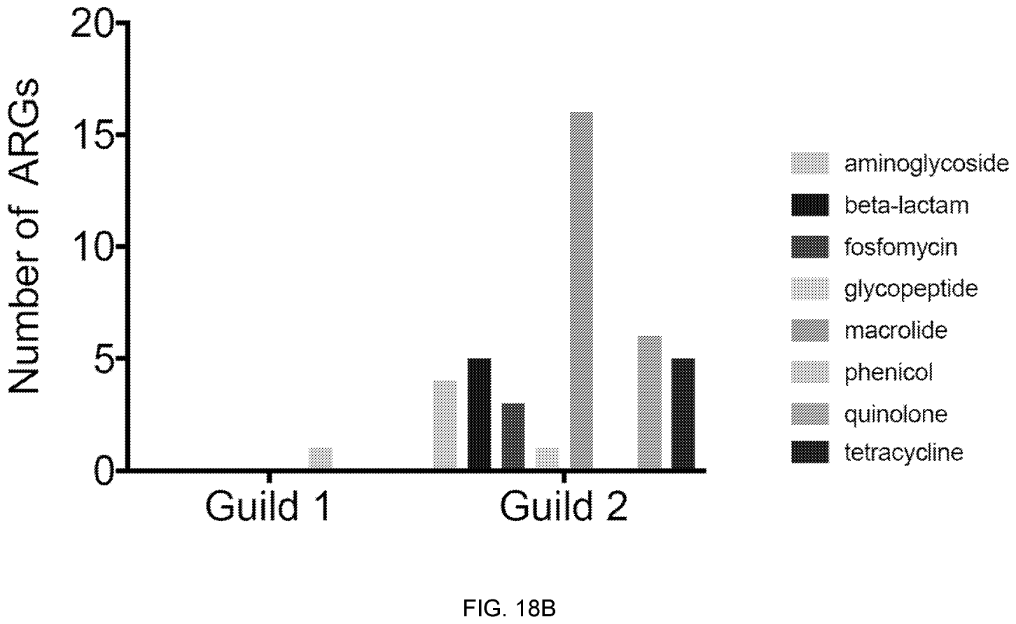

These 141 genomes were also highly prevalent among participants, as 140 of them were in >90%, and 104 were in 100% of the 74 individuals in the W group (FIG. 15). These 141 genomes were also mostly predominant members of the gut microbiota as the abundance of 111 of them was higher than the median of the 1,845 MAGs. Based on Bray-Curtis distance, beta-diversity analysis showed significant correlations between the profiles of the 141 MAGs and all the 1,845 MAGs, as evidenced by Mantel test (R2=0.62, P=0.001) and Procrustes analysis (P=0.001) (FIG. 16, FIG. 5C, D). These indicate that the variations of the 141 MAGs contributed to the major variations of the whole gut microbial community across the 3 time points.

Bacteria which are positively correlated with each other and show robust co-occurrence behavior can be recognized as ecological guilds [5]. The 141 genomes organized themselves into two guilds and genomes in each guild were highly interconnected with positive correlations. Fifty genomes were in Guild 1 and 91 genomes were in Guild 2 (FIG. 6B,). The correlations between the 141 genomes were calculated using FastSpar, n=67 patients. All significant correlations with P<0.001 were included. The co-abundance network within the 141 genomes were visualized. Edges between nodes represent correlations. Red and blue colors indicate positive and negative correlations, respectively. The color of the node represents the members in the two guilds: green for Guild 1 and purple for Guild 2. All the genomes in Guild 1 were from the phylum Firmicutes whereas those in Guild 2 were from 5 different phyla, including Firmicutes, Bacteroidota, Proteobacteria, Actinobacteriota and Fusobacteriota. There were only negative edges between the two Guilds, indicating a competitive relationship. Members of Guild 1 increased its abundance from M0 to M3 and then decreased from M3 to M15 while members of Guild 2 showed an opposite changing pattern (FIG. 6B). Thus, members within each guild had robust cooperative relationships, while competitive relationships existed between the two guilds. The seesaw network with the 141 nodes are in two polarizing clusters. In the network, edges between nodes represent correlations. Red and blue colors indicate positive and negative correlations, respectively. Our data showed that the two guilds of the 141 genomes formed a stable seesaw network existed in all three ecological networks before and after the high fiber intervention and at one-year follow-up in the W group. The co-abundance networks in the U group were constructed based on the 141 genomes and the seesaw network was also observed at each time point. The correlations between the genomes were calculated using FastSpar at each timepoint of U group. All significant correlations with P<0.001 were included. The networks are visualized by lines between nodes represent correlations, and red and blue colors indicating positive and negative correlations, respectively. Node size indicates the average abundance of the genomes across the samples of patients with three timepoints data in U group n=28. The color of the node represents the members in the two Guilds: green for Guild 1 and purple for Guild 2. The percentage of correlations followed the pattern in the seesaw network of the microbiome signature (i.e., positive edges within each guild, negative edges between the 2 guilds) was in yellow, and the ratio of correlations that were negative within each guild and positive between the guilds was in black of the 100% stacked bar.

This indicates that the detection of the stable seesaw networked genomes did not depend on the high fiber intervention, but possibly an inherent signature of the human gut microbiome. Functionality of the metagenomes of the two competing guilds modulates host metabolic phenotypes

It was then determined whether the balance between the two competing guilds could be modulated by dietary fibers and how that affect the host metabolic phenotypes. The total abundance of Guild 1 increased and Guild 2 decreased significantly from M0 to M3. At M15, Guild 1 decreased to a level similar to that at M0, and Guild 2 bounced back but remained lower than M0. Subsequently, high fiber intervention significantly increased the Guild 1 to Guild 2 ratio from M0 to M3. At one-year follow-up, the ratio significantly decreased and was not different from baseline (FIG. 7A). Overall, mantel test with the Manhattan distance based on all the 43 bio-clinical parameters and the Bray-Curtis distance based on the 141 genomes showed that the clinical outcomes were significantly correlated with the variations of the seesaw networked genomes (R2=0.11, P=1×10-4). The associations between the 141 genomes and each host bio-clinical parameter was explored using machine leaning algorithms. Random Forest regression via leave-one-out cross-validation based on the 141 genomes predicted 41 out of the 43 bio-clinical parameters with significant Pearson's correlation coefficient between the predicted and measured values that ranged from 0.11 to 0.44 (FIG. 7B). These results showed that the 141 genomes as two competing guilds in a seesaw network constitutes an important microbiome signature for T2DM and the related metabolic phenotypes.

To explore the genetic basis underlying the association between the dynamic changes of the seesaw networked microbiome signature and the response of the host's metabolic phenotypes, genome-centric analysis of the metagenomes of the two competing guilds was performed. As the balance between the two guilds can be shifted by dietary fibers, carbohydrate-active enzyme (CAZy)-encoding genes and genes encoding key enzymes in short-chain fatty acids (SCFAs) production to compare the genetic capacity for carbohydrate utilization between the two guilds were identified. Compared with genomes in Guild 2, those in Guild 1 had higher proportion of CAZy genes for arabinoxylan (P<0.001), cellulose (P<0.01) and lower proportion of CAZy genes for inulin utilization (P<0.01) (FIG. 7C). There was no difference in genes for starch, pectin, and mucin utilization between the two guilds. Our previous study showed that gut microbiota benefited patients with T2DM via acetic and butyric acid production from carbohydrate fermentation [16]. Among the terminal genes for the butyrate biosynthetic pathways from both carbohydrates (i.e., but and buk) and proteins (i.e., atoA/D and 4Hbt), the copy number of but was significantly higher in Guild 1 and there was no difference in the other terminal genes between the two guilds (FIG. 7C). More than one-third of the genomes in Guild 1 harbored but gene while less than 5% genomes in Guild 2 had this gene (Fisher's exact test P<0.001). Compared with Guild 2, Guild 1 also trended higher in its genetic capacity for acetate production (P=0.06) but a lower genetic capacity for propionate production (P<0.05) (FIG. 7C). These results show that Guild 1 had significantly higher genetic capacity for utilizing complex plant polysaccharides and producing acetate and butyrate than Guild 2.

From the perspective of pathogenicity, 21 out of the 1,845 MAGs encoded 750 virulence factor (VF) genes. Among the 21 VF-encoding genomes, 3 were in Guild 1 while 18 were in Guild 2. Three out of the 50 genomes in Guild 1 had one VF gene involved in antiphagocytosis. In Guild 2, 18 out of the 91 genomes encoded 747 VF genes across 15 different VF classes i.e., acid resistance, adherence, antiphagocytosis, biofilm formation, efflux pump, endotoxin, invasion, iron uptake, manganese uptake, motility, nutritional factor, protease, regulation, secretion system, and toxin (FIG. 7C, 18A). Notably, 98.53% of all the VF genes in Guild 2 were harbored in 8 genomes (1 in Enterobacter kobei, 2 in Escherichia flexneri, 3 in Escherichia coli and 2 in Klebsiella). The highly enriched genes for virulence factors in genomes of Guild 2 (P<2.2×10-16, Fisher's Exact test) indicates that this guild may play an important role in aggravating the metabolic disease phenotypes. In terms of antibiotic resistance genes (ARG), in Guild 1, only 1 genome (2.00% of the genomes in this guild) harbored a copy of an ARG related to phenicol (FIG. 5C, S18B). In Guild 2, 17 genomes (18.68% of the genomes in this guild) encode 40 ARGs for resistance to 7 different antibiotic classes i.e., aminoglycosides, beta-lactam, fosfomycin, glycopeptide, quinolone, macrolide and tetracycline. Thus, Guild 2 may serve as a reservoir of ARGs for horizontal transfer to opportunistic pathogens.

Taken together, our data showed that the two competing guilds had distinct genetic capacity with Guild 1 being potentially beneficial and Guild 2 detrimental. Promoting Guild 1 with dietary fibers shifted the balance between the two guilds that led to more favorable metabolic outcomes.

### The Stably Networked Microbiome Signature Exists in Cohorts Across Ethnicity and Geography

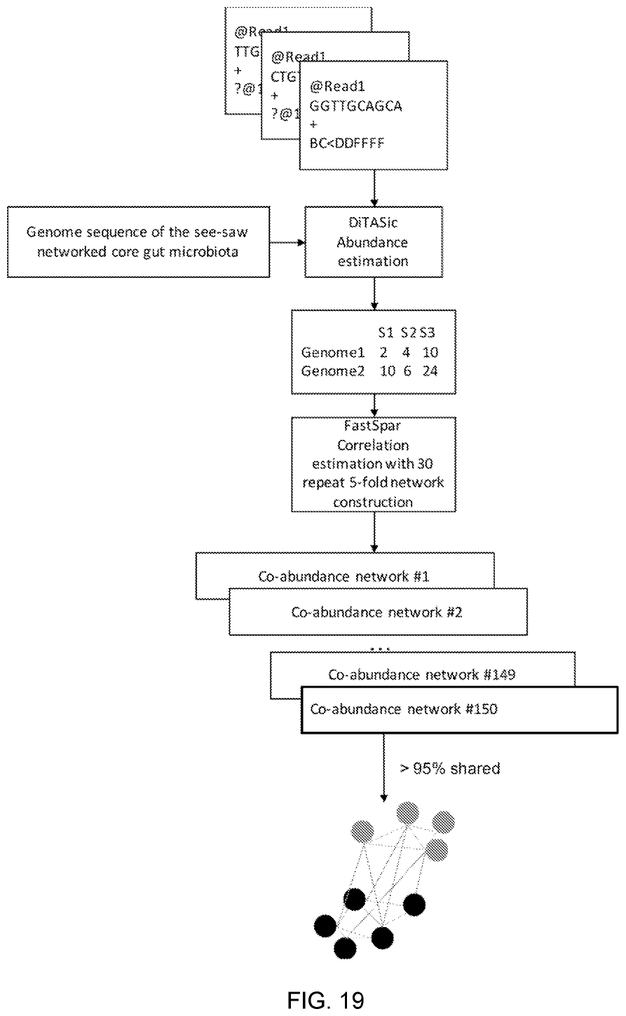

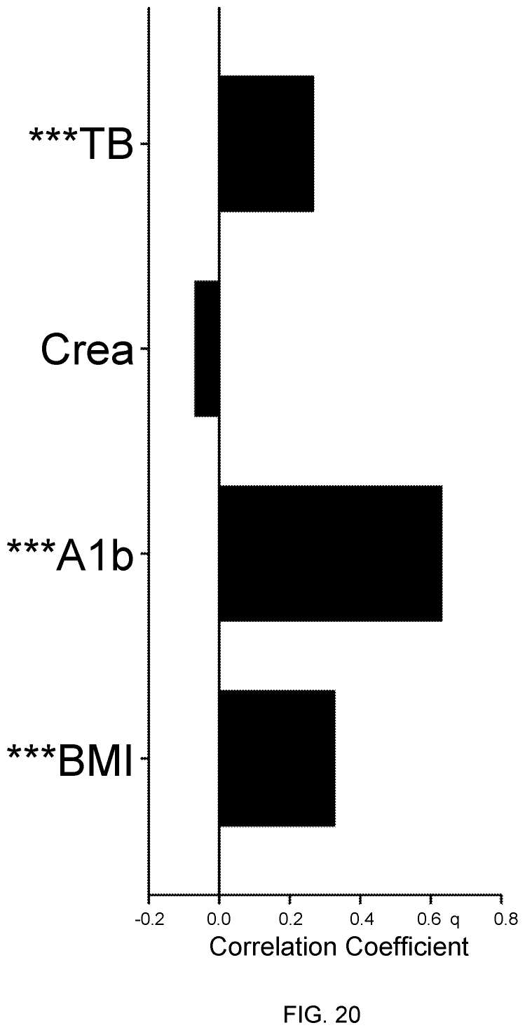

It was then asked whether these 141 genomes that were organized as two competing guilds in a stable seesaw network may be a common microbiome signature for different diseases in other independent metagenomically studied cohorts. To answer this question, the 141 identified genomes were used as reference genomes to retrieve their abundance from an independent T2DM study [22](FIG. 19). In this validation dataset, the reference genomes accounted for 32.93% of the total abundance, and 135 of them were constructed into a co-abundance network in which 99.21% of the total edges followed the pattern in the seesaw network of the microbiome signature (i.e., positive edges within each guild and negative edges between the 2 guilds). This further supported the existence of a similar seesaw network in T2DM patients. Moreover, in the 136 healthy controls of the same study [22], the reference genomes accounted for 35.29% of the total abundance, and 128 genomes were constructed into a co-abundance network in which 98.60% of the total edges of the network were in agreement of the seesaw model. In the context of beta diversity based on Bray-Curtis distance, the microbiome signature showed significant differences between T2DM patients, and the healthy controls based on the abundance matrix of the reference genomes. The composition of the c microbiome signature was different between control and patients in each dataset in the Principal Coordinates Analysis plot based on Bray-Curtis distance. 95% confidence ellipses were projected for control and patients respectively. The p values of the PERMANOVA test were indicated. Additionally, random forest regression models were trained using the abundance matrix of the genomes in the microbiome signature and the phenotype data and found that the predicted values of BMI, fasting insulin and HbA1c from the models were significantly correlated with the measured values (FIG. 20). A random forest model was trained to see whether patients and controls could be differentially classified. Receiver operating characteristic curve analysis showed a moderate prediction power with area under the curve (AUC) of 0.70 by a leave-one-out cross-validation. Thus, it was found that a seesaw networked microbiome signature not only exist but also maintained a similar relationship with the host metabolic phenotypes in an independent T2DM study.

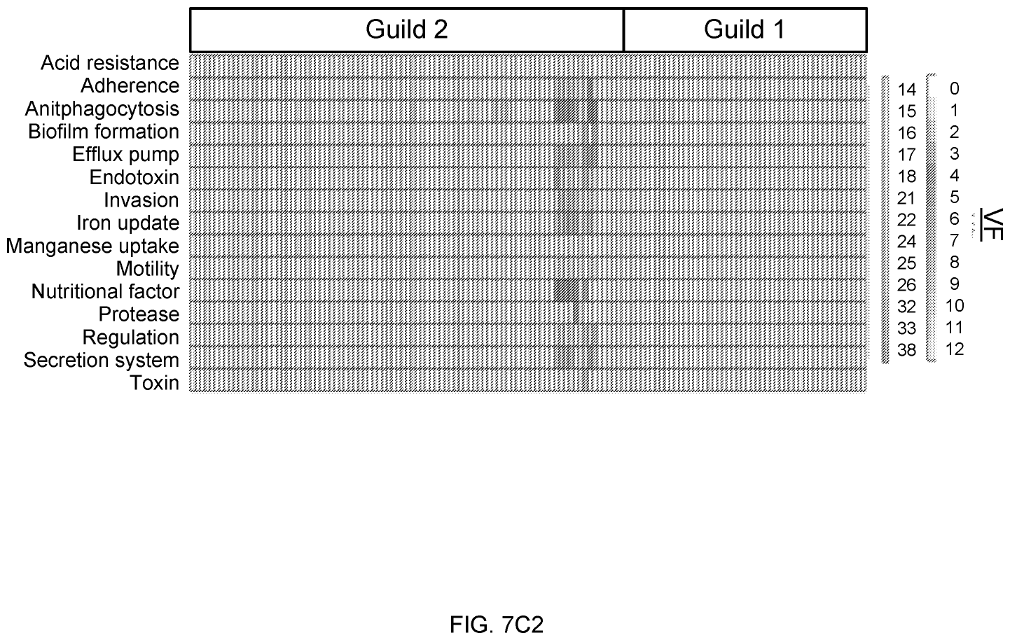

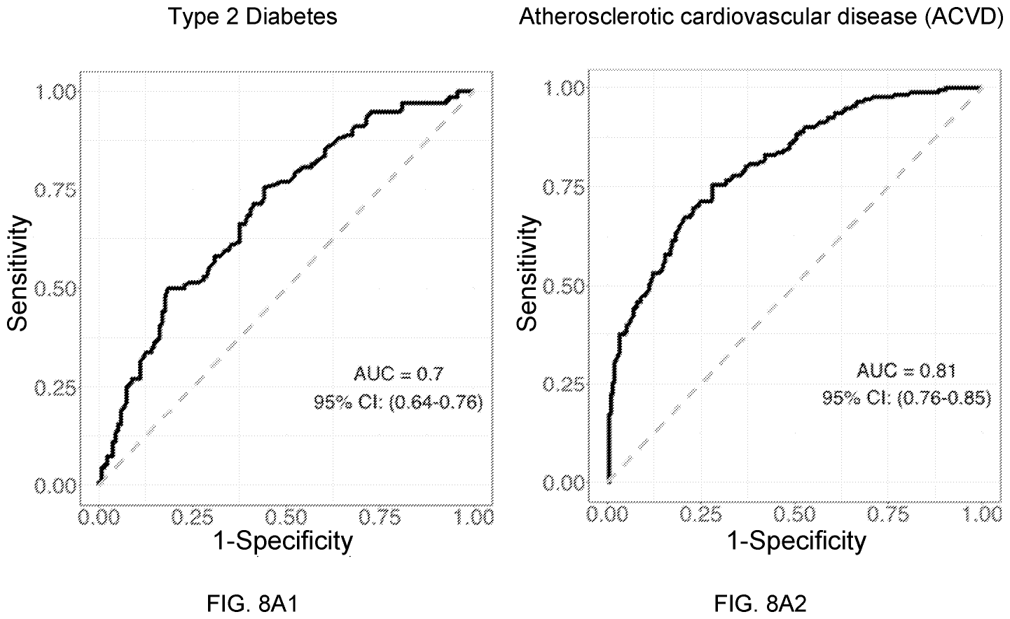

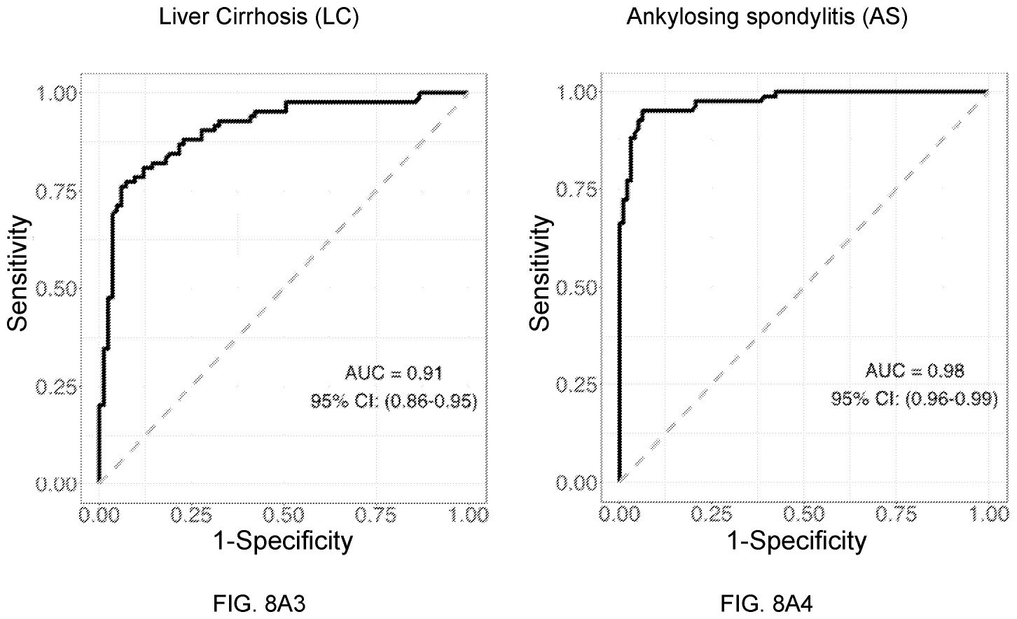

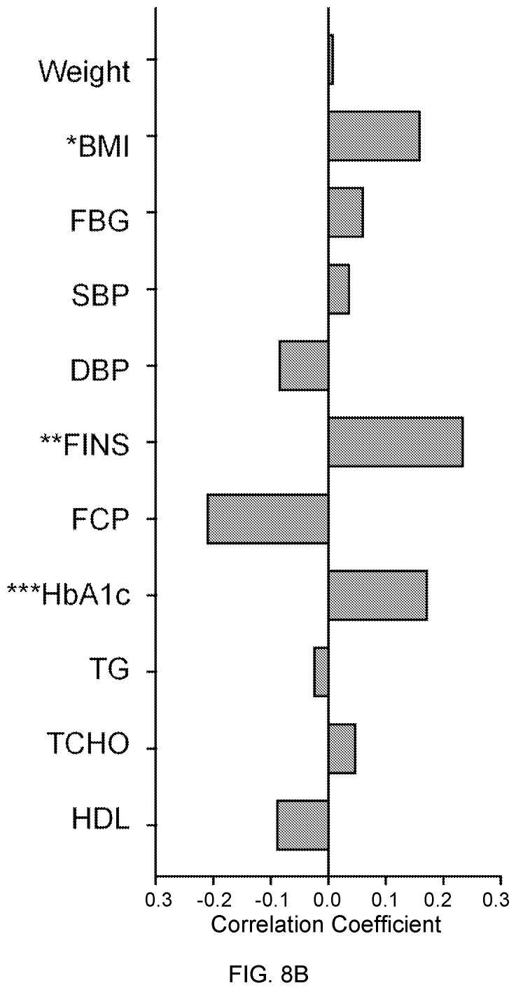

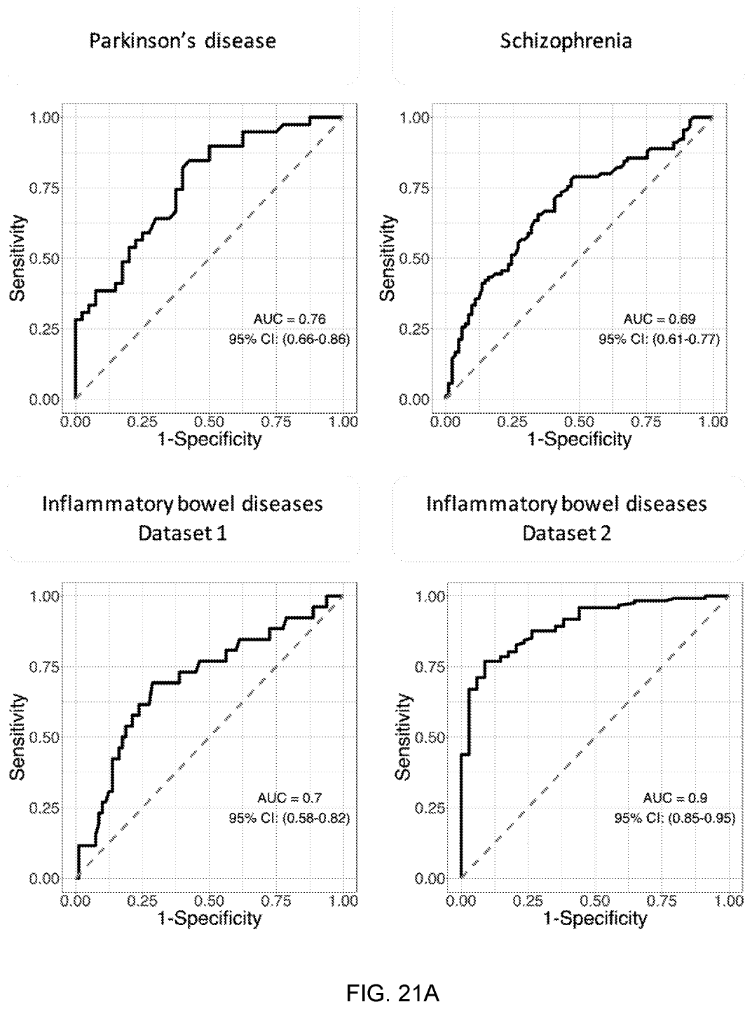

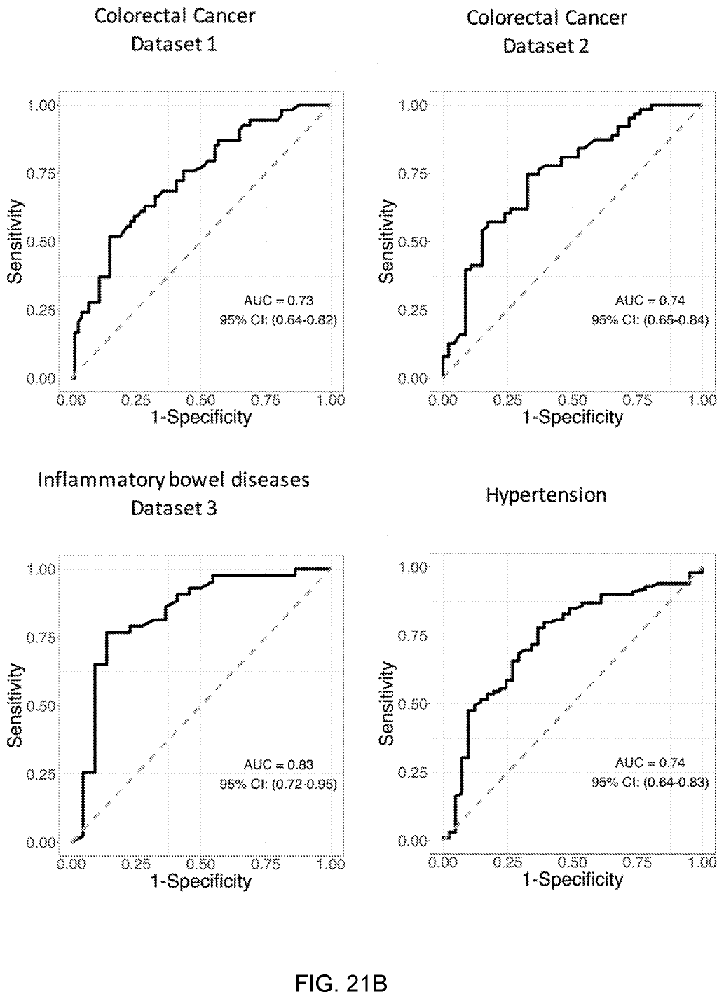

It was further hypothesized that the seesaw networked microbiome signature represents an inherent feature of human gut microbiome, disruption of which may be related to diseases other than T2DM. First, the same validation analysis in metagenomic datasets of case-control studies on three different types of diseases, including ACVD23 (a chronic metabolic disease), LC24 (a liver disease) and AS25 (an autoimmune disease) was performed. Members of the two competing Guilds in the seesaw networked microbiome signature showed similar ecological interactions in four independent human gut metagenomic datasets. The correlations between the genomes were calculated using FastSpar. All significant correlations (P≤0.001) belonged to seesaw model (positive correlations within Guilds and negative correlations between Guilds) were included. The networks were visualized by lines between nodes represent correlations, and red and blue colors indicate positive and negative correlations, respectively. The color of the node represents the members in the two seesaw Groups: green for Guild 1 and purple for Guild 2. The percentage of correlations followed the pattern in the seesaw networked microbiome signature (i.e., positive edges within each guild, negative edges between the 2 guilds) was in yellow, and the ratio of correlations that were negative within each guild and positive between the guilds was in black of the 100% stacked bar. In ACVD patients and their controls, the reference genomes from the microbiome signature accounted for 32.73% and 36.22% of the total abundance respectively, and 139 genomes from the patients and 137 genomes from the controls were constructed into co-abundance networks with 94.33% and 98.49% of the total edges respectively in agreement with the seesaw model. The reference genomes from the microbiome signature accounted for 33.84%, 35.83% and 41.02% of the total abundance in the metagenomic datasets of the healthy control (the studies on LC and AS employed the same control cohort), LC and AS patients respectively. 117, 125 and 123 reference genomes were constructed into co-abundance networks with 100%, 98.68% and 88.54% of the total edges in agreement with the seesaw network model in the metagenomic datasets of the healthy control, LC and AS patients respectively. In the PCoA plot based on Bray-Curtis distance, the microbiome signature showed significant differences between control and patients in all 3 datasets. For the LC study, random forest models were trained using the abundance matrix of the reference genomes and the phenotype data and found that the predicted values of total bilirubin, albumin level and BMI based on the models were significantly correlated with the measured values (FIG. 21). Compared with the T2DM dataset [22], the Random Forest classifier based on the microbiome signature showed better prediction power in distinguishing case from control for ACVD (AUC=0.81), LC (AUC=0.91) and AS (AUC=0.98) (FIG. 8A). To further confirm the relevance of the microbiome signature to human diseases, genomes from the microbiome signature were detected in datasets from more disease types and across different ethnicity and geography. These datasets included hypertension (Chinese cohort), IBD (American cohort and Dutch cohort), CRC (Chinese cohort and Australian cohort), schizophrenia (Chinese cohort), and PD (Chinese cohort). On average, the reference genomes accounted for 31.82%±4.05% (mean±s.d.) of the total abundance of the whole microbiota community in the datasets. The microbiome signature was shown to perform predictive classification between case and control in the metagenomic dataset from studies on hypertension [26](AUC=0.74), IBD (AUC=0.70 for IBD dataset 127, AUC=0.90 for IBD dataset 228 and AUC=0.83 for IBD dataset 328), CRC (AUC=0.73 for CRC dataset 129 and AUC=0.74 for CRC dataset 230), schizophrenia (AUC=0.69), and PD31 (AUC=0.76) (FIG. 22). These results showed the existence of our microbiome signature in healthy controls and various patient populations across ethnicity and geography from independent studies. The associations between the 141 genomes and host phenotypes and their discriminative power as biomarkers to classify controls vs. patients with various types of diseases indicate that these seesaw networked genomes organized in two guilds represent a common microbiome signature associated with widely different human disease phenotypes.

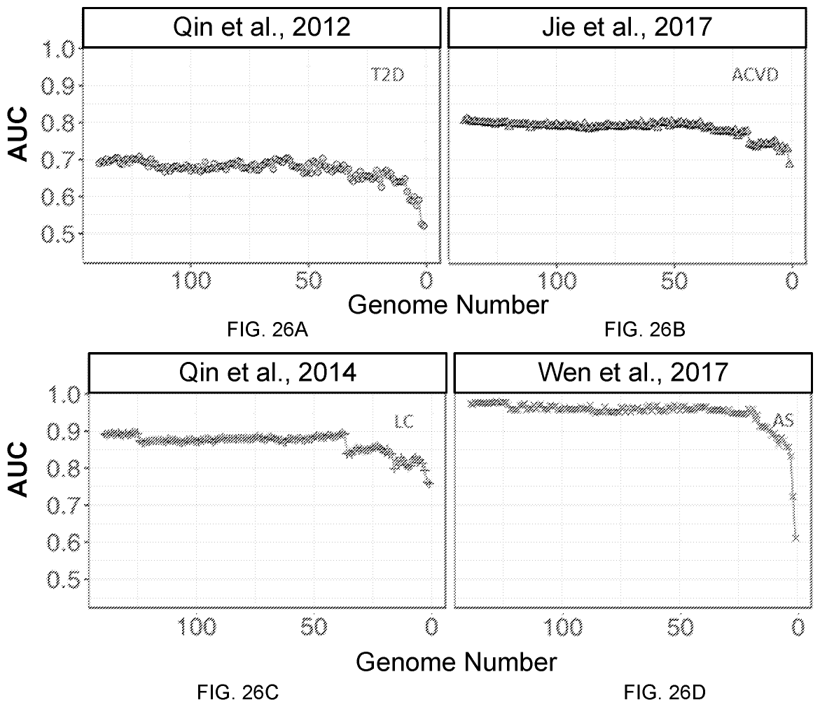

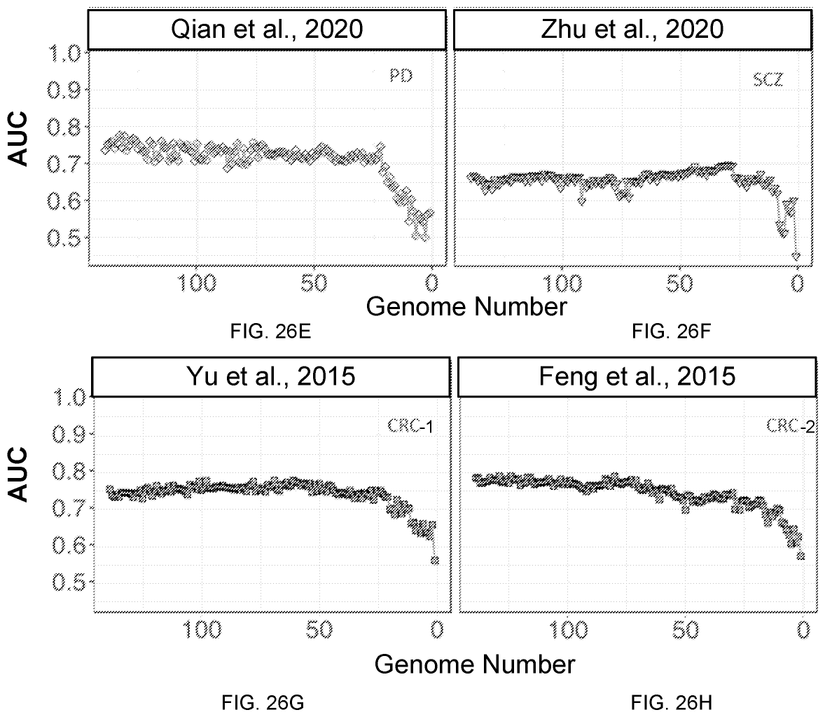

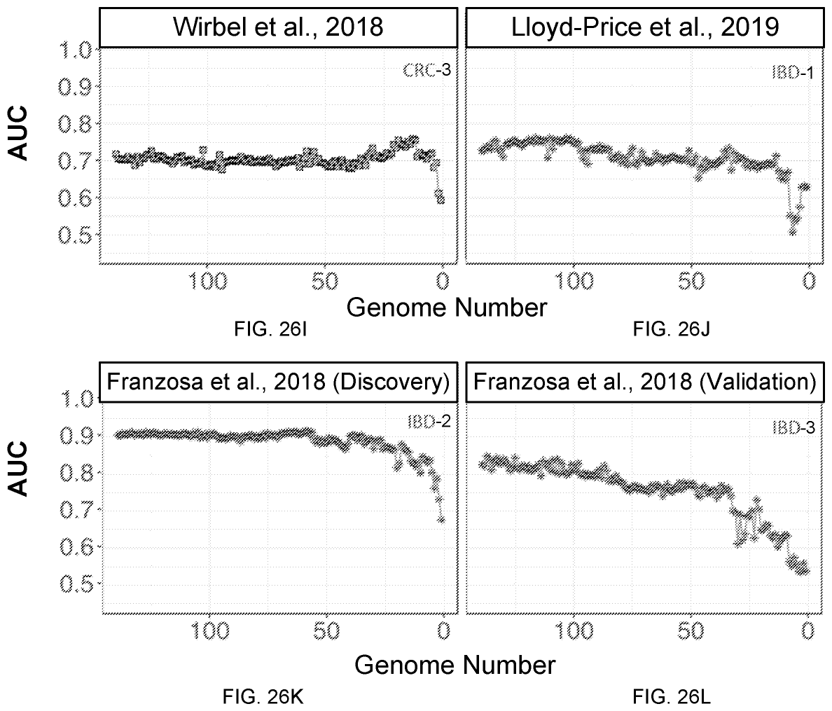

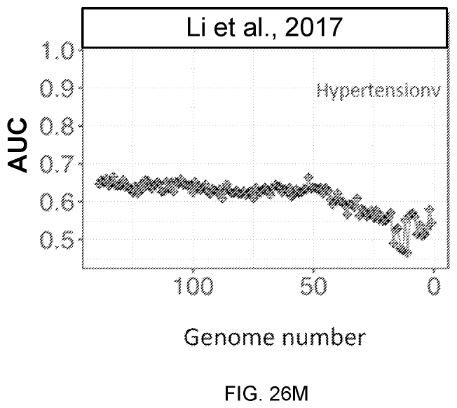

The classification performance with different numbers of genomes selected by degree based backward selection for eight types of diseases were validated. Random Forest regression models for eight different types of diseases were constructed based on 13 datasets obtained from 13 publications T2D (FIG. 26A), ACVD (FIG. 26B), LC (FIG. 26C), AS (FIG. 26D), PD (FIG. 26E), SCZ (FIG. 26F), CRC-1, CRC-2, CRC-3 (FIG. 26G-26I), IBD-1, IBD-2, IBD-3 (FIG. 26J-26L), hypertension (FIG. 26M). Those abundance reads associated with the 141 identified genomes were recruited to classify healthy subjects vs. patients. For each classification model, all the selected genomes are ranked by their degree (connectivity). The genomes with relative low degree are progressively removed. The corresponding prediction power with area under the curve AUC is calculated after the removal of each genome. The prediction power starts dropping when less than about 30 higher degree genomes were utilized to classify the healthy subjects vs. patients.

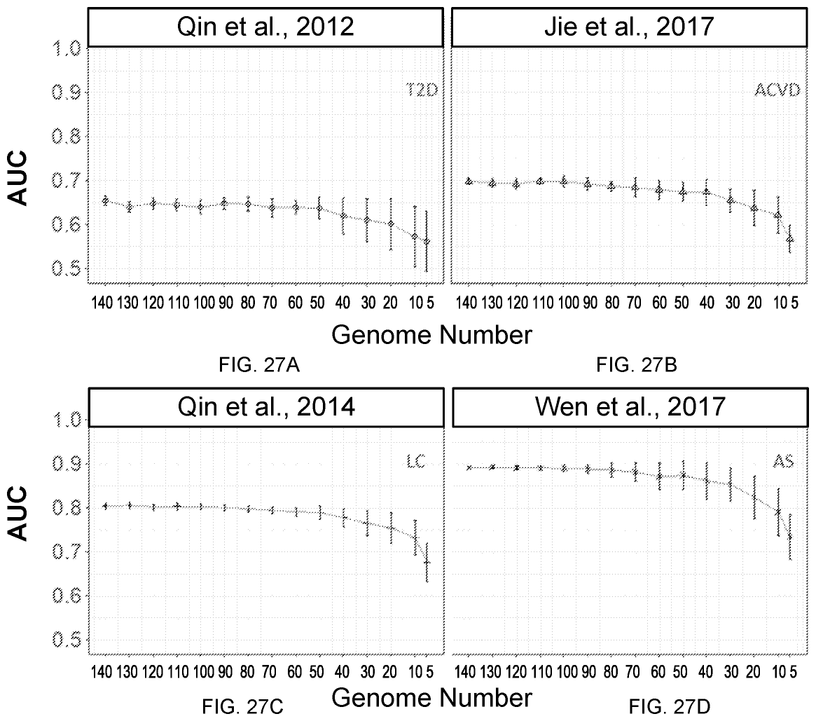

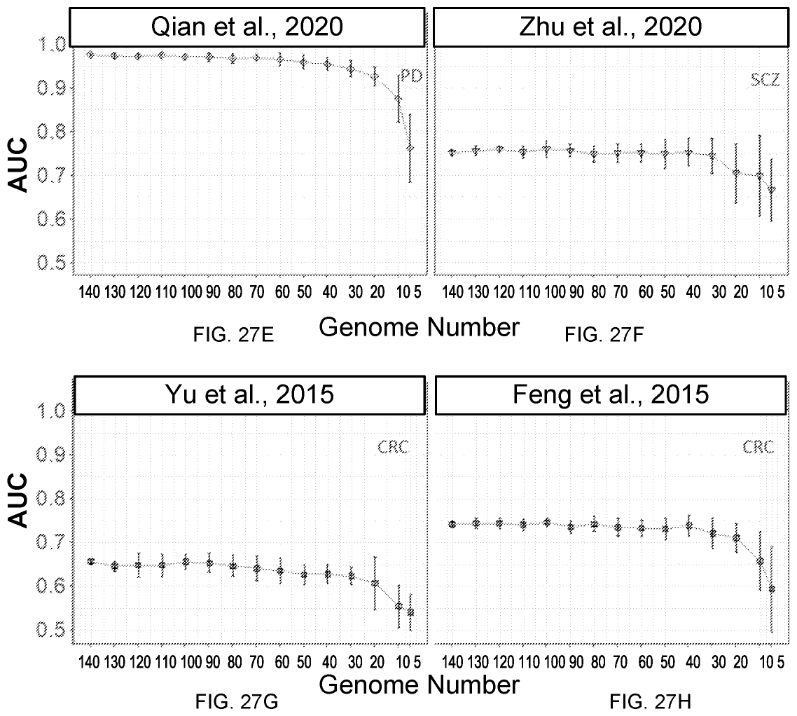

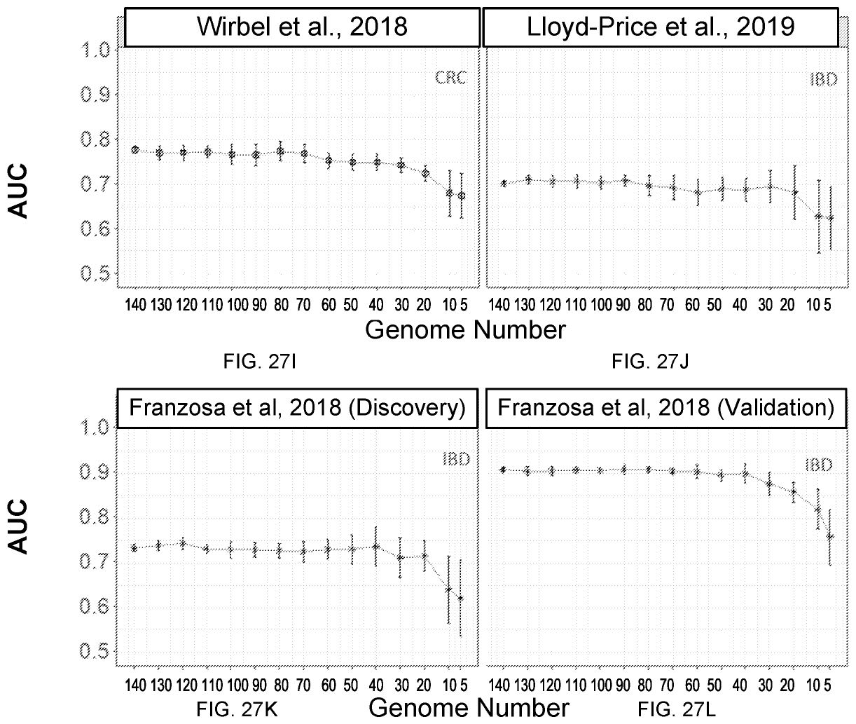

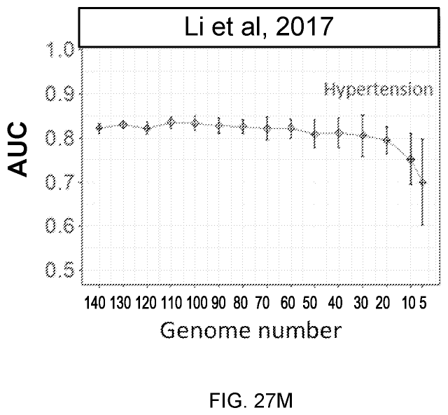

The classification performance with different numbers of genomes selected randomly for eight types of diseases is further validated. Random Forest regression models for eight different types of diseases were constructed based on 13 datasets obtained from 13 publications T2D (FIG. 27A), ACVD (FIG. 27B), LC (FIG. 27C), AS (FIG. 27D), PD (FIG. 27E), SCZ (FIG. 27F), CRC-1, CRC-2, CRC-3 (FIG. 27G-27I), MD-i, IBD-2, IBD-3 (FIG. 27J-27L), hypertension (FIG. 27M). Those abundance reads associated with the 141 identified genomes were recruited to classify healthy subjects versus patients. For each classification model, different numbers of randomly selected genomes are utilized for the prediction of the performance of classification. The corresponding prediction power with area under the curve AUC is calculated for each set of randomly selected genomes. The prediction power starts dropping when less than about 30 randomly selected genomes were utilized to classify the healthy subjects vs. patients.

### Discussion

As described herein, a genome-based, reference-free, and ecological interaction-focused approach led to the identification of a stable seesaw-like network of two competing guilds of genomes, whose changes were associated with a wide range of host phenotypes in patients with T2DM. Moreover, Random Forest models based on these genomes predictively classified case and control across a wide range of diseases, indicating that these genomes may form a common microbiome signature that exists in populations of widely different ethnicity, geography, and disease status.

Genomes in this common microbiome signature are organized in a seesaw-like network that has both cooperative and competitive interactions. Though cooperative ecological networks can be efficient, it creates dependency and the potential for mutual downfall that may bring destabilizing effect on human gut microbiome. This destabilizing effect of cooperation can be dampened by introducing ecological competition in the network [32]. Thus, a seesaw-like network with both cooperative and competitive interactions may represent a stable microbiome structure [32]. Interestingly, although the seesaw-like network is stable, the weight of the two ends i.e., the abundances of Guild 1 and Guild 2 are modifiable and such changes are associated with host health. When large amount of complex fiber became available, Guilds 1 and 2 showed no change in membership nor the nature of interactions with each other but experienced dramatic shifts in guild-level abundance in a competing manner. Members in Guild 1 have higher genetic capacity for degrading complex plant polysaccharides and produce beneficial metabolites including SCFAs which may suppress populations of pathobionts in Guild 216. Members of Guild 2 need to be kept low since their overgrowth may jeopardize host health by increasing inflammation, etc. [33]. However, pathobionts in Guild 2 cannot be eliminated, e.g., they may serve as the necessary agents that train our immune system from early on in our life [34, 35]. Therefore, the balance between Guild 1 and Guild 2 becomes critical in determining whether the gut microbiome supports health or aggravate diseases. This seesaw-like network between Guilds 1 and 2 allows the genomes in our common microbiome signature to readily respond to changes of external energy input to the gut microbial ecosystem and mediate its impact on host health, while simultaneously maintains its structural integrity. Such structural integrity may be key to ensuring long-term ecological stability of the gut microbiome and its ability to provide essential health-relevant functions to the host.

Such a seesaw networked structure may have been stabilized by natural selection over a long history of co-evolution between microbiomes and their hosts [18, 36]. Such a selection pressure may have been exerted by dietary fibers that only interact directly with gut microbes as external energy source [37,38]. Studies on coprolites showed that dietary fiber intake was much higher in ancient humans and only reduced significantly in the past 150 years [39, 40](130 g/d of plant fiber intake in prehistoric diet [41] vs. a median intake of 12-14 g/d in the modern American diet [42]). Such a high fiber intake over evolutionary history may have favored beneficial bacteria in Guild 1 because their higher genetic capacity to utilize plant polysaccharides as an external energy supply enables them to gain competitive advantage over pathobionts in Guild 2 in the gut microbial ecosystem [43]. Akin to tall trees as the foundation species for a closed forest, Guild 1 may work as the “foundation guild” for stabilizing a healthy gut microbiome and keeping the pathobionts at bay [44]. The dominance of Guild 1 over Guild 2 can increase host fitness as shown by the epidemiologically and clinically proven health benefits of dietary fibers in both preventing and alleviating a wide range of chronic conditions [16, 38, 45, 46].

Moreover, the genomes in our seesaw networked common microbiome signature may be considered as part of the core gut microbiome in humans [47, 48]. This is because: 1) they are commonly shared among populations across ethnicity and geography; 2) they show temporal stability not only in membership but also in their interactions with each other and the host; 3) they make up about 10% of the gut microbiome membership but are disproportionally important for shaping the ecological community; 4) they provide essential health-relevant functions to the host; and 5) such a core microbiome organized in a seesaw network may have been established over a long history of co-evolution and becomes the ecological foundation that modulates host health.

The fact that this seesaw-like network can be detected in other independent metagenomic datasets and is shown correlated with different diseases indicates that such an evolutionarily conserved ecological structure may be fundamentally important to human health recovery and maintenance. In addition, the seesaw network structure demonstrated stable relationships both internally within the network and externally with multiple host clinical markers, suggesting that genome-based guilds may serve as robust disease biomarkers. Within the seesaw network, it is the imbalance between the two competing guilds that may play a role as the common biological basis for many human diseases. Targeting this core gut microbiome to restore and maintain dominance of the beneficial guild over the detrimental guild could help reduce disease risk or alleviate symptoms, thus opening a new avenue for chronic diseases management and prevention.

### Materials and Methods

### Clinical Experiment

Study design [16]: This clinical trial, conducted at the Qidong People's Hospital (Jiangsu, China), examined the effect of a high fiber diet in free-living conditions in a cohort of individuals clinically diagnosed T2DM (QIDONG). The study protocol was approved by Ethics Committee of Shanghai General Hospital (2014KY104), and the study was conducted in accordance with the principles of the Declaration of Helsinki. All participants provided written informed consent. The trial was registered in the Chinese Clinical Trial Registry (ChiCTR-IPC-14005346). The study design and participant flow are shown in FIG. 9.

T2DM patients of the Chinese Han ethnicity were recruited for the study (age: 37-70 years; HbA1c: 6.5%-12.0%). More detailed description of inclusion and exclusion criteria were shown in Chinese Clinical Trial registry (chictr.org.cn).

Patients received either a high-fiber diet (WTP diet) as the treatment group (W group) or the usual care (Usual diet) as the control group (U group) for 3 months. Total caloric and macronutrients prescriptions were based on age-specific Chinese Dietary Reference Intakes (Chinese Nutrition Society, 2013). The WTP diet, based on wholegrains, traditional Chinese medicinal foods and prebiotics, included three ready-to-consume pre-prepared foods [16]. The usual diet including standard dietary and exercise advice was made according to the Chinese Diabetes Society guidelines for T2DM [49]. Patients in W group were provided with the WTP diet to perform a self-administered intervention at home for three months, while patients in U group accepted the usual care. W group stopped WTP diet intervention at the end of the third month (at M3). Then W and U continued a one-year follow-up (M15). A meal-based food frequency questionnaire and 24-h dietary recall were used to calculate nutrient intake based on the China Food Composition 200950. Patients in both groups continued with their antidiabetic medications according to their physician prescriptions.

FIGS. 22A and 22B collectively illustrate clinical parameters during intervention in the W and U group. The data are showed as mean±S.E.M (N). Friedman test followed by Nemenyi post-hoc test was used for intra-group comparisons, means with the same letter (a, b, or c) are not significantly different, with different letters are significantly different (P<0.05). Mann-Whitney test (two-sided) was used for comparisons between W and U at the same time point. FBG, fasting blood glucose; MTT Glucose AUC, area under the curve (AUC) of glucose in meal tolerance test; MTT C-Peptide AUC, area under the curve (AUC) of C-Peptide in meal tolerance test; HOMA-IR=1.5+FBG*Fasting-C-Peptide/2800; HOMA-β=0.27*Fasting-C-Peptide/(FBG—3.5); BMI, body mass index; BD, body weight; SBP, systolic blood pressure; DBP, diastolic blood pressure; WC, waist circumference; HP, hip circumference; WHR, waist to hip ratio; TNF-α, tumor necrosis factor-α; WBC, white blood cell count; CRP, C-reactive protein; LBP, lipopolysaccharide-binding protein; TC, total cholesterol; TG, triglyceride; Lpa, lipoprotein a; HDL, high-density lipoprotein; APOA, apolipoprotein A; LDL, low-density lipoprotein; APOB, apolipoprotein B; GFR, glomerular filtration rate; CysC, Cystatin C; ACR, urinary microalbumin to creatinine ratio; IMT, intima-media thickness; DAN, diabetic autonomic neuropathy score; MHR, mean heart rate; SDNN, standard deviation of NN intervals; SDANN, standard deviation of the average NN intervals calculated over 5 minutes; SDNNIndex, mean of standard deviation of NN intervals for 5-minute segments; rMSSD, root-mean-square of the differences of successive NN intervals; pNN50, percentage of the interval differences of successive NN intervals greater than 50 ms; TP, total power; VLF, very low frequency power; LF, low frequency power; HF, high frequency power; DPN, diabetic peripheral neuropathy score.

Before a 2-week run-in period, all participants attended a lecture on diabetes intervention and improvements and received diabetes education and metabolic assessments. 119 eligible individuals were enrolled based on the inclusion and exclusion criteria and assigned into two groups in a 2:1 ratio (n=79 in W group, n=40 in U group) determined by SAS software.

Physical examinations were carried out at M0, M3 and M15 in Qidong People's Hospital (Jiangsu, China), respectively. Sample collection instructions were provided to the participants at the day before. The participants provided the feces and first early morning urine as requested. After collecting fasting venous blood sample, a 3-h meal tolerance test (Chinese buns containing 75 g of available carbohydrates; MTT test) was conducted and the postprandial venous blood samples at 30, 60, 120, 180 min were collected. All the blood samples were centrifuged at 3000 rpm for 20 min at 4° C. after standing at room temperature for 30 min to obtain serum. The fasting blood serum were divided into two parts, one used for hospital tests and the other used for lab tests. The feces, urine and serum samples were stored in the dry ice immediately then transported to lab and frozen at −80° C. Subsequently, anthropometric markers and diabetic complication indexes were measured. Ewing test [51] and 24-h dynamic electrocardiogram were conducted to estimate diabetic autonomic neuropathy (DAN). B-mode carotid ultrasound was conducted to estimate atherosclerosis. Michigan Neuropathy Screening Instrument [52] was conducted to estimate diabetic peripheral neuropathy (DPN). In addition, A meal-based food frequency questionnaire and the 24-h dietary review were recorded for nutrient intake calculation. Besides, the drug use was self-reported.

The fasting venous blood was used to measure HbA1c, fasting blood glucose, fasting insulin, fasting C-Peptide, C-reactive protein (CRP), blood routine examination, blood biochemical examination and five analytes of thyroid. The venous blood samples at 30, 60, 120, 180 min of MTT were used to measure the postprandial blood glucose, insulin and C-Peptide. The fasting early morning urine was used to measure the routine urine examination and urinary microalbumin creatinine ratio. The measurements above were completed at Qidong People's Hospital. Fasting venous blood was used to quantify TNF-α (R&D Systems, MN, USA), lipopolysaccharide-binding protein (Hycult Biotech, PA, USA), leptin (P&C, PCDBH0287, China) and adiponectin (P&C, PCDBH0016, China) by enzyme-linked immunosorbent assays (ELISAs) at Shanghai Jiao Tong University.

The homeostatic model assessments of insulin resistance (HOMA-IR) and islet β-cell function (HOMA-β) were calculated based on fasting blood glucose (mmol/L) and fasting C-Peptide (pmol/L)53: HOMA-IR=1.5+FBG*Fasting-C-Peptide/2800;

HOMA-p=0.27*Fasting-C-Peptide/(FBG−3.5). Glomerular Filtration Rate was estimated by formula GFR (ml/min per 1.73 m2)=186*Scr−1.154*age−0.203*0.742 (if female)*1.233 (if Chinese)54, where Scr (serum creatinine) is in mg/dl and age is in years.

### Gut Microbiome Analysis

Metagenomic sequencing. DNA was extracted from fecal samples using the methods as previously described [17]. Metagenomic sequencing was performed using Illumina Hiseq 3000 at GENEWIZ Co. (Beijing, China). Cluster generation, template hybridization, isothermal amplification, linearization, and blocking denaturing and hybridization of the sequencing primers were performed according to the workflow specified by the service provider. Libraries were constructed with an insert size of approximately 500 bp followed by high-throughput sequencing to obtain paired-end reads with 150 bp in the forward and reverse directions.

Data quality control. Prinseq [55] was used to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2 [56] using --reorder --no-hd --no-contain --dovetail).

De novo assembly, abundance calculation and taxonomic assignment of genomes. De novo assembly was performed for each sample by using IDBA_UD [57](--step 20 --mink 20 --maxk 100 --min_contig 500 --pre_correction). The assembled contigs were further binned using MetaBAT [58](--minContig 1500 --superspecific -B 20). The quality of the bins was assessed using CheckM [59]. Bins had completeness>95%, contamination<5% and strain heterogeneity<5% were retained as high-quality draft genomes. The assembled high-quality draft genomes were further dereplicated by using dRep [60]. DiTASiC [61] was used to calculate the abundance of the genomes in each sample, estimated counts with P-value<0.05 were removed, and all samples were downsized to 36 million reads (One sample with read mapping ratio<25%, which could not be well represented by the high quality genomes, were removed in further analysis). Taxonomic assignment of the genomes was performed by using GTDB-Tk [62].

Gut microbiome functional analysis. Prokka [63] was used to annotate the genomes. KEGG Orthologue (KO) IDs were assigned to the predicted protein sequences in each genome by HMMSEARCH against KOfam using KofamKOALA [64]. Antibiotic resistance genes were predicted using ResFinder [65] with default parameters. The identification of virulence factors was based on the core set of Virulence Factors of Pathogenic Bacteria Database (VFDB [66], download July 2020). The predicted proteins sequences were aligned to the reference sequence in VFDB using BLASTP (best hist with E-value<le-5, identity>80% and query coverage>70%). Genes encoding carbohydrate-active enzymes (CAZys) were identified using dbCAN (releasee 6.0) [67], and the best-hit alignment was retained. The antibiotic resistance genes were identified by ResFinder [68]. Genes encoding formate-tetrahydrofolate ligase, propionyl-CoA:succinate-CoA transferase, propionate CoA-transferase, 4Hbt, AtoA, AtoD, Buk and But were identified as described previously [16].

Gut microbiome network construction and analysis. Fastspar [69] was used to calculate the correlations between the genomes with 1,000 permutations and the correlations with P≤0.001 were remained for further analysis. In W group, prevalent genomes shared by more than 75% of the samples at every timepoint were used to construct the co-abundant network at each timepoint. The networks were visualized with Cystoscape v3.8.1 [70]. The layout of the nodes and edges was determined by Edge-weighted Spring Embedded Layout with correlation coefficient as weight. The links between the nodes are treated as metal springs attached to the pair of nodes. The correlation coefficient was used as the function to determine the repulsion and attraction of the spring [70]. The layout algorithm sets the position of the nodes to minimize the sum of forces in the network. The robust stable edges were defined as the unchanged positive/negative correlations between the same two genomes across all the 3 networks. Stable genome pairs were cluster based on robust positive (set as 1) and negative (set as -1) edges with UPGMA clustering. iTOL71 was used to integrate and visualize the clustering tree, taxonomy information and abundance changes of the 141 genomes.

Validation in other independent cohorts. Independent metagenomic datasets from 4 case control studies were included to validate the commonality of the seesaw network core. These datasets were from 136 control and 136 T2DM individuals in Qin et al., 201222; 171 control and 214 atherosclerotic cardiovascular disease individuals in Jie et al., 201723; 83 control and 84 liver cirrhosis individuals in Qin et al., 201424; and 83 control and 97 ankylosing spondylitis individuals in Wen et al., 201725. (Table S8). DiTASiC was used to calculate the abundance of the 141 genomes in each sample, estimated counts with P-value<0.05 were removed and further converted to relative abundance divided by the total number of reads. Fastspar was used to calculate the correlations between the genomes with 1,000 permutations and the correlations with P<0.001 were remained for construing the networks. 30 repeat 5-fold cross-validation was used and the correlations shared by more than 95% of the 150 networks constructed from the cross-validation process were remained in the final network.

### Statistical Analysis.

Statistical analysis was performed in the R environment (R version 3.6.1). Friedman test followed by Nemenyi post-hoc test was used for intra-group comparisons. Mann-Whitney test was used for comparisons between W and U at the same time point. Pearson Chi-square tests was performed to compare the differences of categorical data between groups or timepoints. PERMANOVA test (9,999 permutations) was used to compare the groups of gut microbiota structure. P value less than 0.05 was accepted as statistical significance.

Mann-Whitney test and fisher exact test were used to compare the functions between Guild 1 and Guild 2. Random Forest with leave-one-out cross-validation was used to perform regression and classification analysis based on the microbiome signature and clinical parameters/groups.

To test whether genome abundance values for the 141 microbiomes identified above could be used to predict human health, random forest classifiers were trained based on microbiota datasets obtained for diseased and healthy controls in at least one study of each of Parkinson's disease, schizophrenia, inflammatory bowel disease, colorectal cancer, and hypertension. FIGS. 21A and 21B collectively illustrate the discriminative power microbiome signature as biomarkers to classify healthy subjects vs. patients in datasets on more diseases across ethnicity and geography. The microbiome signature supports predictive classification models for 8 other independent datasets. The area under the ROC curve (AUC) of the Random Forest classifier based on the 141 genomes in the microbiome signature to classify control and patients in each dataset. Leave-one-out cross validation was applied. Parkinson's Disease (1): Control n=40, Parkinson's Disease n=39; Schizophrenia (2): Control=81, Schizophrenia n=90; Colorectal Cancer (CRC) Dataset 1 (3): Control n=54, CRC n=74; Colorectal Cancer (CRC) Dataset 2 (4): Control n=63, CRC n=46; Inflammatory bowel disease (IBD) Dataset 1 (5): Control n=26, IBD n=80; Inflammatory bowel disease (IBD) Dataset 2 (6): Control n=34, IBD n=121; ; Inflammatory bowel disease (IBD) Dataset 3 (7): Control n=22, IBD n=43. Hypertension (8): Control n=41, hypertension n=99. 1=Qian, Y. W. et al. Gut metagenomics-derived genes as potential biomarkers of Parkinson's disease. Brain 143, 2474-2489 (2020). 2=Zhu, F. et al. Metagenome-wide association of gut microbiome features for schizophrenia. Nat Commun 11, 1612, doi:10.1038/s41467-020-15457-9 (2020). 3=Yu, J. et al. Metagenomic analysis of faecal microbiome as a tool towards targeted non-invasive biomarkers for colorectal cancer. Gut 66, 70-+, doi:DOI 10.1136/gutjnl-2015-309800 (2017). 4=Feng, Q. et al. Gut microbiome development along the colorectal adenoma-carcinoma sequence. Nature communications 6, 6528, doi:10.1038/ncomms7528 (2015). 5=Lloyd-Price, J. et al. Multiomics of the gut microbial ecosystem in inflammatory bowel diseases. Nature 569, 655-662, doi:10.1038/s41586-019-1237-9 (2019). 6=Franzosa, E. A. et al. Gut microbiome structure and metabolic activity in inflammatory bowel disease. Nature microbiology 4, 293-305, doi:10.1038/s41564-018-0306-4 (2019). 7=Li, J. et al. Gut microbiota dysbiosis contributes to the development of hypertension. Microbiome 5, 14, doi:10.1186/s40168-016-0222-x (2017).

Because the respective microbiota guilds are highly interrelated, it was hypothesized that the information in relative abundance values would be highly correlated, such that fewer than all of the genomic abundance values would provide suitable power for classification. As a first test, multiple random forest classifiers were trained based on microbiota datasets obtained for diseased and healthy controls in at least one study of each of type-2 diabetes (T2D), atherosclerotic cardiovascular disease (ACVD), liver cirrhosis (LC), ankylosing spondylitis (AS), Parkinson's disease (PD), schizophrenia (SCZ), colorectal cancer (CRC), inflammatory bowel diseases (IBD), and hypertension. The first random forest classifier trained for each disorder used genomic data for all 141 genomes in Table 1. Each subsequent classifier was trained on one less genome, with the drop out order of genomes determined by the degree of connectivity of the genome within the guilds (the number of connections the genome makes with other genomes in the guilds) in ascending order, as shown in Table 5.

TABLE 5
Order of genome drop-out.

| ID | Degree |
|---|---|
| 1W065.34 | 1 |
| 2W027.13 | 1 |
| 2W073.18 | 1 |
| 2W073.55 | 1 |
| 3U033.37 | 1 |
| 3W052.32 | 1 |
| 3W062.32 | 1 |
| 3W073.49 | 1 |
| 1U002.48 | 1 |
| 2W066.63 | 1 |
| 2W043.36 | 1 |
| 2W043.20 | 1 |
| 2W001.17 | 1 |
| 1U002.41 | 1 |
| 1W071.19 | 1 |
| 1W078.21 | 1 |
| 1W080.50 | 1 |
| 2U001.17 | 1 |
| 2U037.18 | 1 |
| 2W002.29 | 1 |
| 2W004.25 | 1 |
| 2W011.21 | 1 |
| 2W054.20 | 1 |
| 3W028.33 | 1 |
| 3W049.14 | 1 |
| 3W079.9 | 1 |
| 3W080.19 | 1 |
| 1U027.33 | 1 |
| 1U024.11 | 1 |
| 1W023.20 | 1 |
| 1U021.14 | 1 |
| 1W026.10 | 1 |
| 1U014.18 | 1 |
| 2W067.16 | 1 |
| 1W079.48 | 1 |
| 2U033.43 | 2 |
| 2W023.30 | 2 |
| 2W034.32 | 2 |
| 2W067.69 | 2 |
| 3U005.26 | 2 |
| 1W011.28 | 2 |
| 1W001.34 | 2 |
| 1W037.27 | 2 |
| 1W072.11 | 2 |
| 2U017.6 | 2 |
| 2W001.42 | 2 |
| 2W050.17 | 2 |
| 3U038.19 | 2 |
| 3W042.11 | 2 |
| 3W074.2 | 2 |
| 1U017.10 | 2 |
| 1U015.20 | 2 |
| 1W023.6 | 2 |
| 1U036.17 | 2 |
| 2W007.23 | 3 |
| 2W080.51 | 3 |
| 3U010.20 | 3 |
| 1W062.68 | 3 |
| 2W037.10 | 3 |
| 1W023.15 | 3 |
| 1W071.47 | 4 |
| 2W029.13 | 4 |
| 3U024.38 | 4 |
| 3W072.38 | 4 |
| 1U038.6 | 4 |
| 2U037.6 | 4 |
| 2W025.8 | 4 |
| 2W026.44 | 4 |
| 3W039.10 | 4 |
| 3W054.11 | 4 |
| 1U030.25 | 5 |
| 1W059.36 | 5 |
| 2W074.14 | 5 |
| 1U030.5 | 5 |
| 1U035.55 | 5 |
| 2W012.75 | 5 |
| 2W048.49 | 5 |
| 2W080.58 | 5 |
| 3U040.11 | 5 |
| 3W023.3 | 5 |
| 3W055.19 | 5 |
| 1U015.30 | 5 |
| 1U036.46 | 6 |
| 2W009.30 | 6 |
| 1W006.13 | 6 |
| 2W067.20 | 6 |
| 3W002.16 | 6 |
| 1U036.9 | 6 |
| 2U017.37 | 7 |
| 2W001.22 | 7 |
| 2W027.27 | 7 |
| 2W062.21 | 7 |
| 1W070.8 | 7 |
| 2U008.16 | 7 |
| 1U009.16 | 8 |
| 1U014.38 | 8 |
| 2U001.37 | 8 |
| 3W067.18 | 8 |
| 1U017.15 | 8 |
| 3W015.22 | 9 |
| 1W037.25 | 10 |
| 1W071.30 | 10 |
| 2W064.32 | 10 |
| 2W065.60 | 10 |
| 3U007.7 | 10 |
| 3U020.14 | 10 |
| 1U001.8 | 10 |
| 3W048.18 | 11 |
| 2U001.12 | 11 |
| 1W075.10 | 13 |
| 1W075.14 | 14 |
| 3W080.64 | 14 |
| 1U017.16 | 15 |
| 1U038.18 | 16 |
| 1W030.27 | 17 |
| 3W036.5 | 17 |
| 2U033.18 | 18 |
| 2W005.9 | 19 |
| 1W047.42 | 20 |
| 2U038.12 | 20 |
| 2U004.52 | 21 |
| 1U003.28 | 21 |
| 1W032.17 | 22 |
| 2W053.46 | 22 |
| 2W003.7 | 23 |
| 1W042.8 | 24 |
| 1U017.25 | 25 |
| 1W036.28 | 25 |
| 3U023.7 | 25 |
| 1W012.66 | 27 |
| 1W038.31 | 27 |
| 1U035.49 | 28 |
| 3W036.6 | 28 |
| 1W033.3 | 31 |
| 1W033.9 | 31 |
| 2U038.5 | 31 |
| 2W075.14 | 31 |
| 1U028.21 | 32 |
| 1W032.19 | 32 |
| 2U037.14 | 32 |
| 3W042.9 | 41 |

The performance of each model was then determined as AUC for a ROC curve and platted as shown in FIG. 26. As shown in FIG. 26, fewer than all of the 141 genomes was required to adequately power a clinical model of disease state. In fact, in most, if not all cases, models trained with only the 10-15 most connected genomes were adequately powered for clinical use (e.g., having an AUC of 0.65 or greater).

Next, it was determined how many genomes chosen at random from the 141 identified genomes were sufficient to power a model having clinical usefulness. Briefly, multiple random forest classifiers were trained based on microbiota datasets obtained for diseased and healthy controls in at least one study of each of type-2 diabetes (T2D), atherosclerotic cardiovascular disease (ACVD), liver cirrhosis (LC), ankylosing spondylitis (AS), Parkinson's disease (PD), schizophrenia (SCZ), colorectal cancer (CRC), inflammatory bowel diseases (IBD), and hypertension. Specifically, for each dataset, 10 classifiers were trained using randomly selected sets of 5, 10, 20, 30, 40, 50, 60, 70, 80, 90, 100, 110, 120, 130, and 140 genomes from the 141 genomes identified in Table 1 (150 total models per data set). The average AUC from ROC curves for each set of x randomly selected genomes was determined and plotted in FIG. 27. As shown in FIG. 27, fewer than all of the 141 genomes was required to adequately power a clinical model of disease state. In fact, in most, if not all cases, models trained with only 15-20 randomly selected genomes were adequately powered for clinical use (e.g., having an AUC of 0.65 or greater).

### Example 2—Identification of Microbiome Signature for Human Diseases

To investigate the microbiome signature of seven different types of disease: type-2 diabetes (T2D), schizophrenia (SCZ), atherosclerotic cardiovascular disease (ACVD), liver cirrhosis (LC), inflammatory bowel diseases (IBD), colorectal cancer (CRC), ankylosing spondylitis (AS), were obtained from 11 metagenomic datasets from publications (Qin, J. et al. A metagenome-wide association study of gut microbiota in type 2 diabetes. Nature 490, 55-60, doi:10.1038/naturel 1450 (2012). Jie, Z. et al. The gut microbiome in atherosclerotic cardiovascular disease. Nat Commun 8, 845, doi:10.1038/s41467-017-00900-1 (2017). Qin, N. et al. Alterations of the human gut microbiome in liver cirrhosis. Nature 513, 59-64, doi:10.1038/nature13568 (2014). Wen, C. et al. Quantitative metagenomics reveals unique gut microbiome biomarkers in ankylosing spondylitis. Genome Biol 18, 142, doi:10.1186/s13059-017-1271-6 (2017). Zhu, F. et al. Metagenome-wide association of gut microbiome features for schizophrenia. Nat Commun 11, 1612, doi:10.1038/s41467-020-15457-9 (2020). Yu, J. et al. Metagenomic analysis of faecal microbiome as a tool towards targeted non-invasive biomarkers for colorectal cancer. Gut 66, 70-+, doi:DOI 10.1136/gutjnl-2015-309800 (2017). Feng, Q. et al. Gut microbiome development along the colorectal adenoma-carcinoma sequence. Nature communications 6, 6528, doi:10.1038/ncomms7528 (2015). Lloyd-Price, J. et al. Multi-omics of the gut microbial ecosystem in inflammatory bowel diseases. Nature 569, 655-662, doi:10.1038/s41586-019-1237-9 (2019). Franzosa, E. A. et al. Gut microbiome structure and metabolic activity in inflammatory bowel disease. Nature microbiology 4, 293-305, doi:10.1038/s41564-018-0306-4 (2019)). To achieve genome-level resolution, non-redundant high-quality draft genomes were reconstructed (HQMAGs, two HQMAGs were collapsed into one if the average nucleotide identity, ANI, between them was >99%) from the metagenomic datasets.

To facilitate the identification of genome pairs that keep their ecological interactions stable between the case and control group, a co-abundance network was constructed for each case based on the abundance matrix of the HQMAGs representing the prevalent microbes. Co-abundance network is a data-driven way to investigate ecological interactions between microbes across habitats. Prevalent HQMAGs among the biological samples for each indication were selected for network construction. In each case cohort and its corresponding control cohort, for example, a type-2 diabetes group and a healthy subject cohort, pairwise correlations of all possible genome pairs were calculated among these prevalent HQMAGs based on their abundance and constructed seven co-abundance networks. The networks were represented by order S, i.e., the total number of nodes (HQMAGs), and their size L, i.e., the total number of edges (correlations). Fastspar, a rapid and scalable correlation estimation tool for microbiome study, was used to calculate the correlations between the genomes with 1,000 permutations at each time point based on the abundances of the genomes across the patients and the correlations with P≤0.001 were retained for further analysis. The networks were visualized with Cystoscape v3.8.176. The layout of the nodes and edges was determined by Edge-weighted Spring Embedded Layout using the correlation coefficient as weights. The links between the nodes are treated as metal springs attached to the pair of nodes. The correlation coefficient was used to determine the repulsion and attraction of the spring. The layout algorithm sets the position of the nodes to minimize the sum of forces in the network. Differences between the co-abundance networks of the case and control cohort were observed.

Genomes were considered as having robust and stable ecological relationship if a genome pair keeps the same ecological interaction between a case cohort and a control cohort. Robust stable edges were defined by unchanged positive/negative correlations between the same two genomes between a case cohort and a control cohort. Stable genome pairs were clustered based on robust positive (set as 1) and negative (set as -1) edges with average clustering. iTOL77, an online tool, was used for display, manipulation, and annotation for various trees, to integrate and visualize the clustering tree, taxonomy information, and abundance changes of the genomes.

Genome pairs having no correlations or no stable ecological interactions are removed from analysis. Prevalent HQMAGs having positive correlations and negative correlations are selected for further analysis. For each case cohort and its corresponding control cohort, the largest interconnected HQMAG group was identified from all available interconnected HQMAG groups (C1, C2, . . . ), and the HQMAGs that have no interactions with the largest interconnected HQMAG group are removed from further analysis.

The remaining HQMAGs, which included genome pairs with stable correlations were further defined as genomes with stable ecological interactions (GSEIs) and became our microbiome signature candidates. The GSEIs had significantly higher degree, betweenness centrality, eigenvector centrality, closeness centrality and stress centrality than the rest of the genomes in the networks. These suggest that the GSEIs can be considered as the core nodes of the networks as they were highly connected not only within themselves but also with other nodes.

Bacteria which are positively correlated with each other and show robust co-occurrence behavior can be recognized as ecological guilds. The 141 GSEIs organized themselves into two competing guilds and genomes in each guild were highly interconnected with positive correlations. Members within each guild had robust cooperative relationships, while competitive relationships existed between the two guilds.

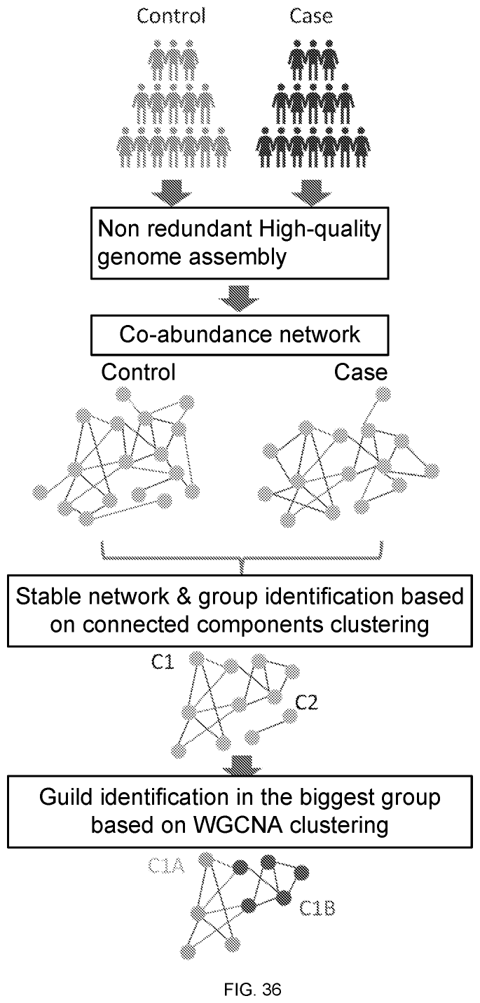

The flow of identifying microbiome signature from a case cohort and a control cohort is illustrated in FIG. 36.

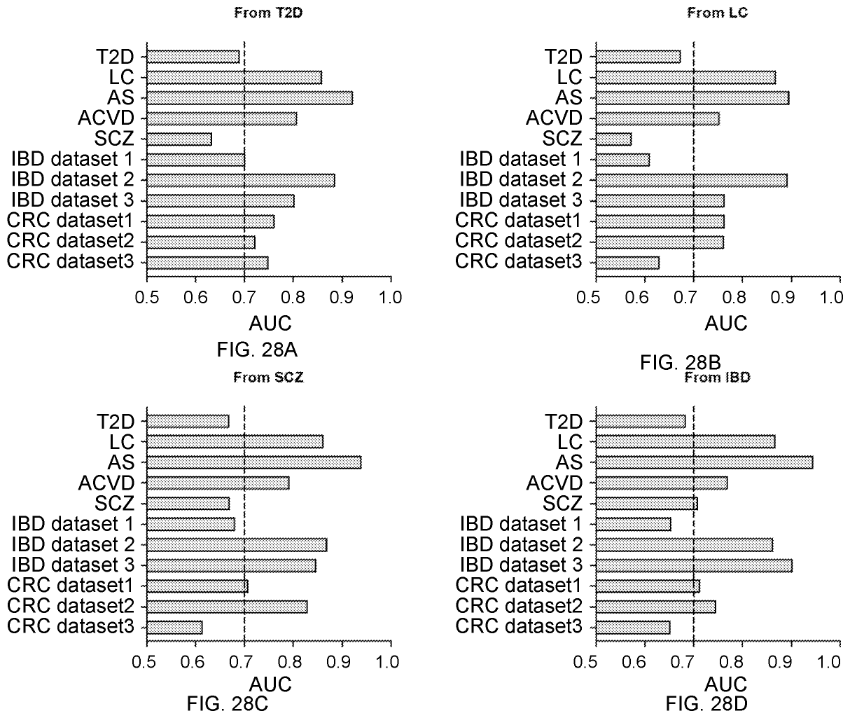

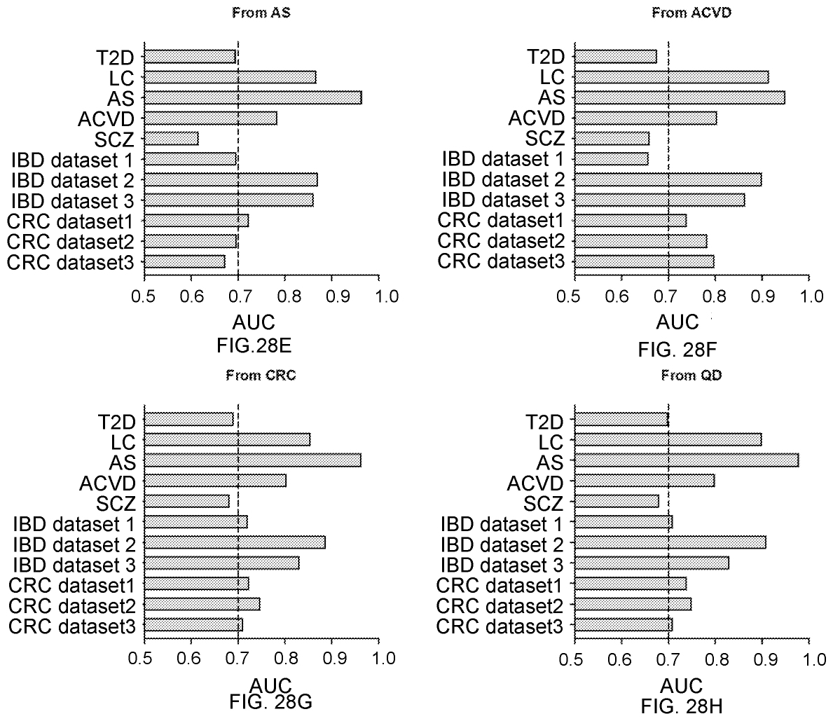

The members from two competing guilds identified from QD and various types of diseases including type-2 diabetes (T2D), schizophrenia (SCZ), atherosclerotic cardiovascular disease (ACVD), liver cirrhosis (LC), inflammatory bowel diseases (IBD), colorectal cancer (CRC), ankylosing spondylitis (AS) were used to predict their capacity to classify case and control across different studies through Random Forest classifiers. The results for microbiome signature from T2D (FIG. 28A), LC (FIG. 28B), SCZ (FIG. 28C), IBD (FIG. 28D), AS (FIG. 28E), ACVD (FIG. 28F), CRC (FIG. 28G), and QD (FIG. 28H) are plotted based on AUC value. FIG. 28 shows all microbiome signature have the capacity to classify case and control across different studies. The exact classification capacity varies between different studies and between different sets of microbiome signatures.

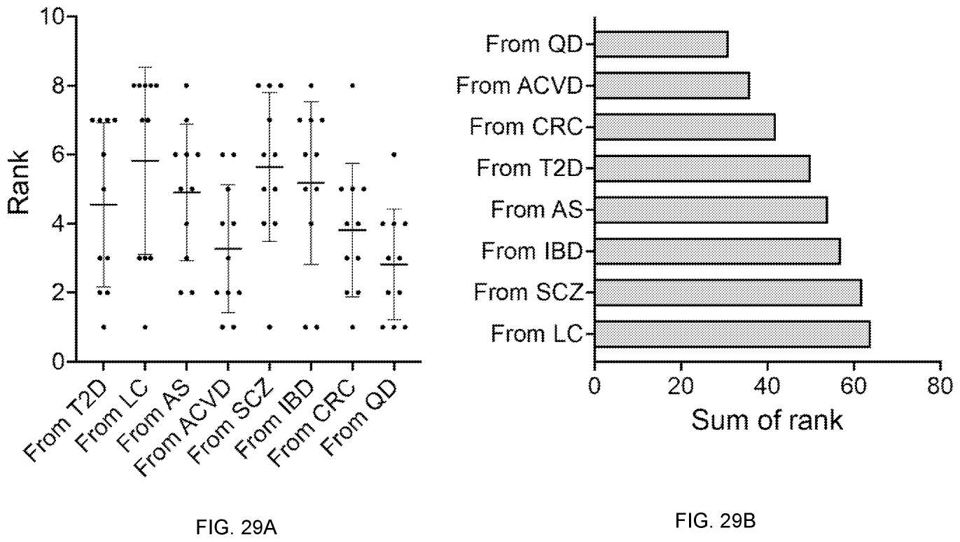

The classification capacity of the eight sets of microbiome signature was ranked based on their performance across 11 datasets. The eight sets of microbiome signature obtained from QD and from various diseases cases: T2D, LC, SCZ, IBD, AS, ACVD, CRC are ranked according to their performance in classifying case and control for each of the dataset. The rank values assigned to each set of signature microbiome is plotted in FIG. 29A. FIG. 29B shows the sum of the ranks for each set of microbiome signatures. The lower the rank is, the better the classification performance is. The microbiome signature obtained from QD has the best performance to classify the healthy subjects vs. patients across 11 datasets.

### Example 3—Identification of Combined Core Microbiome Signature from Combined Pool of Genomes

### 1. Combined Pool of Genomes

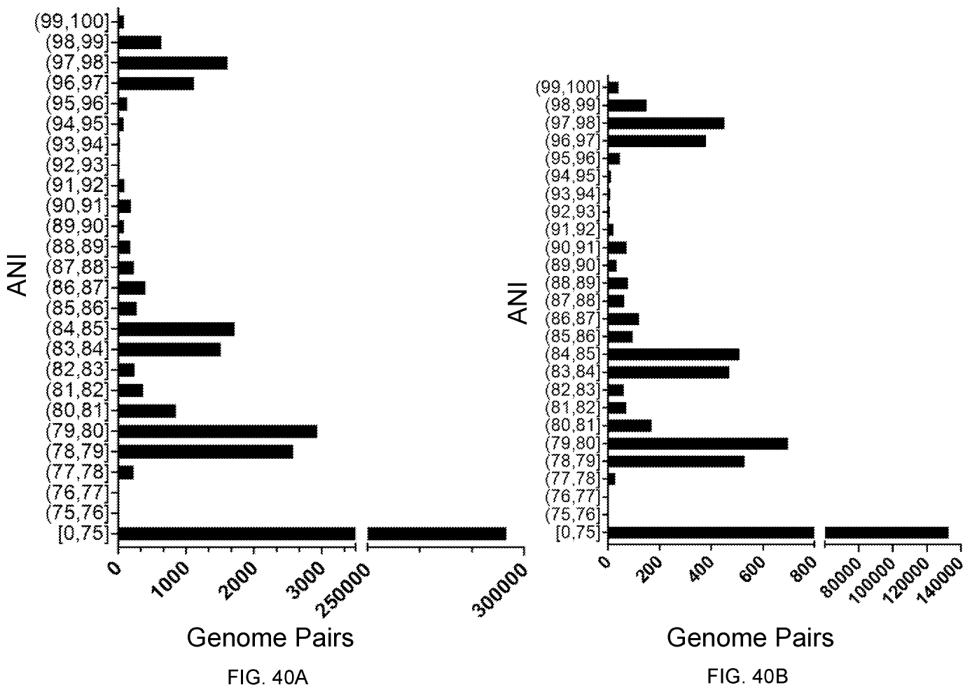

921 genomes (genomes in the two competing guilds found in QD and seven types of diseases including T2D, LC, SCZ, IBD, AS, ACVD, CRC) were further dereplicated by using dRep. Two genomes were collapsed into one if the average nucleotide identity, ANI, between them was >99%. 788 non-redundant genomes were obtained. The genome pairwise ANI comparison was performed for the 310,078 genome pairs among the 788 genomes. The ANI distribution was shown in FIG. 40A. The ANI comparison between the genomes assigned into two competing guilds: Guild 1 and Guild 2, was further studied. After removing genomes with inconsistent guild assignment from the 788 genomes, Guild 1 has 311 genomes, and Guild 2 has 440 genomes. The genome pairs between Guild 1 and Guild 2 were calculated by multiplying the total number of genomes in Guild 1, 440, by the total number of genomes of Guild 2, 331. The ANI distribution for the 136,840 genome pairs was shown in FIG. 40B.

DiTASiC, which applied kallisto for pseudo-alignment and a generalized linear model for resolving shared reads among genomes, was used to calculate the abundance of the genomes in each sample, estimated counts with P-value>0.05 were removed.

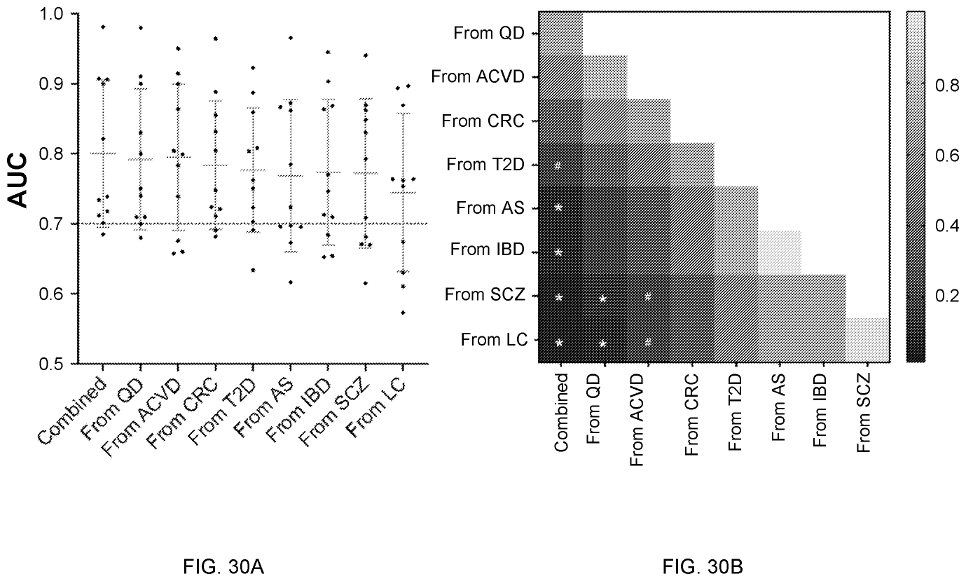

A machine learning classifier based on a Random Forest algorithm was trained to compare the capacity of the combined 788 genomes in classifying patients and control with the individual set of microbiome signature obtained from QD and various diseases cases including T2D, LC, SCZ, IBD, AS, ACVD, CRC. The area under the ROC curve (AUC) of the Random Forest classifier based on the combined pool or individual microbiome signature to classify control and patients in each dataset are shown in FIG. 30A. FIG. 30B shows the significance of intra-group comparison. Friedman test followed by Dunn's post hoc was performed for the analysis (#BH adjusted P<0.1, *BH adjusted P<0.05). Overall, Combined pool has the best capacity to classify case and control across different studies.

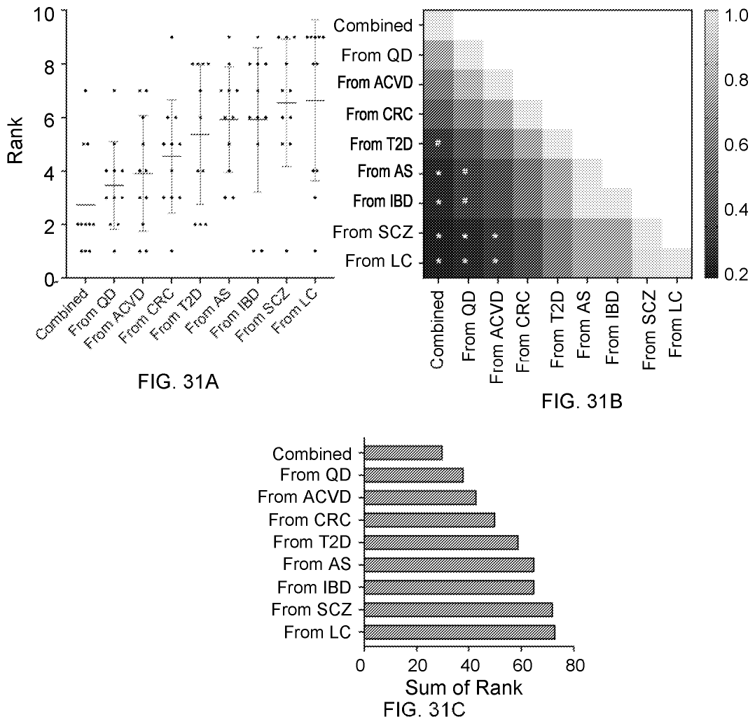

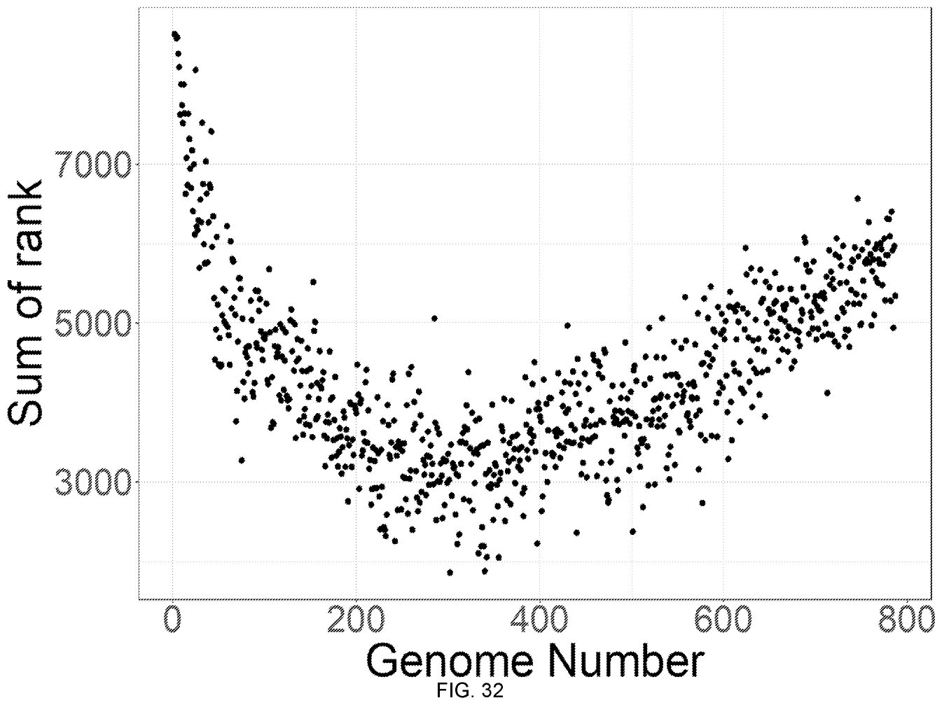

The classification performance of each model was further ranked. The nine sets of microbiome signature are ranked according to their performance in classifying case and control across 11 datasets. The rank values assigned to each set of signature microbiome are plotted FIG. 31A. FIG. 31B shows the significance of intra-group comparison. FIG. 31C shows the sum of the ranking values for each set of microbiome signatures. Kruskal-Wallis test followed by Dunn's post hoc was performed for the analysis (#BH adjusted P<0.1, *BH adjusted P<0.05). The results confirms that the microbiome signature obtained from the combined pool has the best performance to classify the healthy subjects vs. patients across 11 datasets.

### 2. Combined Core Pool of Genomes

The combined core pool of genomes from the combined 788 genomes was selected through the steps set out below. Random Forest classification based on a combined 788 genomes are performed for each dataset. Each of the 788 genome is ranked based on its importance for each dataset. A summed rank is obtained by adding up the value of ranks across 11 datasets and all 788 genomes are ranked again based on the summed value. The most important genome across 11 dataset gets the lowest summed rank value (Table 3).

TABLE 3
Ranking of Genome importance

| | | | | | | summed |
|---|---|---|---|---|---|---|
| Genome | Dataset 1 | Dataset 2 | Dataset 3 | . . . | Dataset 11 | rank |
| 1 | D1R1 | D2R1 | D3R1 | . . . | D11R1 | SR1 |
| 2 | D1R2 | D2R2 | D3R2 | . . . | D11R2 | SR2 |
| 3 | D1R3 | D2R3 | D3R3 | . . . | D11R3 | SR3 |
| . . . | . . . | . . . | . . . | . . . | . . . | . . . |
| 788 | D1R788 | D2R788 | D3R788 | . . . | D11R788 | SR788 |

Starting from the least important genome, every genome one by one is removed from each dataset based on order of importance. The classification performance (AUCs) is calculated for the remaining numbers of genomes after each round of removal by Random Forest model and all the genome numbers are ranked based on AUC values. The ranking values for each genome number across 11 datasets is summed (Table 4).

TABLE 4
Rank genome number based on AUC

| Genome | | | | | | summed |
|---|---|---|---|---|---|---|
| number | Dataset 1 | Dataset 2 | Dataset 3 | . . . | Dataset 11 | rank |
| 787 | D1R1_auc | D2R1_auc | D3R1_auc | D4R1_auc | D11R1_auc | SR1_auc |
| 786 | D1R2_auc | D2R2_auc | D3R2_auc | D4R2_auc | D11R2_auc | SR2_auc |
| 777 | D1R3_auc | D2R3_auc | D3R3_auc | D4R3_auc | D11R3_auc | SR3_acu |
| . . . | . . . | . . . | . . . | . . . | . . . | . . . |
| 2 | D1R786_auc | D2R786_auc | D3R786_auc | D4R786_auc | D11R786_auc | SR786_auc |

The sum of the ranking values for each genome number across 11 datasets is plotted in FIG. 32. 302 genomes achieved lowest summed AUC ranks. After removing 18 genomes which exhibit inconsistent C1A and C1B assignment, 284 genomes remained as the combined core pool genomes.

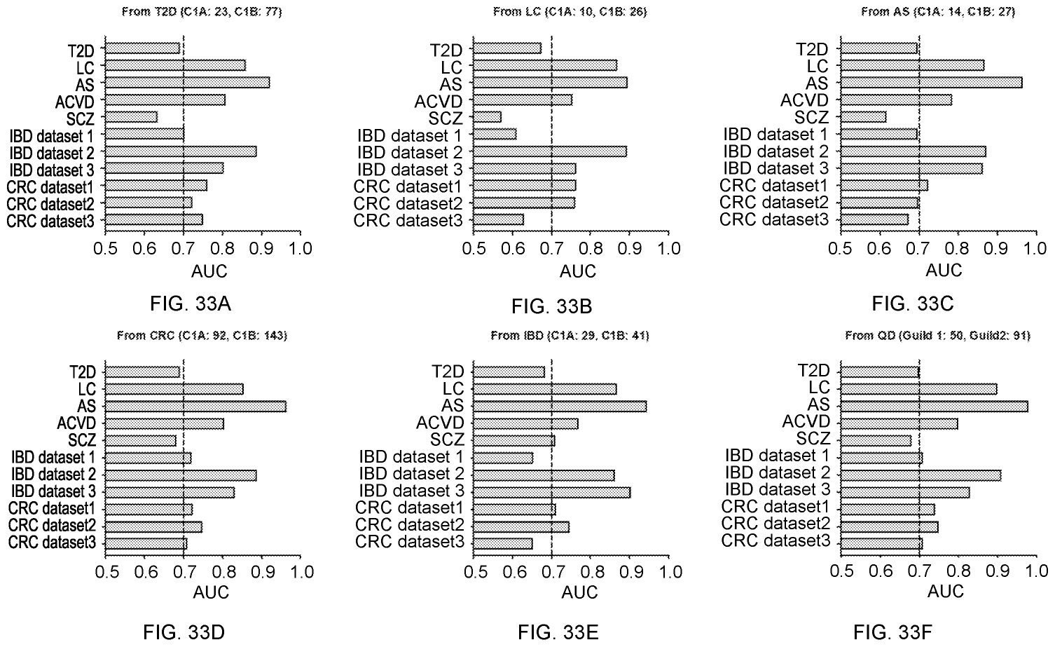

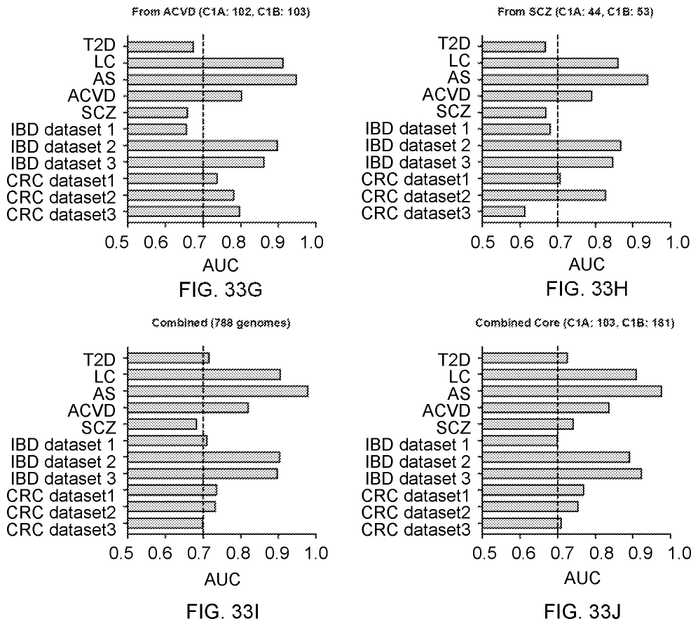

The classification capacity of the two competing guilds identified from: T2D (FIG. 33A), LC (FIG. 33B), AS (FIG. 33C), CRC (FIG. 33D), IBD (FIG. 33E), QD (FIG. 33F), AVCD (FIG. 33G), SCZ (FIG. 33H), combined pool (FIG. 33I), and the combined core pool (FIG. 33J) were compared to each other. The identified microbiome signature for each condition is utilized to classify control and patients in each dataset using Random Forest classifiers. FIG. 31 shows all microbiome signature have the capacity to classify case and control across different studies.

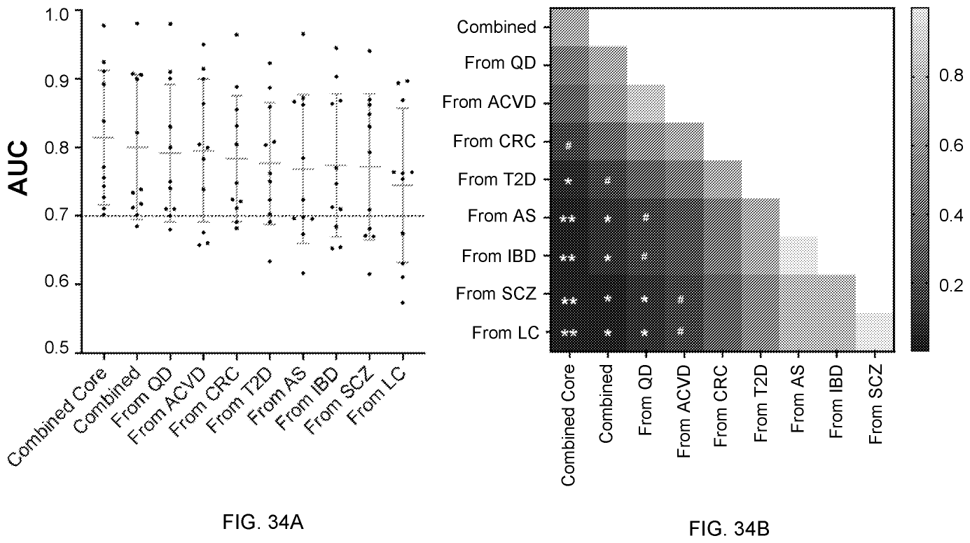

As illustrated in FIG. 34, the capacity of the combined core pool has the best capacity to classify case and control across different studies. The area under the ROC curve (AUC) of the Random Forest classifier based on the combined core pool, the combined pool or individual microbiome signature to classify control and patients in each dataset are compared in FIG. 34A. FIG. 34B shows the significance of intra-group comparison. Friedman test followed by Dunn's post hoc was performed for the analysis (#BH adjusted P<0.1, *BH adjusted P<0.05, **BH adjusted P<0.01).

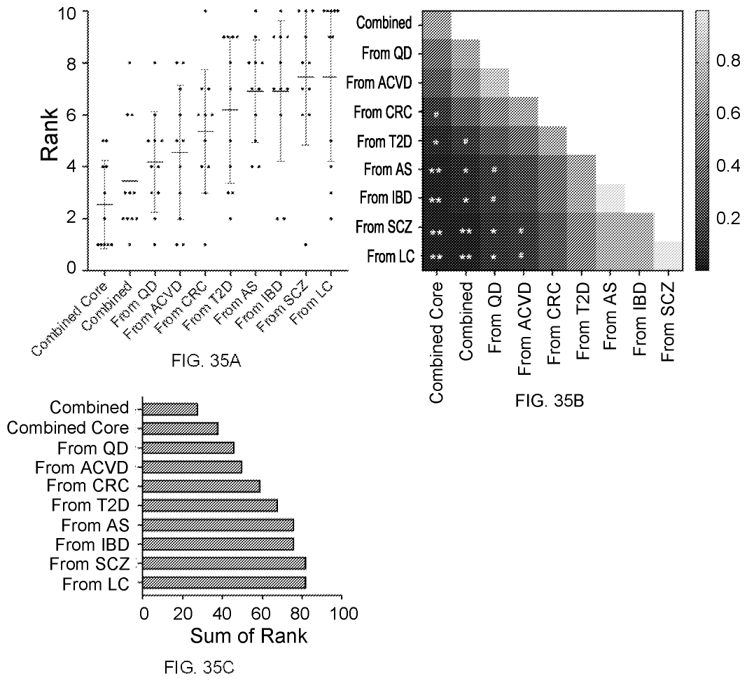

The classification performance of the microbiome signature is ranked based on AUC values across 11 datasets. All the rank values assigned to each set of signature microbiome are plotted FIG. 35A. FIG. 35B shows the significance of intra-group comparison. FIG. 35C shows the sum of the ranking values for each set of microbiome signatures. Kruskal-Wallis test followed by Dunn's post hoc was performed for the analysis (#BH adjusted P<0.1, *BH adjusted P<0.05, **BH adjusted P<0.01). Those results confirm that the microbiome signature obtained from the combined core pool has the best performance to classify the healthy subjects vs. patients across different datasets.

### Example 4—Universal Random Forest Classification Models Based on the 284 Core Genomes in the Seesaw Networked Two Competing Guilds

25 metagenomic datasets covering case-control studies on 15 different diseases (type-2 diabetes (T2D), hypertension (HT), schizophrenia (SCZ), atherosclerotic cardiovascular disease (ACVD), liver cirrhosis (LC), inflammatory bowel diseases (IBD), colorectal cancer (CRC), ankylosing spondylitis (AS), Parkinson's disease (PD), Multiple Sclerosis (MS), Gaucher disease type II (GDII), COVID-19 (COV), Behcet's disease (BD), autism spectrum disorder (ASD), and pancreatic cancer (PC)) were used to train random forest classification models for each dataset using the abundance of the same 284 core genomes present in our seesaw network of two competing guilds: the foundation guild and the pathogen guild. The models enabled us to distinguish between case and control in each of the 25 metagenomic datasets tested using leave-one-out cross-validation, with most AUCs above 0.7.

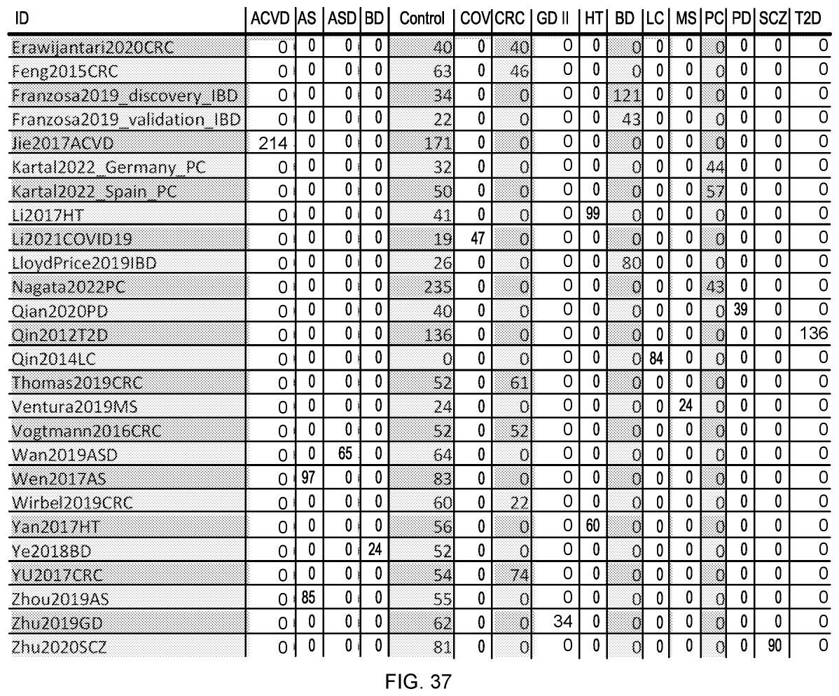

To classify case vs control, the case and control samples from the 25 datasets that corresponded to 15 various diseases (FIG. 37) were combined, considering patients with any disease as cases. All samples in the combined data set were split into two cohorts: 80% for training and 20% for testing. The 80% samples were used as training set to build Random Forest classification model based on the abundance of the 284 combined core genomes (control, n=1285; Case, n=1424; 10-fold Cross Validation). The 20% samples were used as testing set to get probability score from the Random Forest classification model based on the abundance of the 284 combined core genomes (Control, n=319; Case, n=356).

As shown in FIG. 38 A1, training set resulted in an AUC of 0.74 to classify case vs. control. The best cutoff value is 0.5028, the specificity value is 0.7275, and the sensitivity value is 0.6374. As shown in FIG. 38 B1, test set yielded an AUC of 0.76 to classify case vs. control. The best cutoff value is 0.531, the specificity value is 0.6489, and the sensitivity value is 0.7492. The model generated a significantly higher probability score for case than control, which were observed in both of the training set (FIG. 38A2, FIG. 38A3) and testing set (FIG. 38B2, FIG. 38B3). Accordingly, a universal model that differentiates between disease and control can be trained using the identified microorganism genomes described herein.

The success of Random Forest models based on the 284 core genomes in our seesaw networked two competing guilds suggests that the biological signals associated with these genomes are robustly detectable despite the variations introduced by all kinds of confounding factors, ranging from biological to technological. The further refinement and testing of our universal models will make a significant contribution to translational metagenomics.

### Example 5—Repeated Training for Universal Random Forest Classification Models Based on the 284 Core Genomes in the Seesaw Networked Two Competing Guilds

The 25 metagenomic datasets covering case-control studies on 15 different diseases were utilized to construct Random Forest classification models with randomly selected number of genomes out of the 284 core genomes.

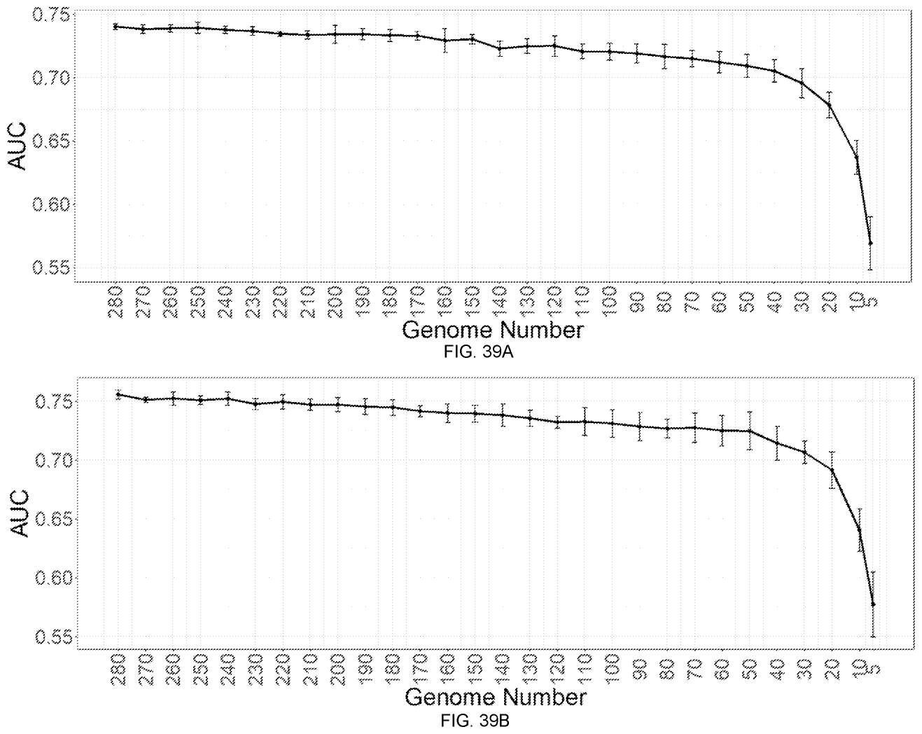

Briefly, multiple random forest classifiers were trained based on microbiota datasets obtained for diseased and healthy controls in at least one study of each of type-2 diabetes (T2D), atherosclerotic cardiovascular disease (ACVD), liver cirrhosis (LC), ankylosing spondylitis (AS), Parkinson's disease (PD), schizophrenia (SCZ), colorectal cancer (CRC), inflammatory bowel diseases (IBD), and hypertension. Specifically, datasets were randomly divided into 80% for training the RF model and 20% for testing. For each dataset, 10 classifiers were trained using randomly selected sets of 5, 10, 20, 30, 40, 50, 60, 70, 80, 90, 100, 110, 120, 130, 140, 150, 160, 170, 180, 190, 200, 210, 220, 230, 240, 250, 260, 270 and 280 genomes from the 284 genomes identified in Table 2 (290 total models per data set). The average AUC from ROC curves for each set of x randomly selected genomes was determined and plotted in FIG. 39. As shown in FIG. 39, fewer than all of the 284 genomes was required to adequately power a clinical model of disease state. In fact, in most, if not all cases, models trained with only 15-20 randomly selected genomes were adequately powered for clinical use (e.g., having an AUC of 0.65 or greater).

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in FIG. 1, and/or as described in FIG. 2. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.
1 Zhao, L. The gut microbiota and obesity: from correlation to causality. Nat Rev Microbiol 11, 639-647, doi:10.1038/nrmicro3089 (2013).
2 Fan, Y. & Pedersen, O. Gut microbiota in human metabolic health and disease. Nat Rev Microbiol, doi:10.1038/s41579-020-0433-9 (2020).
3 Zhang, C. & Zhao, L. Strain-level dissection of the contribution of the gut microbiome to human metabolic disease. Genome Med 8, 41, doi:10.1186/s13073-016-0304-1 (2016).
4 Vacca, M. et al. The Controversial Role of Human Gut Lachnospiraceae. Microorganisms 8, doi:10.3390/microorganisms8040573 (2020).
5 Wu, G., Zhao, N., Zhang, C., Lam, Y. Y. & Zhao, L. Guild-based analysis for understanding gut microbiome in human health and diseases. Genome Medicine 13, 22, doi:10.1186/s13073-021-00840-y (2021).
6 Dominguez-Bello, M. G., Godoy-Vitorino, F., Knight, R. & Blaser, M. J. Role of the microbiome in human development. Gut 68, 1108-1114, doi:10.1136/gutjnl-2018-317503 (2019).
7 Kundu, P., Blacher, E., Elinav, E. & Pettersson, S. Our Gut Microbiome: The Evolving Inner Self Cell 171, 1481-1493, doi:10.1016/j.cell.2017.11.024 (2017).
8 O'Hara, A. M. & Shanahan, F. The gut flora as a forgotten organ. Embo Rep 7, 688-693, doi:10.1038/sj.embor.7400731 (2006).
9 Koh, A. & Backhed, F. From Association to Causality: the Role of the Gut Microbiota and Its Functional Products on Host Metabolism. Mol Cell 78, 584-596, doi:10.1016/j.molcel.2020.03.005 (2020).
10 Sanna, S. et al. Causal relationships among the gut microbiome, short-chain fatty acids and metabolic diseases. Nat Genet 51, 600-+, doi:10.1038/s41588-019-0350-x (2019).
11 Meijnikman, A. S., Gerdes, V. E., Nieuwdorp, M. & Herrema, H. Evaluating Causality of Gut Microbiota in Obesity and Diabetes in Humans. Endocr Rev 39, 133-153, doi:10.1210/er.2017-00192 (2018).
12 Tierney, B. T., Tan, Y., Kostic, A. D. & Patel, C. J. Gene-level metagenomic architectures across diseases yield high-resolution microbiome diagnostic indicators. Nat Commun 12, 2907, doi:10.1038/s41467-021-23029-8 (2021).
13 Wang, J. & Jia, H. Metagenome-wide association studies: fine-mining the microbiome. Nat Rev Microbiol 14, 508-522, doi: 10.1038/nrmicro.2016.83 (2016).
14 Duvallet, C., Gibbons, S. M., Gurry, T., Irizarry, R. A. & Alm, E. J. Meta-analysis of gut microbiome studies identifies disease-specific and shared responses. Nat Commun 8, 1784, doi:10.1038/s41467-017-01973-8 (2017).
15 Jackson, M. A. et al. Gut microbiota associations with common diseases and prescription medications in a population-based cohort. Nat Commun 9, 2655, doi:10.1038/s41467-018-05184-7 (2018).
16 Zhao, L. et al. Gut bacteria selectively promoted by dietary fibers alleviate type 2 diabetes. Science 359, 1151-1156, doi:10.1126/science.aao5774 (2018).
17 Zhang, C. H. et al. Dietary Modulation of Gut Microbiota Contributes to Alleviation of Both Genetic and Simple Obesity in Children. Ebiomedicine 2, 968-984, doi:10.1016/j.ebiom.2015.07.007 (2015).
18 Foster, K. R., Chluter, J. S., Oyte, K. Z. C. & Rakoff-Nahoum, S. The evolution of the host microbiome as an ecosystem on a leash. Nature 548, 43-51, doi:10.1038/nature23292 (2017).
19 Sommer, F., Anderson, J. M., Bharti, R., Raes, J. & Rosenstiel, P. The resilience of the intestinal microbiota influences health and disease. Nat Rev Microbiol 15, 630-638, doi:10.1038/nrmicro.2017.58 (2017).
20 Poisot, T. & Gravel, D. When is an ecological network complex?Connectance drives degree distribution and emerging network properties. PeerJ 2, e251, doi:10.7717/peerj.251 (2014).
21 Barabasi, A. L. Network science. Philos Trans A Math Phys Eng Sci 371, 20120375, doi:10.1098/rsta.2012.0375 (2013).
22 Qin, J. et al. A metagenome-wide association study of gut microbiota in type 2 diabetes. Nature 490, 55-60, doi:10.1038/nature11450 (2012).
23 Jie, Z. et al. The gut microbiome in atherosclerotic cardiovascular disease. Nat Commun 8, 845, doi:10.1038/s41467-017-00900-1 (2017).
24 Qin, N. et al. Alterations of the human gut microbiome in liver cirrhosis. Nature 513, 59-64, doi:10.1038/nature13568 (2014).
25 Wen, C. et al. Quantitative metagenomics reveals unique gut microbiome biomarkers in ankylosing spondylitis. Genome Biol 18, 142, doi:10.1186/s13059-017-1271-6 (2017).
26 Li, J. et al. Gut microbiota dysbiosis contributes to the development of hypertension. Microbiome 5, 14, doi:10.1186/s40168-016-0222-x (2017).
27 Lloyd-Price, J. et al. Multi-omics of the gut microbial ecosystem in inflammatory bowel diseases. Nature 569, 655-662, doi:10.1038/s41586-019-1237-9 (2019).
28 Franzosa, E. A. et al. Gut microbiome structure and metabolic activity in inflammatory bowel disease. Nat Microbiol 4, 293-305, doi:10.1038/s41564-018-0306-4 (2019).
29 Yu, J. et al. Metagenomic analysis of faecal microbiome as a tool towards targeted non-invasive biomarkers for colorectal cancer. Gut 66, 70-+, doi:DOI 10.1136/gutjnl-2015-309800 (2017).
30 Feng, Q. et al. Gut microbiome development along the colorectal adenoma-carcinoma sequence. Nat Commun 6, 6528, doi:10.1038/ncomms7528 (2015).
31 Qian, Y. W. et al. Gut metagenomics-derived genes as potential biomarkers of Parkinson's disease. Brain 143, 2474-2489 (2020).
32 Coyte, K. Z., Schluter, J. & Foster, K. R. The ecology of the microbiome: Networks, competition, and stability. Science 350, 663-666, doi:10.1126/science.aad2602 (2015).
33 Kamada, N., Seo, S. U., Chen, G. Y. & Nunez, G. Role of the gut microbiota in immunity and inflammatory disease. Nat Rev Immunol 13, 321-335, doi:10.1038/nri3430 (2013).
34 Vatanen, T. et al. Variation in Microbiome LPS Immunogenicity Contributes to Autoimmunity in Humans. Cell 165, 1551, doi:10.1016/j.cell.2016.05.056 (2016).
35 Bach, J. F. The hygiene hypothesis in autoimmunity: the role of pathogens and commensals. Nat Rev Immunol 18, 105-120, doi:10.1038/nri.2017.111 (2018).
36 Risely, A. Applying the core microbiome to understand host-microbe systems. Journal of Animal Ecology 89, 1549-1558 (2020).
37 Makki, K., Deehan, E. C., Walter, J. & Backhed, F. The Impact of Dietary Fiber on Gut Microbiota in Host Health and Disease. Cell Host Microbe 23, 705-715, doi:10.1016/j.chom.2018.05.012 (2018).
38 Reynolds, A. et al. Carbohydrate quality and human health: a series of systematic reviews and meta-analyses. The Lancet 393, 434-445 (2019).
39 Eaton, S. B. The ancestral human diet: what was it and should it be a paradigm for contemporary nutrition ?P Nutr Soc 65, 1-6, doi:10.1079/Pns2005471 (2006).
40 Jew, S., AbuMweis, S. S. & Jones, P. J. Evolution of the human diet: linking our ancestral diet to modern functional foods as a means of chronic disease prevention. J Med Food 12, 925-934, doi:10.1089/jmf.2008.0268 (2009).
41 Spiller, G. A. & Amen, R. J. Topics in dietary fiber research. (Springer, 1978).
42 Thompson, H. J. & Brick, M. A. Perspective: Closing the dietary fiber gap: An ancient solution for a 21st century problem. Advances in Nutrition 7, 623-626 (2016).
43 Deehan, E. C. et al. Modulation of the gastrointestinal microbiome with nondigestible fermentable carbohydrates to improve human health. Microbiology spectrum 5, 5.5. 04 (2017).
44 Prevey, J. S., Germino, M. J. & Huntly, N. J. Loss of foundation species increases population growth of exotic forbs in sagebrush steppe. Ecol Appl 20, 1890-1902, doi:10.1890/09-0750.1 (2010).
45 Anderson, J. W. et al. Health benefits of dietary fiber. Nutr Rev 67, 188-205, doi:10.1111/j.1753-4887.2009.00189.x (2009).
46 Kaczmarczyk, M. M., Miller, M. J. & Freund, G. G. The health benefits of dietary fiber: beyond the usual suspects of type 2 diabetes mellitus, cardiovascular disease and colon cancer. Metabolism 61, 1058-1066, doi:10.1016/j.metabol.2012.01.017 (2012).
47 Risely, A. Applying the core microbiome to understand host-microbe systems. J Anim Ecol 89, 1549-1558, doi:10.1111/1365-2656.13229 (2020).
48 Berg, G. et al. Microbiome definition re-visited: old concepts and new challenges. Microbiome 8, 103, doi:10.1186/s40168-020-00875-0 (2020).
49 Society, C. D. China guideline for type 2 diabetes (2013 edition). Chin J Diabetes 22 (2014).
50 yuexin, Y., guangya, W. & xingchang, P. China Food Composition (Book 1, Beijing Medical Univ. Press, ed. 2, 2009). (2009).
51 Ewing, D. & Clarke, B. Diagnosis and management of diabetic autonomic. British Medical Journal 285 (1982).
52 Feldman, E. L. et al. A Practical Two-Step Quantitative Clinical and Electrophysiological Assessment for the Diagnosis and Staging of Diabetic Neuropathy. Diabetes Care 17, 1281-1289 (1994).
53 Li, X., Zhou, Z. G., Qi, H. Y., Chen, X. Y. & Huang, G. Replacement of insulin by fasting C-peptide in modified homeostasis model assessment to evaluate insulin resistance and islet beta cell function. Zhong Nan Da Xue Xue Bao Yi Xue Ban 29, 419-423 (2004).
54 Ma, Y. C. et al. Modified glomerular filtration rate estimating equation for Chinese patients with chronic kidney disease. J Am Soc Nephrol 17, 2937-2944, doi:10.1681/ASN.2006040368 (2006).
55 Schmieder, R. & Edwards, R. Quality control and preprocessing of metagenomic datasets. Bioinformatics 27, 863-864, doi:10.1093/bioinformatics/btr026 (2011).
56 Langmead, B. & Salzberg, S. L. Fast gapped-read alignment with Bowtie 2. Nat Methods 9, 357-359, doi:10.1038/nmeth.1923 (2012).
57 Peng, Y., Leung, H. C., Yiu, S. M. & Chin, F. Y. IDBA-UD: a de novo assembler for single-cell and metagenomic sequencing data with highly uneven depth. Bioinformatics 28, 1420-1428, doi:10.1093/bioinformatics/bts174 (2012).
58 Kang, D. D., Froula, J., Egan, R. & Wang, Z. MetaBAT, an efficient tool for accurately reconstructing single genomes from complex microbial communities. PeerJ 3, e1165, doi:10.7717/peerj.1165 (2015).
59 Parks, D. H., Imelfort, M., Skennerton, C. T., Hugenholtz, P. & Tyson, G. W. CheckM: assessing the quality of microbial genomes recovered from isolates, single cells, and metagenomes. Genome Res 25, 1043-1055, doi:10.1101/gr.186072.114 (2015).
60 Olm, M. R., Brown, C. T., Brooks, B. & Banfield, J. F. dRep: a tool for fast and accurate genomic comparisons that enables improved genome recovery from metagenomes through de-replication. ISME J 11, 2864-2868, doi:10.1038/ismej.2017.126 (2017).
61 Fischer, M., Strauch, B. & Renard, B. Y. Abundance estimation and differential testing on strain level in metagenomics data. Bioinformatics 33, i124-i132, doi:10.1093/bioinformatics/btx237 (2017).
62 Chaumeil, P. A., Mussig, A. J., Hugenholtz, P. & Parks, D. H. GTDB-Tk: a toolkit to classify genomes with the Genome Taxonomy Database. Bioinformatics, doi:10.1093/bioinformatics/btz848 (2019).
63 Seemann, T. Prokka: rapid prokaryotic genome annotation. Bioinformatics 30, 2068-2069, doi:10.1093/bioinformatics/btu153 (2014).
64 Aramaki, T. et al. KofamKOALA: KEGG Ortholog assignment based on profile HM4 and adaptive score threshold. Bioinformatics 36, 2251-2252, doi:10.1093/bioinformatics/btz859 (2020).
65 Zankari, E. et al. Identification of acquired antimicrobial resistance genes. J Antimicrob Chemother 67, 2640-2644, doi:10.1093/jac/dks261 (2012).
66 Liu, B., Zheng, D., Jin, Q., Chen, L. & Yang, J. VFDB 2019: a comparative pathogenomic platform with an interactive web interface. Nucleic Acids Res 47, D687-D692, doi:10.1093/nar/gky1080 (2019).
67 Yin, Y. et al. dbCAN: a web resource for automated carbohydrate-active enzyme annotation. Nucleic Acids Res 40, W445-451, doi:10.1093/nar/gks479 (2012).
68 Bortolaia, V. et al. ResFinder 4.0 for predictions of phenotypes from genotypes. J Antimicrob Chemother 75, 3491-3500, doi: 10.1093/jac/dkaa345 (2020).
69 Watts, S. C., Ritchie, S. C., Inouye, M. & Holt, K. E. FastSpar: rapid and scalable correlation estimation for compositional data. Bioinformatics 35, 1064-1066, doi:10.1093/bioinformatics/bty734 (2019).
70 Shannon, P. et al. Cytoscape: a software environment for integrated models of biomolecular interaction networks. Genome Res 13, 2498-2504, doi:10.1101/gr.1239303 (2003).
71 Letunic, I. & Bork, P. Interactive Tree Of Life (iTOL) v4: recent updates and new developments. Nucleic Acids Res 47, W256-W259, doi:10.1093/nar/gkz239 (2019).

What is claimed is:

1. A method of identifying a set of gut microorganisms, comprising:
at a computer system having one or more processors, and memory storing one or more programs for execution by the one or more processors:
A) obtaining, in electronic form, for each respective subject in a first plurality of subjects having a first state of a biological characteristic, a corresponding plurality of genomic abundance values comprising, for each respective gut microorganism in a plurality of gut microorganisms, a corresponding value for the abundance of the genome of the respective gut microorganism in a biological sample from the gut of the respective subject;
B) obtaining, in electronic form, for each respective subject in a second plurality of subjects having a second state of a biological characteristic, a corresponding plurality of genomic abundance values comprising, for each respective gut microorganism in the plurality of gut microorganisms, a corresponding value for the abundance of the genome of the respective gut microorganism in a biological sample from the gut of the respective subject;
C) computing a first plurality of similarity metrics from the corresponding pluralities of genomic abundance values across the first plurality of subjects, wherein:
the first plurality of similarity metrics comprises a first corresponding similarity metric for each unique pair of gut microorganisms in the plurality of gut microorganisms, and
the first corresponding similarity metric quantifies a similarity between (i) a corresponding first vector formed by the corresponding genomic abundance values of the first microorganism in the unique pair of gut microorganisms across the first plurality of subjects and (ii) a corresponding second vector formed by the corresponding genomic abundance values of the second microorganism in the unique pair of gut microorganisms across the first plurality of subjects;
D) computing a second plurality of similarity metrics using the corresponding genomic abundance values for the second plurality of subjects, wherein:
the second plurality of similarity metrics comprises a second corresponding similarity metric for each unique pair of gut microorganisms in the plurality of gut microorganisms, and
the second corresponding similarity metric quantifies a similarity between (i) a corresponding second vector formed by the corresponding genomic abundance values of the first microorganism in the unique pair of gut microorganisms across the second plurality of subjects and (ii) a corresponding second vector formed by the corresponding genomic abundance values of the second microorganism in the unique pair of gut microorganisms across the second plurality of subjects;
E) determining a set of unique pairs of gut microorganisms in the plurality of gut microorganisms based on the first plurality of similarity metrics and the second plurality of similarity metrics, wherein, for each respective unique pair of gut microorganisms in the set of unique pairs of gut microorganisms:
the first corresponding similarity metric and the second corresponding similarity metric both indicate a statistically significant positive correlation between the abundance of the first gut microorganism and the abundance of the second gut microorganism in the respective unique pair of gut microorganisms, or
the first corresponding similarity metric and the second corresponding similarity metric both indicate a statistically significant negative correlation between the abundance of the first gut microorganism and the abundance of the second gut microorganism in the respective unique pair of gut microorganisms; and
F) identifying a set of gut microorganisms comprising respective gut microorganisms represented in the set of unique pairs of gut microorganisms.

2. The method of claim 1, wherein:
the obtaining A) comprises:
(i) obtaining, in electronic form, for each respective subject in the first plurality of subjects, a corresponding first plurality of at least 100,000 nucleic acid sequences for genomic DNA from a corresponding biological sample from the gut of the respective subject, and
(ii) determining, for each respective subject in the first plurality of subjects, the corresponding genomic abundance value for each respective gut microorganism in the plurality of gut microorganisms from the corresponding first plurality of at least 100,000 nucleic acid sequences; and
the obtaining B) comprises:
(i) obtaining, in electronic form, for each respective subject in the second plurality of subjects, a corresponding second plurality of at least 100,000 nucleic acid sequences for genomic DNA from a corresponding biological sample from the gut of the respective subject, and
(ii) determining, for each respective subject in the second plurality of subjects, the corresponding genomic abundance value for each respective gut microorganism in the plurality of gut microorganisms from the corresponding second plurality of at least 100,000 nucleic acid sequences.

3. The method of claim 2, wherein:
the determining A) (ii) comprises, for each respective subject in the first plurality of subjects:
assembling a corresponding first plurality of gut microorganism genomes by metagenomic de novo sequence assembly from the corresponding first plurality of at least 100,000 nucleic acid sequences, and
calculating, for each respective gut microorganism genome in the corresponding first plurality of gut microorganism genomes, a corresponding genomic abundance of the respective gut microorganism genome; and
the determining B) (ii) comprises, for each respective subject in the second plurality of subjects:
assembling a corresponding second plurality of gut microorganism genomes by metagenomic de novo sequence assembly from the corresponding second plurality of at least 100,000 nucleic acid sequences, and
calculating, for each respective gut microorganism genome in the corresponding second plurality of gut microorganism genomes, a corresponding genomic abundance of the respective gut microorganism genome.

4. The method of claim 2, wherein:
the determining A) (ii) comprises, for each respective subject in the first plurality of subjects:
assigning each respective nucleic acid sequence in the corresponding first plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding first plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism; and
the determining B) (ii) comprises, for each respective subject in the first plurality of subjects:
assigning each respective nucleic acid sequence in the corresponding second plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding second plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

5. The method according to any one of claims 2-4, further comprising:
sequencing, for each respective subject in the first plurality of subjects, genomic DNA from the corresponding biological sample from the gut of the respective subject, thereby obtaining the corresponding first plurality of at least 100,000 nucleic acid sequences; and
sequencing, for each respective subject in the second plurality of subjects, genomic DNA from the corresponding biological sample from the gut of the respective subject, thereby obtaining the corresponding second plurality of at least 100,000 nucleic acid sequences.

6. The method according to any one of claims 1-5, wherein:
the first state of the biological characteristic is the absence of a disease or disorder and the second state of the biological characteristic is the presence of the disease or disorder;
the first state of the biological characteristic is a first severity of a disease or disorder and the second state of the biological characteristic is a second severity of the disease or disorder;
the first state of the biological characteristic is an untreated disease or disorder and the second state of the biological characteristic is a treated disease or disorder;
the first state of the biological characteristic is a disease or disorder treated with a first therapy and the second state of the biological characteristic is a disease or disorder treated with a second therapy;
the first state of the biological characteristic is a first level of a nutrient in a diet and the second state of the biological characteristic is a second level of a nutrient in a diet; or
the first state of the biological characteristic is a first age and the second state of the biological characteristic is a second age.

7. The method according to any one of claims 1-6, wherein the plurality of gut microorganisms comprises at least 20 gut microorganisms selected from Table 1, Table 2, or FIG. 42 A-42XX.

8. The method according to any one of claims 1-7, wherein:
for each respective subject in the first plurality of subjects, the biological sample from the gut of the respective subject is a fecal sample; and
for each respective subject in the second plurality of subjects, the biological sample from the gut of the respective subject is a fecal sample.

9. The method according to any one of claims 1-8, wherein the first corresponding similarity metric and the second similarity metric are both a Pearson correlation coefficient, an intraclass correlation coefficient, or a rank correlation coefficient.

10. The method according to any one of claims 1-9, wherein a statistically significant positive correlation has a P-value of less than 0.001.

11. The method according to any one of claims 1-10, wherein the set of gut microorganisms comprises all respective gut microorganisms represented in the set of unique pairs of gut microorganisms.

12. The method according to any one of claims 1-10, wherein the identifying F) comprises:
clustering the respective gut microorganisms represented in the set of unique pairs of gut microorganisms into one of more networks, wherein each respective connected network comprising a corresponding plurality of nodes and a corresponding set of one or more edges, wherein:
each respective node in the corresponding plurality of nodes represents a unique gut microorganism represented in the set of unique pairs of gut microorganisms,
each respective edge in the corresponding set of one or more edges connects two nodes representing a respective unique pair of gut microorganisms in the set of unique pairs of gut microorganisms, and
each respective node in the corresponding plurality of nodes is connected to at least one other respective node in the plurality of nodes through a respective edge in the corresponding set of one or more edges; and
identifying the respective network in the one or more networks comprising the most nodes, thereby identifying the set of gut microorganisms represented by the corresponding plurality of nodes in the respective network.

13. The method according to any one of claims 1-12, wherein the set of gut microorganisms comprises at least 20 gut microorganisms selected from Table 1, Table 2, or FIG. 42 A-42XX.

14. A method of training a model for evaluating human health, comprising:
at a computer system having one or more processors, and memory storing one or more programs for execution by the one or more processors:
A) obtaining, in electronic form, for each respective training subject in a plurality of training subjects:
(i) a corresponding plurality of genomic abundance values comprising, for each respective gut microorganism in a plurality of gut microorganisms, a corresponding value for the abundance of the genome of the respective gut microorganism in a corresponding biological sample from the gut of the respective training subject, and
(ii) a corresponding state of a biological characteristic of the respective training subject;
B) inputting, for each respective training subject in the plurality of training subjects, information about the respective training subject into a model comprising a plurality of parameters, wherein the model applies the plurality of parameters to the information through at least 10,000 computations to obtain a corresponding output for the respective training subject from the model, wherein:
the corresponding output comprises an indication of the corresponding state of the biological characteristic of the respective training subject, and
the information about the respective training subject comprises the corresponding genomic abundance value for each respective gut microorganism in the plurality of gut microorganisms, and
the plurality of gut microorganisms are selected from Table 1, Table 2, or FIG. 42 A-42XX; and
C) adjusting the plurality of parameters based on, for each respective training subject in the first plurality of training subjects, one or more differences between (i) the corresponding output from the model and (ii) the corresponding state of the biological characteristic of the respective training subject.

15. The method of claim 14, wherein the obtaining A) comprises, for each respective training subject in the plurality of training subjects:
(i) obtaining, in electronic form, a corresponding plurality of at least 100,000 nucleic acid sequences for genomic DNA from the corresponding biological sample from the gut of the respective training subject; and
(ii) determining, for each respective gut microorganism in the plurality of gut microorganisms, the corresponding value for the abundance of the genome of the respective gut microorganism from the corresponding first plurality of at least 100,000 nucleic acid sequences.

16. The method of claim 15, wherein the determining A) (ii) comprises, for each respective training subject in the plurality of training subjects:
assembling, in electronic form, a corresponding plurality of gut microorganism genomes by metagenomic de novo sequence assembly from the corresponding plurality of at least 100,000 nucleic acid sequences, and
calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

17. The method of claim 15, wherein the determining A) (ii) comprises, for each respective subject in the plurality of training subjects:
assigning each respective nucleic acid sequence in the corresponding plurality of at least 100,000 sequences to a respective gut microorganism in the plurality of gut microorganisms, thereby generating, for each respective gut microorganism in the plurality of gut microorganism, a corresponding count of respective nucleic acid sequences in the corresponding plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

18. The method according to any one of claims 15-17, further comprising sequencing, for each respective subject in the plurality of training subjects, genomic DNA from the corresponding biological sample from the gut of the respective training subject, thereby obtaining the corresponding plurality of at least 100,000 nucleic acid sequences.

19. The method according to any one of claims 14-18, wherein the plurality of gut microorganisms comprises at least 20 gut microorganisms selected from Table 1, Table 2, or FIG. 42 A-42XX.

20. The method of any one of claims 14-19, wherein the plurality of gut microorganisms comprises at least 20 microorganisms selected from those microorganisms in Table 1, Table 2, or FIG. 42 A-42XX having a connectivity of at least 2.

21. The method according to any one of claims 14-20, wherein for each respective subject in the plurality of training subjects, the biological sample from the gut of the respective subject is a fecal sample from the respective training subject.

22. The method according to any one of claims 14-21, wherein the biological characteristic is a disease or disorder, a therapy administered to the subject, or a diet of the subject.

23. The method of claim 22, wherein the disease or disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD).

24. The method of claim 22, wherein the disease or disorder is cancer.

25. The method of any one of claims 14-24, wherein the indication of the corresponding state of the biological characteristic is a class output of a respective state, in a plurality of possible states, of the biological characteristic.

26. The method of any one of claims 14-24, wherein the indication of the corresponding state of the biological characteristic is a probability output for the corresponding state of the biological characteristic.

27. The method of any one of claims 14-26, wherein the model is a neural network algorithm, a support vector machine algorithm, a Naive Bayes algorithm, a nearest neighbor algorithm, a boosted trees algorithm, a Random Forest algorithm, a convolutional neural network algorithm, a decision tree algorithm, a regression algorithm, or a clustering algorithm.

28. The method of any one of claims 14-27, wherein the plurality of parameters is at least 1000, at least 10,000, at least 15,000, at least 50,000, at least 100,000, at least 250,000, at least 500,000, or at least 1,000,000 parameters.

29. The method of any one of claims 14-28, wherein the model applies the plurality of parameters to the information through at least 25,000, at least 50,000, at least 100,000, at least 250,000, at least 500,000, or at least 1,000,000 computations to obtain a corresponding output for the respective training subject from the model.

30. A method for evaluating the health of a subject, comprising:
at a computer system having one or more processors, and memory storing one or more programs for execution by the one or more processors:
A) obtaining, in electronic form, a plurality of genomic abundance values comprising, for each respective gut microorganism in a plurality of at least 20 gut microorganisms selected from Table 1, Table 2, or FIG. 42 A-42XX, a corresponding abundance value for the genome of the respective species of gut bacteria, in the plurality of at least 20 gut microorganisms, in a biological sample from the subject; and
B) inputting the plurality of genomic abundance values into a model comprising a plurality of parameters, wherein the model applies the plurality of parameters to the plurality of genomic abundance values through at least 10,000 computations to generate as output from the model an indication {e.g., a class output or probability output} of the health of the subject.

31. The method of claim 30, wherein the obtaining A) comprises:
(i) obtaining, in electronic form, a plurality of at least 100,000 nucleic acid sequences {Include support in the spec for minimum numbers, maximum numbers, and ranges of NA sequences} for genomic DNA from the biological sample from the gut of the subject; and
(ii) determining, for each respective gut microorganism in the plurality of gut microorganisms, the corresponding value for the abundance of the genome of the respective gut microorganism from the plurality of at least 100,000 nucleic acid sequences.

32. The method of claim 31, wherein the determining A) (ii) comprises:
assembling, in electronic form, a corresponding plurality of gut microorganism genomes by metagenomic de novo sequence assembly from the plurality of at least 100,000 nucleic acid sequences, and
calculating, for each respective gut microorganism in the plurality of gut microorganisms, the corresponding value for the abundance of the genome of the respective gut microorganism based on the prevalence of respective nucleic acid sequences, in the plurality of at least 100,000 nucleic acid sequences, used to assemble a respective gut microorganism genome in the plurality of gut microorganism genomes corresponding to the respective gut microorganism.

33. The method of claim 31, wherein the determining A) (ii) comprises:
assigning each respective nucleic acid sequence in the plurality of at least 100,000 sequences to a respective gut microorganism in the plurality of gut microorganisms, thereby generating, for each respective gut microorganism in the plurality of gut microorganism, a corresponding count of respective nucleic acid sequences in the plurality of nucleic acid sequences assigned to the respective gut microorganism, and
determining, for each respective gut microorganism in the plurality of gut microorganisms, the corresponding genomic abundance value for the respective gut microorganism based on the corresponding count of respective nucleic acid sequences assigned to the respective gut microorganism.

34. The method according to any one of claims 31-33, further comprising sequencing genomic DNA from the biological sample from the gut of the subject, thereby obtaining the plurality of at least 100,000 nucleic acid sequences.

35. The method of any one of claims 30-34, wherein the plurality of gut microorganisms comprises at least 20 microorganisms selected from those microorganisms in Table 1, Table 2, or FIG. 42 A-42XX having a connectivity of at least 2.

36. The method according to any one of claims 30-35, wherein the biological sample from the gut of the subject is a fecal sample.

37. The method according to any one of claims 30-36, wherein the indication of the health of the subject is an indication of a biological characteristic, wherein the biological characteristic is a disease or disorder, a therapy administered to the subject, or a diet of the subject.

38. The method of claim 37, wherein the disease or disorder is selected from the group consisting of type-2 diabetes, hypertension, schizophrenia, atherosclerotic cardiovascular disease (ACVD), liver cirrhosis (LC), inflammatory bowel diseases (IBD), colorectal cancer (CRC), ankylosing spondylitis (AS), and Parkinson's disease (PD).

39. The method of claim 37, wherein the disease or disorder is cancer.

40. The method of any one of claims 30-39, wherein the indication of the health of the subject is a class output of a respective state, in a plurality of possible states, of the health of the subject.

41. The method of any one of claims 30-39, wherein the indication of the health of the subject is a probability output for the corresponding state of the health of the subject.

42. The method of any one of claims 30-41, wherein the model is a neural network algorithm, a support vector machine algorithm, a Naive Bayes algorithm, a nearest neighbor algorithm, a boosted trees algorithm, a Random Forest algorithm, a convolutional neural network algorithm, a decision tree algorithm, a regression algorithm, or a clustering algorithm.

43. The method of any one of claims 30-42, wherein the plurality of parameters is at least 1000, at least 10,000, at least 15,000, at least 50,000, at least 100,000, at least 250,000, at least 500,000, or at least 1,000,000 parameters.

44. The method of any one of claims 30-43, wherein the model applies the plurality of parameters to the information through at least 25,000, at least 50,000, at least 100,000, at least 250,000, at least 500,000, or at least 1,000,000 computations to obtain a corresponding output for the respective training subject from the model.

45. A computer system, comprising:
one or more processors; and
a non-transitory computer-readable medium including computer-executable instructions that, when executed by the one or more processors, cause the processors to perform the method according to any one of claims 1-44.

46. A non-transitory computer-readable storage medium having stored thereon program code instructions that, when executed by a processor, cause the processor to perform the method according to any one of claims 1-44.
